SEQUENCE LISTING (50 sequences)

SEQ ID NO: 1 - DNA, 1265 bp, Artificial Sequence
  Artificial polynucleotide
gcaagagacacagtcctgggaaagtctgccggctatccagacaattataaaaatgtgtctcccaaggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgaaagctcaaggccctctgccacagcgccctcctcacacagataggaaacaaagaaagattcataagagtcaggtggtcttggcctggtgggcttaagctccttcaggaatctgttctcccatcacatctcatctccccaaaggtggcacagctacctcagcatggtgccctccatcgcttctctcatattcactatacaagttgtttgtaagttttcatcaaaatattgttaaggggcgaagacgtcctcccctcaatgtgttagcagaagagcaagaactgataagctattgtttttgtgccaaagtgtttatgaaaacactcagtcaccccttatttaaaaatatttattgctatattttatactcatgaaagtacatgagcctatttatatttatttattttctatttattataatatttcttatcagatgaatttgaaacattttgaaacataccttattttgtggttctaataaagtaatgttatca

SEQ ID NO: 2 - DNA, 1840 bp, ArtificialSequence
  Artificial polynucleotide
gcacatcagaccaggcagctcgcagcaaagcaagatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaggatgcaacgttggaaaggaaagaaaagtggaagacattaaggaagaaaaatttaaactcaggatggaagagtcccccaaaagctgtcttctgcttggttggctttttccagttttcctaagttcatcatgacacctttgctgatttctacatgtaaatgttaaatgcccgcagagccagggagctaatgtatgcatagatattctagcattccacttggccttatgctgttgaaatatttaagtaatttatgtatttattaatttatttctgcatttcacatttgtataccaagatgtattgaatatttcatgtgctcgtggcctgatccactgggaccaggccctattatgcaaattgtgagcttgttatcttcttcaacagctcttcaatcagggctgcgtaggtacattagcttttgtgacaaccaataagaacataatattctgacacaagcagtgttacatatttgtgaccagtaaagacataggtggtatttggagacatgaagaagctgtaaagttgactctgaagagtttagcactagtttcaacaccaagaaagactttttagaagtgatattgataagaaaccagggccttctttagaagggtacctaaatttaaaagaattttgaaaggctgggtatcggtggtatatgcttttaattccagcactcaggagaccaaggcaggcagatctctgtgagtttgaggacagcctggtgtacagagggagttccagcacagccagtgccacacagaaattctgtctcaaaaacaattaaaaaaaaaaaaaa

SEQ ID NO: 3 - protein, 215 aa, ArtificialSequence
  Artificial polypeptide
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 4 - protein, 335 aa, ArtificialSequence
  Artificial polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 5 - protein, 121 aa, ArtificialSequence
  Artificial polypeptide
MAEVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTV

SEQ ID NO: 6 - DNA, 363 bp, ArtificialSequence
  Artificial polynucleotide
atggccgaggtgcagctggtggagagcggcggcggcctggtgcagcccggcggcagcctgaggctgagctgcgccgccagcggcttcaccttcagcaactacaccatgaactgggtgaggcaggcccccggcaagggcctggagtgggtgagcgccatcagcggcagcggcggcagcacctactacgccgacagcgtgaagggcaggttcaccatcagcagggacaacagcaagaacaccctgtacctgcagatgaacagcctgagggccgaggacaccgccgtgtactactgcgccaaggacaggtacagccaggtgcactacgccctggactactggggccagggcaccctggtgaccgtg

SEQ ID NO: 7 - protein, 59 aa, ArtificialSequence
  Artificial polypeptide
DIQMTQSPDSLPVTPGEPASISCRSSQSLLHSNGYNYLDWYLQKAGQSPQLLIYLGSNR

SEQ ID NO: 8 - DNA, 339 bp, ArtificialSequence
  Artificial polynucleotide
gacatccagatgacccagtcccccgactccctgcccgtgacccccggcgagcccgcctccatctcctgccggtcctcccagtccctgctgcactccaacggctacaactacctggactggtacctgcagaaggccggccagtccccccagctgctgatctacctgggctccaaccgggcctccggcgtgcccgaccggttctccggctccggctccggcaccgacttcaccctgaagatctcccgggtggaggccgaggacgtgggcgtgtactactgccagcagtactacaaccaccccaccaccttcggccagggcaccaagctggagatcaagcgg

SEQ ID NO: 9 - protein, 474 aa, ArtificialSequence
  Artificial polypeptide
MGQSRYLLFLATLALLNHLSLAMAEVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - DNA, 1431 bp, ArtificialSequence
  Artificial polynucleotide
gccaccatgggccagagcaggtacctgctgttcctggccaccctggccctgctgaaccacctgagcctggccatggccgaggtgcagctggtggagagcggcggcggcctggtgcagcccggcggcagcctgaggctgagctgcgccgccagcggcttcaccttcagcaactacaccatgaactgggtgaggcaggcccccggcaagggcctggagtgggtgagcgccatcagcggcagcggcggcagcacctactacgccgacagcgtgaagggcaggttcaccatcagcagggacaacagcaagaacaccctgtacctgcagatgaacagcctgagggccgaggacaccgccgtgtactactgcgccaaggacaggtacagccaggtgcactacgccctggactactggggccagggcaccctggtgaccgtggccgccaagaccaccgcccccagcgtgtaccccctggcccccgtgtgcggcgacaccaccggcagcagcgtgaccctgggctgcctggtgaagggctacttccccgagcccgtgaccctgacctggaacagcggcagcctgagcagcggcgtgcacaccttccccgccgtgctgcagagcgacctgtacaccctgagcagcagcgtgaccgtgaccagcagcacctggcccagccagagcatcacctgcaacgtggcccaccccgccagcagcaccaaggtggacaagaagatcgagcccaggggcctcaccatcaagccctgccccccctgcaagtgccccgcccccaacctgctgggcggccccagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgcccagacccagacccacagggaggactacaacagcaccctgagggtggtgagcgccctgcccatccagcaccaggactggatgagcggcaaggagttcaagtgcaaggtgaacaacaaggacctgcccgcccccatcgagaggaccatcagcaagcccaagggcagcgtgagggcctcccaggtgtacgtgctgcccccccccgaggaggagatgaccaagaagcaggtgaccctgacctgcatggtgaccgacttcatgcccgaggacatctacgtggagtggaccaacaacggcaagaccgagctgaactacaagaacaccgagcccgtgctggacagcgacggcagctacttcatgtacagcaagctgagggtggagaagaagaactgggtggagaggaacagctacagctgcagcgtggtgcacgagggcctgcacaaccaccacaccaccaagagcttcagcaggacccccggcaagtaa

SEQ ID NO: 11 - protein, 241 aa, ArtificialSequence
  Artificial polypeptide
MGQSRYLLFLATLALLNHLSLADIQMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - DNA, 732 bp, ArtificialSequence
  Artificial polynucleotide
gccaccatgggccagtcccggtacctgctgttcctggccaccctggccctgctgaaccacctgtccctggccgacatccagatgacccagtcccccgactccctgcccgtgacccccggcgagcccgcctccatctcctgccggtcctcccagtccctgctgcactccaacggctacaactacctggactggtacctgcagaaggccggccagtccccccagctgctgatctacctgggctccaaccgggcctccggcgtgcccgaccggttctccggctccggctccggcaccgacttcaccctgaagatctcccgggtggaggccgaggacgtgggcgtgtactactgccagcagtactacaaccaccccaccaccttcggccagggcaccaagctggagatcaagcgggccgacgccgcccccaccgtgtccatcttccccccctcctccgagcagctgacctccggcggcgcctccgtggtgtgcttcctgaacaacttctaccccaaggacatcaacgtgaagtggaagatcgacggctccgagcggcagaacggcgtgctgaactcctggaccgaccaggactccaaggactccacctactccatgtcctccaccctgaccctgaccaaggacgagtacgagcggcacaactcctacacctgcgaggccacccacaagacctccacctcccccatcgtgaagtccttcaaccggaacgagtgctaa

SEQ ID NO: 13 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 14 - DNA, 806 bp, ArtificialSequence
  Artificial polynucleotide
agcagggggcgctgtgtgtaccgagaatacgagaatacctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaaaaaaaaaaaaaaaaaaaaa

SEQ ID NO: 15 - protein, 180 aa, ArtificialSequence
  Artificial polypeptide
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 16 - protein, 10 aa, ArtificialSequence
  Artificial polypeptide
GFIFSYFDMA

SEQ ID NO: 17 - protein, 16 aa, ArtificialSequence
  Artificial polypeptide
SISPDGSIPYYRDSVK

SEQ ID NO: 18 - protein, 11 aa, ArtificialSequence
  Artificial polypeptide
RSYGGYSELDY

SEQ ID NO: 19 - protein, 120 aa, ArtificialSequence
  Artificial polypeptide
DVQLVESGGGLVQPGRSLKLSCAASGFIFSYFDMAWVRQAPTKGLEWVASISPDGSIPYYRDSVKGRFTVSRENAKSSLYLQMDSLRSEDTATYYCARRSYGGYSELDYWGQGVMVTVSS

SEQ ID NO: 20 - protein, 11 aa, ArtificialSequence
  Artificial polypeptide
QASQDIGNWLA

SEQ ID NO: 21 - protein, 7 aa, ArtificialSequence
  Artificial polypeptide
GSTSLAD

SEQ ID NO: 22 - protein, 8 aa, ArtificialSequence
  Artificial polypeptide
LQAYGAPW

SEQ ID NO: 23 - protein, 107 aa, ArtificialSequence
  Artificial polypeptide
DIQMTQSPASLSASLEEIVTITCQASQDIGNWLAWYHQKPGKSPQLLIYGSTSLADGVPSRFSGSSSGSQYSLKISRLQVEDIGIYYCLQAYGAPWTFGGGTKLELK

SEQ ID NO: 24 - protein, 255 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 25 - DNA, 20 bp, ArtificialSequence
  Artificial polynucleotide
gaagtccgctgttcttttcc

SEQ ID NO: 26 - DNA, 20 bp, ArtificialSequence
  Artificial polynucleotide
ttgacttccgttcttcagtg

SEQ ID NO: 27 - DNA, 20 bp, ArtificialSequence
  Artificial polynucleotide
gctgcaactgcatccatatc

SEQ ID NO: 28 - DNA, 20 bp, ArtificialSequence
  Artificial polynucleotide
aggagcccttttagaccttt

SEQ ID NO: 29 - DNA, 38 bp, ArtificialSequence
  Artificial polynucleotide
cgcgtaaagcatctctacggtggtcctaatagtgcatg

SEQ ID NO: 30 - DNA, 30 bp, ArtificialSequence
  Artificial polynucleotide
cactattaggaccaccgtcgagatgcttta

SEQ ID NO: 31 - protein, 154 aa, Artificial Sequence
  Artificial polypeptide
MTMITPSLVPSSDPLVTAASVLEFALLIRLTIGQAVVSTQSTGGGLVQPGRSLRLSCAASGFTLDDYGMHWVRQAPGKGLEWVSGISWNSDSIGYVDSVKGRFTISRDNAKNSLYLQMNSLRVEDTALYYCVKDISGWYSFDYWGQGTLVTVSS

SEQ ID NO: 32 - DNA, 462 bp, Artificial Sequence
  Artificial polynucleotide
atgaccatgattacgccaagcttggtaccgagctcggatccactagtaacggccgccagtgtgctggaattcgcccttctaatacgactcactatagggcaagcagtggtatcaacgcagagtacggggggaggcttggtacagcctggcaggtccctgagactctcctgtgcagcctctggattcacccttgatgattatggcatgcactgggtccggcaagctccagggaagggcctggagtgggtctcaggtattagttggaatagtgatagtataggctatgtggactctgtgaagggccgattcaccatctccagagacaacgccaagaactccctgtatctgcaaatgaacagtctgagagttgaggacacggccttgtattactgtgtaaaagatattagtggctggtacagctttgactactggggccagggaaccctggtcaccgtctcctca

SEQ ID NO: 33 - protein, 127 aa, Artificial Sequence
  Artificial polypeptide
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 34 - DNA, 381 bp, Artificial Sequence
  Artificial polynucleotide
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaa

SEQ ID NO: 35 - protein, 139 aa, Artificial Sequence
  Artificial polypeptide
MEWGPCWVFLVVILEGVQCGVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCARGVYHNGWSFFDYWGQGTLTVSS

SEQ ID NO: 36 - DNA, 420 bp, Artificial Sequence
  Artificial polynucleotide
atggagtgggggccgtgctgggttttccttgttgttattttagaaggtgtccagtgtggggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactatgcagactctgtgaagggccgattcaccatctccagagacaatgccaagaactcactgtatctgcaaatgaacagcctgagagacgaggacacggctgtgtattactgtgcgagaggagtgtatcacaatggctggtccttctttgactactggggccagggaaccctactcaccgtctcctca

SEQ ID NO: 37 - protein, 219 aa, Artificial Sequence
  Artificial polypeptide
MDMRVLAQLLGLLLLCFPGARCDIQMTQSPSSLSASVGNRVTITCRASQDISSWLAWYQQKPEKAPKSLIYAASSLQSGVPSRFSGSGSGMDMRVLAQLLGLLLLCFPGARCDIQMTQSPSSLSASVGNRVTITCRASQDISSWLAWYQQKPEKAPKSLIYAASSLQSGVPSRFSGSGSGDFTLTISSLQPEDFATYYCQQYNSYPLTFGQGTRLEIKR

SEQ ID NO: 38 - DNA, 390 bp, Artificial Sequence
  Artificial polynucleotide
atggacatgagggtcctcgctcagctcctggggctcctgctgctctgtttcccaggtgccagatgtgacatccagatgacccagtccccatcctcactgtctgcatctgtaggaaacagagtcaccattacttgtcgggcgagtcaggatattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccccctcaccttcggccaagggacacgactggagattaaacga

SEQ ID NO: 39 - protein, 136 aa, Artificial Sequence
  Artificial polypeptide
MDTLCSTLLLLTIPSWVLSQITLKESGPTLVKPTQTLTLTCTFSGFSLSTSGVGVGWIRQPPGKALEWLALIYWDDHSPYSPSLKSRLTITKDTSKNQVVLTMTNMDPVDTATYYCARTRGAFDIWGQGTMVTVSS

SEQ ID NO: 40 - DNA, 408 bp, Artificial Sequence
  Artificial polynucleotide
atggacactctttgctccacgctcctgctgctgaccatcccttcatgggtcttgtcccagatcaccttgaaggagtctggtcctacgctggtgaaacccacacagaccctcacgctgacctgcaccttctctggattctcactcagcactagtggagtgggtgtgggctggatccgtcagcccccaggaaaggccctggagtggcttgcactcatttattgggatgatcatagcccctacagcccatctctgaagagcaggctcaccatcaccaaggacacctccaaaaaccaggtggtccttacaatgaccaacatggaccctgtggacacagccacatattactgtgcacgcacccggggggcttttgatatctggggccaagggacaatggtcaccgtctcttca

SEQ ID NO: 41 - protein, 127 aa, Artificial Sequence
  Artificial polypeptide
MEAPAQLLFLLLLWLPDTTGEIVLTQSPATLSLSPGERATLSCRASQGVSSYLAWYQQKPGQAPRLLIYDASNRATGIPARFSGSGPGTDFTLTISSLEPEDFAVYYCQQRSNWHPTFGQGTKVEIK

SEQ ID NO: 42 - DNA, 401 bp, Artificial Sequence
  Artificial polynucleotide
atggaagccccagcgcagcttctcttcctcctgctactctggctcccagataccaccggagaaattgtgttgacacagtctccagccaccctgtctttgtctccaggggaaagagccaccctctcctgcagggccagtcagggtgttagcagctacttagcctggtaccagcagaaacctggccaggctcccaggctcctcatctatgatgcatccaacagggccactggcatcccagccaggttcagtggcagtgggcctgggacagacttcactctcaccatcagcagcctagagcctgaagattttgcagtttattactgtcagcagcgtagcaactggcatccgacgttcggccaagggaccaaggtggaaatcaaacgaactgtggctgcaccatc

SEQ ID NO: 43 - protein, 140 aa, Artificial Sequence
  Artificial polypeptide
MDTLCSTLLLLTIPSWVLSQITLKESGPTLVKPKQTLTLTCTFSGFSLSTSGMGVGWIRQPPGKALEWLAVIYWDDHQLYSPSLKSRLTITKDTSKNQVVLTMTNMDPVDTATYYCAHRRGAFQHWGQGTLVTVSSASTK

SEQ ID NO: 44 - DNA, 422 bp, Artificial Sequence
  Artificial polynucleotide
atggacacactttgctccacgctcctgctgctgaccatcccttcatgggtcttgtcccagatcaccttgaaggagtctggtcctacgctagtgaagcccaaacagaccctcacgctgacctgcaccttctctggattctcactcagcactagtggaatgggtgtgggctggatccgtcagcccccaggaaaggccctggagtggcttgcagtcatttattgggatgatcatcaactctacagtccatctctgagagcaggctcaccatcaccaaggacacctccaaaaaccaggtggtccttacaatgaccaacatggaccctgtggacacagccacatattactgtgcacacagacgaggggccttccagcactggggccagggcaccctggtcaccgtctcctcagcttccaccaagggc

SEQ ID NO: 45 - protein, 129 aa, Artificial Sequence
  Artificial polypeptide
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYDSSLTFGGGTKVEIKRT

SEQ ID NO: 46 - DNA, 447 bp, Artificial Sequence
  Artificial polynucleotide
atggagtgggggccgtgctgggttttccttgttgttattttagaaggtgtccagtgtgggatggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatgatagctcgctcactttcggcggagggaccaaggtggagatcaaacgaact

SEQ ID NO: 47 - protein, 136 aa, Artificial Sequence
  Artificial polypeptide
MDTLCSTLLLLTIPSWVLSQITLKESGPTLVKPTQTLTLSCTFSGFSLSTSGVGVGWIRQPPGKALEWLALIHWDDAERYSPSLKSRLTITKDTSKNQVVLTMTNMDLVDTATYYCAHTRGAFDIWGQGTMVTVSS

SEQ ID NO: 48 - DNA, 408 bp, Artificial Sequence
  Artificial polynucleotide
atggacacactttgctccacgctcctgctgctgaccatcccttcatgggtcttgtcccagatcaccttgaaggagtctggtcctacgctggtgaaacccacacagaccctcacgctgtcctgcaccttctctgggttctcactcagcactagtggagtgggtgtgggctggatccgtcagcccccaggaaaggccctggaatggcttgcactcattcattgggatgatgctgagcgctacagtccatctctgaagagcaggctcaccatcaccaaggacacctccaaaaaccaggtggtccttacaatgaccaacatggaccttgtggacacagccacatattactgtgcacacacccggggggcttttgatatctggggccaagggacaatggtcaccgtctcttca

SEQ ID NO: 49 - protein, 127 aa, Artificial Sequence
  Artificial polypeptide
METPAQLLFLLLLWLPDTTGEIVLTQSPGTLSLSPGERAILSCRASQSVSSSFLAWYQQKPGQAPRLLIYGAFSRATGIPDRFSGSGSGTDFTLTISRLEPEDFAVYYCQQYDSSRTFGQGTKVEIK

SEQ ID NO: 50 - DNA, 381 bp, Artificial Sequence
  Artificial polynucleotide
atggaaaccccagcgcagcttctcttcctcctgctactctggctcccagataccaccggagaaattgtgttgacgcagtctccaggcaccctgtctttgtctccaggggaaagagccatcctctcctgcagggccagtcagagtgttagcagcagcttcttagcctggtaccaacagaaacctggccaggctcccaggctcctcatctatggtgcatttagcagggccactggcatcccagacaggttcagtggcagtgggtctgggacagacttcactctcaccatcagcagactggagcctgaagattttgcagtgtattactgtcagcagtatgatagctcacggacgttcggccaggggaccaaggtggaaatcaaa

(19) United States
(12) Patent Application Publication
(10) Pub. No.: US 2022/0265820 A1
(43) Pub. Date: Aug. 25, 2022

(54) TUMOR IMMUNOTHERAPY USING SINDBIS VIRAL VECTORS AND AGONIST MONOCLONAL ANTIBODIES

(71) Applicant: New York University, New York, NY (US)

(72) Inventors: Daniel MERUELO, Scarborough, NY (US); Iris SCHERWITZL, New York, NY (US); Silvana OPP, New York, NY (US); Minjun YU, Dobbs Ferry, NY (US); Alicia HURTADO-MARTINEZ, New York, NY (US); Christine PAMPENO, New York, NY (US)

(21) Appl. No.: 17/625,326

(22) PCT Filed: Jul. 8, 2020
(86) PCT No.: PCT/US2020/041116

§ 371 Date: Jan. 6, 2022

Related U.S. Application Data
(60) Provisional application No. 62/871,675, filed on Jul. 8, 2019.

(51) Int. Cl. A61K 39/395; A61K 48/00; A61P 35/00; A61K 39/00; C07K 16/28

(52) U.S. Cl. CPC A61K 39/3955; A61K 48/0083; A61P 35/00; A61K 39/001188; C07K 2317/75; A61K 2039/505; A61K 2039/55538; C12N 2770/36143; C12N 2770/36134; C07K 16/2878

(57) ABSTRACT

The present disclosure provides compositions and methods for the treatment of cancer. More specifically, the present disclosure provides compositions and methods utilizing a combination of an oncolytic viruses, such as Sindbis virus, and antibodies directed against a co-stimulatory molecule or to an immune system agonist molecule, such as anti-OX40 antibodies and anti-4-1BB antibodies.

### CROSS-REFERENCE TO RELATED APPLICATIONS

This application claims the benefit of U.S. Provisional Application No. 62/871,675, filed on Jul. 8, 2019, the contents of which is hereby incorporated by reference in their entirety.

### INCORPORATION-BY-REFERENCE OF SEQUENCE LISTING

The instant application contains a Sequence Listing which has been submitted in ASCII format via EFS-Web and is hereby incorporated by reference in its entirety. Said ASCII copy, created on Jul. 3, 2020, is named “27522-0225PCT Sequence Listing_ST25.txt” and is 34 kilobytes in size.

### FIELD OF THE INVENTION

The present disclosure describes compositions and methods directed to treating cancer where the compositions include utilizing oncolytic viruses, such as Sindbis virus, and antibodies directed against a co-stimulatory molecule or to an immunesystem agonist molecule, such as OX-40 and 4-1BB (CD137).

### BACKGROUND OF THE INVENTION

Immune checkpoint modulation has shown remarkable promise in treating cancer. Although, high response rates with immune checkpoint blockade have been documented in patients with highly immunogenic tumors, often the proportion of patients that respond to treatment is still low. Major challenges to overcome are the lack of T cell infiltration into the tumor microenvironment as well as the immunosuppressive nature of the tumor, which inhibits the intratumoral immune response. Further, tumors tend to quickly escape the immune response by mutating or losing the expression of drug targets or tumor antigens targeted by the immune response. Thus there is a need in the art for compositions and methods that overcome these limitations. The present disclosure addresses these needs.

### SUMMARY OF THE INVENTION

The present disclosure provides methods for treating cancer in a subject in need thereof comprising administering to the subject a therapeutically effective amount of (a) a oncolytic viral vector and (b) an antibody directed against a co-stimulatory molecule or a nucleic acid encoding same; or an antibody to an immunesystem agonist molecule or a nucleic acid encoding same.

The oncolytic viral vector can be a Sindbis viral vector. The Sindbis viral vector can be replication defective. The Sindbis viral vector can comprise at least one nucleic acid encoding a therapeutic protein. The Sindbis viral vector can comprise at least one nucleic acid encoding an immunostimulatory or an immunomodulatory protein. The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12. The Sindbis viral vector can comprise at least one nucleic acid encoding LacZ, Flue or GFP.

The antibody can be an anti-OX40 antibody, an anti-4-1BB antibody, an anti-CD28 antibody, an anti-GITR antibody, an anti-CD137 antibody, an anti-CD37 antibody, an anti-HVEM antibody, or a combination thereof.

The Sindbis viral vector and the antibody can induce an immune response in a tumor associated antigen (TAA) nonspecific manner. The induced and nonspecific immune response can be a first immune response. The first immune response can be followed by a secondary immuneresponse. The secondary immune response can be the result of one or more TAAs released from the dead tumor cells. The secondary immune response can comprise memory T cells directed against one or more TAAs released from the dead tumor cells.

The present disclosure also provides methods for treating cancer in a subject in need thereof comprising administering to the subject a therapeutically effective amount of (a) a Sindbis viral vector comprising a nucleic acid encoding interleukin-12 and (b) an anti-OX40 monoclonal antibody or a nucleic acid encoding same, thereby treating cancer in the subject.

The Sindbis viral vector can be replication defective. The Sindbis viral vector can comprise the nucleic acid encoding interleukin-12 and can further comprise the nucleic acid encoding the anti-OX40 monoclonal antibody. The method can comprise administering a Sindbis viral vector comprising the nucleic acid encoding interleukin-12 and administering a Sindbis viral vector comprising the nucleic acid encoding the anti-OX40 monoclonal antibody. The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit (IL-12 β, IL-12, p40 subunit) GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise a nucleic acid encoding interleukin-12 alpha subunit of amino acid sequence of GenBank accession no. AAA39292.1 and comprise a nucleic acid encoding interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector can comprise a nucleic acid encoding a human anti-OX40 variable heavy chain comprising the amino acid sequence of SEQ ID NO: 31. The nucleic acid sequence encoding the human anti-OX40 variable heavy chain is SEQ ID NO: 32. The Sindbis viral vector can comprise a nucleic acid encoding a human anti-OX40 variable light chain comprising the amino acid sequence of SEQ ID NO: 33. The nucleic acid sequence encoding the human anti-OX40 variable light chain is SEQ ID NO: 34.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 31. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 32. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 33. The nucleic acid sequence encoding the human anti-OX40 variable light chain that is at least 70%, 75%, 80%, 85%, 90%, 95%, 96%, 97%, 98%, 99% or 100% identical to SEQ ID NO: 34.

The Sindbis viral vector can comprise a nucleic acid encoding a human anti-OX40 variable heavy chain comprising the amino acid sequence of SEQ ID NO: 35. The nucleic acid sequence encoding the human anti-OX40 variable heavy chain is SEQ ID NO: 36. The Sindbis viral vector can comprise a nucleic acid encoding a human anti-OX40 variable light chain comprising the amino acid sequence of SEQ ID NO: 37. The nucleic acid sequence encoding the human anti-OX40 variable light chain is SEQ ID NO: 38.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 35. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 36. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 37. The nucleic acid sequence encoding the human anti-OX40 variable light chain that is at least 70%, 75%, 80%, 85%, 90%, 95%, 96%, 97%, 98%, 99% or 100% identical to SEQ ID NO: 38.

The Sindbis viral vector can comprise a nucleic acid encoding a human anti-OX40 variable heavy chain comprising the amino acid sequence of SEQ ID NO: 39. The nucleic acid sequence encoding the human anti-OX40 variable heavy chain is SEQ ID NO: 40. The Sindbis viral vector can comprise a nucleic acid encoding a human anti-OX40 variable light chain comprising the amino acid sequence of SEQ ID NO: 41. The nucleic acid sequence encoding the human anti-OX40 variable light chain is SEQ ID NO: 42.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 39. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 40. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 41. The nucleic acid sequence encoding the human anti-OX40 variable light chain that is at least 70%, 75%, 80%, 85%, 90%, 95%, 96%, 97%, 98%, 99% or 100% identical to SEQ ID NO: 42.

The Sindbis viral vector can comprise a nucleic acid encoding a human anti-OX40 variable heavy chain comprising the amino acid sequence of SEQ ID NO: 43. The nucleic acid sequence encoding the human anti-OX40 variable heavy chain is SEQ ID NO: 44. The Sindbis viral vector can comprise a nucleic acid encoding a human anti-OX40 variable light chain comprising the amino acid sequence of SEQ ID NO: 45. The nucleic acid sequence encoding the human anti-OX40 variable light chain is SEQ ID NO: 46.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 43. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 44. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 45. The nucleic acid sequence encoding the human anti-OX40 variable light chain that is at least 70%, 75%, 80%, 85%, 90%, 95%, 96%, 97%, 98%, 99% or 100% identical to SEQ ID NO: 46.

The Sindbis viral vector can comprise a nucleic acid encoding a human anti-OX40 variable heavy chain comprising the amino acid sequence of SEQ ID NO: 47. The nucleic acid sequence encoding the human anti-OX40 variable heavy chain is SEQ ID NO: 48. The Sindbis viral vector can comprise a nucleic acid encoding a human anti-OX40 variable light chain comprising the amino acid sequence of SEQ ID NO: 49. The nucleic acid sequence encoding the human anti-OX40 variable light chain is SEQ ID NO: 50.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 47. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 48. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 49. The nucleic acid sequence encoding the human anti-OX40 variable light chain that is at least 70%, 75%, 80%, 85%, 90%, 95%, 96%, 97%, 98%, 99% or 100% identical to SEQ ID NO: 50.

The Sindbis viral vector and the anti-OX40 monoclonal antibody can be administered sequentially or concurrently. The Sindbis viral vector can be administered systemically. The anti-OX40 monoclonal antibody can be administered systemically. Both the Sindbis viral vector and the anti-OX40 monoclonal antibody, or a nucleic acid encoding same, can be administered systemically. The Sindbis viral vector can be administered parenterally. The anti-OX40 monoclonal antibody can be administered parenterally. Both the Sindbis viral vector and the anti-OX40 monoclonal antibody, or a nucleic acid encoding same, can be administered parenterally. The Sindbis viral vector can be administered intraperitoneally. The anti-OX40 monoclonal antibody can be administered intraperitoneally. Both the Sindbis viral vector and the anti-OX40 monoclonal antibody, or a nucleic acid encoding same, can be administered intraperitoneally.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

The present disclosure further provides a Sindbis viral vector comprising a nucleic acid encoding interleukin-12 and a nucleic acid encoding an anti-OX40 monoclonal antibody. The present disclosure also provides a composition comprising (a) a Sindbis viral vector comprising a nucleic acid encoding interleukin-12 and (b) an anti-OX40 monoclonal antibody or a nucleic acid encoding same. The present disclosure also provides a composition comprising (a) a Sindbis viral vector comprising a nucleic acid encoding interleukin-12 and (b) a Sindbis viral vector comprising a nucleic acid encoding an anti-OX40 monoclonal antibody.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit (IL-12 β, IL-12, p40 subunit) GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise a nucleic acid encoding interleukin-12 alpha subunit of amino acid sequence of GenBank accession no. AAA39292.1 and comprise a nucleic acid encoding interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector can comprise a nucleic acid encoding a human anti-OX40 variable heavy chain comprising the amino acid sequence of SEQ ID NO: 31. The nucleic acid sequence encoding the human anti-OX40 variable heavy chain is SEQ ID NO: 32. The Sindbis viral vector can comprise a nucleic acid encoding a human anti-OX40 variable light chain comprising the amino acid sequence of SEQ ID NO: 33. The nucleic acid sequence encoding the human anti-OX40 variable light chain is SEQ ID NO: 34.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 31. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 32. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 33. The nucleic acid sequence encoding the human anti-OX40 variable light chain that is at least 70%, 75%, 80%, 85%, 90%, 95%, 96%, 97%, 98%, 99% or 100% identical to SEQ ID NO: 34.

The Sindbis viral vector can comprise a nucleic acid encoding a human anti-OX40 variable heavy chain comprising the amino acid sequence of SEQ ID NO: 35. The nucleic acid sequence encoding the human anti-OX40 variable heavy chain is SEQ ID NO: 36. The Sindbis viral vector can comprise a nucleic acid encoding a human anti-OX40 variable light chain comprising the amino acid sequence of SEQ ID NO: 37. The nucleic acid sequence encoding the human anti-OX40 variable light chain is SEQ ID NO: 38.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 35. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 36. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 37. The nucleic acid sequence encoding the human anti-OX40 variable light chain that is at least 70%, 75%, 80%, 85%, 90%, 95%, 96%, 97%, 98%, 99% or 100% identical to SEQ ID NO: 38.

The Sindbis viral vector can comprise a nucleic acid encoding a human anti-OX40 variable heavy chain comprising the amino acid sequence of SEQ ID NO: 39. The nucleic acid sequence encoding the human anti-OX40 variable heavy chain is SEQ ID NO: 40. The Sindbis viral vector can comprise a nucleic acid encoding a human anti-OX40 variable light chain comprising the amino acid sequence of SEQ ID NO: 41. The nucleic acid sequence encoding the human anti-OX40 variable light chain is SEQ ID NO: 42.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 39. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 40. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 41. The nucleic acid sequence encoding the human anti-OX40 variable light chain that is at least 70%, 75%, 80%, 85%, 90%, 95%, 96%, 97%, 98%, 99% or 100% identical to SEQ ID NO: 42.

The Sindbis viral vector can comprise a nucleic acid encoding a human anti-OX40 variable heavy chain comprising the amino acid sequence of SEQ ID NO: 43. The nucleic acid sequence encoding the human anti-OX40 variable heavy chain is SEQ ID NO: 44. The Sindbis viral vector can comprise a nucleic acid encoding a human anti-OX40 variable light chain comprising the amino acid sequence of SEQ ID NO: 45. The nucleic acid sequence encoding the human anti-OX40 variable light chain is SEQ ID NO: 46.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 43. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 44. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 45. The nucleic acid sequence encoding the human anti-OX40 variable light chain that is at least 70%, 75%, 80%, 85%, 90%, 95%, 96%, 97%, 98%, 99% or 100% identical to SEQ ID NO: 46.

The Sindbis viral vector can comprise a nucleic acid encoding a human anti-OX40 variable heavy chain comprising the amino acid sequence of SEQ ID NO: 47. The nucleic acid sequence encoding the human anti-OX40 variable heavy chain is SEQ ID NO: 48. The Sindbis viral vector can comprise a nucleic acid encoding a human anti-OX40 variable light chain comprising the amino acid sequence of SEQ ID NO: 49. The nucleic acid sequence encoding the human anti-OX40 variable light chain is SEQ ID NO: 50.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 47. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 48. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 49. The nucleic acid sequence encoding the human anti-OX40 variable light chain that is at least 70%, 75%, 80%, 85%, 90%, 95%, 96%, 97%, 98%, 99% or 100% identical to SEQ ID NO: 50.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The present disclosure also provides methods for treating cancer in a subject in need thereof comprising administering to the subject a therapeutically effective amount of a Sindbis viral vector comprising a nucleic acid encoding interleukin-12 and a Sindbis viral vector comprising a nucleic acid encoding NY-ESO-1, thereby treating cancer in the subject. The present disclosure also provides methods for treating cancer in a subject in need thereof comprising administering to the subject a therapeutically effective amount of a Sindbis viral vector comprising a nucleic acid encoding interleukin-12 and a nucleic acid encoding NY-ESO-1, thereby treating cancer in the subject.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit (IL-12 β, IL-12, p40 subunit) GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise a nucleic acid encoding interleukin-12 alpha subunit of amino acid sequence of GenBank accession no. AAA39292.1 and comprise a nucleic acid encoding interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The Sindbis viral vector comprising a nucleic acid encoding interleukin-12 and the Sindbis viral vector comprising a nucleic acid encoding NY-ESO-1 can be administered sequentially or concurrently. The Sindbis viral vector comprising a nucleic acid encoding interleukin-12 can be administered systemically. The Sindbis viral vector comprising a nucleic acid encoding NY-ESO-1 can be administered systemically. Both the Sindbis viral vector comprising a nucleic acid encoding interleukin-12 and the Sindbis viral vector comprising a nucleic acid encoding NY-ESO-1 can be administered systemically. The Sindbis viral vector comprising a nucleic acid encoding interleukin-12 can be administered parenterally. The Sindbis viral vector comprising a nucleic acid encoding NY-ESO-1 can be administered parenterally. Both the Sindbis viral vector comprising a nucleic acid encoding interleukin-12 and the Sindbis viral vector comprising a nucleic acid encoding NY-ESO-1 can be administered parenterally. The Sindbis viral vector comprising a nucleic acid encoding interleukin-12 can be administered intraperitoneally. The Sindbis viral vector comprising a nucleic acid encoding NY-ESO-1 can be administered intraperitoneally. Both the Sindbis viral vector comprising a nucleic acid encoding interleukin-12 and the Sindbis viral vector comprising a nucleic acid encoding NY-ESO-1 can be administered intraperitoneally.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

The present disclosure further provides a Sindbis viral vector comprising a nucleic acid encoding interleukin-12 and a nucleic acid encoding NY-ESO-1. The present disclosure also provides a composition comprising (a) a Sindbis viral vector comprising a nucleic acid encoding interleukin-12 and (b) a Sindbis viral vector comprising a nucleic acid encoding NY-ESO-1.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit (IL-12 β, IL-12, p40 subunit) GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise a nucleic acid encoding interleukin-12 alpha subunit of amino acid sequence of GenBank accession no. AAA39292.1 and comprise a nucleic acid encoding interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The present disclosure provides methods for treating cancer in a subject in need thereof comprising administering to the subject a therapeutically effective amount of (a) a Sindbis viral vector comprising a nucleic acid encoding NY-ESO-1 and (b) an anti-OX40 monoclonal antibody or a nucleic acid encoding same, thereby treating cancer in the subject.

The Sindbis viral vector can be replication defective. The Sindbis viral vector can comprise the nucleic acid encoding NY-ESO-1 and can further comprise the nucleic acid encoding the anti-OX40 monoclonal antibody. The method can comprise administering a Sindbis viral vector comprising the nucleic acid encoding NY-ESO-1 and administering a Sindbis viral vector comprising the nucleic acid encoding the anti-OX40 monoclonal antibody. The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector can comprise a nucleic acid encoding a human anti-OX40 variable heavy chain comprising the amino acid sequence of SEQ ID NO: 31. The nucleic acid sequence encoding the human anti-OX40 variable heavy chain is SEQ ID NO: 32. The Sindbis viral vector can comprise a nucleic acid encoding a human anti-OX40 variable light chain comprising the amino acid sequence of SEQ ID NO: 33. The nucleic acid sequence encoding the human anti-OX40 variable light chain is SEQ ID NO: 34.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 31. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 32. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 33. The nucleic acid sequence encoding the human anti-OX40 variable light chain that is at least 70%, 75%, 80%, 85%, 90%, 95%, 96%, 97%, 98%, 99% or 100% identical to SEQ ID NO: 34.

The Sindbis viral vector can comprise a nucleic acid encoding a human anti-OX40 variable heavy chain comprising the amino acid sequence of SEQ ID NO: 35. The nucleic acid sequence encoding the human anti-OX40 variable heavy chain is SEQ ID NO: 36. The Sindbis viral vector can comprise a nucleic acid encoding a human anti-OX40 variable light chain comprising the amino acid sequence of SEQ ID NO: 37. The nucleic acid sequence encoding the human anti-OX40 variable light chain is SEQ ID NO: 38.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 35. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 36. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 37. The nucleic acid sequence encoding the human anti-OX40 variable light chain that is at least 70%, 75%, 80%, 85%, 90%, 95%, 96%, 97%, 98%, 99% or 100% identical to SEQ ID NO: 38.

The Sindbis viral vector can comprise a nucleic acid encoding a human anti-OX40 variable heavy chain comprising the amino acid sequence of SEQ ID NO: 39. The nucleic acid sequence encoding the human anti-OX40 variable heavy chain is SEQ ID NO: 40. The Sindbis viral vector can comprise a nucleic acid encoding a human anti-OX40 variable light chain comprising the amino acid sequence of SEQ ID NO: 41. The nucleic acid sequence encoding the human anti-OX40 variable light chain is SEQ ID NO: 42.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 39. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 40. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 41. The nucleic acid sequence encoding the human anti-OX40 variable light chain that is at least 70%, 75%, 80%, 85%, 90%, 95%, 96%, 97%, 98%, 99% or 100% identical to SEQ ID NO: 42.

The Sindbis viral vector can comprise a nucleic acid encoding a human anti-OX40 variable heavy chain comprising the amino acid sequence of SEQ ID NO: 43. The nucleic acid sequence encoding the human anti-OX40 variable heavy chain is SEQ ID NO: 44. The Sindbis viral vector can comprise a nucleic acid encoding a human anti-OX40 variable light chain comprising the amino acid sequence of SEQ ID NO: 45. The nucleic acid sequence encoding the human anti-OX40 variable light chain is SEQ ID NO: 46.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 43. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 44. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 45. The nucleic acid sequence encoding the human anti-OX40 variable light chain that is at least 70%, 75%, 80%, 85%, 90%, 95%, 96%, 97%, 98%, 99% or 100% identical to SEQ ID NO: 46.

The Sindbis viral vector can comprise a nucleic acid encoding a human anti-OX40 variable heavy chain comprising the amino acid sequence of SEQ ID NO: 47. The nucleic acid sequence encoding the human anti-OX40 variable heavy chain is SEQ ID NO: 48. The Sindbis viral vector can comprise a nucleic acid encoding a human anti-OX40 variable light chain comprising the amino acid sequence of SEQ ID NO: 49. The nucleic acid sequence encoding the human anti-OX40 variable light chain is SEQ ID NO: 50.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 47. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 48. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 49. The nucleic acid sequence encoding the human anti-OX40 variable light chain that is at least 70%, 75%, 80%, 85%, 90%, 95%, 96%, 97%, 98%, 99% or 100% identical to SEQ ID NO: 50.

The Sindbis viral vector and the anti-OX40 monoclonal antibody can be administered sequentially or concurrently. The Sindbis viral vector can be administered systemically. The anti-OX40 monoclonal antibody can be administered systemically. Both the Sindbis viral vector and the anti-OX40 monoclonal antibody, or a nucleic acid encoding same, can be administered systemically. The Sindbis viral vector can be administered parenterally. The anti-OX40 monoclonal antibody can be administered parenterally. Both the Sindbis viral vector and the anti-OX40 monoclonal antibody, or a nucleic acid encoding same, can be administered parenterally. The Sindbis viral vector can be administered intraperitoneally. The anti-OX40 monoclonal antibody can be administered intraperitoneally. Both the Sindbis viral vector and the anti-OX40 monoclonal antibody, or a nucleic acid encoding same, can be administered intraperitoneally.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

The present disclosure further provides a Sindbis viral vector comprising a nucleic acid encoding NY-ESO-1 and a nucleic acid encoding an anti-OX40 monoclonal antibody. The present disclosure also provides a composition comprising (a) a Sindbis viral vector comprising a nucleic acid encoding NY-ESO-1 and (b) an anti-OX40 monoclonal antibody or a nucleic acid encoding same. The present disclosure also provides a composition comprising (a) a Sindbis viral vector comprising a nucleic acid encoding NY-ESO-1 and (b) a Sindbis viral vector comprising a nucleic acid encoding an anti-OX40 monoclonal antibody. The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The present disclosure also provides methods for treating cancer in a subject in need thereof comprising administering to the subject a therapeutically effective amount (a) a Sindbis viral vector and (b) an anti-4-1BB (CD137) monoclonal antibody or a nucleic acid encoding same, thereby treating cancer in the subject. The present disclosure further provides in vitro or ex vivo methods for treating cancer or assessing the treatment of cancer in a subject comprising contacting a biological sample from the subject with (a) a Sindbis viral vector and (b) an anti-4-1BB monoclonal antibody or a nucleic acid encoding same. The Sindbis viral vector does not comprise an endogenous nucleic acid encoding any protein.

The Sindbis viral vector is replication defective. The Sindbis viral vector can comprise a nucleic acid sequence encoding a therapeutic protein, an immunostimulatory protein or an immunomodulatory protein. The Sindbis viral vector can comprise the nucleic acid encoding the therapeutic protein, an immunostimulatory protein or an immunomodulatory protein and further comprise the nucleic acid encoding the anti-4-1BB monoclonal antibody. The Sindbis viral vector can comprise a nucleic acid sequence encoding LacZ (lac operon structural gene lacZ encoding β-galactosidase), Flue (firefly luciferase) or GFP (green fluorescent protein). The Sindbis viral vector can comprise the nucleic acid encoding LacZ, Flue or GFP and further comprise the nucleic acid encoding the anti-4-1BB monoclonal antibody.

The Sindbis viral vector can comprise a nucleic acid encoding an anti-4-1BB antibody heavy chain comprising the heavy chain complementarity determining region 1 (HCDR1), HCDR2 and HCDR3 amino acid sequences of SEQ ID NOs: 16, 17 and 18, respectively. The Sindbis viral vector can comprise the nucleic acid encoding an anti-4-1BB antibody heavy chain comprising the amino acid sequence of SEQ ID NO: 19. The Sindbis viral vector can comprise a nucleic acid encoding an anti-4-1BB antibody heavy chain comprising the light chain complementarity determining region 1 (LCDR1), LCDR2 and LCDR3 amino acid sequences of SEQ ID NOs: 20, 21 and 22, respectively. The Sindbis viral vector can comprise the nucleic acid encoding an anti-4-1BB antibody light chain comprising the amino acid sequence of SEQ ID NO: 23.

The Sindbis viral vector can comprise the nucleic acid encoding anti-4-1BB antibody heavy chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody light chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody heavy chain amino acid sequence that binds to a target antigen of the amino acid sequence of SEQ ID NO: 24. The Sindbis viral vector can comprise the nucleic acid encoding anti-4-1BB antibody light chain amino acid sequence that binds to a target antigen of the amino acid sequence of SEQ ID NO: 24.

The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12. The anti-4-1BB antibody can be urelumab, utomilumab or a combination thereof. The anti-4-1BB antibody can be InVivoMAb anti-mouse 4-1BB (BioXCell, Clone: LOB12.3, Cat. No. BE0169).

The Sindbis viral vector and the anti-4-1BB monoclonal antibody can be administered sequentially or concurrently. The Sindbis viral vector can be administered systemically. The anti-4-1BB monoclonal antibody can be administered systemically. Both the Sindbis viral vector and the anti-4-1BB monoclonal antibody, or a nucleic acid encoding same, can be administered systemically. The Sindbis viral vector can be administered parenterally. The anti-4-1BB monoclonal antibody can be administered parenterally. Both the Sindbis viral vector and the anti-4-1BB monoclonal antibody, or a nucleic acid encoding same, can be administered parenterally. The Sindbis viral vector can be administered intraperitoneally. The anti-4-1BB monoclonal antibody can be administered intraperitoneally. Both the Sindbis viral vector and the anti-4-1BB monoclonal antibody, or a nucleic acid encoding same, can be administered intraperitoneally.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against 4-1BB or an anti-4-1BB monoclonal antibody, as described herein, can be a full length antibody against 4-1BB antibody or an antigen binding fragment thereof, wherein the fragment retains binding to the 4-1BB receptor on a cell surface. An “antigen-binding fragment” of an anti-4-1BB antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is a lymphoma. In one preferred aspect, the cancer is a B cell lymphoma.

The present disclosure provides a Sindbis viral vector comprising a nucleic acid encoding a therapeutic protein, an immunostimulatory protein or an immunomodulatory protein and a nucleic acid encoding an anti-4-1BB monoclonal antibody. The present disclosure provides a Sindbis viral vector comprising a nucleic acid encoding encoding LacZ, Flue or GFP and a nucleic acid encoding an anti-4-1BB monoclonal antibody. The present disclosure also provides a composition comprising (a) a Sindbis viral vector comprising a nucleic acid encoding a therapeutic protein, an immunostimulatory protein or an immunomodulatory protein and (b) a Sindbis viral vector comprising a nucleic acid encoding an anti-4-1BB monoclonal antibody. The present disclosure also provides a composition comprising (a) a Sindbis viral vector comprising a nucleic acid encoding LacZ, Flue or GFP and (b) a Sindbis viral vector comprising a nucleic acid encoding an anti-4-1BB monoclonal antibody. The present disclosure further provides a composition comprising (a) a Sindbis viral vector comprising a nucleic acid encoding a therapeutic protein, an immunostimulatory protein or an immunomodulatory protein and (b) an anti-4-1BB monoclonal antibody or a nucleic acid encoding same. The present disclosure provides a composition comprising (a) a Sindbis viral vector comprising a nucleic acid encoding encoding LacZ, Flue or GFP and (b) an anti-4-1BB monoclonal antibody or a nucleic acid encoding same.

The Sindbis viral vector can comprise a nucleic acid encoding an anti-4-1BB antibody heavy chain comprising the heavy chain complementarity determining region 1 (HCDR1), HCDR2 and HCDR3 amino acid sequences of SEQ ID NOs: 16, 17 and 18, respectively. The Sindbis viral vector can comprise the nucleic acid encoding an anti-4-1BB antibody heavy chain comprising the amino acid sequence of SEQ ID NO: 19. The Sindbis viral vector can comprise a nucleic acid encoding an anti-4-1BB antibody heavy chain comprising the light chain complementarity determining region 1 (LCDR1), LCDR2 and LCDR3 amino acid sequences of SEQ ID NOs: 20, 21 and 22, respectively. The Sindbis viral vector can comprise the nucleic acid encoding an anti-4-1BB antibody light chain comprising the amino acid sequence of SEQ ID NO: 23.

The Sindbis viral vector can comprise the nucleic acid encoding anti-4-1BB antibody heavy chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody light chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody heavy chain amino acid sequence that binds to a target antigen of the amino acid sequence of SEQ ID NO: 24. The Sindbis viral vector can comprise the nucleic acid encoding anti-4-1BB antibody light chain amino acid sequence that binds to a target antigen of the amino acid sequence of SEQ ID NO: 24.

The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12.

An antibody of the present disclosure, or a fragment thereof, can be derived from any species, including, but not limited to, a human, a mouse, a rat, a hamster, a dog, a rabbit, a frog, a sheep, a goat, a cow, a horse, a pig, a bird, a donkey, a chicken, a camel, a llama, a dromedary, an alpaca, a shark, a bovine and a turtle. In some aspects, an antibody of the present disclosure, or a fragment thereof, is derived from a human. In some aspects, an antibody of the present disclosure, or a fragment thereof, is derived from a mouse. In some aspects, an antibody of the present disclosure, or a fragment thereof, is derived from a camel, a llama or an alpaca. In some aspects, an antibody of the present disclosure, or a fragment thereof, is derived from a shark. In some aspects, an antibody of the present disclosure, or a fragment thereof, of the present disclosure is a chimeric antibody that is derived from two or more of the aforementioned species. In a non-limiting example, an antibody of the present disclosure, or fragment thereof, can be a chimeric antibody that is derived from a human and a mouse. In some aspects, an antibody of the present disclosure, or a fragment thereof, can be derived from any species other than human and can be further humanized using standard methods known in the art as to reduce the immunogenicity of the antibody.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against 4-1BB or an anti-4-1BB monoclonal antibody, as described herein, can be a full length antibody against 4-1BB antibody or an antigen binding fragment thereof, wherein the fragment retains binding to the 4-1BB receptor on a cell surface. An “antigen-binding fragment” of an anti-4-1BB antibody, as described herein can include any fragment selected from the group consisting of Fv, Fav, F(ab′)2, Fab′, dsFv, scFv, sc(Fv)2, scFv-CH3, scFv-Fc, and diabody fragments.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF DRAWINGS

FIG. 1A-1D. SV.IL12 induces a modest therapeutic efficacy and increases OX40 expression on CD4 T cells. FIG. 1A depicts treatment schema. BALB/c mice were given intraperitoneal (i.p.) injections of SV, IL-12 (50 ng), or SV.IL12 at various times after injection of 7×10⁴ CT.26.Fluc on day 0. FIG. 1B depicts survival plots of control and treated mice bearing CT26.Fluc tumors. The x-axis shows days of treatment and y-axis shows percentage survival. Statistical significance between SV.IL12 and all other groups was determined with the Mantel-Cox test. Results are representatives of at least two independent experiments. FIG. 1C-D depict effect on OX40 expression of treatment of CT26 tumor-bearing mice with SV, IL-12 (50 ng), or SV.IL12 on 4 consecutive days (days 1, 2, 3, and 4). On day 7, spleens were excised and a single-cell suspension was stained and analyzed by flow cytometry. As controls, naive and untreated (control) tumor-bearing mice were used. FIG. 1C depicts percentage of OX40 expression by CD4 T cells (left), regulatory T cells (TREG; middle), and CD8 T cells (right). The x-axis shows the various treatment groups and the y-axis shows percentage of OX40+ cells.

FIG. 1D depicts representative flow cytometry plots indicating OX40 staining in different T cell subsets. Bars represent means and each symbol represents an individual mouse. Statistical significance was determined with the Kruskal-Wallis test followed by the Dunns' test or the Mann-Whitney test. Results are representatives of at least two independent experiments.

FIG. 2A-2C. SV infects monocytes/macrophages in mediastinal lymph nodes and quickly activates T cells. FIG. 2A depicts that tumor free mice were treated i.p. with SV expressing the firefly luciferase (Fluc) protein. 4 hours later, bioluminescent images were taken to monitor Fluc expression from SV. To determine the source of the signal, the mediastinal lymph nodes (LN) and adipose tissue were extracted and imaged separately. FIG. 2B depicts percentage of GFP expression by Ly6G-CD11b+F4/80+ cells. FIG. 2C depicts percentage of CD69 expression by CD4 (left graph) and CD8 (right graph) T cells. Tumor free mice were treated i.p. with SV expressing GFP for 4 consecutive days. On day 5, mediastinal and inguinal LN were extracted and a single cell suspension was stained and analyzed by flow cytometry. As control, naïve mice were used. Statistical significance was determined with the Mann-Whitney test. Results are representatives of at least two independent experiments.

FIG. 3A-3D. IL-12 and IFN-γ production derived from SV.IL12 infection. FIG. 3A depicts IL-12 levels in supernatant of infected cells measured by ELISA. 5×10⁵ MyC-CaP cells were infected with SV.IL12 at various MOI (10; 1; 0′1) for 2 hours. As control, MyC-CaP cells were infected with SV or left uninfected (mock). SV was washed away and replaced with fresh media. After 24 hours incubation, supernatant was collected and IL-12 was measured by ELISA. FIG. 3B depicts IL-12 levels in plasma was measured by ELISA. FIG. 3C depicts percentage of Tbet expression by CD4 T cells in cell suspensions from mediastinal LN stained and analyzed by flow cytometry. FIG. 3D depicts IFNγ enzyme-linked immunospot analysis of splenocytes from control and treated mice as indicated (n=3-10 mice per group). Tumor bearing mice were treated with SV.IL12 on 4 consecutive days (days 1; 2; 3; 4). As control, naïve, untreated (control), IL-12 (50 ng) and SV treated mice were used. On day 7, plasma, spleen and mediastinal LN were collected from each mice. Statistical significance was determined with the Kruskal-Wallis test followed by the he Dunns' test or Mann-Whitney test. Results are representatives of at least two independent experiments.

FIG. 4A-4B. SV infectivity of MyC-CaP.Fluc tumors. MyC-CaP.Fluc and CT.26.Fluc cells were challenged with serially diluted single round replication SV.GFP (10⁻¹-10⁻⁴) and incubated overnight. 16 hours post infection the percentage of GFP-positive cells was analyzed by flow cytometer for each dilution. FIG. 4A depicts representative flow cytometry plots of GFP positive MyC-CaP.Fluc and CT.26.Fluc cells per dilution and uninfected controls are shown. FIG. 4B depicts plotted infectivity curve of GFP-positive cells.

FIG. 5A-5E. SV.IL12 in combination with anti-OX40 antibody cures established tumors in vivo. FIG. 5A depicts the experimental protocol for the prostate and colon cancer model. FVB/NJ or BALB/c mice were given an i.p. injection of SV.IL12 and/or anti-OX40 at various times after injection of 10⁵ MyC-CaP.Fluc or 7×10⁴ CT26.Fluc cells on day 0, respectively. FIG. 5B depicts CT26.Fluc tumor growth curves shown as fold changes relative to the luminescence on day 0 of the same mouse. Each line represents an individual mouse. Left graphs: control (n=14) (top) and SV.IL12 (n=20) (bottom). Right graphs: anti-OX40 (n=10) (top) and SV.IL12+anti-OX40 (n=11) (bottom). FIG. 5C depicts the representative bioluminescence images of control and treated CT26.Fluc-bearing mice. FIG. 5D depicts survival plots of control and treated mice bearing peritoneally disseminated CT26.Fluc tumors. FIG. 5E depicts survival plots of control and treated mice bearing peritoneally disseminated MyC-CaP.Fluc tumors. Statistical significances between SV.IL12+anti-OX40 and anti-OX40 or SV.IL12 were determined with the Mantel-Cox test. Results are representatives of at least two independent experiments.

FIG. 6A-6B. Tumor growth of MyC-CaP.Fluc tumor bearing mice during treatment. FVB/NJ mice were given injection of SV.IL12 and/or anti-OX40 intraperitoneally (i.p.) at various times after injection of 10⁵ MyC-CaP.Fluc cells on day 0. FIG. 6A depicts tumor growth curves are shown as fold changes relative to the luminescence on day 0 of the same mouse. Each line represents an individual mouse. Left graphs: Control (n=10) (top) and SV.IL12 (n=10) (bottom). Right graphs: aOX40 (n=10) (top) and SV.IL12+aOX40 (n=10) (bottom). FIG. 6B depicts representative bioluminescence images of control and treated CT.26.Fluc bearing mice. Results are representatives of at least two independent experiments.

FIG. 7A-7D. The therapeutic efficacy of SV.IL12 in combination with anti-OX40 is maintained at reduced treatment regimen in CT26.Fluc bearing mice. FIG. 7A depicts the treatment schema. BALB/c mice were given i.p. injection of SV.IL12 (day 1 and 8) and/or anti-OX40 (day 2 and 9) of 7×10⁴ CT26.Fluc on day 0. FIG. 7B depicts representative bioluminescence images of control and treated CT26.Fluc bearing mice. FIG. 7C depicts tumor growth curves are shown as fold changes relative to the luminescence on day 0 of the same mouse. Each line represents an individual mouse. Left graphs: Control (n=14) (top) and SV.IL12 (n=15) (bottom). Right graphs: aOX40 (n=17) (top) and SV.IL12+aOX40 (n=15) (bottom). FIG. 7D depicts survival plots of control and treated mice bearing CT26.Fluc tumors. Statistical significance between SV.IL12+aOX40 and SV.IL12 was determined with the Mantel-Cox test. Results are representatives of at least two independent experiments.

FIG. 8A-8B. Therapeutic efficacy of SV.IL12 in combination with anti-OX40 is dependent on CD4 and CD8 T cells. FIG. 8A depicts that mice injected with anti-CD4 (0.4 mg) or anti-CD8 (0.1 mg) depleting antibody. As a control, rat IgG2b (0.4 mg) isotype control was used. The frequency of CD4 and CD8 T cells were assessed by flow cytometry in splenocytes after 24, 48, 72 and 96 hours. FIG. 8B depicts that BALB/c mice were inoculated with 7×10⁴ CT.26.Fluc on day −4. Depletion antibody anti-CD4 or anti-CD8 were injected i.p. on day −3, 1, 5, 9, 13 and 17. Mice were left untreated (control) or were treated with SV.IL12 and anti-OX40 on day 4 and 11. Tumor growth curves are shown as fold changes relative to the luminescence on day 0 of the same mouse. Each line represents an individual mouse.

FIG. 9A-9I. Combination therapy markedly changes the transcriptome signature of T cells favoring effector T cells with a Th1 type phenotype. FIGS. 9A-9I depict RNA sequencing of T cells isolated from spleens derived from untreated tumor bearing mice (control) compared with mice treated with SV.IL12 and/or anti-OX40 on day 7. FIG. 9A depicts principal component analysis (PCA) of normalized read counts from the CT26.Fluc tumor model. FIG. 9B depicts PCA of normalized read counts from the MyC-CaP.Fluc tumor model.

FIG. 9C depicts MA plots of differentially expressed genes (DEG; >2-fold) in T cells of control versus anti-OX40 treated mice (top graph), SV.IL12 treated mice (middle graph) or SV.IL12+anti-OX40 treated mice (bottom graph) in the CT.26 model. Significantly (p<0.05) upregulated and downregulated DEG are depicted in red or blue, respectively. FIG. 9D depicts Pathway and network analysis based on DEG in T cells isolated from CT26.Fluc-bearing mice treated with combination therapy. Downregulated (blue) and upregulated (red) pathways are shown, respectively. FIG. 9E depicts Heatmap analysis of selected genes based on normalized read counts linked to T cell differentiation and activation as well as T cell lineage transcription factors. FIGS. 9F-I depict data from tumor bearing mice that were treated with SV.IL12 and/or anti-OX40. As control, naïve and untreated (control) tumor bearing mice were used. On day 7, spleens were excised and a single cell suspension was stained and analyzed by flow cytometry. FIG. 9F depicts percentage of CD44 or Ki-67 expression by T cells from the CT26.Fluc tumor model. FIG. 9G depicts percentage of CD44 or Ki-67 expression by T cells from the MyC-CaP.Fluc tumor model. FIG. 9H depicts the percentage of ICOS and T-bet expression by CD4 T cells.

FIG. 9I depicts representative flow cytometry plots ICOS and T-bet expression by CD4 T cells. Bars represent means and each symbol represent an individual mouse. Statistical significance was determined with the Kruskal-Wallis test followed by the Dunns' test. Results are representatives of at least two independent experiments.

FIG. 10A-10B. Combination therapy induces systemic CD4 and CD8 T cell activation. Tumor bearing mice were left untreated or treated with SV.IL12 and/or anti-OX40. On day 7, spleens were excised and a single cell suspension was stained and analyzed by flow cytometry. As control, naïve and untreated (control) tumor bearing mice were used. FIGS. 10A and 10B depict representative flow cytometry plots of CD44 and Ki-67 expression on CD4 and CD8 T cells in the CT.26.Fluc and MyC-CaP.Fluc tumor model, respectively.

FIG. 11A-11G. SV.IL12 in combination with anti-OX40 promotes metabolic reprogramming of T cells. Tumor bearing mice were left untreated or treated with SV.IL12 and/or anti-OX40. T cells were isolated from spleens on day 7 or otherwise indicated. FIG. 11A depicts selected gene set enrichment analysis (GSEA) of oxidative phosphorylation and glycolysis pathways based on DEG in control versus SV+anti-OX40. FIG. 11B depicts mitochondrial respiration assessed by measuring the median values of oxygen consumption rates (OCR) in T cells of indicated groups using an extracellular flux analyzer. Oligomycin, FCCP, Antimycin A and Rotenone were injected as indicated to identify energetic mitochondrial phenotypes. FIG. 11C depicts Mitotracker Green FM staining of T cells from indicated groups using flow cytometry. FIG. 11D depicts Mitotracker Deep Red FM staining of T cells from indicated groups using flow cytometry. FIG. 11E depicts western blot of c-Myc protein expression in T cells of control or mice treated with anti-OX40, SV.IL12 or SV.IL12+anti-OX40. GAPDH (bottom) is loading control. FIG. 11F depicts baseline extracellular acidification rates (ECAR) in T cells of indicated groups derived from the CT26.Fluc and MyC-CaP.Fluc tumor models. FIG. 11G depicts energy profile (OCR versus ECAR) of T cells from naïve or CT26.Fluc bearing mice treated with SV.IL12+anti-OX40 on day 7, 14 and 40. Error bars indicate SEM. Results are representatives of at least two independent experiments in FIGS. 111B-G.

FIG. 12A-12D. Combination therapy rewires T cells metabolically. FIGS. 12A and 12B depict the metabolic activity of T cells isolated from spleens of mice bearing CT.26.Fluc or MyC-CaP.Fluc tumor respectively, on day 7. Baseline OCR (left) and respiratory capacity (right) was measured in T cells of indicated groups using an extracellular flux analyzer. FIG. 12C depicts representative flow cytometry plots of Mitotracker Green FM and Mitotracker Deep Red staining in CD4 and CD8 T cells isolated from spleens of CT26.Fluc tumor bearing mice. FIG. 12D depicts energy profile (OCR versus ECAR) of T cells from naïve (bottom graph) or CT26.Fluc bearing mice treated with SV.IL12+anti-OX40 (top graph) on day 7, 14 and 30. Data represent the mean of three different experiments. Bars represent means±SEM (A, B, D). Results are representatives of at least two independent experiments.

FIG. 13A-13G. Reprogrammed T cells in SV+anti-OX40 treated mice display enhanced CD4 mediated cytokine production and anti-tumor activity. Tumor bearing mice were left untreated or treated with SV.IL12 and/or anti-OX40. Spleens were excised on day 7 for further analysis. FIG. 13A depicts Heatmap analysis of selected genes based on normalized read counts linked to cytokine expression and enhanced anti-tumor activity. FIG. 13B depicts RNA sequencing performed on isolated T cells from CT26.Fluc tumor-bearing mice. FIG. 13C depicts Interferon-gamma (IFN-γ enzyme-linked immunospot analysis of splenocytes from control and treated mice as indicated (n=5-10 mice per group). Additionally, IFN-γ enzyme-linked immunospot analysis was measured in splenocytes depleted of CD4 or CD8 T cells in the CT26.Fluc (Top) and MyC-CaP.Fluc (Bottom) tumor models. FIG. 13D depicts representative flow cytometry plots of T-bet and granzyme B (GrB) expression by CD4 T cells from indicated groups using flow cytometry. FIG. 13E depicts Percentage of T-bet and granzyme B (GrB) expression by CD4 T cells from indicated groups using flow cytometry of FIG. 13D. FIG. 13F depict cytotoxic activity of T cells from control and treated mice (n=5-10 mice per group) co-cultured at an effector-to-target cell ratio of 10:1 with either CT26.FLUC. FIG. 13G depicts MyC-CaP.Fluc tumor cell lines for 2 days. Additionally, T cells were depleted of CD4 or CD8 T cells and co-cultured as previously described. Cytotoxic activity was assessed based on viability of tumor cells, which was determined by measuring the luciferase activity and is shown relative to naïve T cells. Bars represent means±SEM in FIGS. 13B, 13F and 13G, and each symbol represent an individual mouse in FIG. 13E. Statistical significance was determined with the Kruskal-Wallis test followed by the he Dunns' test. Results are representatives of at least two independent experiments.

FIG. 14A-14F. CD8 T cells show enhanced cytotoxic potential in mice treated with SV.IL12 and anti-OX40. Tumor bearing mice were left untreated or were treated with SV.IL12 with or without anti-OX40. Mice were sacrificed on day 7 to analyze the T cell immune response in spleen. FIGS. 14A-14B depict percentage of granzyme B and T-bet expression, by CD8 T cells from CT26.Fluc tumor bearing mice. FIGS. 14C-14D depict percentage of granzyme B and T-bet expression, respectively by CD8 T cells from MyC-CaP.Fluc tumor bearing mice. FIGS. 14B and 14D depict representative flow cytometry plots. FIGS. 14E and 14F depict percentage of NKG2D (left graph) or T-bet (right graph) expression by CD8 T cells in the mice bearing CT26.Fluc and MyC-CaP.Fluc tumor, respectively. Bars represent means and each symbol represent an individual mouse. Statistical significance was determined with the Kruskal-Wallis test followed by the he Dunns' test. Results are representatives of at least two independent experiments.

FIG. 15A-15F. Mice treated with SV.IL12 in combination with anti-OX40 display enhanced T cell migration and intratumoral T cell activity. CT26.Fluc bearing mice were left untreated or were treated with SV.IL12 and/or anti-OX40. On day 7, spleens were excised and a single cell suspension was stained and analyzed by flow cytometry. FIG. 15A depicts percentage of CXCR3 expression by CD4 (left graph) and CD8 (right graph) T cells. FIG. 15 B depicts representative flow cytometry plots. Tumors were harvested after 2 weeks of treatment from control and treated mice. FIG. 15C depicts intratumoral gene expression of CXCL9 (Top) and CXCL10 (bottom) analyzed by real time PCR. Data are normalized to GAPDH. FIG. 15D depicts intratumoral T cell immune responses from indicated groups that were assessed by flow cytometry. Percentage of CD4 expression by T cells (left graph), Ki-67 expression (middle graph) and granzyme B expression (right graph) by CD4 T cells. FIG. 15E depicts multiplex immunofluorescence staining of tumors isolated from CT26.Fluc tumor bearing mice. FIG. 15F depicts multiplex immunofluorescence staining of tumors isolated from MyC-CaP.Fluc tumor bearing mice. Representative images of T cell infiltration are shown for control as well as anti-OX40, SV.IL12 and SV.IL12+anti-OX40. Proteins of interest were stained and are indicated by color in each image: K-i67 (red), CD3 (green), CD8 (magenta) and DAPI nuclear staining appears in blue. Bars represent means±SEM in FIG. 15C, and each symbol represent an individual mouse in FIGS. 15A and 15D). Statistical significance was determined with the Kruskal-Wallis test followed by the Dunns' test. Results are representatives of at least two independent experiments

FIG. 16A-16D. T cells show enhanced migration into tumors and exert anti-tumor activity in mice treated with SV.IL12+anti-OX40. Tumor bearing mice were left untreated or were treated with SV.IL12 with or without anti-OX40. Mice were sacrificed on day 7 and 14 to analyze the T cell immune response in spleen. FIG. 16A-16C depict percentage of CXCR3 expression by CD4 (left graph) and CD8 (right graph) T cells measured by flow cytometry in the CT26.Fluc tumor model on day 14, in the MyC-CaP.Fluc tumor model on day 7 (B) and in the MyC-CaP.Fluc tumor model on day 14, respectively. FIG. 16D depicts percentage of CD8 expression by T cells (left graph), Ki-67 expression (middle graph) and granzyme B expression (right graph) by CD8 T cells. Tumors were harvested after 2 weeks of treatment from control and treated mice. T cell immune responses from indicated groups were assessed by flow cytometry. Bars represent means and each symbol represent an individual mouse. Statistical significance was determined with the Kruskal-Wallis test followed by the he Dunns' test. Results are representatives of at least two independent experiments.

FIG. 17. Combination therapy stimulates granzyme B expression in MyC-CaP.Fluc tumors. FIG. 17 depicts tumors stained by multiplex immunofluorescence. MyC-CaP.Fluc tumors were harvested after 2 weeks of treatment from control and treated mice. Representative images are shown for untreated (control), anti-OX40, SV.IL12 and SV.IL12+anti-OX40 treated mice. Proteins of interest were stained and are indicated by color in each image: F4/80 (red) and granzyme B (green). DAPI nuclear staining appears in blue.

FIG. 18A-18B. SV.IL12 triggers innate immune response and induces iNOS expression in MyC-CaP.Fluc tumors. MyC-CaP.Fluc tumors in FIG. 18A and CT26.Fluc tumors in FIG. 18B, were harvested after 2 weeks of treatment from control and treated mice. Tumors were stained by multiplex immunofluorescence. Representative images are shown for untreated (control), anti-OX40, SV.IL12 and SV.IL12+anti-OX40 treated mice. Proteins of interest were stained and are indicated by color in each image: iNOS (Cyan), Arginase 1 (green), and CD11b (magenta). DAPI nuclear staining appears in blue.

FIG. 19. Treatment schema of C57/B16 (female) mice re-injected with Alm5-2Fluc-17 tumor. Alm5-2Fluc-17 tumor reinjection was done from 9 C57/B16 mice in 80 mice (16 cages) on day 0. Treatment started on day 9 after cells implantation. Antibodies (250 ug/dose) treatment (blue dots) was done 3 times/week for 3 weeks starting at day 10 after cells re-injected. Sindbis Vector was administered 4 days/week for 4 weeks (red dots) starting day 9 after cells (mornings). IVIS imaging was done on indicated days after tumor implantation.

FIG. 20. Combination of IL-12 and anti-OX40 expressed by Sindbis viral vectors synergistically enhances survival of subjects with established tumors. FIG. 20. depicts Percentage survival rate of C57/B16 (female) mice re-injected with Alm5-2Fluc-17 tumor and treated with SV.IL12 vector; SV.IL-12 vector and anti-OX40 IgG; RepOX40IgG_Rep-IL12 (fragmented SV expressing OX-40 IgG and fragmented SV expressing IL-12, 50% mix of both vectors) or RepOX40IgG_SV-IL12 (fragmented SV expressing OX-40 IgG and full length SV expressing IL-12, 50% mix of both vectors), with n=5 mice in each treatment group. Untreated mice were used as a control. The mice were re-injected with tumor cells and treated according to the scheme in FIG. 19.

FIG. 21A-21C. A20 lymphoma cells were SV infection resistant. FIG. 21A depicts A20 cells and BHK cells were infected with SV carrying GFP overnight. GFP expression was observed under fluorescent microscope. FIG. 21B depicts SV-GFP infectivity to BHK cells was verified by flow cytometry. FIG. 21C depicts SV-GFP infectivity to A20 cells in vivo were measured by flow cytometry. 10⁷ A20 cells (express CD45.2) were inoculated to CByJ.SJL(B6)-Ptprca/J (CD45.1 BALB/C) mice. Recipient mice were treated with SV-GFP 4 days later. GFP expression was measured the next day.

FIG. 22A-22C. Sindbis virus (SV) and α4-1BB combination completely cured BALB/C mice A20 lymphoma. FIG. 21A depicts representative bioluminescence images of groups as indicated. Intensity scale, day 0, 7, 21, min: 400, max: 7000; day 14, min: 100, max: 1000; day 28, min: 3000, max: 50000. FIG. 2B depicts tumor growth measured by relative firefly luciferase (fLuc) activity (normalized to day 0 fLuc activity). Untreated, n=16; SV, n=18; α4-1BB Ab, n=13; SV plus α4-1BB Ab, n=13. FIG. 2C depicts survival curve of all groups (the ratio is shown as survived number/total number).

FIG. 23A-23E. SV alone and SV plus α4-1BB mAb stimulated cell cycle progression, cytokine production, and activation. FIG. 23A depicts the numbers of significant differential (SD) expressed genes (upregulated and downregulated) of SV vs. untreated are as indicated. SD expressed genes were selected based on Deseq2 analysis (q<0.05), |Log 2FC|≥1. FIG. 23B depicts the enrichment scores for gene cluster of cell cycle for SV vs. untreated, SV+α4-1BB vs. untreated and SV+α4-1BB vs. SV respectively (“cell cycle” is the gene cluster with the highest enrichment score for these 3 comparisons). FIG. 23C depicts the heat map representing SD expressed cytokine and chemokine genes (left, SV vs. untreated; right, SV+α4-1BB vs. α4-1BB, Log 2FC≥1). Expression values are shown by Z-score. Genes are hierarchically clustered by one minus Pearson correlation. Red arrow, Ccl8, IL4, IL13 and IL21 expression. FIG. 23D depicts the percentage of CD69+ T cells from all groups on day 2 after starting treatment was measured by flow cytometry. FIG. 23E depicts GSEA enrichment plot of KEGG (SV+α4-1BB vs. untreated) TCR receptor signaling pathway. *, p<0.05; **, p<0.01, *** p<0.001.

FIG. 24A-24C. SV infection enhanced cell cycle progression and migration. FIG. 24A depicts DAVID KEGG analysis. FIG. 24B depicts GSEA enrichment plot of KEGG (SV vs. Untreated) cell cycle pathway (SV vs. Untreated). FIG. 24C depicts cell movement pathway was significantly enhanced by IPA (SV vs. Untreated).

FIG. 25A-25B. Significant differential (SD) upregulated genes are clustered by DAVID analysis. FIG. 25A depicts enrichment score of gene clusters for SV+a 4-1BB vs Untreated. FIG. 25B depicts enrichment score of gene clusters for SV+a 4-1BB vs SV.

FIG. 26A-26D. Untreated group had low ratio of T cells and high ratio of regulatory T cells on day 28. FIGS. 26A-26C depict the frequency of CD4, CD8, and Treg respectively measured by flow cytometry. FIG. 26D depicts the Treg/CD8 ratio as indicated.

FIG. 27A-27D. Sindbis virus plus α4-1BB combination induced higher cytotoxicity. FIG. 27A depicts splenocytes mixed with fLuc-A20 lymphoma cells according to the ratio as indicated (splenocytes:lymphoma cells). Cytotoxicity corresponds to the reduction of normalized Luc activity (fLuc activity of A20 lymphoma cells only is normalized to 1). SV+ tumor, α4-1BB+ tumor, SV+α4-1BB+ tumor: tumor inoculated mice. SV, α4-1BB, SV+α4-1BB: mice without tumor inoculation. FIG. 27B depicts splenocytes harvested from all groups after 7 days treatment. The percentage of NKG2D+ cells was measured by flow cytometry (CD8 T cell gated). FIG. 27C depicts the percentage of granzyme B+ and perforin+ cells was measured by flow cytometry (CD8 T cell gated). FIG. 27D depicts cytotoxicity associated genes upregulated in SV+α4-1BB treated group. The heat map depicts the relative expression level of cytotoxicity associated genes. Expression values are shown by Z-score. Genes are hierarchically clustered by one minus Pearson correlation (day 7). Red square, granzyme b and perforin expression. Red arrow, Ifng and Stat4 expression. **, p<0.01; ****,p<0.0001.

FIG. 28A-28F. Sindbis virus plus α4-1BB combination induced Th1 differentiation and IFNγ production. FIG. 28A depicts IFNγ Elispot analysis of splenocytes harvested at day 2, 7, 14 and 28 from all groups as indicated. Upper panel, IFNγ Elispot image on day 7 after treatment. 1, 2, 3: three individual mice. Lower panel, IFNγ spots number from indicated groups over the course of treatment (2×10⁵ splenocytes per well). No stimulator was added. FIG. 28B depicts IFNγ production from CD4/CD8 T cell population in splenocytes and purified CD4/CD8 T cells. All groups were cultured in media for 5 h in the presence of brefeldin A. FIG. 28C depicts IFNγ production from purified CD4 T cells at different stimulation conditions. FIG. 28D depicts upregulated Th1 pathway gene set under SV, α4-1BB and SV+α4-1BB stimulation. Expression values are shown by Z-score. Genes are hierarchically clustered by one minus Pearson correlation (day 7). FIG. 28E depicts T-bet expression for all groups as indicated. FIG. 28F depicts EOMES expression for all groups as indicated. CD8 T cell gated. FIGS. 28E and 28F, day 7 after treatment. *, p<0.05; **, p<0.01, ****, p<0.0001.

FIG. 29 depicts IFNγ production from splenocytes of all groups with or without tumor inoculation on day 7 after treatment was measured by Elispot. With tumor: tumor was inoculated on day 0. Without tumor: tumor was not inoculated. No stimulator was added in Elispot assay.

FIG. 30A-30B. IFNγ production measurement. FIG. 30A, IFNγ production (at day 7) by all groups, as indicated, was measured by Elispot. FIG. 30B, IFNγ production of purified T cells (CD8 T cell portion) on day 7 after treatment was measured by flow cytometry.

FIG. 31A-31I. SV and α4-1BB mAb stimulated chemotaxis, adhesion and enhanced T cell infirtration and activation in tumor. FIG. 31A depicts heat map of the expression pattern of SV+α4-1BB upregulated chemokine and chemokine receptor genes (Expression values are shown by Z-score.) Genes are hierarchically clustered by one minus Pearson correlation (day 7). FIG. 31B depicts the percentage of CCR5+ cells was measured by flow cytometry (day 7). FIGS. 31C and 31D depict the percentage of CD11a+ and ICAM-1+ cells, respectively measured by flow cytometry. FIG. 31E depicts the relative expression of CD11a (ltgal) and ICAM-1 was shown by heat map measured by RNA-Seq. Expression values are shown by Z score. FIG. 31F depicts the percentage of OX40+ and ICOS+ T cells were measured by flow cytometry. *, p<0.05; **, p<0.01; ***, p<0.001; ****, p<0.0001. FIG. 31G depicts the frequency of CD3 and CD8 T cells to total harvested cells from tumor measured by flow cytometry. FIG. 31H depicts the CD8/Treg ratio of tumor infiltrated T cells. FIG. 31I depicts the percentage of granzyme B+CD8 T cells as indicated. *, p<0.05; **, p<0.01; ***, p<0.001; ****, p<0.0001.

FIG. 32A-32E. The phenotype of tumor infiltrated T cells. FIGS. 32A-32E depict the percentage of Ki67+, Foxp3+, T-bet+, EOMES+, NKG2D+ T cells respectively, measured by flow cytometry.

FIG. 33A-33C. Sindbis virus plus α4-1BB synergistically enhanced T cell glycolysis and oxidative phosphorylation. FIG. 33A depicts GSEA enrichment plot of KEGG (SV+α4-1BB vs. untreated) glycolysis pathway. FIG. 33B depicts the canonical pathways of SV plus α4-1BB Ab stimulation clustered by IPA. Red square, oxidative phosphorylation. FIG. 33C depicts both oxygen consumption rate (oxidative phosphorylation) and Extracellular Acidification Rate (glycolysis) measured by seahorse XFe24. All groups are as indicated (n=4).

FIG. 34A-34B. SV plus low dose α4-1BB mAb cured A20 tumor bearing mice. FIG. 34A depicts Bioluminescence images of mice showing tumor load in A20 tumor bearing mice treated with SV plus low dose α4-1BB mAb, as compared to control (untreated) and SV alone. FIG. 34B depicts tumor growth (Relative Luciferase activity) in each treatment group as indicated. Each line is a single mice.

FIG. 35A-35D. Cured mice are completely protected from A20 lymphoma rechallenge. FIG. 35A depicts Bioluminescence images of groups, previously treated as indicated, were re-challenged with A20 lymphoma cells. FIG. 35B depicts IFNγ production from purified T cells of all groups(To SV+α4-1BB, 4 months after treatment finished), in the absence or presence of A20 tumor cells (5×104 per well), was measured by Elispot assay. FIG. 35C depicts cytotoxicity assay was performed the same as FIG. 27A. Left 2 panels, total splenocytes were used. Right, purified T cells were used. Left upper, A20 Fluc cells and left lower, CT26 Fluc cells were used for co-culture. FIG. 35D depicts significant differential (SD) upregulated gene sets are clustered by DAVID KEGG analysis. *, p<0.05; **, p<0.01; ****, p<0.0001

FIG. 36. Combination of NY-ESO-1 and IL-12 expressed by separate Sindbis viral vectors synergistically enhances survival of subjects with established tumors. FIG. 36 depicts the percentage survival rate of C57/B16 (albino-female) mice re-injected with Alm5-2Fluc-17 tumor cells and treated with SV-IL-12, SV-NY-ESO-1 or a 50% mixture of both the vectors in one injection, SV-IL-12 and SV-NY-ESO-1, as indicated. Untreated mice were used as control. A total of n=5 mice in each group were tested for percentage survival days after tumor transplantation

FIG. 37. Combination of NY-ESO-1 and IL-12 expressed by the same Sindbis viral vectors synergistically enhances survival of subjects with established tumors. FIG. 37 depicts the percentage survival rate of C57/B16 (albino-female) mice re-injected with Alm5-2Fluc-17 tumor cells and treated with SV-IL-12, SV-NY-ESO-1 or a Sindbis viral vector expressing both IL-12 and NY-ESO-1 (SV-NYESO-SGP2-IL12), as indicated. Untreated mice were used as control. A total of n=5 mice in each group were tested for percentage survival days after tumor transplantation.

FIG. 38. pSP6-R_IL12 Sindbis replicon vector expressing IL12 a and b subunits. FIG. 38 depicts plasmid map with SP6, promoter for in vitro transcription; Replicase, SV RNA polymerase; Psg, subgenomic promoter for IL12 expression; linker, joins IL12 a and b subunits; AmpR, ampicillin resistance gene. Numbers show nucleotide positions of genes in the replicon plasmid.

FIG. 39. Sindbis Repicon vector expressing full length antibody to OX40 IgG2a. FIG. 39 depicts plasmid map with T7 promoter for in vitro transcription; Replicase, SV RNA polymerase; Psg subgenomic promoter for expression of anti-OX40 heavy chain IgG2a; 2Psg, second subgenomic promoter for expression of light chain anti-OX40. AmpR, ampicillin resistance gene; ColE1, plasmid origin of replication. Numbers show nucleotide positions of genes in the replicon plasmid.

FIG. 40. Sindbis replicon vector expressing single chain antibody to OX40. FIG. 40 depicts plasmid map with T7, promoter for in vitro transcription; Replicase, SV RNA polymerase; Psg, subgenomic SV promoter; IL12 signal peptide, signal peptide fused to the sequence encoding anti-OX40 single chain antibody; AmpR, ampicillin resistance gene; ColE1, plasmid origin of replication. Numbers show nucleotide positions of genes in the replicon plasmid.

FIG. 41. Sindbis Replicon Vector expressing NY-ESO-1. FIG. 41 depicts plasmid map with T7, promoter for in vitro transcription; Replicase, SV RNA polymerase; Psg, subgenomic promoter for transcription; Hu NY-ESO-1, coding sequence for human NY-ESO-1 tumor associated antigen, Poly A, poly A tail transcribed onto NY-ESO mRNA; AmpR, ampicillin resistant gene; ColE, plasmid origin of replication. Numbers show nucleotide positions of genes in the replicon plasmid.

FIG. 42. pT7StuIR1-FcOX40L_T2A_NY-ESO1. Sindbis replicon vector expressing the OX40 Ligand fused with the Fc receptor sequence and NY-ESO-1. FIG. 42 depicts plasmid map with T7, promoter for in vitro transcription; Replicase, SV RNA polymerase; Psg, subgenomic SV promoter; FcOX40L coding sequence; T2A, termination peptide sequence; NY-ESO-1, coding sequence; AmpR, ampicillin resistance; ColE1, plasmid origin of replication. Numbers show nucleotide positions of genes in the replicon plasmid.

### DETAILED DESCRIPTION OF THE INVENTION

Oncolytic virus (OV) therapy has become a novel immunotherapeutic approach to treat cancer. A rationale for oncolytic virus is that they can infect and lyse the tumor cell. They have been made to selectively replicate in tumor cells either through the direction of tumor specific promoters or through direct intratumoral administration. Most OVs encounter a number of barriers to systemic administration. Once lysed by OVs, tumor cells release tumor associated antigens (TAAs) that can stimulate cytotoxic T cells. OV infection also induces an inflammatory response that helps to trigger an immune anti-tumor response. Several OV clinical trials are underway and have shown promising results. However, whether OV therapy can effectively treat tumors that they are unable to infect remains an unresolved limitation.

Sindbis virus (SV) belongs to alphavirus genus and is one type of OV. Although it does not lyse infected tumor cells, it can cause their apoptotic death. It offers several important benefits. SV is known as one of the least virulent alphaviruses with clinical signs and symptoms usually unapparent. It has been estimated that there are 17 times more subclinical than symptomatic SV infections. In general, when symptoms do occur in humans they consist of a self-limiting, mild, febrile disease with vesicular exanthema and arthralgia from which most patients recover within 14 days. The disease is in part self-limiting because SV is an RNA virus that does not integrate in the host genome and hence its presence is transitory. The lack of an integrative step in its replication cycle also avoids insertional mutagenesis risks. In addition, SV vectors of the present disclosure were generated from the laboratory strain AR339, which is not known to cause disease in humans. These vectors were further attenuated by rendering them replication-defective.

SV vectors can target tumors systemically and can reach metastatic tumor cells throughout the body. They can target tumors without infecting normal tissues. However, susceptibility to infection by SV vectors depends on a number of factors including laminin receptor expression and distribution, as well as, defects in IFN signaling in tumors. The present disclosure demonstrates that SV vectors can effectively help cure tumors that they are unable to infect and further demonstrates that the combination antibodies and SV vectors provide a surprising synergistic therapeutic effect against cancer.

The present disclosure provides methods for treating cancer in a subject in need thereof comprising administering to the subject a therapeutically effective amount of (a) a oncolytic viral vector and (b) an antibody directed against a co-stimulatory molecule or a nucleic acid encoding same; or an antibody to an immune system agonist molecule or a nucleic acid encoding same.

The oncolytic viral vector can be a Sindbis viral vector. The Sindbis viral vector can be replication defective. Sindbis viral vectors were produced as described in U.S. Pat. No. 8,093,021 (incorporated herein by reference in its entirety). The Sindbis viral vector can comprise at least one nucleic acid encoding a therapeutic protein. The Sindbis viral vector can comprise at least one nucleic acid encoding an immunostimulatory or an immunomodulatory protein. The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12. The Sindbis viral vector can comprise at least one nucleic acid encoding LacZ, Flue or GFP.

The antibody can be an anti-OX40 antibody, an anti-4-1BB antibody, an anti-CD28 antibody, an anti-GITR antibody, an anti-CD137 antibody, an anti-cd37 antibody, an anti-HVEM antibody, or a combination thereof.

The Sindbis viral vector and the antibody can induce an immune response in a tumor associated antigen (TAA) nonspecific manner. The induced and nonspecific immune response can be a first immune response. The first immune response can be followed by a secondary immuneresponse. The secondary immune response can be the result of one or more TAAs released from the dead tumor cells. The secondary immune response can comprise memory T cells directed against one or more TAAs released from the dead tumor cells.

The present disclosure also provides methods for treating cancer in a subject in need thereof comprising administering to the subject a therapeutically effective amount of (a) a Sindbis viral vector comprising a nucleic acid encoding interleukin-12 and (b) an anti-OX40 monoclonal antibody or a nucleic acid encoding same, thereby treating cancer in the subject.

The Sindbis viral vector can be replication defective. The Sindbis viral vector can comprise the nucleic acid encoding interleukin-12 and can further comprise the nucleic acid encoding the anti-OX40 monoclonal antibody. The method can comprise administering a Sindbis viral vector comprising the nucleic acid encoding interleukin-12 and administering a Sindbis viral vector comprising the nucleic acid encoding the anti-OX40 monoclonal antibody.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit of GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The nucleic acid encoding interleukin-12 alpha subunit (IL-12 α, IL-12, p35 subunit) comprises the nucleic acid sequence of SEQ ID NO: 1 shown in the following Table.

The nucleic acid encoding interleukin-12 alphasubunit
(IL-12α, IL-12, p35 subunit)

| | |
|---|---|
| 1 | gcaagagaca cagtcctggg aaagtctgcc ggctatccag acaattataa aaatgtgtct |
| 61 | cccaaggtca gcgttccaac agcctcaccc tcggcatcca gcagctcctc tcagtgccgg |
| 121 | tccagcatgt gtcaatcacg ctacctcctc tttttggcca cccttgccct cctaaaccac |
| 181 | ctcagtttgg ccagggtcat tccagtctct ggacctgcca ggtgtcttag ccagtcccga |
| 241 | aacctgctga agaccacaga tgacatggtg aagacggcca gagaaaaact gaaacattat |
| 301 | tcctgcactg ctgaagacat cgatcatgaa gacatcacac gggaccaaac cagcacattg |
| 361 | aagacctgtt taccactgga actacacaag aacgagagtt gcctggctac tagagagact |
| 421 | tcttccacaa caagagggag ctgcctgccc ccacagaaga cgtctttgat gatgaccctg |
| 481 | tgccttggta gcatctatga ggacttgaag atgtaccaga cagagttcca ggccatcaac |
| 541 | gcagcacttc agaatcacaa ccatcagcag atcattctag acaagggcat gctggtggcc |
| 601 | atcgatgagc tgatgcagtc tctgaatcat aatggcgaga ctctgcgcca gaaacctcct |
| 661 | gtgggagaag cagaccctta cagagtgaaa atgaagctct gcatcctgct tcacgccttc |
| 721 | agcacccgcg tcgtgaccat caacagggtg atgggctatc tgagctccgc ctgaaagctc |
| 781 | aaggccctct gccacagcgc cctcctcaca cagataggaa acaaagaaag attcataaga |
| 841 | gtcaggtggt cttggcctgg tgggcttaag ctccttcagg aatctgttct cccatcacat |
| 901 | ctcatctccc caaaggtggc acagctacct cagcatggtg ccctccatcg cttctctcat |
| 961 | attcactata caagttgttt gtaagttttc atcaaaatat tgttaagggg cgaagacgtc |
| 1021 | ctcccctcaa tgtgttagca gaagagcaag aactgataag ctattgtttt tgtgccaaag |
| 1081 | tgtttatgaa aacactcagt caccccttat ttaaaaatat ttattgctat attttatact |
| 1141 | catgaaagta catgagccta tttatattta tttattttct atttattata atatttctta |
| 1201 | tcagatgaat ttgaaacatt ttgaaacata ccttattttg tggttctaat aaagtaatgt |
| 1261 | tatca (SEQ ID NO: 1) |

The nucleic acid encoding interleukin-12 beta subunit (IL-12 β, IL-12, p40 subunit) comprises the nucleic acid sequence of SEQ ID NO: 2 shown in the following Table.

The nucleic acid encoding interleukin-12 beta subunit
(IL-12β, IL-12, p40 subunit)

| | |
|---|---|
| 1 | gcacatcaga ccaggcagct cgcagcaaag caagatgtgt cctcagaagc taaccatctc |
| 61 | ctggtttgcc atcgttttgc tggtgtctcc actcatggcc atgtgggagc tggagaaaga |
| 121 | cgtttatgtt gtagaggtgg actggactcc cgatgcccct ggagaaacag tgaacctcac |
| 181 | ctgtgacacg cctgaagaag atgacatcac ctggacctca gaccagagac atggagtcat |
| 241 | aggctctgga aagaccctga ccatcactgt caaagagttt ctagatgctg gccagtacac |
| 301 | ctgccacaaa ggaggcgaga ctctgagcca ctcacatctg ctgctccaca agaaggaaaa |
| 361 | tggaatttgg tccactgaaa ttttaaaaaa tttcaaaaac aagactttcc tgaagtgtga |
| 421 | agcaccaaat tactccggac ggttcacgtg ctcatggctg gtgcaaagaa acatggactt |
| 481 | gaagttcaac atcaagagca gtagcagttc ccctgactct cgggcagtga catgtggaat |
| 541 | ggcgtctctg tctgcagaga aggtcacact ggaccaaagg gactatgaga agtattcagt |
| 601 | gtcctgccag gaggatgtca cctgcccaac tgccgaggag accctgccca ttgaactggc |
| 661 | gttggaagca cggcagcaga ataaatatga gaactacagc accagcttct tcatcaggga |
| 721 | catcatcaaa ccagacccgc ccaagaactt gcagatgaag cctttgaaga actcacaggt |
| 781 | ggaggtcagc tgggagtacc ctgactcctg gagcactccc cattcctact tctccctcaa |
| 841 | gttctttgtt cgaatccagc gcaagaaaga aaagatgaag gagacagagg aggggtgtaa |
| 901 | ccagaaaggt gcgttcctcg tagagaagac atctaccgaa gtccaatgca aaggcgggaa |
| 961 | tgtctgcgtg caagctcagg atcgctatta caattcctca tgcagcaagt gggcatgtgt |
| 1021 | tccctgcagg gtccgatcct aggatgcaac gttggaaagg aaagaaaagt ggaagacatt |
| 1081 | aaggaagaaa aatttaaact caggatggaa gagtccccca aaagctgtct tctgcttggt |
| 1141 | tggctttttc cagttttcct aagttcatca tgacaccttt gctgatttct acatgtaaat |
| 1201 | gttaaatgcc cgcagagcca gggagctaat gtatgcatag atattctagc attccacttg |
| 1261 | gccttatgct gttgaaatat ttaagtaatt tatgtattta ttaatttatt tctgcatttc |
| 1321 | acatttgtat accaagatgt attgaatatt tcatgtgctc gtggcctgat ccactgggac |
| 1381 | caggccctat tatgcaaatt gtgagcttgt tatcttcttc aacagctctt caatcagggc |
| 1441 | tgcgtaggta cattagcttt tgtgacaacc aataagaaca taatattctg acacaagcag |
| 1501 | tgttacatat ttgtgaccag taaagacata ggtggtattt ggagacatga agaagctgta |
| 1561 | aagttgactc tgaagagttt agcactagtt tcaacaccaa gaaagacttt ttagaagtga |
| 1621 | tattgataag aaaccagggc cttctttaga agggtaccta aatttaaaag aattttgaaa |
| 1681 | ggctgggtat cggtggtata tgcttttaat tccagcactc aggagaccaa ggcaggcaga |
| 1741 | tctctgtgag tttgaggaca gcctggtgta cagagggagt tccagcacag ccagtgccac |
| 1801 | acagaaattc tgtctcaaaa acaattaaaa aaaaaaaaaa (SEQ ID NO: 2) |

The Sindbis viral vector can comprise the nucleic acid encoding interleukin-12 alpha subunit of amino acid sequence of GenBank accession no. AAA39292.1 and interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise the nucleic acid encoding the interleukin-12 alpha subunit of amino acid sequence of GenBank accession no. AAA39292.1 and a nucleic acid encoding the interleukin-12 beta subunit of amino acid sequence of GenBank accession no. AAA39296.1. The amino acid sequence of the interleukin-12 alpha subunit comprises the amino acid sequence of SEQ ID NO: 3 shown in the following Table.

The amino acid sequence of the interleukin-12 alpha subunit

| | |
|---|---|
| 1 | MCQSRYLLFL ATLALLNHLS LARVIPVSGP ARCLSQSRNL LKTTDDMVKT AREKLKHYSC |
| 61 | TAEDIDHEDI TRDQTSTLKT CLPLELHKNE SCLATRETSS TTRGSCLPPQ KTSLMMTLCL |
| 121 | GSIYEDLKMY QTEFQAINAA LQNHNHQQII LDKGMLVAID ELMQSLNHNG ETLRQKPPVG |
| 181 | EADPYRVKMK LCILLHAFST RVVTINRVMG YLSSA (SEQ ID NO: 3) |

The amino acid sequence of the interleukin-12 beta subunit comprises the amino acid sequence of SEQ ID NO: 4 shown in the following Table.

The amino acid sequence of the interleukin-12 beta subunit

| | |
|---|---|
| 1 | MCPQKLTISW FAIVLLVSPL MAMWELEKDV YVVEVDWTPD APGETVNLTC DTPEEDDITW |
| 61 | TSDQRHGVIG SGKTLTITVK EFLDAGQYTC HKGGETLSHS HLLLHKKENG IWSTEILKNF |
| 121 | KNKTFLKCEA PNYSGRFTCS WLVQRNMDLK FNIKSSSSSP DSRAVTCGMA SLSAEKVTLD |
| 181 | QRDYEKYSVS CQEDVTCPTA EETLPIELAL EARQQNKYEN YSTSFFIRDI IKPDPPKNLQ |
| 241 | MKPLKNSQVE VSWEYPDSWS TPHSYFSLKF FVRIQRKKEK MKETEEGCNQ KGAFLVEKTS |
| 301 | TEVQCKGGNV CVQAQDRYYN SSCSKWACVP CRVRS (SEQ ID NO: 4) |

The Sindbis viral vector can comprise a nucleic acid encoding an interleukin-12 alpha subunit that is at least 70%, 75%, 80%, 85%, 90%, 95%, 96%, 97%, 98%, 99% or 100% identical to amino acid sequence of SEQ ID NO: 3 and a nucleic acid encoding an interleukin-12 alpha subunit that is at least 70%, 75%, 80%, 85%, 90%, 95%, 96%, 97%, 98%, 99% or 100% identical to amino acid sequence of SEQ ID NO: 4.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding an anti-OX40 variable heavy chain comprising the amino acid sequence of SEQ ID NO:5 shown in the following Table.

anti-OX40 variable heavy chain amino acid (mouse anti-OX40
variable heavy chain)

| | |
|---|---|
| 1 | MAEVQLVESG GGLVQPGGSL RLSCAASGFT FSNYTMNWVR QAPGKGLEWV |
| 51 | SAISGSGGST YYADSVKGRF TISRDNSKNT LYLQMNSLRA EDTAVYYCAK |
| 101 | DRYSQVHYAL DYWGQGTLVT V (SEQ ID NO: 5) |

The nucleic acid sequence encoding the anti-OX40 variable heavy chain comprises the nucleic acid sequence of SEQ ID NO:6 shown in the following Table.

anti-OX40 variable heavy chain nucleic acid
(mouse anti-OX40 variable heavy chain)

5′atggccgaggtgcagctggtggagagcggcggcggcctggtgcagcc
cggcggcagcctgaggctgagctgcgccgccagcggcttcaccttcagc
aactacaccatgaactgggtgaggcaggcccccggcaagggcctggagt
gggtgagcgccatcagcggcagcggcggcagcacctactacgccgacag
cgtgaagggcaggttcaccatcagcagggacaacagcaagaacaccctg
tacctgcagatgaacagcctgagggccgaggacaccgccgtgtactact
gcgccaaggacaggtacagccaggtgcactacgccctggactactgggg
ccagggcaccctggtgaccgtg 3′ (SEQ ID NO: 6)

The Sindbis viral vector can comprise a nucleic acid encoding an anti-OX40 variable light chain comprising the amino acid sequence of SEQ ID NO:7 shown in the following Table.

anti-OX40 variable light chain amino acid
(mouse anti-OX40 variable light chain)
(SEQ ID NO: 7)

| | |
|---|---|
| 1 | DIQMTQSPDS LPVTPGEPAS ISCRSSQSLL HSNGYNYLDW |
| | YLQKAGQSPQ |
| 51 | LLIYLGSNR |

The nucleic acid sequence encoding the anti-OX40 variable light chain comprises the nucleic acid sequence of SEQ ID NO:8 shown in the following Table.

anti-OX40 variable light chain nucleic acid
(mouse anti-OX40 variable light chain)
(SEQ ID NO: 8)
5′gacatccagatgacccagtcccccgactccctgcccgtgacccccggc
gagcccgcctccatctcctgccggtcctcccagtccctgctgcactccaa
cggctacaactacctggactggtacctgcagaaggccggccagtcccccc
agctgctgatctacctgggctccaaccgggcctccggcgtgcccgaccgg
ttctccggctccggctccggcaccgacttcaccctgaagatctcccgggt
ggaggccgaggacgtgggcgtgtactactgccagcagtactacaaccacc
ccaccaccttcggccagggcaccaagctggagatcaagcgg-3′

The Sindbis viral vector can comprise a nucleic acid encoding an anti-OX40 variable heavy chain of amino acid sequence of SEQ ID NO: 5 and a nucleic acid encoding an anti-OX40 variable light chain of amino acid sequence of SEQ ID NO: 7. The Sindbis viral vector can comprise a nucleic acid encoding an anti-OX40 variable heavy chain that is at least 70%, 75%, 80%, 85%, 90%, 95%, 96%, 97%, 98%, 99% or 100% identical to amino acid sequence of SEQ ID NO: 5 and a nucleic acid encoding an anti-OX40 variable light chain that is at least 70%, 75%, 80%, 85%, 90%, 95%, 96%, 97%, 98%, 99%˜ or 100% identical to amino acid sequence of SEQ ID NO: 7.

The Sindbis viral vector can comprise a nucleic acid encoding an anti-OX40 antibody heavy chain comprising the amino acid sequence of SEQ ID NO:9 shown in the following Table.

anti-OX40 antibody heavy chain amino acid sequence
(mouse anti-OX40 IgG2a antibody heavy chain)
(SEQ ID NO: 9)
MGQSRYLLFLATLALLNHLSLAMAEVQLVESGGGLVQPGGSLRLSCAASG
FTFSNYTMNWVRQAPGKGLEWVSAISGSGGSTYYADSVKGRFTISRDNSK
NTLYLQMNSLRAEDTAVYYCAKDRYSQVHYALDYWGQGTLVTVAAKTTAP
SVYPLAPVCGDTTGSSVTLGCLVKGYFPEPVTLTWNSGSLSSGVHTFPAV
LQSDLYTLSSSVTVTSSTWPSQSITCNVAHPASSTKVDKKI [symbol]
PAPNLLGGPSVFIFPPKIKDVLMISLSPIVTCVVV
DVSEDDPDVQISWFVNNVEVHTAQTQTHREDYNSTLRVVSALPIQHQDWM
SGKEEKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEK
KNWVERNSYSCSVVHEGLHNHHTTKSFSRTPGK

The nucleic acid sequence encoding the anti-OX40 antibody heavy chain comprises the nucleic acid sequence of SEQ ID NO: 10 shown in the following Table.

anti-OX40 antibody heavy chain nucleic acid sequence (mouse anti-OX40 IgG2a
antibody heavy chain)
(SEQ ID NO: 10)

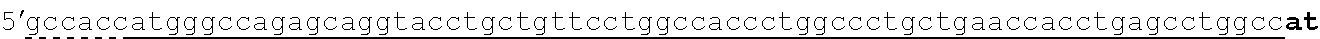

ggccgaggtgcagctggtggagagcggcggcggcctggtgcagcccggcggcagcctgaggctgagctgcgccgcc
agcggcttcaccttcagcaactacaccatgaactgggtgaggcaggcccccggcaagggcctggagtgggtgagcg
ccatcagcggcagcggcggcagcacctactacgccgacagcgtgaagggcaggttcaccatcagcagggacaacag
caagaacaccctgtacctgcagatgaacagcctgagggccgaggacaccgccgtgtactactgcgccaaggacagg
tacagccaggtgcactacgccctggactactggggccagggcaccctggtgaccgtggccgccaagaccaccgccc
ccagcgtgtaccccctggcccccgtgtgcggcgacaccaccggcagcagcgtgaccctgggctgcctggtgaaggg
ctacttccccgagcccgtgaccctgacctggaacagcggcagcctgagcagcggcgtgcacaccttccccgccgtg
ctgcagagcgacctgtacaccctgagcagcagcgtgaccgtgaccagcagcacctggcccagccagagcatcacct

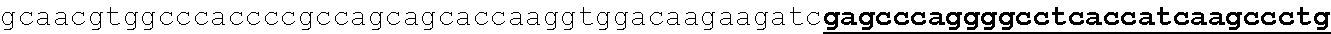

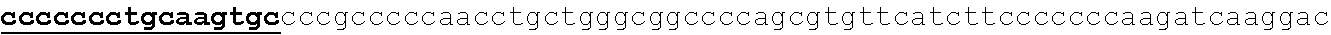

gtgctgatgatcagcctgagccccatcgtgacctgcgtggtggtggacgtgagcgaggacgaccccgacgtgcaga
tcagctggttcgtgaacaacgtggaggtgcacaccgcccagacccagacccacagggaggactacaacagcaccct
gagggtggtgagcgccctgcccatccagcaccaggactggatgagcggcaaggagttcaagtgcaaggtgaacaac

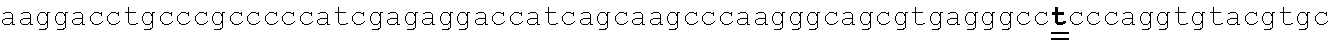

tgcccccccccgaggaggagatgaccaagaagcaggtgaccctgacctgcatggtgaccgacttcatgcccgagga
catctacgtggagtggaccaacaacggcaagaccgagctgaactacaagaacaccgagcccgtgctggacagcgac
ggcagctacttcatgtacagcaagctgagggtggagaagaagaactgggtggagaggaacagctacagctgcagcg
tggtgcacgagggcctgcacaaccaccacaccaccaagagcttcagcaggacccccggcaagtaa-3′

In SEQ ID NOs: 9 and 10, the underlined residues indicate IL-2 signal peptide; the Bold residues indicate variable antigen binding region; the non-underlined residues indicate mouse heavy chain IgG2a constant region, GB Accession BC080671; and the bold and underlined residues indicate the Hinge and disulfide bond region. The double underlined residue in SEQ ID NO: 9 indicates change from C to T to remove ApaI site. The dotted underlined residues in SEQ ID NO: 9 indicate Kozak sequence.

The Sindbis viral vector can comprise a nucleic acid encoding an anti-OX40 antibody light chain comprising the amino acid sequence of SEQ ID NO:11 shown in the following Table.

anti-OX40 antibody light chain amino acid sequence
(mouse anti-OX40 IgG2a antibody light chain)
(SEQ ID NO: 11)
MGQSRYLLFLATLALLNHLSLADIQMTQSPDSLPVTPGEPASISCRSSQS
LLHSNGYNYLDWYLQKAGQSPQLLIYLGSNRASGVPDRFSGSGSGTDFTL
KISRVEAEDVGVYYCQQYYNHPTTFGQGTKLEIKRADAAPTVSIFPPSSE
QLTSGGASVVCFLNNFYPKDINVKWKIDGSERQNGVLNSWTDQDSKDSTY
SMSSTLTLTKDEYERHNSYTCEATHKTSTSPIVKSFNRNEC

The nucleic acid sequence encoding the mouse anti-OX40 antibody light chain comprises the nucleic acid sequence of SEQ ID NO 12 shown in the following Table

anti-OX40 antibody light chain nucleic acid sequence (mouse anti-OX40 IgG2a antibody light
chain)
(SEQ ID NO: 12)

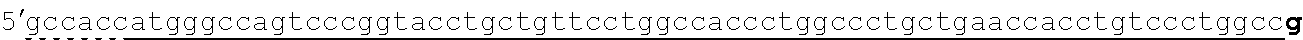

acatccagatgacccagtcccccgactccctgcccgtgacccccggcgagcccgcctccatctcctgccggtcct
cccagtccctgctgcactccaacggctacaactacctggactggtacctgcagaaggccggccagtccccccagc
tgctgatctacctgggctccaaccgggcctccggcgtgcccgaccggttctccggctccggctccggcaccgact
tcaccctgaagatctcccgggtggaggccgaggacgtgggcgtgtactactgccagcagtactacaaccacccca
ccaccttcggccagggcaccaagctggagatcaagcgggccgacgccgcccccaccgtgtccatcttccccccct
cctccgagcagctgacctccggcggcgcctccgtggtgtgcttcctgaacaacttctaccccaaggacatcaacg
tgaagtggaagatcgacggctccgagcggcagaacggcgtgctgaactcctggaccgaccaggactccaaggact
ccacctactccatgtcctccaccctgaccctgaccaaggacgagtacgagcggcacaactcctacacctgcgagg
ccacccacaagacctccacctcccccatcgtgaagtccttcaaccggaacgagtgctaa-3′

In SEQ ID NOs: 11 and 12, the underlined residues indicate IL-2 signal peptide; the Bold residues indicate variable antigen binding region; the non-underlined residues indicate light constant region, GB Accession BC091750.1. The dotted underlined residues in SEQ ID NO: 12 indicate Kozak sequence.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise a nucleic acid encoding an anti-OX40 antibody heavy chain with an amino acid sequence that binds to a target antigen of the amino acid sequence of SEQ ID NO:13 shown in the following Table.

amino acid sequence of the Target antigen
(Target Antigen: OX40/CD134, Receptor for
TNFSF4/OX40L/GP34)
(SEQ ID NO: 13)

| | |
|---|---|
| 1 | MCVGARRLGR GPCAALLLLG LGLSTVTGLH CVGDTYPSND |
| | RCCHECRPGN |
| 51 | GMVSRCSRSQ NTVCRPCGPG FYNDVVSSKP CKPCTWCNLR |
| | SGSERKQLCT |
| 101 | ATQDTVCRCR AGTQPLDSYK PGVDCAPCPP GHFSPGDNQA |
| | CKPWTNCTLA |
| 151 | GKHTLQPASN SSDAICEDRD PPATQPQETQ GPPARPITVQ |
| | PTEAWPRTSQ |
| 201 | GPSTRPVEVP GGRAVAAILG LGLVLGLLGP LAILLALYLL |
| | RRDQRLPPDA |
| 251 | HKPPGGGSFR TPIQEEQADA HSTLAKI |

The Sindbis viral vector can comprise a nucleic acid encoding a human anti-OX40 antibody variable heavy chain comprising the amino acid sequence of SEQ ID NO: 31 shown in the following Table.

Human anti-OX40 antibody, 1-12Z5
(ATCC No. PTA-7216) Heavy chain variable region
amino acid sequence. Leader sequence in bold.
(SEQ ID NO: 31)

| | |
|---|---|
| 1 | MTMITPSLVP SSDPLVTAAS VLEFALLIRL TIGQAVVSTQ |
| | STGGGLVQPG RSLRLSCAAS |
| 61 | GFTLDDYGMH WVRQAPGKGL EWVSGISWNS DSIGYVDSVK |
| | GRFTISRDNA KNSLYLQMNS |
| 121 | LRVEDTALYY CVKDISGWYS FDYWGQGTLV TVSS |

The nucleic acid sequence encoding the human anti-OX40 antibody heavy chain variable region comprises the nucleic acid sequence of SEQ ID NO: 32 shown in the following Table.

Human anti-OX40 antibody 1-12Z5
(ATCC No. PTA-7216) Heavy chain variable region
nucleic acid sequence
(SEQ ID NO: 32)
5′-atgaccatgattacgccaagcttggtaccgagctcggatccactagt
aacggccgccagtgtgctggaattcgcccttctaatacgactcactatag
ggcaagcagtggtatcaacgcagagtacggggggaggcttggtacagcct
ggcaggtccctgagactctcctgtgcagcctctggattcacccttgatga
ttatggcatgcactgggtccggcaagctccagggaagggcctggagtggg
tctcaggtattagttggaatagtgatagtataggctatgtggactctgtg
aagggccgattcaccatctccagagacaacgccaagaactccctgtatct
gcaaatgaacagtctgagagttgaggacacggccttgtattactgtgtaa
aagatattagtggctggtacagctttgactactggggccagggaaccctg
gtcaccgtctcctca-3′

The Sindbis viral vector can comprise a nucleic acid encoding a human anti-OX40 antibody variable light chain comprising the amino acid sequence of SEQ ID NO: 33 shown in the following Table.

Human anti-OX40 antibody 1-12Z5
(ATCC No. PTA-7216) Light chain variable region
amino acid sequence
(SEQ ID NO: 33)

| | |
|---|---|
| 1 | MEAPAQLLFL LLLWLPDTTG EIVLTQSPAT LSLSPGERAT |
| | LSCRASQSVS SYLAWYQQKP |
| 61 | GQAPRLLIYD ASNRATGIPA RFSGSGSGTD FTLTISSLEP |
| | EDFAVYYCQQ RSNWPITFGQ |
| 121 | GTRLEIK |

The nucleic acid sequence encoding the human anti-OX40 antibody light chain variable region comprises the nucleic acid sequence of SEQ ID NO: 34 shown in the following Table.

Human anti-OX40 antibody 1-12Z5
(ATCC No. PTA-7216) Light chain variable region
nucleic acid sequence
(SEQ ID NO: 34)
5′atggaagccccagctcagcttctcttcctcctgctactctggctccca
gataccaccggagaaattgtgttgacacagtctccagccaccctgtcttt
gtctccaggggaaagagccaccctctcctgcagggccagtcagagtgtta
gcagctacttagcctggtaccaacagaaacctggccaggctcccaggctc
ctcatctatgatgcatccaacagggccactggcatcccagccaggttcag
tggcagtgggtctgggacagacttcactctcaccatcagcagcctagagc
ctgaagattttgcagtttattactgtcagcagcgtagcaactggccgatc
accttcggccaagggacacgactggagattaaa-3′

The Sindbis viral vector can comprise a nucleic acid encoding a human anti-OX40 antibody variable heavy chain comprising the amino acid sequence of SEQ ID NO: 35 shown in the following Table.

Human anti-OX40 antibody 112F32
(ATCC No. PTA-7217) Heavy chain variable region
amino acid sequence
(SEQ ID NO: 35)

| | |
|---|---|
| 1 | MEWGPCWVFL VVILEGVQCG VQLVESGGGL VQPGGSLRLS |
| | CAASGFTFSS YSMNWVRQAP |
| 61 | GKGLEWVSYI SSSSSTIYYA DSVKGRFTIS RDNAKNSLYL |
| | QMNSLRDEDT AVYYCARGVY |
| 121 | HNGWSFFDYW GQGTLLTVSS |

The nucleic acid sequence encoding the human anti-OX40 antibody heavy chain variable region comprises the nucleic acid sequence of SEQ ID NO: 36 shown in the following Table.

Human anti-OX40 antibody 112F32
(ATCCNo. PTA-7217) Heavy chain variable region
nucleic acid sequence
(SEQ ID NO: 36)
5′atggagtgggggccgtgctgggttttccttgttgttattttagaaggt
gtccagtgtggggtgcagctggtggagtctgggggaggcttggtacagcc
tggggggtccctgagactctcctgtgcagcctctggattcaccttcagta
gctatagcatgaactgggtccgccaggctccagggaaggggctggagtgg
gtttcatacattagtagtagtagtagtaccatatactatgcagactctgt
gaagggccgattcaccatctccagagacaatgccaagaactcactgtatc
tgcaaatgaacagcctgagagacgaggacacggctgtgtattactgtgcg
agaggagtgtatcacaatggctggtccttctttgactactggggccaggg
aaccctactcaccgtctcctca-3′

The Sindbis viral vector can comprise a nucleic acid encoding a human anti-OX40 antibody variable light chain comprising the amino acid sequence of SEQ ID NO: 37 shown in the following Table.

Human anti-OX40 antibody 112F32
(ATCC No. PTA-7217) Light chain variable region
amino acid sequence.
(SEQ ID NO: 37)

| | |
|---|---|
| 1 | MDMRVLAQLL GLLLLCFPGA RCDIQMTQSP SSLSASVGNR |
| | VTITCRASQD ISSWLAWYQQ |
| 61 | KPEKAPKSLI YAASSLQSGV PSRFSGSGSG TDFTLTISSL |
| | QPEDFATYYC QQYNSYPLTF |
| 121 | GQGTRLEIKR |

The nucleic acid sequence encoding the human anti-OX40 antibody light chain variable region comprises the nucleic acid sequence of SEQ ID NO: 38 shown in the following Table.

Human anti-OX40 antibody 12F32 LV
(ATCC No. PTA-7217) Light chain variable region
nucleic acid sequence
(SEQ ID NO: 38)
5′-atggacatgagggtcctcgctcagctcctggggctcctgctgctctg
tttcccaggtgccagatgtgacatccagatgacccagtccccatcctcac
tgtctgcatctgtaggaaacagagtcaccattacttgtcgggcgagtcag
gatattagcagctggttagcctggtatcagcagaaaccagagaaagcccc
taagtccctgatctatgctgcatccagtttgcaaagtggggtcccatcaa
ggttcagcggcagtggatctgggacagatttcactctcaccatcagcagc
ctgcagcctgaagattttgcaacttattactgccaacagtataatagtta
ccccctcaccttcggccaagggacacgactggagattaaacga-3′

The Sindbis viral vector can comprise a nucleic acid encoding a human anti-OX40 antibody variable heavy chain comprising the amino acid sequence of SEQ ID NO: 39 shown in the following Table.

Human anti-OX40 antibody 112Y1 31
(ATCC NO. PTA-7218) Heavy chain variable region
amino acid sequence
(SEQ ID NO: 39)

| | |
|---|---|
| 1 | MDTLCSTLLL LTIPSWVLSQ ITLKESGPTL VKPTQTLTLT |
| | CTFSGFSLST SGVGVGWIRQ |
| 61 | PPGKALEWLA LIYWDDHSPY SPSLKSRLTI TKDTSKNQVV |
| | LTMTNMDPVD TATYYCARTR |
| 121 | GAFDIWGQGT MVTVSS |

The nucleic acid sequence encoding the human anti-OX40 antibody heavy chain variable region comprises the nucleic acid sequence of SEQ ID NO: 40 shown in the following Table.

Human anti-OX40 antibody 112Y1 31
(ATCC NO. PTA-7218) Heavy chain variable region
nucleic acid sequence
(SEQ ID NO: 40)
5′-atggacactctttgctccacgctcctgctgctgaccatcccttcatg
ggtcttgtcccagatcaccttgaaggagtctggtcctacgctggtgaaac
ccacacagaccctcacgctgacctgcaccttctctggattctcactcagc
actagtggagtgggtgtgggctggatccgtcagcccccaggaaaggccct
ggagtggcttgcactcatttattgggatgatcatagcccctacagcccat
ctctgaagagcaggctcaccatcaccaaggacacctccaaaaaccaggtg
gtccttacaatgaccaacatggaccctgtggacacagccacatattactg
tgcacgcacccggggggcttttgatatctggggccaagggacaatggtca
ccgtctcttca-3′

The Sindbis viral vector can comprise a nucleic acid encoding a human anti-OX40 antibody variable light chain comprising the amino acid sequence of SEQ ID NO: 41 shown in the following Table.

| Human anti-OX40 antibody 112Y1 31 (ATCC NO. PTA-7218) Light chain | |
|---|---|
| variable region amino acid sequence | |
| (SEQ ID NO: 41) | |
| 1 | MEAPAQLLFL LLLWLPDTTG EIVLTQSPAT LSLSPGERAT LSCRASQGVS SYLAWYQQKP |
| 61 | GQAPRLLIYD ASNRATGIPA RFSGSGPGTD FTLTISSLEP EDFAVYYCQQ RSNWHPTFGQ |
| 121 | GTKVEIK |

The nucleic acid sequence encoding the human anti-OX40 antibody light chain variable region comprises the nucleic acid sequence of SEQ ID NO: 42 shown in the following Table.

Human anti-OX40 antibody 112Y1 31 (ATCC NO.
PTA-7218) Light chain variable region nucleic
acid sequence
(SEQ ID NO: 42)
5′-atggaagccccagcgcagcttctcttcctcctgctactctggctccc
agataccaccggagaaattgtgttgacacagtctccagccaccctgtctt
tgtctccaggggaaagagccaccctctcctgcagggccagtcagggtgtt
agcagctacttagcctggtaccagcagaaacctggccaggctcccaggct
cctcatctatgatgcatccaacagggccactggcatcccagccaggttca
gtggcagtgggcctgggacagacttcactctcaccatcagcagcctagag
cctgaagattttgcagtttattactgtcagcagcgtagcaactggcatcc
gacgttcggccaagggaccaaggtggaaatcaaacgaactgtggctgcac
catc-3′

The Sindbis viral vector can comprise a nucleic acid encoding a human anti-OX40 antibody variable heavy chain comprising the amino acid sequence of SEQ ID NO: 43 shown in the following Table.

| Human anti-OX40 antibody 112V8 (ATCC NO. PTA-7219) Heavy chain | |
|---|---|
| variable region amino acid sequence | |
| (SEQ ID NO: 43) | |
| 1 | MDTLCSTLLL LTIPSWVLSQ ITLKESGPTL VKPKQTLTLT CTFSGFSLST SGMGVGWIRQ |
| 61 | PPGKALEWLA VIYWDDHQLY SPSLKSRLTI TKDTSKNQVV LTMTNMDPVD TATYYCAHRR |
| 121 | GAFQHWGQGT LVTVSSASTK |

The nucleic acid sequence encoding the human anti-OX40 antibody heavy chain variable region comprises the nucleic acid sequence of SEQ ID NO: 44 shown in the following Table.

Human anti-OX40 antibody 112V8 (ATCC NO. PTA-7219)
Heavy chain variable region nucleic acid sequence
(SEQ ID NO: 44)
5′-atggacacactttgctccacgctcctgctgctgaccatcccttcatg
ggtcttgtcccagatcaccttgaaggagtctggtcctacgctagtgaagc
ccaaacagaccctcacgctgacctgcaccttctctggattctcactcagc
actagtggaatgggtgtgggctggatccgtcagcccccaggaaaggccct
ggagtggcttgcagtcatttattgggatgatcatcaactctacagtccat
ctctgalgagcaggctcaccatcaccaaggacacctccaaaaaccaggtg
gtccttacaatgaccaacatggaccctgtggacacagccacatattactg
tgcacacagacgaggggccttccagcactggggccagggcaccctggtca
ccgtctcctcagcttccaccaagggc-3′

The Sindbis viral vector can comprise a nucleic acid encoding a human anti-OX40 antibody variable light chain comprising the amino acid sequence of SEQ ID NO: 45 shown in the following Table.

| Human anti-OX40 antibody 112V8 (ATCC NO. PTA-7219) Light chain | |
|---|---|
| variable region amino acid sequence | |
| (SEQ ID NO: 45) | |
| 1 | METPAQLLFL LLLWLPDTTG EIVLTQSPGT LSLSPGERAT LSCRASQSVS SSYLAWYQQK |
| 61 | PGQAPRLLIY GASSRATGIP DRFSGSGSGT DFTLTISRLE PEDFAVYYCQ QYDSSLTFGG |
| 121 | GTKVEIKRT |

The nucleic acid sequence encoding the human anti-OX40 antibody light chain variable region comprises the nucleic acid sequence of SEQ ID NO: 46 shown in the following Table.

Human anti-OX40 antibody 112V8 (ATCC NO. PTA-7219)
Light chain variable region nucleic acid sequence
(SEQ ID NO: 46)
5′-atggagtgggggccgtgctgggttttccttgttgttattttagaagg
tgtccagtgtgggatggaaaccccagcgcagcttctcttcctcctgctac
tctggctcccagataccaccggagaaattgtgttgacgcagtctccaggc
accctgtctttgtctccaggggaaagagccaccctctcctgcagggccag
tcagagtgttagcagcagctacttagcctggtaccagcagaaacctggcc
aggctcccaggctcctcatctatggtgcatccagcagggccactggcatc
ccagacaggttcagtggcagtgggtctgggacagacttcactctcaccat
cagcagactggagcctgaagattttgcagtgtattactgtcagcagtatg
atagctcgctcactttcggcggagggaccaaggtggagatcaaacgaac
t-3′

The Sindbis viral vector can comprise a nucleic acid encoding a human anti-OX40 antibody variable heavy chain comprising the amino acid sequence of SEQ ID NO: 47 shown in the following Table.

| Human anti-OX40 antibody 112Y55 (ATCC No. PTA-7220) Heavy chain | |
|---|---|
| variable region amino acid sequence | |
| (SEQ ID NO: 47) | |
| 1 | MDTLCSTLLL LTIPSWVLSQ ITLKESGPTL VKPTQTLTLS CTFSGFSLST SGVGVGWIRQ |
| 61 | PPGKALEWLA LIHWDDAERY SPSLKSRLTI TKDTSKNQVV LTMTNMDLVD TATYYCAHTR |
| 121 | GAFDIWGQGT MVTVSS |

The nucleic acid sequence encoding the human anti-OX40 antibody heavy chain variable region comprises the nucleic acid sequence of SEQ ID NO: 48 shown in the following Table.

Human anti-OX40 antibody 112Y55 (ATCC No.
PTA-7220) Heavy chain variable region nucleic
acid sequence
(SEQ ID NO: 48)
5′-atggacacactttgctccacgctcctgctgctgaccatcccttcatg
ggtcttgtcccagatcaccttgaaggagtctggtcctacgctggtgaaac
ccacacagaccctcacgctgtcctgcaccttctctgggttctcactcagc
actagtggagtgggtgtgggctggatccgtcagcccccaggaaaggccct
ggaatggcttgcactcattcattgggatgatgctgagcgctacagtccat
ctctgaagagcaggctcaccatcaccaaggacacctccaaaaaccaggtg
gtccttacaatgaccaacatggaccttgtggacacagccacatattactg
tgcacacacccggggggcttttgatatctggggccaagggacaatggtca
ccgtctcttca-3′

The Sindbis viral vector can comprise a nucleic acid encoding a human anti-OX40 antibody variable light chain comprising the amino acid sequence of SEQ ID NO: 49 shown in the following Table.

| Human anti-OX40 antibody 112Y55 (ATCC No. PTA-7220) Light chain | |
|---|---|
| variable region amino acid sequence | |
| (SEQ ID NO: 49) | |
| 1 | METPAQLLFL LLLWLPDTTG EIVLTQSPGT LSLSPGERAI LSCRASQSVS SSFLAWYQQK |
| 61 | PGQAPRLLIY GAFSRATGIP DRFSGSGSGT DFTLTISRLE PEDFAVYYCQ QYDSSRTFGQ |
| 121 | GTKVEIK |

The nucleic acid sequence encoding the human anti-OX40 antibody light chain variable region comprises the nucleic acid sequence of SEQ ID NO: 50 shown in the following Table.

Human anti-OX40 antibody 112Y55 (ATCC No.
PTA-7220) Light chain variable region nucleic
acid sequence
(SEQ ID NO: 50)
5′-atggaaaccccagcgcagcttctcttcctcctgctactctggctccc
agataccaccggagaaattgtgttgacgcagtctccaggcaccctgtctt
tgtctccaggggaaagagccatcctctcctgcagggccagtcagagtgtt
agcagcagcttcttagcctggtaccaacagaaacctggccaggctcccag
gctcctcatctatggtgcatttagcagggccactggcatcccagacaggt
tcagtggcagtgggtctgggacagacttcactctcaccatcagcagactg
gagcctgaagattttgcagtgtattactgtcagcagtatgatagctcacg
gacgttcggccaggggaccaaggtggaaatcaaa-3′

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 IgG2a antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 IgG2a antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and a mouse anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise a nucleic acid encoding a human anti-OX40 variable heavy chain comprising the amino acid sequence of SEQ ID NO: 31. The nucleic acid sequence encoding the human anti-OX40 variable heavy chain is SEQ ID NO: 32. The Sindbis viral vector can comprise a nucleic acid encoding a human anti-OX40 variable light chain comprising the amino acid sequence of SEQ ID NO: 33. The nucleic acid sequence encoding the human anti-OX40 variable light chain is SEQ ID NO: 34.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 31. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 32. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 33. The nucleic acid sequence encoding the human anti-OX40 variable light chain that is at least 70%, 75%, 80%, 85%, 90%, 95%, 96%, 97%, 98%, 99% or 100% identical to SEQ ID NO: 34.

The Sindbis viral vector can comprise a nucleic acid encoding a human anti-OX40 variable heavy chain comprising the amino acid sequence of SEQ ID NO: 35. The nucleic acid sequence encoding the human anti-OX40 variable heavy chain is SEQ ID NO: 36. The Sindbis viral vector can comprise a nucleic acid encoding a human anti-OX40 variable light chain comprising the amino acid sequence of SEQ ID NO: 37. The nucleic acid sequence encoding the human anti-OX40 variable light chain is SEQ ID NO: 38.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 35. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 36. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 37. The nucleic acid sequence encoding the human anti-OX40 variable light chain that is at least 70%, 75%, 80%, 85%, 90%, 95%, 96%, 97%, 98%, 99% or 100% identical to SEQ ID NO: 38.

The Sindbis viral vector can comprise a nucleic acid encoding a human anti-OX40 variable heavy chain comprising the amino acid sequence of SEQ ID NO: 39. The nucleic acid sequence encoding the human anti-OX40 variable heavy chain is SEQ ID NO: 40. The Sindbis viral vector can comprise a nucleic acid encoding a human anti-OX40 variable light chain comprising the amino acid sequence of SEQ ID NO: 41. The nucleic acid sequence encoding the human anti-OX40 variable light chain is SEQ ID NO: 42.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 39. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 40. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 41. The nucleic acid sequence encoding the human anti-OX40 variable light chain that is at least 70%, 75%, 80%, 85%, 90%, 95%, 96%, 97%, 98%, 99% or 100% identical to SEQ ID NO: 42.

The Sindbis viral vector can comprise a nucleic acid encoding a human anti-OX40 variable heavy chain comprising the amino acid sequence of SEQ ID NO: 43. The nucleic acid sequence encoding the human anti-OX40 variable heavy chain is SEQ ID NO: 44. The Sindbis viral vector can comprise a nucleic acid encoding a human anti-OX40 variable light chain comprising the amino acid sequence of SEQ ID NO: 45. The nucleic acid sequence encoding the human anti-OX40 variable light chain is SEQ ID NO: 46.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 43. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 44. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 45. The nucleic acid sequence encoding the human anti-OX40 variable light chain that is at least 70%, 75%, 80%, 85%, 90%, 95%, 96%, 97%, 98%, 99% or 100% identical to SEQ ID NO: 46.

The Sindbis viral vector can comprise a nucleic acid encoding a human anti-OX40 variable heavy chain comprising the amino acid sequence of SEQ ID NO: 47. The nucleic acid sequence encoding the human anti-OX40 variable heavy chain is SEQ ID NO: 48. The Sindbis viral vector can comprise a nucleic acid encoding a human anti-OX40 variable light chain comprising the amino acid sequence of SEQ ID NO: 49. The nucleic acid sequence encoding the human anti-OX40 variable light chain is SEQ ID NO: 50.

The Sindbis viral vector can comprise a nucleic acid encoding a human anti-OX40 variable heavy chain comprising the amino acid sequence that is at least 70%, 75%, 80%, 85%, 90%, 95%, 96%, 97%, 98%, 99% or 100% identical to SEQ ID NO: 47. The nucleic acid sequence encoding the human anti-OX40 variable heavy chain that is at least 70%, 75%, 80%, 85%, 90%, 95%, 96%, 97%, 98%, 99% or 100% identical to is SEQ ID NO: 48. The Sindbis viral vector can comprise a nucleic acid encoding a human anti-OX40 variable light chain comprising the amino acid sequence that is at least 70%, 75%, 80%, 85%, 90%, 95%, 96%, 97%, 98%, 99% or 100% identical to SEQ ID NO: 49. The nucleic acid sequence encoding the human anti-OX40 variable light chain that is at least 70%, 75%, 80%, 85%, 90%, 95%, 96%, 97%, 98%, 99% or 100% identical to SEQ ID NO: 50.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector and the anti-OX40 monoclonal antibody can be administered sequentially or concurrently. The Sindbis viral vector can be administered systemically. The anti-OX40 monoclonal antibody can be administered systemically. Both the Sindbis viral vector and the anti-OX40 monoclonal antibody, or a nucleic acid encoding same, can be administered systemically. The Sindbis viral vector can be administered parenterally. The anti-OX40 monoclonal antibody can be administered parenterally. Both the Sindbis viral vector and the anti-OX40 monoclonal antibody, or a nucleic acid encoding same, can be administered parenterally. The Sindbis viral vector can be administered intraperitoneally. The anti-OX40 monoclonal antibody can be administered intraperitoneally. Both the Sindbis viral vector and the anti-OX40 monoclonal antibody, or a nucleic acid encoding same, can be administered intraperitoneally.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

Sindbis Viral Vector and Anti-OX40 Monoclonal Antibody

The present disclosure further provides a Sindbis viral vector comprising a nucleic acid encoding interleukin-12 and a nucleic acid encoding an anti-OX40 monoclonal antibody. The present disclosure also provides a composition comprising (a) a Sindbis viral vector comprising a nucleic acid encoding interleukin-12 and (b) an anti-OX40 monoclonal antibody or a nucleic acid encoding same. The present disclosure also provides a composition comprising (a) a Sindbis viral vector comprising a nucleic acid encoding interleukin-12 and (b) a Sindbis viral vector comprising a nucleic acid encoding an anti-OX40 monoclonal antibody.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit of GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise the nucleic acid encoding interleukin-12 alpha subunit of amino acid sequence of GenBank accession no. AAA39292.1 and interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The present disclosure also provides methods for treating cancer in a subject in need thereof comprising administering to the subject a therapeutically effective amount of (a) a Sindbis viral vector comprising a nucleic acid encoding interleukin-12 and (b) an anti-OX40 monoclonal antibody or a nucleic acid encoding same, thereby treating cancer in the subject.

The Sindbis viral vector can be replication defective. The Sindbis viral vector can comprise the nucleic acid encoding interleukin-12 and can further comprise the nucleic acid encoding the anti-OX40 monoclonal antibody. The method can comprise administering a Sindbis viral vector comprising the nucleic acid encoding interleukin-12 and administering a Sindbis viral vector comprising the nucleic acid encoding the anti-OX40 monoclonal antibody.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit of GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise the nucleic acid encoding interleukin-12 alpha subunit of amino acid sequence of GenBank accession no. AAA39292.1 and interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The Sindbis viral vector and the anti-OX40 monoclonal antibody can be administered sequentially or concurrently. The Sindbis viral vector can be administered systemically. The anti-OX40 monoclonal antibody can be administered systemically. Both the Sindbis viral vector and the anti-OX40 monoclonal antibody, or a nucleic acid encoding same, can be administered systemically. The Sindbis viral vector can be administered parenterally. The anti-OX40 monoclonal antibody can be administered parenterally. Both the Sindbis viral vector and the anti-OX40 monoclonal antibody, or a nucleic acid encoding same, can be administered parenterally. The Sindbis viral vector can be administered intraperitoneally. The anti-OX40 monoclonal antibody can be administered intraperitoneally. Both the Sindbis viral vector and the anti-OX40 monoclonal antibody, or a nucleic acid encoding same, can be administered intraperitoneally.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

Sindbis virus can be administered at least one time, at least two times, at least three times, at least four times or at least five times per week. Sindbis virus can be administered for at least one week, at least two weeks, at least three weeks, or at least four weeks. Sindbis virus can be administered from 10⁶-10⁹ TU/mL. Preferably, Sindbis virus can be administered from 10⁶-10⁹ TU/mL.

An anti-OX40 monoclonal antibody can be administered at least one time, at least two times, at least three times, at least four times or at least five times per week. An anti-OX40 monoclonal antibody can be administered for at least one week, at least two weeks, at least three weeks, or at least four weeks. An anti-OX40 monoclonal antibody can be administered from 25 μg-500 μg, 25 μg-450 μg, 50 μg-400 μg, from 50 μg-350 μg, from 50 μg-300 μg, from 50 μg-250 μg, from 50 μg-200 μg, from 50 μg-150 μg or from 50 μg-100 μg. An anti-OX40 monoclonal antibody can be administered at 250 μg. An anti-OX40 monoclonal antibody can be administered at 250 μg once a week for one week. An anti-OX40 monoclonal antibody can be administered at 250 μg once a week for two weeks. An anti-OX40 monoclonal antibody can be administered at 250 μg once a week for three weeks. An anti-OX40 monoclonal antibody can be administered at 250 μg three times a week for one week. An anti-OX40 monoclonal antibody can be administered at 250 μg three times a week for two weeks. An anti-OX40 monoclonal antibody can be administered at 250 μg three times a week for three weeks.

The results provided in the instant disclosure demonstrate that administration of the a Sindbis virus expressing IL-12 (SV.IL12) markedly increases the expression of OX40 on CD4 T cells and demonstrate that administration of a combination of SV.IL12 and anti-OX40 monoclonal antibody resulted in complete tumor regression in colon cancer, prostate cancer and ovarian cancer in vivo models and led to a greater than 60% survival rate (in some instances to a greater than 90% survival rate). This combined therapeutic effect was dramatically more effective when compared to either SV.IL12 or anti-OX40 monoclonal antibody treatment alone. These results also confirm that the oncolytic activity of the Sindbis virus is not required to induce a robust and effective anti-tumor response.

The results provided in the instant disclosure demonstrate that the combination of SV.IL12 or anti-OX40 monoclonal antibody treatment markedly changes the transcriptome signature of T cells and favors the differentiation of terminal effector T cells (e.g., effector T cells with a Th1 type phenotype). In particular, pathways upregulated by the combination treatment were dominated by DNA replication, chromosomal organization and cell cycle regulation, but also included various metabolic and immunological processes, such as mitochondrial respiration, nucleotide metabolism and adaptive immune responses. Specifically, only T cells from combined therapy expressed the gene signature of terminally differentiated effector T cells, which are characterized by high expression of the killer lectin-like receptor (KLRG1) and low expression of the interleukin 7 receptor (IL-7R). Furthermore, genes encoding products associated with the differentiation and function of effector cells, such as Batf Id2, Tbet, Gzmb and Ifng, were also highly expressed in T cells following combination therapy. Furthermore, CD4 T cells also expressed a marked anti-tumor effector phenotype (ICOS*Tbet*) which was on average 2 to 3-fold higher during combined therapy compared with SV.IL12 or anti-OX40 treatment.

The tumor microenvironment can be a very challenging milieu for an effector T cell as it is characterized by hypoxia, acidosis and low levels of nutrient sources such as glucose and glutamine. Even if T cell activation and initiation of effector function is allowed, T cells may be unable to generate the bioenergetics intermediates necessary to carry out effector function in the tumor microenvironment. Thus, providing a metabolic support for T cells is crucial for the success of cancer treatments. The results provided in the instant disclosure demonstrate that the combination of SV.IL12 or anti-OX40 monoclonal antibody promotes metabolic reprogramming of T cells. Specifically, the basal rate of oxygen consumption (OCR) was enhanced and spare respiratory capacity was dramatically increased in T cells following combination treatment. The combination also induced elevated protein expression of c-MYC as well as rate of extracellular acidification (ECAR). Collectively, these results show that SV.IL12 induces enhanced oxidative phosphorylation in CD8 T cells and the combination treatment is required to push CD4 T cells towards glycolysis by increasing the protein expression of c-MYC. Thus, the combination of SV.IL12 or anti-OX40 monoclonal antibody metabolically rewires T cells to an energetic state using both metabolic pathways, oxidative phosphorylation and glycolysis.

The results provided in the instant disclosure demonstrate that metabolic reprogrammed T cells display enhanced CD4 mediated cytokine production and anti-tumor activity following treatment with the combination of SV.IL12 and anti-OX40 monoclonal antibody. Specifically, genes encoding pro-inflammatory cytokines ifng and il2 were upregulated in T cells and the secretion of interferon-γ (IFNγ) by splenocytes was increased following combination treatment. Additional, the levels of the cytotoxic proteases, granzyme A and B, were upregulated following combination treatment. Further, granzyme B positive cells were detected in CD8 as well as CD4 T cells, indicating the presence of cytotoxic CD4 T cells following combination treatment. In addition, tumor growth was markedly reduced when co-cultured with splenocytes from mice receiving combined therapy. Surprisingly, tumor growth inhibition was mediated by CD4 T cells. Together, these results clearly show that T cells from combined therapy elicit enhanced anti-tumor and functional activity, such as granzyme B and IFNγ production driven by CD4 T cells.

The results provided in the instant disclosure demonstrate that treatment with the combination of SV.IL12 and anti-OX40 monoclonal antibody results in enhanced T cell migration and intratumoral T cell immunity. Specifically, CXCR3 levels were significantly upregulated on CD4 T cells following combination therapy. In contrast, CXCR3 expression on CD8 T cells only appeared later on in treatment, indicating that CD4 T cells are first recruited to the inflamed site followed by CD8 T cells. Combination therapy also enhanced the production of CXCL9 and CXCL10 in the tumor microenvironment, indicating that CXCR3 positive T cells migrate to the tumor site. These results clearly show that the combination of SV.IL12 and anti-OX40 monoclonal antibody alter the tumor microenvironment by facilitating T cell infiltration via modulation of the CXCR3/CXCL9-11 axis. Not only did combination therapy increase T cell infiltration but CD4 as well as CD8 T cells also demonstrated enhanced functional activity in the tumor, as judged by the Ki-67 and granzyme B expression. These results indicate that the presence of activated T cells in the tumor microenvironment exert anti-tumor activity which inhibits tumor growth. Enhanced iNOS production was also demonstrated in tumors treated with combination therapy. Interestingly, the amount of iNOS inversely correlated with arginase1 production, indicating a repolarization of tumor associated macrophages from the M2-like (pro-tumor) into Ml-like (anti-tumor) phenotype during combination therapy.

Thus, the data provided herein clearly shows that even in absence of direct Sindbis virus infectivity, SV.IL12 in combination with an anti-OX40 monoclonal antibody alter the tumor microenvironment by enhancing T cell infiltration and intratumoral T cell immunity, especially against low immunogenic tumors. The synergistic therapeutic efficacy of the systemic administration of the combination is driven by T cell modulation and reprogramming of its metabolic state, in order to enhance the anti-tumor response in the periphery and in the tumor microenvironment. Furthermore, the use of Sindbis virus allows these metabolically reprogrammed T cells to better infiltrate the tumor microenvironment, which is crucial for an adequate immunotherapy.

Sindbis Viral Vector and Anti-4-1BB Monoclonal Antibody

The present disclosure also provides methods for treating cancer in a subject in need thereof comprising administering to the subject a therapeutically effective amount (a) a Sindbis viral vector and (b) an anti-4-1BB monoclonal antibody or a nucleic acid encoding same, thereby treating cancer in the subject. The present disclosure further provides in vitro or ex vivo methods for treating cancer or assessing the treatment of cancer in a subject comprising contacting a biological sample from the subject with (a) a Sindbis viral vector and (b) an anti-4-1BB monoclonal antibody or a nucleic acid encoding same. Preferably, the Sindbis viral vector does not comprise an endogenous nucleic acid encoding any protein. Sindbis viral vectors were produced as described in U.S. Pat. No. 8,093,021 (incorporated herein by reference in its entirety).

The Sindbis viral vector is replication defective. The Sindbis viral vector can comprise a nucleic acid sequence encoding a therapeutic protein, an immunostimulatory protein or an immunomodulatory protein. The Sindbis viral vector can comprise the nucleic acid encoding the therapeutic protein, an immunostimulatory protein or an immunomodulatory protein and further comprise the nucleic acid encoding the anti-4-1BB monoclonal antibody. The Sindbis viral vector can comprise a nucleic acid sequence encoding LacZ (lac operon structural gene lacZ encoding β-galactosidase), Flue (firefly luciferase) or GFP (green fluorescent protein). The Sindbis viral vector can comprise the nucleic acid encoding LacZ, Flue or GFP and further comprise the nucleic acid encoding the anti-4-1BB monoclonal antibody.

The Sindbis viral vector can comprise a nucleic acid encoding an anti-4-1BB antibody heavy chain comprising the heavy chain complementarity determining region 1 (HCDR1), HCDR2 and HCDR3 amino acid sequences as follows: HCDR1: GFIFSYFDMA (SEQ ID NO: 16), HCDR2: SISPDGSIPYYRDSVK (SEQ ID NO: 17) and HCDR3: RSYGGYSELDY (SEQ ID NO: 18).

The Sindbis viral vector can comprise a nucleic acid encoding an anti-4-1BB heavy chain comprising the amino acid sequence of SEQ ID NO:19 shown in the following Table.

anti-4-1BB heavy chain amino acid
(SEQ ID NO: 19)
DVQLVESGGGLVQPGRSLKLSCAASGFIFSYEDMAWVRQAPTKGLEWVAS
ISPDGSIPYYRDSVKGRFTVSRENAKSSLYLQMDSLRSEDTATYYCARRS
YGGYSELDYWGQGVMVTVSS.

The Sindbis viral vector can comprise a nucleic acid encoding an anti-4-1BB antibody light chain comprising the light chain complementarity determining region 1 (LCDR1), LCDR2 and LCDR3 amino acid sequences as follows: LCDR1: QASQDIGNWLA (SEQ ID NO: 20), LCDR2: GSTSLAD (SEQ ID NO: 21) and LCDR3: LQAYGAPW (SEQ ID NO: 22).

The Sindbis viral vector can comprise a nucleic acid encoding an anti-4-1BB light chain comprising the amino acid sequence of SEQ ID NO:23 shown in the following Table.

anti-4-1BB light chain amino acid
(SEQ ID NO: 23)
DIQMTQSPASLSASLEEIVTITCQASQDIGNWLAWYHQKPGKSPQLLIYG
STSLADGVPSRFSGSSSGSQYSLKISRLQVEDIGIYYCLQAYGAPWTEGG
GTKLELK

The Sindbis viral vector can comprise a nucleic acid encoding anti-4-1BB antibody heavy chain CDR1, CDR2 and CDR3 amino acid sequences that binds to a target antigen comprising the amino acid sequence of SEQ ID NO:24 shown in the following Table.

| anti-4-1BB target antigen | |
|---|---|
| (SEQ ID NO: 24) | |
| 1 | MGNNCYNVVV IVLLLVGCEK VGAVQNSCDN CQPGTFCRKY NPVCKSCPPS |
| 51 | TFSSIGGQPN CNICRVCAGY FRFKKFCSST HNAECECIEG FHCLGPQCTR |
| 101 | CEKDCRPGQE LTKQGCKTCS LGTFNDQNGT GVCRPWTNCS LDGRSVLKTG |
| 151 | TTEKDVVCGP PVVSFSPSTT ISVTPEGGPG GHSLQVLTLF LALTSALLLA |
| 201 | LIFITLLFSV LKWIRKKFPH IFKQPFKKTT GAAQEEDACS CRCPQEEEGG |
| 251 | GGGYEL |

The Sindbis viral vector can comprise a nucleic acid encoding a 4-1BB heavy chain that is at least 70%, 75%, 80%, 85%, 90%, 95%, 96%, 97%, 98%, 99% or 100% identical to amino acid sequence of SEQ ID NO: 19 and a nucleic acid encoding a 4-1BB light chain that is at least 70%, 75%, 80%, 85%, 90%, 95%, 96%, 97%, 98%, 99% or 100% identical to amino acid sequence of SEQ ID NO: 23.

The Sindbis viral vector can comprise the nucleic acid encoding anti-4-1BB antibody light chain CDR1, CDR2 and CDR3 amino acid sequences that binds to a target antigen (4-1BB antigen) of the amino acid sequence of (SEQ ID NO: 24). The Sindbis viral vector can comprise the nucleic acid encoding anti-4-1BB antibody heavy chain amino acid sequence that binds to a target antigen (4-1BB antigen) of the amino acid sequence of (SEQ ID NO: 24). The Sindbis viral vector can comprise the nucleic acid encoding anti-4-1BB antibody light chain amino acid sequence that binds to a target antigen (4-1BB antigen) of the amino acid sequence of (SEQ ID NO: 24).

The Sindbis viral vector can comprise a nucleic acid encoding an anti-4-1BB antibody heavy chain comprising the heavy chain complementarity determining region 1 (HCDR1), HCDR2 and HCDR3 amino acid sequences of SEQ ID NOs: 16, 17 and 18, respectively. The Sindbis viral vector can comprise the nucleic acid encoding an anti-4-1BB antibody heavy chain comprising the amino acid sequence of SEQ ID NO: 19. The Sindbis viral vector can comprise a nucleic acid encoding an anti-4-1BB antibody heavy chain comprising the light chain complementarity determining region 1 (LCDR1), LCDR2 and LCDR3 amino acid sequences of SEQ ID NOs: 20, 21 and 22, respectively. The Sindbis viral vector can comprise the nucleic acid encoding an anti-4-1BB antibody light chain comprising the amino acid sequence of SEQ ID NO: 23.

The Sindbis viral vector can comprise the nucleic acid encoding anti-4-1BB antibody heavy chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody light chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody heavy chain amino acid sequence that binds to a target antigen of the amino acid sequence of SEQ ID NO: 24. The Sindbis viral vector can comprise the nucleic acid encoding anti-4-1BB antibody light chain amino acid sequence that binds to a target antigen of the amino acid sequence of SEQ ID NO: 24.

The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12. The anti-4-1BB antibody can be urelumab, utomilumab or a combination thereof. The anti-4-1BB antibody can be InVivoMAb anti-mouse 4-1BB (BioXCell, Clone: LOB12.3, Cat. No. BE0169).

The Sindbis viral vector and the anti-4-1BB monoclonal antibody can be administered sequentially or concurrently. The Sindbis viral vector can be administered systemically. The anti-4-1BB monoclonal antibody can be administered systemically. Both the Sindbis viral vector and the anti-4-1BB monoclonal antibody, or a nucleic acid encoding same, can be administered systemically. The Sindbis viral vector can be administered parenterally. The anti-4-1BB monoclonal antibody can be administered parenterally. Both the Sindbis viral vector and the anti-4-1BB monoclonal antibody, or a nucleic acid encoding same, can be administered parenterally. The Sindbis viral vector can be administered intraperitoneally. The anti-4-1BB monoclonal antibody can be administered intraperitoneally. Both the Sindbis viral vector and the anti-4-1BB monoclonal antibody, or a nucleic acid encoding same, can be administered intraperitoneally.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against 4-1BB or an anti-4-1BB monoclonal antibody, as described herein, can be a full length antibody against 4-1BB antibody or an antigen binding fragment thereof, wherein the fragment retains binding to the 4-1BB receptor on a cell surface. An “antigen-binding fragment” of an anti-4-1BB antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is a lymphoma. In one preferred aspect, the cancer is a B cell lymphoma.

The present disclosure provides a Sindbis viral vector comprising a nucleic acid encoding a therapeutic protein, an immunostimulatory protein or an immunomodulatory protein and a nucleic acid encoding an anti-4-1BB monoclonal antibody. The present disclosure provides a Sindbis viral vector comprising a nucleic acid encoding LacZ, Flue or GFP and a nucleic acid encoding an anti-4-1BB monoclonal antibody. The present disclosure also provides a composition comprising (a) a Sindbis viral vector comprising a nucleic acid encoding a therapeutic protein, an immunostimulatory protein or an immunomodulatory protein and (b) a Sindbis viral vector comprising a nucleic acid encoding an anti-4-1BB monoclonal antibody. The present disclosure also provides a composition comprising (a) a Sindbis viral vector comprising a nucleic acid encoding LacZ, Flue or GFP and (b) a Sindbis viral vector comprising a nucleic acid encoding an anti-4-1BB monoclonal antibody. The present disclosure further provides a composition comprising (a) a Sindbis viral vector comprising a nucleic acid encoding a therapeutic protein, an immunostimulatory protein or an immunomodulatory protein and (b) an anti-4-1BB monoclonal antibody or a nucleic acid encoding same. The present disclosure provides a composition comprising (a) a Sindbis viral vector comprising a nucleic acid encoding encoding LacZ, Flue or GFP and (b) an anti-4-1BB monoclonal antibody or a nucleic acid encoding same.

The Sindbis viral vector can comprise a nucleic acid encoding an anti-4-1 in antibody heavy chain comprising the heavy chain complementarity determining region 1 (HCDR1), HCDR2 and HCDR3 amino acid sequences of SEQ ID NOs: 16, 17 and 18, respectively. The Sindbis viral vector can comprise the nucleic acid encoding an anti-4-1BB antibody heavy chain comprising the amino acid sequence of SEQ ID NO: 19. The Sindbis viral vector can comprise a nucleic acid encoding an anti-4-1BB antibody heavy chain comprising the light chain complementarity determining region 1 (LCDR1), LCDR2 and LCDR3 amino acid sequences of SEQ ID NOs: 20, 21 and 22, respectively. The Sindbis viral vector can comprise the nucleic acid encoding an anti-4-1BB antibody light chain comprising the amino acid sequence of SEQ ID NO: 23.

The Sindbis viral vector can comprise the nucleic acid encoding anti-4-1BB antibody heavy chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody light chain CDR1, CDR2 and CDR3 amino acid sequences that binds to a target antigen of the amino acid sequence of SEQ ID NO: 24. The Sindbis viral vector can comprise the nucleic acid encoding anti-4-1BB antibody heavy chain amino acid sequence that binds to a target antigen of the amino acid sequence of SEQ ID NO: 24. The Sindbis viral vector can comprise the nucleic acid encoding anti-4-1BB antibody light chain amino acid sequence that binds to a target antigen of the amino acid sequence of SEQ ID NO: 24.

The immunostimulatory or immunomodulatory protein can be IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or any combination thereof. In a preferred aspect, the immunostimulatory or immunomodulatory protein is IL-12. Additional cytokines include IL-I8-IL-36. In addition to CCL17, other chemokines can also be used, including, but not limited to, CCL1-CCL27 and other CC chemokines, CXCLI-CXCL13 and other CXC chemokines, C chemokines, and CX3C chemkines. Cytokine or chemokine receptors and soluble receptors can also be used. Additional immune modulators that can be used include TGF-β and TNFα. In addition, different combinations of the above-mentioned (or alternative) cytokines can be used.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against 4-1BB or an anti-4-1BB monoclonal antibody, as described herein, can be a full length antibody against 4-1BB antibody or an antigen binding fragment thereof, wherein the fragment retains binding to the 4-1BB receptor on a cell surface. An “antigen-binding fragment” of an anti-4-1BB antibody, as described herein can include any fragment selected from the group consisting of Fv, Fav, F(ab′)2, Fab′, dsFv, scFv, sc(Fv)2, scFv-CH3, scFv-Fc, and diabody fragments.

Sindbis virus can be administered at least one time, at least two times, at least three times, at least four times or at least five times per week. Sindbis virus can be administered for at least one week, at least two weeks, at least three weeks, or at least four weeks. Sindbis virus can be administered from 10⁶-10⁹ TU/mL. Preferably, Sindbis virus can be administered from 10⁶-10⁹ TU/mL.

An anti-4-1BB monoclonal antibody can be administered at least one time, at least two times, at least three times, at least four times or at least five times per week. An anti-4-1BB monoclonal antibody can be administered for at least one week, at least two weeks, at least three weeks, or at least four weeks. An anti-4-1BB monoclonal antibody can be administered from 25 μg-500 μg, 25 μg-450 μg, 50 μg-400 μg, from 50 μg-350 μg, from 50 μg-300 μg, from 50 μg-250 μg, from 50 μg-200 μg, from 50 μg-150 μg or from 50 μg-100 μg. An anti-4-1BB monoclonal antibody can be administered at 50 μg. An anti-4-1BB monoclonal antibody can be administered at 50 μg once a week for three weeks. An anti-4-1BB monoclonal antibody can be administered at 250 μg. An anti-4-1BB monoclonal antibody can be administered at 250 μg three times week for two weeks. An anti-4-1BB monoclonal antibody can be administered at 350 μg. An anti-4-1BB monoclonal antibody can be administered at 350 μg three times week for two weeks.

The results provided in the instant disclosure demonstrate that administration of the combination of Sindbis virus and anti-4-1BB monoclonal antibody resulted in complete tumor regression in an lymphoma in vivo model and that this therapeutic effect was dramatically more effective when compared to either Sindbis virus or anti-4-1BB monoclonal antibody treatment alone. Tumor elimination involves a synergistic effect of the combination that significantly boosts T cell cytotoxicity, IFNγ production, T cell proliferation, migration, and glycolysis. As described in more detail below, the data identified the molecular pathways, including upregulated cytokines, chemokines and metabolic pathways in T cells that are triggered by the combined therapy and help to achieve a highly effective anti-tumor response.

The results provided in the instant disclosure demonstrate that administration of the combination of Sindbis virus and anti-4-1BB monoclonal antibody resulted in increased T cell cycle progression, cytokine production and activation. T cell proliferation is critical for an effective anti-tumor response.

The results provided in the instant disclosure demonstrate that administration of the combination of Sindbis virus and anti-4-1BB monoclonal antibody resulted in increased cytotoxicity (e.g., increased cytotoxic T cell function). Specifically, genes such as Gzmb (granzyme B), Prfl (perforin) and Klrkl (NKG2D) are significantly upregulated in T cells (particularly CD8 T cells) following administration of Sindbis virus and anti-4-1BB monoclonal antibody.

The results provided in the instant disclosure demonstrate that administration of the combination of Sindbis virus and anti-4-1BB monoclonal antibody resulted in increased IFNγ production from T cells and Th1 differentiation. The combination of Sindbis virus and anti-4-1BB monoclonal antibody upregulated the expression of STAT4, Ccr5, Cxcr3, Havcr2(Tim3), IL12rbl and Klrcl in T cells, which are required for the development of Th1 cells from naïve CD4+ T cells and IFNγ production. This increase was independent of the presence or absence of TAA. The combination of Sindbis virus and anti-4-1BB monoclonal antibody increased IFNγ production from both CD4 and CD8 T cells (with a larger portion CD4 T cells producing IFNγ) and demonstrated that antigen presenting cells (APCs) are essenTh1tial for helping T cells product IFNγ. The combination of Sindbis virus and anti-4-1BB monoclonal antibody also increased T-bet in T cells. T-bet is a key transcription factor which is essential for type I immune response (IFNγ production, T cell cytotoxicity) and memory T cell differentiation. Thus, this indicates that the combination of Sindbis virus and anti-4-1BB monoclonal antibody boosts the type I immune response, which is critical for controlling tumor growth. The combination of Sindbis virus and anti-4-1BB monoclonal antibody also increased Eomesodermin (EOMES) in T cells. EOMES, another important transcription factor, is upregulated in activated T cells and is essential for memory CD8 T cell development.

The results provided in the instant disclosure demonstrate that administration of the combination of Sindbis virus and anti-4-1BB monoclonal antibody resulted in increased chemotaxis, adhesion and enhanced T cell infiltration and activation in tumors. Specifically, the combination significantly upregulates CD11a and ICAM-1(CD54) in both CD4 and CD8 T cells, which are two adhesion molecules expressed on activated T cells and are essential for the formation of immune synapses between T cells and APCs and are also required for T cell/T cell homotypic aggregation and activation. The combination of Sindbis virus and anti-4-1BB monoclonal antibody also significantly upregulated OX40 and ICOS in T cells. OX40 engagement promotes effector T cell function and survival and ICOS is another key CD4 T cell costimulatory molecule. Tumor infiltrating lymphocytes play a critical anti-tumor role and are an important marker for prognosis. The percentage of CD3 and CD8 T cells increased about two-fold following combination treatment. Thus, these results demonstrate that combination treatment enhanced T cell infiltration, division, activation, cytotoxicity and downregulated the inhibitory Treg population.

The results provided in the instant disclosure demonstrate that administration of the combination of Sindbis virus and anti-4-1BB monoclonal antibody resulted in enhanced T cell glycolysis and oxidative phosphorylation. T cell activation requires a quick consumption of energy through both enhanced glycolysis and oxidative phosphorylation. Metabolic switch is a major feature of T cell activation and memory T cell development. Upregulation of glycolysis genes quickly produce ATP and supports T cell migration and cytotoxicity in hypoxic or acidific microenvironments (such as in and around a tumor). The instant results demonstrate that combination treatment significantly increased both oxygen consumption rate (OCR, represents oxidative phosphorylation) and extracellular acidification rate (ECAR, represents glycolysis). This indicates that both glycolysis and oxidative phosphorylation are activated in combination treated T cells.

The results provided in the instant disclosure demonstrate that mice cured by the administration of the combination of Sindbis virus and anti-4-1BB monoclonal antibody are completely protected from cancer rechallenge demonstrating that these mice acquired long lasting antitumor immunity.

The conventional view of oncolytic virus therapy against tumors is that it requires selective infection of cancer cells resulting in the induction of cancer cell lysis and apoptosis. Tumor specific antigens (TAAs), released from dead tumor cells, attract and further stimulate an antitumor immune response. The data presented herein demonstrates that encoding a TAA is not necessary for the combination of Sindbis virus and anti-4-1BB monoclonal antibody to be fully successful in eradicating growing tumors.

The quick inhibition of tumor growth is critical for cancer therapy because tumor cells undergo exponentially rapid division. However, the induction of adaptive immunity and establishment of tumor specific immunity takes a long time. An ideal therapy requires an early, quick reduction of tumor burden, and a later induction of anti-tumor specificity that prevents relapse. The data presented herein demonstrates that the combination of Sindbis virus and anti-4-1BB monoclonal antibody treatment induced massive T cell activation due to viral induced immune response. This massive activation helps to control the tumor in a TAA nonspecific manner.

It was shown herein that both NKG2D (KLRKI) and granzyme B are highly expressed under combination treatment. This massive nonspecific activation is critical for controlling tumor growth at an early time point (day 7). This step is also important for inducing anti-tumor specificity that is mediated by TAAs released from dead tumor cells due to nonspecific killing. After tumor regression, T cells from treated animals were able maintain the ability to produce IFNγ and acquired immunological memory to rapidly reject tumor rechallenges. IFNγ production from purified T cells of cured mice was significantly enhanced after encountering tumor cells. This demonstrates that anti-tumor specificity is fully established in cured mice.

The data also shows that Sindbis viral infection of tumor cells, inclusion of dendtric cells and lymphodepletion are not necessary for successful cancer treatment. The omission of these additional features decreases costs, any risks related to toxicity and infection.

Thus, the data provided herein demonstrates that the combination of Sindbis virus and anti-4-1BB monoclonal antibody completely eradicated a B-cell lymphoma in a preclinical mouse model, a result that could not be achieved with either treatment alone. Tumor elimination involves a synergistic effect of the combination that significantly boosts T cell cytotoxicity, IFN-γ production, migration, tumor infiltration and oxidative phosphorylation. In addition, all mice that survived after treatment developed long lasting antitumor immunity.

Sindbis Viral Vector and Sindbis Viral Vector NY-ESO-1

The present disclosure also provides methods for treating cancer in a subject in need thereof comprising administering to the subject a therapeutically effective amount of a Sindbis viral vector comprising a nucleic acid encoding interleukin-12 and a Sindbis viral vector comprising a nucleic acid encoding NY-ESO-1, thereby treating cancer in the subject. The present disclosure also provides methods for treating cancer in a subject in need thereof comprising administering to the subject a therapeutically effective amount of a Sindbis viral vector comprising a nucleic acid encoding interleukin-12 and a nucleic acid encoding NY-ESO-1, thereby treating cancer in the subject.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit of GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise the nucleic acid encoding interleukin-12 alpha subunit of amino acid sequence of GenBank accession no. AAA39292.1 and interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO:14 shown in the following Table.

| Nucleic acid sequence encoding NY-ESO-1 (NM_001327.1) | |
|---|---|
| (SEQ ID NO: 14) | |
| 1 | agcagggggc gctgtgtgta ccgagaatac gagaatacct cgtgggccct gaccttctct |
| 61 | ctgagagccg ggcagaggct ccggagccat gcaggccgaa ggccggggca cagggggttc |
| 121 | gacgggcgat gctgatggcc caggaggccc tggcattcct gatggcccag ggggcaatgc |
| 181 | tggcggccca ggagaggcgg gtgccacggg cggcagaggt ccccggggcg caggggcagc |
| 241 | aagggcctcg gggccgggag gaggcgcccc gcggggtccg catggcggcg cggcttcagg |
| 301 | gctgaatgga tgctgcagat gcggggccag ggggccggag agccgcctgc ttgagttcta |
| 361 | cctcgccatg cctttcgcga cacccatgga agcagagctg gcccgcagga gcctggccca |
| 421 | ggatgcccca ccgcttcccg tgccaggggt gcttctgaag gagttcactg tgtccggcaa |
| 481 | catactgact atccgactga ctgctgcaga ccaccgccaa ctgcagctct ccatcagctc |
| 541 | ctgtctccag cagctttccc tgttgatgtg gatcacgcag tgctttctgc ccgtgttttt |
| 601 | ggctcagcct ccctcagggc agaggcgcta agcccagcct ggcgcccctt cctaggtcat |
| 661 | gcctcctccc ctagggaatg gtcccagcac gagtggccag ttcattgtgg gggcctgatt |
| 721 | gtttgtcgct ggaggaggac ggcttacatg tttgtttctg tagaaaataa aactgagcta |
| 781 | cgaaaaaaaa aaaaaaaaaa aaaaaa |

The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO:15 shown in the following Table.

| Amino acid sequence of NY-ESO-1 (NP_001318.1) | |
|---|---|
| (SEQ ID NO: 15) | |
| 1 | mqaegrgtgg stgdadgpgg pgipdgpggn aggpgeagat ggrgprgaga arasgpggga |
| 61 | prgphggaas glngccrcga rgpesrllef ylampfatpm eaelarrsla qdapplpvpg |
| 121 | vllkeftvsg niltirltaa dhrqlqlsis sclqqlsllm witqcflpvf laqppsgqrr |

The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

A replication defective Sindbis viral vector as described herein can be any replication defective Sindbis viral vector including a replication defective viral vector described, for example, in U.S. Pat. Nos. 7,303,898, 7,306,792, and 8,093,021. Replication defective vectors are preferred for use in the present invention in order to prevent infection of healthy tissues.

The Sindbis viral vector comprising a nucleic acid encoding interleukin-12 and the Sindbis viral vector comprising a nucleic acid encoding NY-ESO-1 can be administered sequentially or concurrently. The Sindbis viral vector comprising a nucleic acid encoding interleukin-12 can be administered systemically. The Sindbis viral vector comprising a nucleic acid encoding NY-ESO-1 can be administered systemically. Both the Sindbis viral vector comprising a nucleic acid encoding interleukin-12 and the Sindbis viral vector comprising a nucleic acid encoding NY-ESO-1 can be administered systemically. The Sindbis viral vector comprising a nucleic acid encoding interleukin-12 can be administered parenterally. The Sindbis viral vector comprising a nucleic acid encoding NY-ESO-1 can be administered parenterally. Both the Sindbis viral vector comprising a nucleic acid encoding interleukin-12 and the Sindbis viral vector comprising a nucleic acid encoding NY-ESO-1 can be administered parenterally. The Sindbis viral vector comprising a nucleic acid encoding interleukin-12 can be administered intraperitoneally. The Sindbis viral vector comprising a nucleic acid encoding NY-ESO-1 can be administered intraperitoneally. Both the Sindbis viral vector comprising a nucleic acid encoding interleukin-12 and the Sindbis viral vector comprising a nucleic acid encoding NY-ESO-1 can be administered intraperitoneally.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

The present disclosure further provides a Sindbis viral vector comprising a nucleic acid encoding interleukin-12 and a nucleic acid encoding NY-ESO-1. The present disclosure also provides a composition comprising (a) a Sindbis viral vector comprising a nucleic acid encoding interleukin-12 and (b) a Sindbis viral vector comprising a nucleic acid encoding NY-ESO-1.

The Sindbis viral vector can comprise the nucleic acid encoding interleukin-12 alpha subunit (IL-12 α, IL-12, p35 subunit) and interleukin-12 beta subunit (IL-12 β, IL-12, p40 subunit). The Sindbis viral vector can comprise the nucleic acid encoding interleukin-12 alpha subunit of GenBank accession no. M86672 and interleukin-12 beta subunit of GenBank accession no. M86671. The nucleic acid encoding interleukin-12 alpha subunit comprises the nucleic acid sequence of SEQ ID NO: 1. The nucleic acid encoding interleukin-12 beta subunit comprises the nucleic acid sequence of SEQ ID NO: 2.

The Sindbis viral vector can comprise the nucleic acid encoding interleukin-12 alpha subunit of amino acid sequence of GenBank accession no. AAA39292.1 and interleukin-12 beta subunit of amino acid sequence of GenBank accession no. AAA39296.1. The amino acid sequence of the interleukin-12 alpha subunit is of amino acid sequence of SEQ ID NO: 3. The amino acid sequence of the interleukin-12 beta subunit is of amino acid sequence of SEQ ID NO: 4.

The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The present disclosure provides methods for treating cancer in a subject in need thereof comprising administering to the subject a therapeutically effective amount of (a) a Sindbis viral vector comprising a nucleic acid encoding NY-ESO-1 and (b) an anti-OX40 monoclonal antibody or a nucleic acid encoding same, thereby treating cancer in the subject.

The Sindbis viral vector can be replication defective. The Sindbis viral vector can comprise the nucleic acid encoding NY-ESO-1 and can further comprise the nucleic acid encoding the anti-OX40 monoclonal antibody. The method can comprise administering a Sindbis viral vector comprising the nucleic acid encoding NY-ESO-1 and administering a Sindbis viral vector comprising the nucleic acid encoding the anti-OX40 monoclonal antibody. The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

The Sindbis viral vector and the anti-OX40 monoclonal antibody can be administered sequentially or concurrently. The Sindbis viral vector can be administered systemically. The anti-OX40 monoclonal antibody can be administered systemically. Both the Sindbis viral vector and the anti-OX40 monoclonal antibody, or a nucleic acid encoding same, can be administered systemically. The Sindbis viral vector can be administered parenterally. The anti-OX40 monoclonal antibody can be administered parenterally. Both the Sindbis viral vector and the anti-OX40 monoclonal antibody, or a nucleic acid encoding same, can be administered parenterally. The Sindbis viral vector can be administered intraperitoneally. The anti-OX40 monoclonal antibody can be administered intraperitoneally. Both the Sindbis viral vector and the anti-OX40 monoclonal antibody, or a nucleic acid encoding same, can be administered intraperitoneally.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

The cancer can be a solid cancer or a liquid/hematologic cancer. The cancer can comprise metastatic cancer. The cancer can comprise a solid tumor. The cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer. In one preferred aspect, the cancer is colon cancer. In one preferred aspect, the cancer is prostate cancer. In one preferred aspect, the cancer is ovarian cancer.

The present disclosure further provides a Sindbis viral vector comprising a nucleic acid encoding NY-ESO-1 and a nucleic acid encoding an anti-OX40 monoclonal antibody. The present disclosure also provides a composition comprising (a) a Sindbis viral vector comprising a nucleic acid encoding NY-ESO-1 and (b) an anti-OX40 monoclonal antibody or a nucleic acid encoding same. The present disclosure also provides a composition comprising (a) a Sindbis viral vector comprising a nucleic acid encoding NY-ESO-1 and (b) a Sindbis viral vector comprising a nucleic acid encoding an anti-OX40 monoclonal antibody. The Sindbis viral vector can comprise the nucleic acid encoding NY-ESO-1 of NCBI Reference accession no. NM_001327.1. The Sindbis viral vector can comprise the nucleic acid encoding NY-ESO-1 of NCBI Reference accession no. NM_001327.1. The nucleic acid encoding NY-ESO-1 comprises the nucleic acid sequence of SEQ ID NO: 14. The Sindbis viral vector can comprise the nucleic acid encoding NY-ESO-1 of amino acid sequence of NCBI Reference accession no. NP_001318.1. The amino acid sequence of the NY-ESO-1 comprises the amino acid sequence of SEQ ID NO: 15.

The Sindbis viral vector can comprise a nucleic acid encoding an anti-OX40 variable heavy chain comprising the amino acid sequence of SEQ ID NO: 5. The nucleic acid sequence encoding the anti-OX40 variable heavy chain is SEQ ID NO: 6. The Sindbis viral vector can comprise a nucleic acid encoding an anti-OX40 variable light chain comprising the amino acid sequence of SEQ ID NO: 7. The nucleic acid sequence encoding the anti-OX40 variable light chain is SEQ ID NO: 8.

The Sindbis viral vector can comprise a nucleic acid encoding a mouse anti-OX40 heavy chain of amino acid sequence of SEQ ID NO; 5 and a nucleic acid encoding a mouse anti-OX40 light chain of amino acid sequence of SEQ ID NO: 7. The Sindbis viral vector can comprise a nucleic acid encoding a mouse anti-OX40 heavy chain that is at least 70%, 75%, 80%, 85%, 90%, 95%, 96%, 97%, 98%, 99% or 100% identical to amino acid sequence of SEQ ID NO: 5 and a nucleic acid encoding a mouse anti-OX40 light chain that is at least 70%, 75%, 80%, 85%, 90%, 95%, 96%, 97%, 98%, 99% or 100% identical to amino acid sequence of SEQ ID NO: 7.

The Sindbis viral vector can comprise a nucleic acid encoding an anti-OX40 antibody heavy chain comprising the amino acid sequence of SEQ ID NO: 9. The nucleic acid sequence encoding the an anti-OX40 antibody heavy chain is SEQ ID NO: 10.

The Sindbis viral vector can comprise a nucleic acid encoding a mouse anti-OX40 antibody light chain comprising the amino acid sequence of SEQ ID No: 11. The nucleic acid sequence encoding the anti-OX40 antibody light chain is SEQ ID NO: 12.

The Sindbis viral vector can comprise a nucleic acid encoding an anti-OX40 antibody heavy chain with an amino acid sequence of SEQ ID NO: 9 and an anti-OX40 antibody light chain with an amino acid sequence of SEQ ID NO: 11. The Sindbis viral vector can comprise a nucleic acid encoding an anti-OX40 antibody heavy chain of SEQ ID NO: 10 and an anti-OX40 antibody light chain with an amino acid sequence of SEQ ID NO: 12.

The Sindbis viral vector can comprise a nucleic acid encoding an anti-OX40 antibody heavy chain with an amino acid sequence that is at least 70%, 75%, 80%, 85%, 90%, 95%, 96%, 97%, 98%, 99% or 100% identical to SEQ ID NO: 9 and an anti-OX40 antibody light chain with an amino acid sequence that is at least 70%, 75%, 80%, 85%, 90%, 95%, 96%, 97%, 98%, 99% or 100% identical to SEQ ID NO: 11. The Sindbis viral vector can comprise a nucleic acid encoding an anti-OX40 antibody heavy chain that is at least 70%, 75%, 80%, 85%, 90%, 95%, 96%, 97%, 98%, 99% or 100% identical to SEQ ID NO: 10 and an anti-OX40 antibody light chain that is at least 70%, 75%, 80%, 85%, 90%, 95%, 96%, 97%, 98%, 99% or 100% identical to SEQ ID NO: 12.

The Sindbis viral vector can comprise the nucleic acid encoding an anti-OX40 antibody heavy chain with an amino acid sequence that binds to a target antigen of the amino acid sequence of SEQ ID NO: 13.

The Sindbis viral vector can comprise a nucleic acid encoding an anti-OX40 antibody variable heavy chain with an amino acid sequence that binds to a target antigen of the amino acid sequence of SEQ ID NO: 13. The Sindbis viral vector can comprise a nucleic acid encoding an anti-OX40 antibody heavy chain with an amino acid sequence that binds to a target antigen of the amino acid sequence of SEQ ID NO: 13. The Sindbis viral vector can comprise a nucleic acid encoding an anti-OX40 antibody variable light chain with an amino acid sequence that binds to a target antigen of the amino acid sequence of SEQ ID NO: 13. The Sindbis viral vector can comprise the nucleic acid encoding an anti-OX40 antibody light chain with an amino acid sequence that binds to a target antigen of the amino acid sequence of SEQ ID NO: 13.

The Sindbis viral vector can comprise a nucleic acid encoding an anti-OX40 antibody variable heavy chain amino acid sequence, and an anti-OX40 antibody variable light chain amino acid sequence, that binds to a target antigen of the amino acid sequence of SEQ ID NO: 13. The Sindbis viral vector can comprise a nucleic acid encoding an anti-OX40 antibody heavy chain amino acid sequence, and an anti-OX40 antibody light chain amino acid sequence, that binds to a target antigen of the amino acid sequence of SEQ ID NO: 13.

A “monoclonal antibody” as disclosed herein, can be a full-length antibody or an antigen binding fragment thereof, wherein the “antigen binding fragment” is a fragment of the full length antibody that retains binding to the target antigen of the said monoclonal antibody. For example, a monoclonal antibody against OX-40 or an anti-OX40 monoclonal antibody, as described herein, can be a full length antibody against OX40 antibody or an antigen binding fragment thereof, wherein the fragment retains binding to the OX40 receptor on a cell surface. An “antigen-binding fragment” of an anti-OX-40 antibody, as described herein can include any fragment selected from the group consisting of Fv, Fav, F(ab′)2, Fab′, dsFv, scFv, sc(Fv)2, scFv-CH3, scFv-Fc, and diabody fragments.

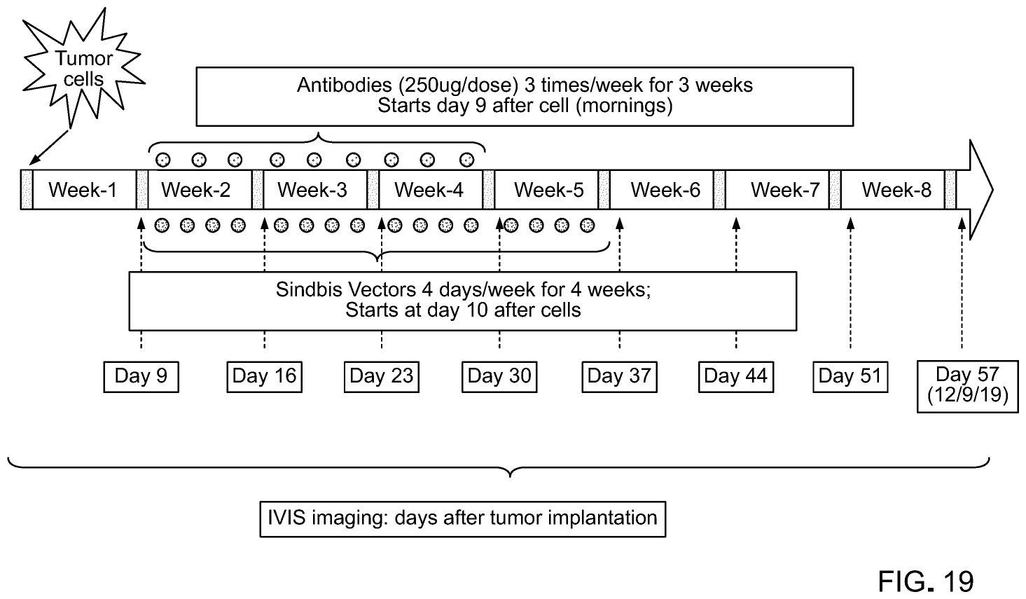

The results provided in the instant disclosure demonstrate that administration of a combination of IL-12 and NY-ESO-1, expressed by separate Sindbis virus vector synergistically enhances the survival rate of a subject bearing an established tumor. The results described herein show that mice transplanted with Alm5-2Fluc-17 ovarian cancer cells by reinjection to establish tumor as depicted in FIG. 19, when treated post-tumor establishment with a SV vector expressing NY-ESO-1 (SVNYESO) showed no enhancement of survival, with a percentage survival rate similar to untreated tumor bearing mice, thereby showing that some tumors are resistant to treatment with SV expressing a TAA, like NY-ESO-1. The results described herein show that treatment of the tumor bearing mice, with a SV expressing NYESO (SV-NYESO_SV-IL12) showed improvement in survival rate. The results show that surprisingly treatment with a 50% mix in one injection of a SV expressing IL-12 (SV-IL-12) and a SV expressing NYESO (SV-NYESO_SV-IL12), demonstrated synergistically enhanced survival as compared to mice treated with the SV-IL-12 or SV-NYESO. The results described herein clearly show the possibility of using a combination of SV vectors expressing IL-12 and NY-ESO-1, for treatment of cancers that may be resistant to treatment with a SV expressing a tumor associated antigen.

The results provided in the instant disclosure demonstrate that administration of a combination of IL-12 and NY-ESO-1, expressed by the same Sindbis virus vector synergistically enhances the survival rate of a subject bearing an established tumor. The results show that mice bearing established tumors of Alm5-2Fluc-17 ovarian cancer cells, when treated with a Sindbis viral vector that expresses both IL-12 and NYESO (SV-NYESO_SGP2_IL12), demonstrated synergistically enhanced survival as compared to mice treated with the SV-IL-12 or SV-NYESO. The results described herein clearly show the possibility of using a single SV vectors expressing both IL-12 and NY-ESO-1, for treatment of cancers that may be resistant to treatment with a SV expressing a tumor associated antigen.

Treating cancer means treating at least one symptom of cancer. Treating at least one symptom of cancer can include any of the following, or any combination thereof: inhibiting tumor growth, reducing tumor size, reducing tumor number, reducing tumor burden, preventing cancer recurrence, preventing metastasis of a primary tumor.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T cell lymphoma, Peripheral T cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

The nucleotide sequences encoding the TAAs to be expressed by a Sindbis viral vector as described herein are well known in the art and can be easily obtained from the literature. For example, the sequence of NY-ESO-1, a testicular antigen aberrantly expressed in human cancers was published in 1997 (http://www.pnas.org/content/94/5/1914.full, Yao-Tseng Chen, Matthew J. Scanlant, Ugur Sahin, Ozlem Tiireci, Ali O. Guret, Solam Tsangt, Barbara Williamsont, Elisabeth Stockertt, Michael Pfreundschuh, and Lloyd J. Old, PNAS 1997.), whereas the Carcinoembryonic antigen sequence was published in 1987 (http://mcb.asm.org/content/7/9/3221.short Isolation and characterization of full-length functional cDNA clones for human carcinoembryonic antigen. N Beauchemin, S. Benchimol, D Cournoyer, A Fuks and C P Stanners, Molecular and Cellular Biology.

Although in mice a single i.p. injection of the SV/TAA as described herein, is sufficient to elicit a detectable CD8+ mediated immune response directed against the tumor, other regimens may be necessary for achieving a maximal response. For example, between 1 and about 8 i.p. injections over a time period of between 1 week and many weeks, with the possibility of injecting one or more booster injections 1 or more years later, may be preferably administered for a maximum effect.

### EXAMPLES

### Example 1: Sindbis with Anti-OX40 Enable Immune Responses to Cold Tumors

This study, investigates the therapeutic efficacy of a replication-deficient oncolytic viral vector called Sindbis Virus. Because Sindbis Virus (SV) is a blood-borne pathogen, vectors from this virus can be administered in the bloodstream via the intravenous (i.v.) and intraperitoneal (i.p.) routes, which greatly facilitates their delivery [Tseng, J C, et al., Nature Biotech., 2004]. Furthermore, SV was genetically modified to be replication-defective by splitting its genome and deleting the packaging signal to block viral assembly after viral replication [Bredenbeek P J, et al., J. Virol. 1993]. This study shows that SV expressing the pro-inflammatory cytokine IL-12 (SV.IL12) activates T cells as well as enhances the expression of OX40 on CD4 T effector cells and, therefore, potentiates efficacy of the agonistic anti-OX40 antibody therapy. The data indicates that combination of SV.IL12 and anti-OX40 activates tumor immunity against low immunogenic tumors through the metabolic rewiring of T cells into highly activated effector cells. Furthermore, SV.IL12 in combination with anti-OX40 induces a marked immune cell infiltration into the tumor microenvironment. Considering that tumors tend to quickly escape the immune response by mutating or losing the expression of drug targets or tumor antigens targeted by the immune response, the treatment approach disclosed herein reduces the risk of developing tumor resistances and offers an attractive and safe strategy to change the immunogenic phenotype of various cancers without prior knowledge of tumor antigens.

The studies presented herein describe several, non-limiting examples of anti-OX-40 antibody, Sindbis viral vector (SV), Sindbis viral vector expressing IL-12 (SV.IL-12), Sindbis viral vector expressing an anti-OX-40 antibody and Sindbis viral vector expressing both IL-12 and an anti-OX-40 antibody. These examples are provided below to further illustrate different features of the present invention. The examples also illustrate useful methodology for practicing the invention. These examples do not and are not intended to limit the claimed invention.

Materials and Methods

Cell Lines

Baby hamster kidney (BHK), BALB/c colon carcinoma [CT26.WT (ATCC® CRL-2638™)] and FVB prostate carcinoma [MyC-CaP (ATCC® CRL-3255™)] cell lines were obtained from the American Type Culture Collection (ATCC). Firefly luciferase (Fluc)-expressing CT26 and MyC-CaP cells (CT26.Fluc and MyC-CaP.Fluc) were generated by stable transfection of pGL4.20_Fluc plasmid.

BHK cells were maintained in minimum essential a-modified media (a-MEM) (Corning CellGro) with 5% fetal bovine serum (FCS, Gibco) and 100 mg/ml penicillin-streptomycin (Corning CellGro). CT26.Fluc and MyC-CaP.Fluc cells were maintained in Dulbecco's modified Eagles medium containing 4.5 g/l Glucose (DMEM, Corning CellGro) supplemented with 10% FCS, 100 mg/ml penicillin-streptomycin, 7.5 μg/ml Puromycin or 400 g/ml Gentamycin, respectively. All cell lines were cultured at 37° C. and 5% C02.

SV Production

SV-LacZ production and titering were done the same as previously described [Scherwitzl I, Mal Ther Oncolytics. 2018]. SV.IL12 and SY.Lacz vectors were produced as previously described [Subramanian A et al., Proc Natl Acad Sci US A. 2005; Leonard W J et al., F1000Res. 2016; Rowell J F et al., J. Immunol. 1999; Metcalf T U et al., J. Virol. 2013]. All SV viral vectors used in these studies are replication-defective. Vectors were produced as previously described. SV.IL12 plasmid used in this study has been published in 2002 [Tseng J C et al., J Natl Cancer Inst. 2002]. To construct a Sindbis viral vector containing genes for interleukin 12 (IL-12), the Sindbis viral vector SinRep/2PSG was first constructed, which contains a secondary subgenomic promoter that is responsive to the Sindbis replicase. Two DNA oligonucleotide primers (sequence 5′ CGCGTAAAGCATCTCTACGGTGGTCCTAATAGTGCATG-3′; SEQ ID NO: 29) and its complementary strand 5′CACTATTAGGACCACCGTCGAGATGCTTTA-3′; SEQ ID NO: 30) containing the subgenomic promoter sequence were annealed and ligated into the MluI and SphI sites of the SinRep plasmid. The murine IL-12 α subunit gene (mp35; ATCC 87596) and the IL-12 β subunit gene (mp40; ATCC 87595) were subcloned into the MluI and the StuI sites of SinRep/2PSG, respectively, to produce the Sin-Rep/IL12 plasmid.

SV empty is the same plasmid without an additional gene of interest (e.g.IL12). SV.Luc was generated as described [Tseng, J C et al., Nature Biotech., 2004]. SV.GFP was generated as published in 2012 [Suzme R et al., Cancer Gene Ther., 2012]. Briefly, plasmids carrying the replicon (e.g. SinRep-IL12 or SinRep-IL-12) or DHBB helper RNAs were linearized with XhoI. In vitro transcription was performed using the mMessage mMachine RNA transcription kit (Ambion). Helper and replicon RNAs were then electroporated into BHK cells and incubated at 37° C. in αMEM supplemented with 10% FCS. After 12 hours, the media was replaced with OPTI-MEM (GIBCO-BRL) supplemented with CaCl₂) (100 mg/l) and cells were incubated at 37° C. After 24 hours, the supernatant was collected, centrifuged to remove cellular debris, and frozen at −80° C. Vectors were titrated as previously described [Tseng J C et al., J. Natl. Can. Inst., 2002].

In Vivo Experiments and Tumor Models

All experiments were performed in accordance with the Institutional Animal Care and Use Committee of New York University Health. Six to 12-week old female BALB/c mice were purchased from Taconic (Germantown, N.Y.) and age matched male FVB/NJ mice were purchased from Jackson Laboratory.

Tumor Inoculation and Animal Studies

Treatment started on day 4 after i.p. inoculation of 7×10⁴ CT26.Fluc cells or 105 cells of MyC-CaP.Fluc in 500 μl OPTI-MEM. For treatments, mice were randomized and SV (10⁷ TU/ml), in a total volume of 500 μl, was injected i.p. into the left side of the animal once for CT26.Fluc and 4 days a week (days 1, 2, 3, 4) for a total of 4 weeks for MyC-CaP.Fluc inoculated mice. The immune checkpoint inhibitor anti-OX40 (clone OX-86, BioXCell) was injected i.p. into the left side of the animal at a dose of 250 μg per injection (1×/week for the CT26.Fluc and 3×/week for MyC-CaP.Fluc tumor bearing mice). Therapeutic efficacy of the treatment was monitored in two ways: tumor luminescence and survival. Noninvasive bioluminescent imaging was performed using the IVIS Spectrum imaging system (Caliper Life Science) at the indicated time points and tumor growth was quantified using the Living Image 3.0 software (Caliper Life Science) as previously described 86. Relative tumor growth for each mouse was calculated dividing total body counts of a given day by total body counts of the first IVIS image. Survival was monitored and recorded daily.

Flow Cytometry

For flow cytometry analysis, spleens and tumors were harvested from mice and processed as previously described [Scherwitzl I, et al., Mol. Ther. Oncol, 2018]. The extracted tumors were chopped into small pieces and incubated with a digestive mix containing RPMI with collagenase IV (50 μg/ml) and DNAse I (20 U/ml) for 1 hour at 37° C. Tumor samples had additional hyaluronidase V (50 μg/ml) in the digestive mix.

Spleens and digested tumors were mashed through a 70-μm strainer before red blood cells were lysed using ammonium-chloride-potassium (ACK) lysis (Gibco). Cells were washed with PBS containing 1% FCS and surface receptors were stained using various antibodies. Fluorochrome-conjugated antibodies against mouse CD3, CD4, CD44, ICOS, OX40, CD69, Foxp3, Granzyme B and Tbet, were purchased from Biolegend. Fluorochrome-conjugated antibodies against mouse CD8a were purchased from BD Biosciences. Mitotracker Deep Red FM, Mitotracker Green and Fluorchrome-conjugated antibodies against CXCR3 and Ki67 were purchased from Thermofisher. Stained cells were fixed with PBS containing 4% Formaldehyde. For intracellular staining, the forkhead box P3 (FOXP3) staining buffer set was used (eBioscience). Flow cytometry analysis was performed on a LSR II machine (BD Bioscience) and data were analyzed using FlowJo (Tree Star).

T Cell Isolation

Total T cells were freshly isolated with the EasySep™ mouse T Cell Isolation Kit. Freshly isolated lymphocytes were depleted of either CD4 or CD8 specific T cells using EasySep™ mouse CD4 and CD8 Positive Selection Kits II. Isolation of T cells and depletions were performed according to the manufacturer's protocols (Stemcell Technologies).

Enzyme-Linked Immunospot (ELISPOT)

Enzyme-linked immunospot was performed as previously described [Scherwitzl I, et al., Mol. Ther. Oncol, 2018]. Splenocytes and T cells were prepared as described for flow cytometry. Mouse IFNγ ELISPOT was performed according to the manufacturer's protocol (BD Bioscience). Lymphocytes (4×105 cells) and isolated (8×104) T cells were plated per well overnight in RPMI supplemented with 10% FCS. No additional stimulus was used in the ELISPOT. As positive control, cells were stimulated with 5 ng/ml PMA+1 g/ml Ionomycin.

Ex Vivo Cytotoxic Assay

T cells were isolated on day 7 and day 14 during treatment. 8×105/ml T cells were co-cultured with CT26.Fluc cells (2×104/ml) or MyC-CaP.Fluc cells (2×104/ml) in a 24 well plate for 2 days in 1 ml RPMI supplemented with 10% FCS. Cells were washed with PBS and lysed with 100 l of M-PER Mammalian Protein Extraction Reagent (Promega) per well. Cytotoxicity was assessed based on the viability of CT26 cells, which was determined by measuring the luciferase activity in each well. Luciferase activity was measured by adding 100 l of Steady-Glo reagent (Promega) to each cell lysate and measuring the luminescence using a GLOMAX portable luminometer (Promega).

CD8+ and CD4+ T-Cell Depletion In Vivo

CD8+ T cells were depleted using anti-CD8 antibody (clone 2.43) (Bio X cell, Lebanon, N.H.). 0.1 mg antibody in 0.2 ml PBS was injected into each mouse, starting 1 day before the first SV treatment, and then every 4 days for 2 weeks. CD4+ T cells were depleted using anti-CD4 antibody (clone GK 1.5) (Bio X cell, Lebanon, N.H.). 0.4 mg were injected into each mouse, starting day 1 before the first treatment. Control mice were injected with PBS and isotype controls.

Quantitative Real-Time PCR

RNA was extracted from tumor samples using RNeasy Kit (Qiagen), followed by cDNA synthesis with the iScript II Kit (Bio-Rad). qRT-PCR was performed using iQ™ SYBR Green Supermix (Biorad) and an StepOne™ Real-Time PCR Detection System (Applied Biosystems). PCR conditions were as follows: 95° C. for 10 min, followed by 40 cycles (94° C. for 30 s, 58° C. for 30 s) of amplification. For quantitation, CT values were normalized to GAPDH and expression was analysed using the 2-ΔΔCT method. Primers for CXCL9, CXCL10 and GAPDH were used. CXCL9 (Forward: GAAGTCCGCTGTTCTTTTCC; SEQ ID NO: 25 Reverse: TTGACTTCCGTTCTTCAGTG; SEQ ID NO: 26), CXCL10 (Forward: GCTGCAACTGCATCCATATC; SEQ ID NO: 27; Reverse: AGGAGCCCTTTTAGACCTTT; SEQ ID NO: 28).

Transcriptome Analysis of T Cells

Total RNA was extracted from freshly isolated T cells on day 7 of treatment from spleens using RNeasy Kit (Qiagen). For each group, 3 BALB/C mice or 3 FVB/J mice were used for biological repeats. RNA-seq was done by NYUMC Genome Center. RNA quality and quantity was analyzed. RNAseq libraries were prepared and loaded on the automated HiSeq 4000 Sequencing System (Illumina) and run as single 50 nucleotide reads.

Alignment and Differential Expression Analysis

Sequences were aligned to the mm10 mouse genome using Bowtie software, Version 1.0.0⁸⁷ [Langmead R et al., Genome Biol. 2009] with two mismatches allowed. Uniquely mapped reads were further processed by removing PCR duplicates with Picard (“Picard Tools.” Broad Institute, GitHub repository. http://broadinstitute.github.io/picard/) MarkDuplicates and transcripts were counted using HTSeq⁸⁸ and differential gene expression was performed between all groups using DESeq [Anders S et al., Genome Biol. 2010]. Differences in gene expression were considered significant if padj<0.05.

GSEA and Enrichment Map Analysis

The network-based method enrichment map 90 was used for gene-set enrichment visualization and interpretation of data. As a follow up analysis of Gene-Set Enrichment Analysis2 (GSEA) [Mootha V K et al., Nat. Genet., 2003] it reduces redundancy and helps in the interpretation of large gene sets and helps to quickly identify major enriched functional themes in the gene expression data. To perform this analysis, we first assigned a unique row identifier for each transcript and obtained differentially expressed genes through DESeq [Anders S et al., Genome Biol. 2010]. These genes were then ranked and GSEA was performed in Gene Pattern 92 server using GSEA pre-ranked module. We then obtained the gene identifiers corresponding to the gene names using the Bioconductor package ‘org.Mm.eg.db’ and the resulting positively and negatively regulated gene identifiers were used to generate enrichment maps in Cytoscape [Shannon P et al., Genome Res. 2003]. Expression heatmap is drawn by Morpheus (https://software.broadinstitute.org/morpheus/). Highest and lowest expression for each gene (row min. and row max.) were displayed as red or blue color, respectively.

Measurement of Oxygen Consumption and Extracellular Acidification Rates of T Cells

T cell metabolic output was measured by Seahorse technology as previously described [Scharping N E et al., Cancer Immunol. Res., 2017]. Purified T cells were plated at 6×10⁵ cells/well in a Seahorse XF24 cell culture microplate. Oxygen consumption rate (OCR) and extracellular acidification rate (ECAR) were measured using an Agilent Seahorse XFe24 metabolic analyzer following the procedure recommended by the manufacturer (Agilent). For the mitochondrial stress test, 1) oligomycin (1 μM), 2) FCCP (1.5 μM) and 3) rotenone (100 nM) and antimycin A (1 μM) were injected sequentially through ports A, B and C.

Immunoblot Analysis

Cells were lysed in M-PER© Mammalian Protein Extraction Reagent according to the manufacturer's protocol. Lysates were separated by SDS-PAGE on 4-12% Bio-Rad gels, transferred to polyvinylidene difluoride (PVDF) membranes, blocked in 5% Milk in TBS buffer with 0.1% Tween-20 (TBST). Primary antibodies to c-Myc (Santa Cruz Biotechnology) and GAPDH (Thermofisher) were added at room temperature or overnight at 4° C. Secondary fluorescent antibodies (IRDye®, LI-COR) were added in 5% Milk in TBST for 1 h at room temperature. Odyssey® Classic Infrared Imaging System was used for visualization.

Histochemistry and Multiplex immunofluorescence (MIF)

Tumors of mice were collected, fixed in 4% PFA for 2 days and embedded in paraffin, sectioned and H&E stained. For Multiplex immunofluorescence staining and imaging, five micron paraffin sections were stained with Akoya Biosciences® Opal™ multiplex automation kit on a Leica BondRX® autostainer, according to the manufacturers' instructions. Prior to incubation with the first primary antibody, sections underwent heat retrieval with Bond Epitope Retrieval Buffer 2 (Leica ER2, AR9640) and blocking. Primary antibodies in Panel 1 were against CD3 (1:200, Biorad, MCA1477T), CD8 (1:2000, Cell Signaling, 98941S), Ki67 (1:200, Abcam, AB16667). Primary antibodies in Panel 2 recognized iNos (1:1000, Genetex, GTX130246), Arg1 (1:750, Genetex, GTX109242), Granzyme B (1:1000, Abcam, AB4059), CD11b (1:10,000, Abcam, AB133357), F480 (1:250, Cell Signaling, 70076S). Each primary antibody was followed by a cocktail of horse radish peroxidase-conjugated secondary antibodies against mouse and rabbit IgG (RTU, Akoya/Perkin Elmer, Cat # ARH1001) and then tyramide mediated signal amplification (TSA) with covalent linkage of the individual Opal fluorophor (each 1:250, Opal 520 (FP1496001KT), 540 (FP1487001KT), 570 (FP1494001KT), 620 (FP1488001KT), 650 (FP1495001KT) or 690 (FP1497001KT), Akoya/Perkin Elmer Cat #'s) to the tissue antigen. Antibodies were subsequently stripped using either ER1 (Leica, AR9961) or ER2 (Leica, AR9640) heat retrieval buffer and the next round of immunostaining initiated. After completion of the sequential incubations and stripping, slides were counterstained with spectral DAPI (Akoya/PerkinElmer, FP1490). Monoplex controls were used to confirm appropriate staining for antibodies integrated into the multiplex panels. Multispectral imaging was performed on a Vectra3 imaging system (Akoya/PerkinElmer) at 20×. The fluorophore emission signatures were captured by a multispectral camera and then unmixed with InForm software (Akoya/PerkinElmer). Autofluorescence, obtained from an unstained slide, was removed from the composites and pseudo-colored images exported as tif files.

Statistical Analysis

Statistical analysis was performed using GraphPad Prism 7.0 as described in Figure legends. All data are shown as mean±s.e.m. Figures were prepared using GraphPad Prism 7, Adobe Photoshop and ImageJ Software. Treated groups were compared, using a one-way analysis using Prism7 (GraphPad Software), to naïve mice. Differences with a P value of <0.05 were considered significant: *P<0.05; **P<0.005; ***P<0.0001.

SV Expressing IL-12 Enhances the Expression of OX40 on CD4 T Cells

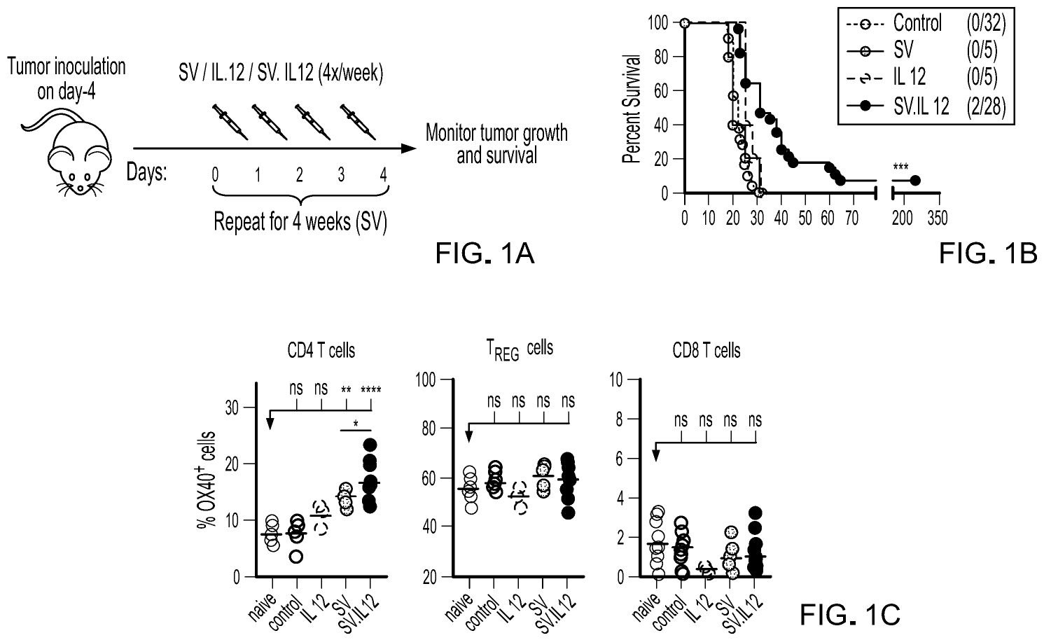

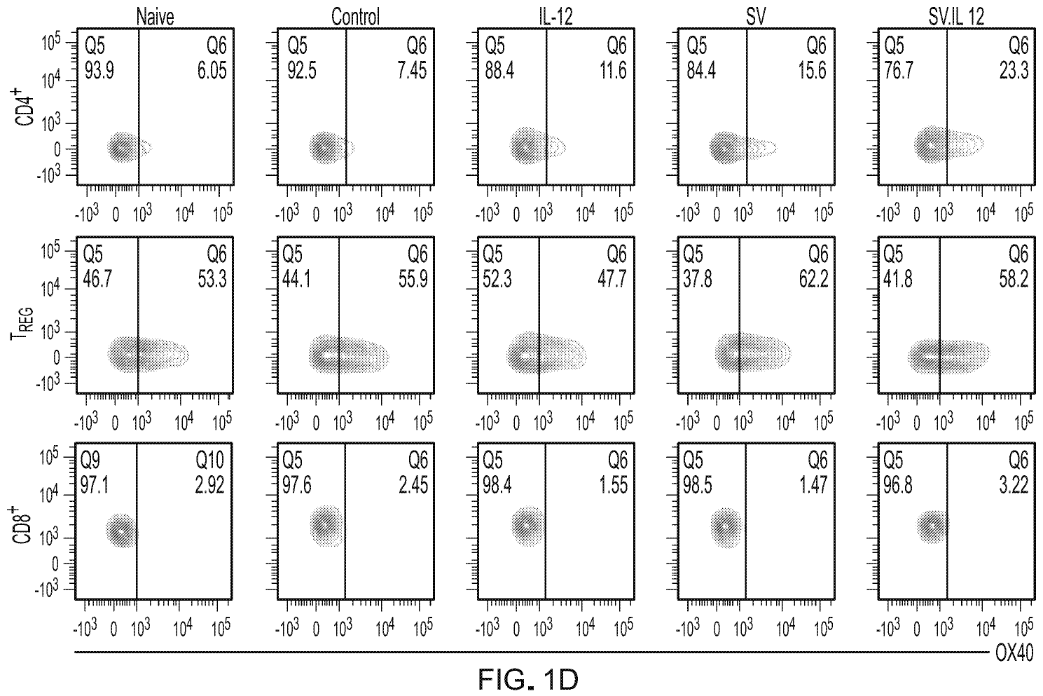

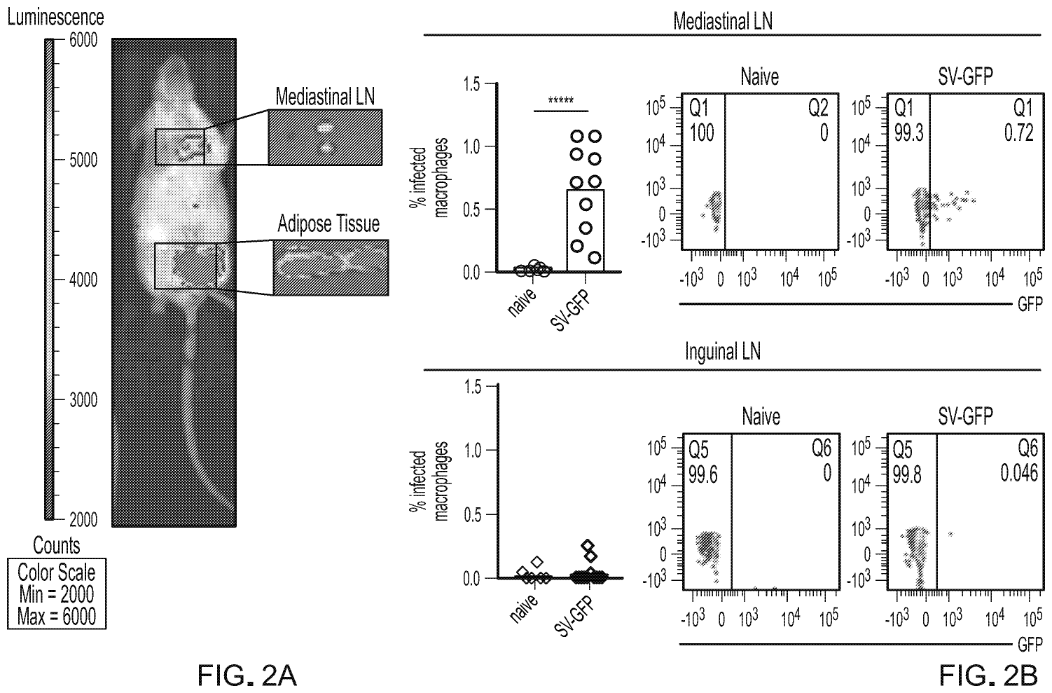

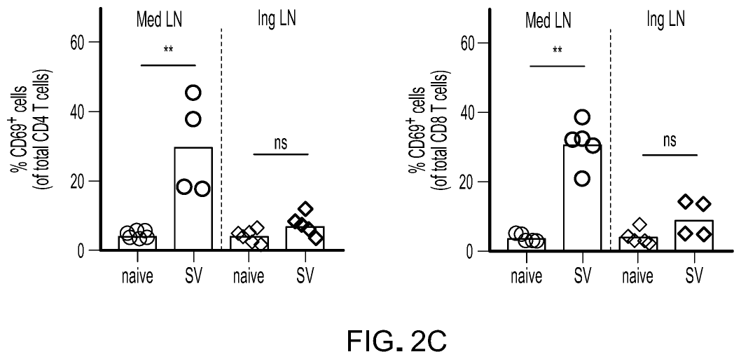

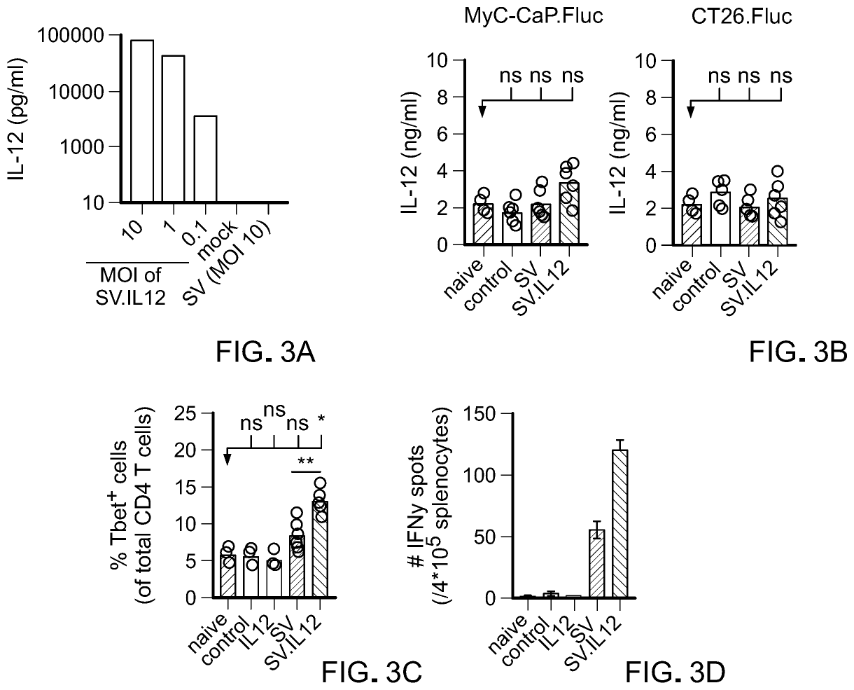

The study described herein investigated the therapeutic effect of SV.IL12 in immune-competent tumor bearing mice (colon cancer; CT26). To exploit SV.IL12 for cancer therapy, tumor cells were i.p. implanted and after tumor establishment (4 days after tumor cell injection [day 0]), SV, SV.IL12 or IL-12 were i.p. injected on 4 consecutive days (day 1, 2, 3 and 4) for a total of 4 weeks (FIGS. 1A and B). While untreated (control), SV and IL-12 treated mice succumb to cancer after 3 weeks, treatment with SV.IL12 slightly prolonged survival time with an overall long-term survival rate of 7.1%. These data suggest that SV expressing IL-12 is needed to induce the observed therapeutic efficacy. Shortly after i.p. injection, SV infects macrophages in mediastinal lymph nodes where T cells get subsequently activated (FIG. 2). Even though SV.IL12 infected cells secrete significant amounts of IL-12 as observed in in vitro experiments (FIG. 3A), i.p. injection of SV.IL12 did not significantly change levels of plasma IL-12 in mice (FIG. 3B). Thus, suggesting that IL-12 produced by SV acts locally and stimulates transduced macrophages (FIG. 2) that present tumor antigens to corresponding T cells and activates them further. That shapes the subsequent anti-tumor immune response, such as promoting the differentiation into Th1 cells as well as increasing IFNγ production (FIGS. 3C and 3D)^{27, 28, 29, 30}. After one week of treatment, we analyzed the T cell response for their expression of inhibitory and activation markers. OX40 was markedly upregulated on CD4 T cells during SV.IL12 treatment, which was mostly among the effector CD4 T cells and less on the regulatory T cells (FIGS. 1C and 1D). Interestingly, SV treatment also induced OX40 upregulation on CD4 T cells but to a lesser extent (FIGS. 1C and 1D). On the basis of the results above and previous studies, reporting a beneficial effect of anti-OX40 in cancer treatment [Aspeslagh S et al., Eur. J Cancer, 2016], it was hypothesized that the agonistic anti-OX40 antibody could augment the therapeutic efficacy of SV.IL12 and help induce anti-tumor immune responses without to require knowledge of the tumor antigens.

Intraperitoneal Delivery of SV.IL12 and Anti-OX40 Antibody Cures Established Cancers

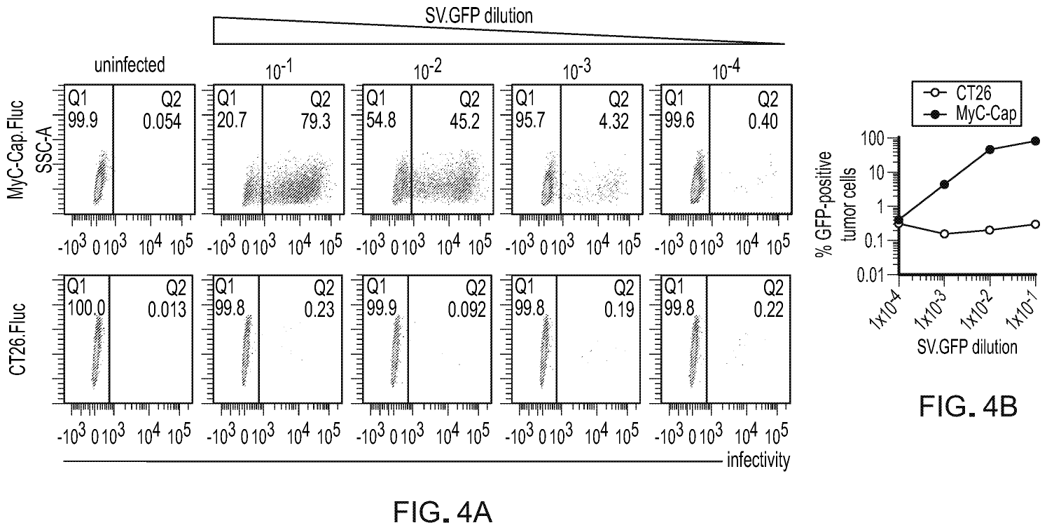

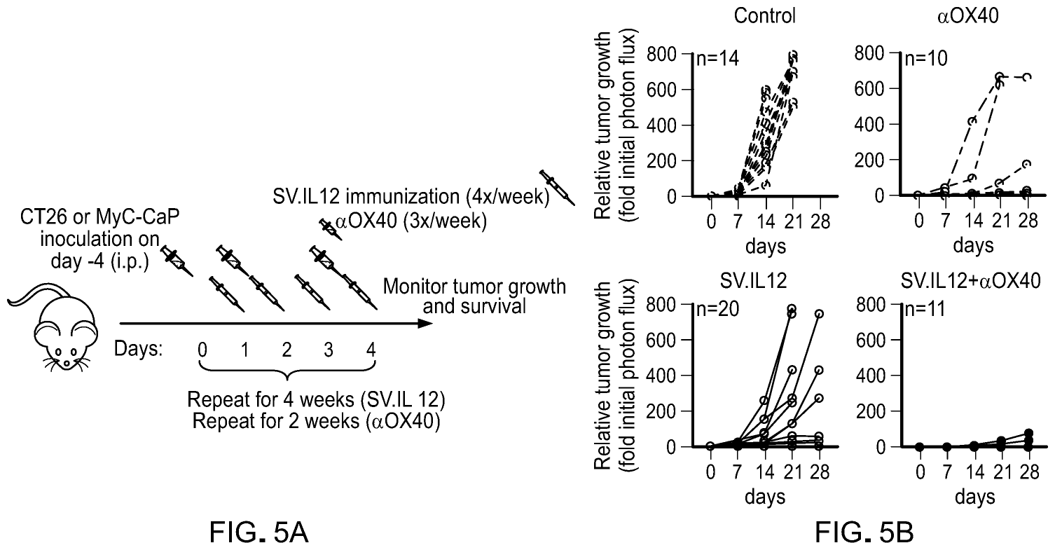

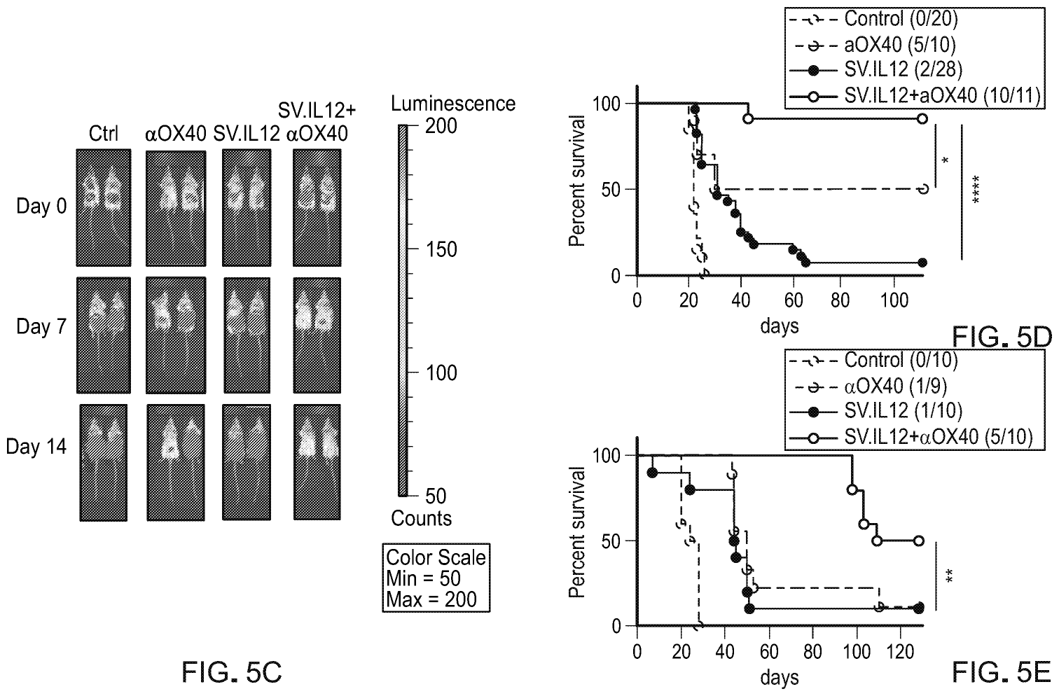

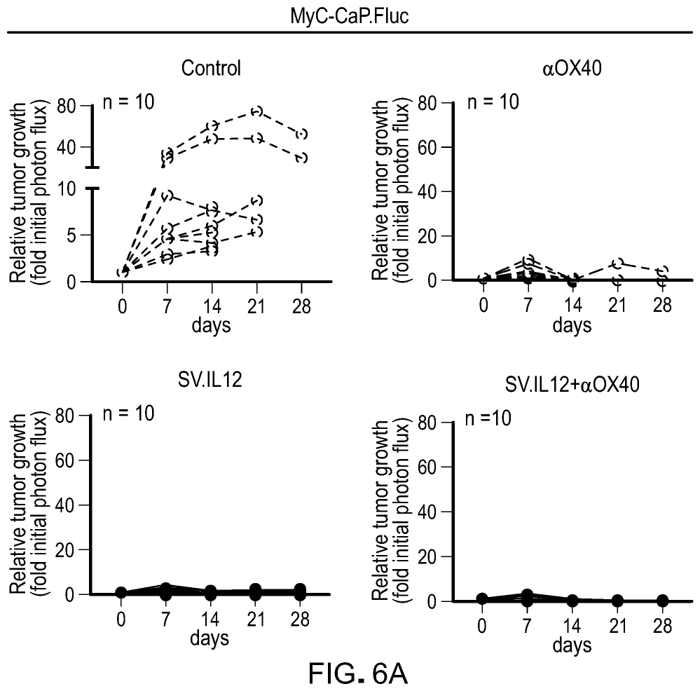

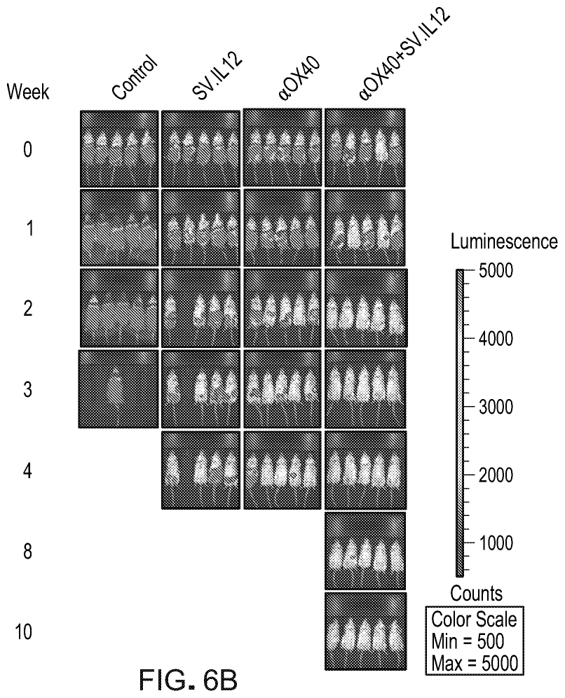

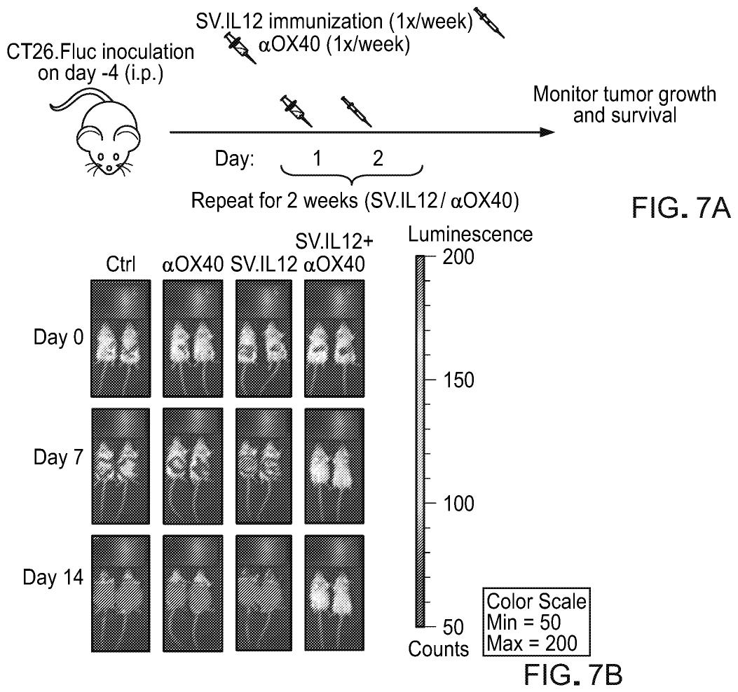

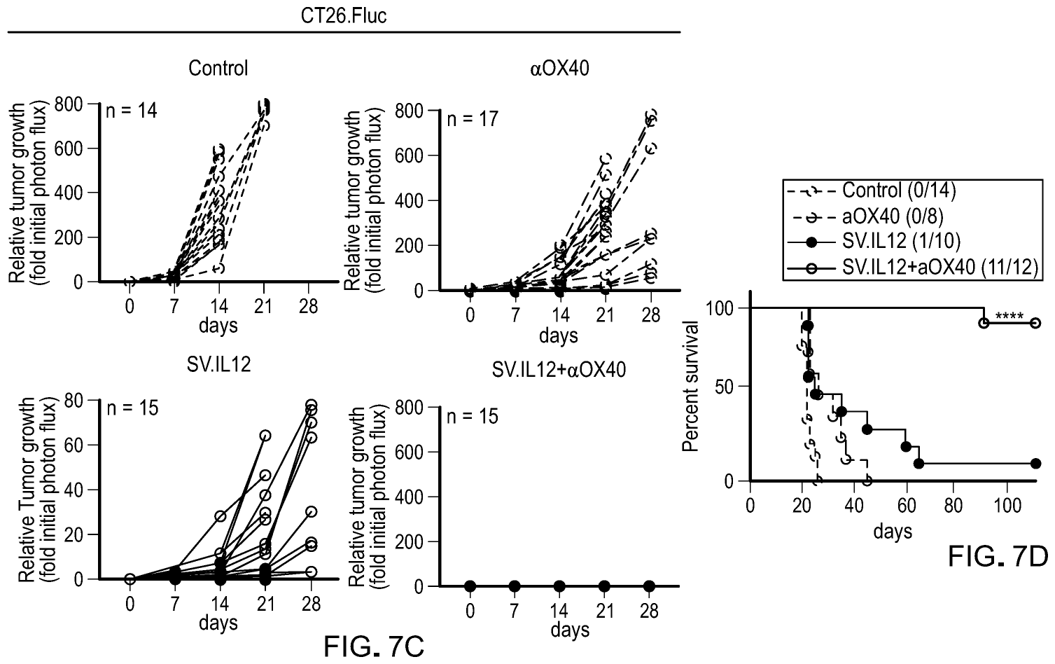

To investigate whether the oncolytic activity of SV.IL12 in combination with anti-OX40 is required for successful anti-cancer therapy, SV non-susceptible (colon cancer; CT26) and susceptible (prostate cancer; MyC-CaP) tumor cell lines were used in this study (FIG. 4) [Granot T et al., Mol. Ther., 2014; Huang P Y et al., Mol. Ther., 2012] Immuno-competent female BALB/c and male FVB/NJ mice were implanted with either CT26 or MyC-CaP tumor cell lines, which expressed the firefly luciferase (Fluc) protein, respectively. This allowed monitoring tumor growth in vivo using noninvasive bioluminescent imaging. Once tumors became established (day 0), mice were treated with SV.IL12 in combination with anti-OX40. SV.IL12 was i.p. injected on 4 consecutive days (day 1, 2, 3 and 4) for a total of 4 weeks (FIG. 5A). Anti-OX40 was injected 3 times a week (day 0, 2 and 4) for a total of 2 weeks. In both tumor models, all untreated animals experienced progressive tumor growth and succumbed to cancer on week 3 (FIGS. 5 and 6). Mice bearing CT26.Fluc or MyC-CaP.Fluc tumors showed some delay in tumor growth when treated with i.p. injected SV.IL12 or anti-OX40 alone but with only a moderate effect on long-term survival (FIGS. 5 and 6). However, the combination of SV.IL12 with anti-OX40 resulted in complete regression of tumors in both tumor models (FIGS. 5 and 6). Tumors occasionally did recur in mice treated with combination therapy after treatment was completed, resulting in a long-term survival rate of 91.6% and 50% in the CT26 and MyC-CaP tumor model, respectively. In conclusion, combination of SV.IL12 with anti-OX40 elicits a strong therapeutic efficacy against two distinct solid tumors. Furthermore, these findings confirm that the oncolytic activity of SV is not required to induce a robust and effective anti-tumor response. Due to the fact that anti-OX40 monotherapy already resulted in a 20-50% survival rate, whether the addition of SV.IL12 would allow reduction of treatment frequencies while still maintaining the strong therapeutic efficacy of combination therapy, was investigated. This is especially important for lowering risks of adverse events as well as being more convenient for patients in clinics. Interestingly, therapeutic efficacy in the CT26.Fluc tumor model was maintained with only one injection per week of SV.IL12 and anti-OX40 (FIG. 7). This is in contrast with MyC-CaP.Fluc tumor bearing mice for which the full treatment regimen was required (data not shown). Thus, in the following experiments mice bearing CT26 tumors were treated with one injection a week whereas MyC-CaP tumor bearing mice received the full treatment regimen.

Combination Therapy Markedly Changes the Transcriptome Signature of T Cells

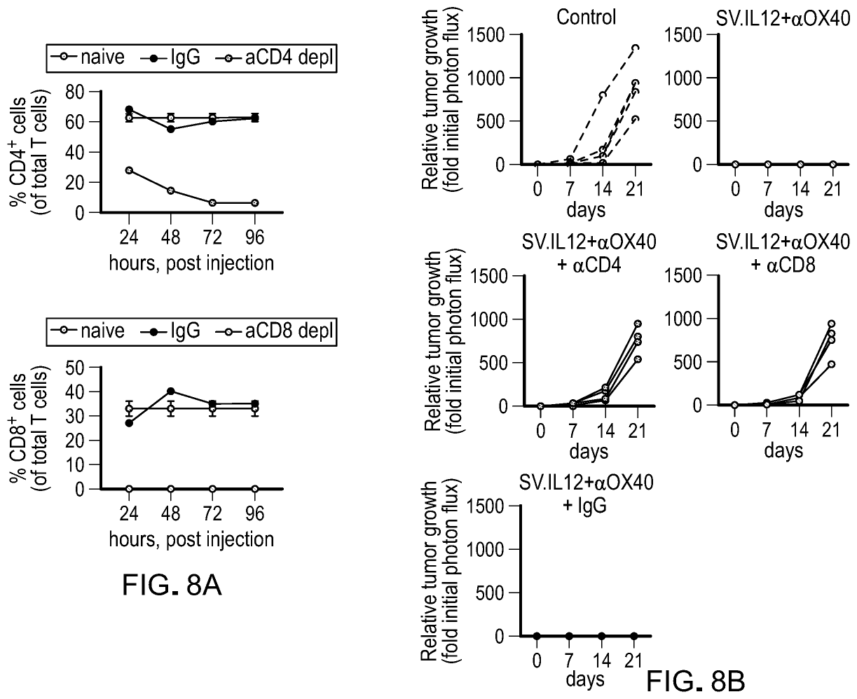

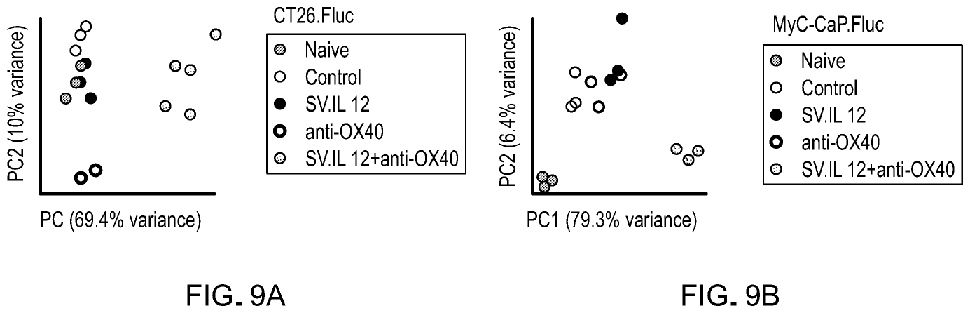

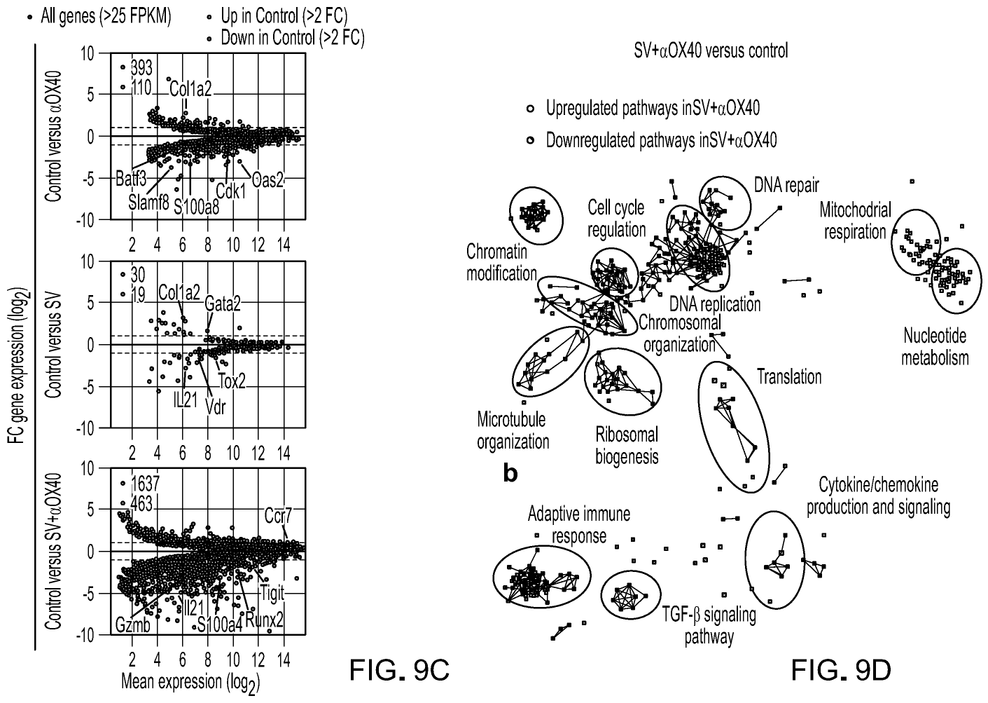

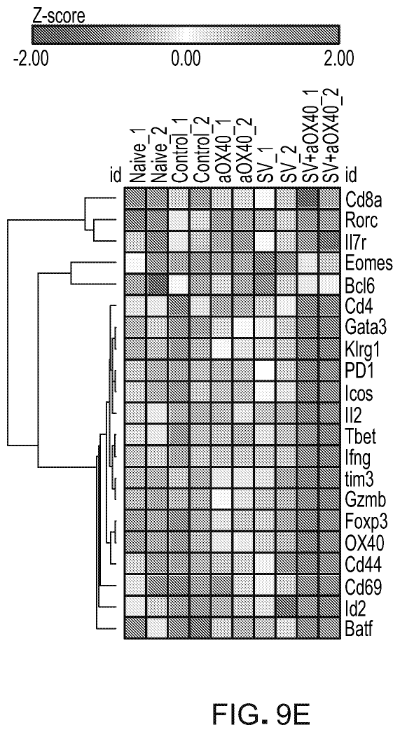

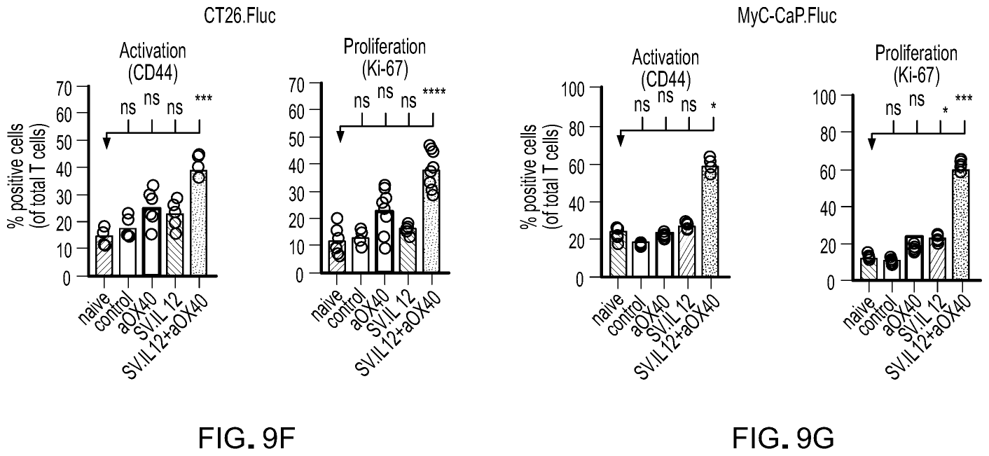

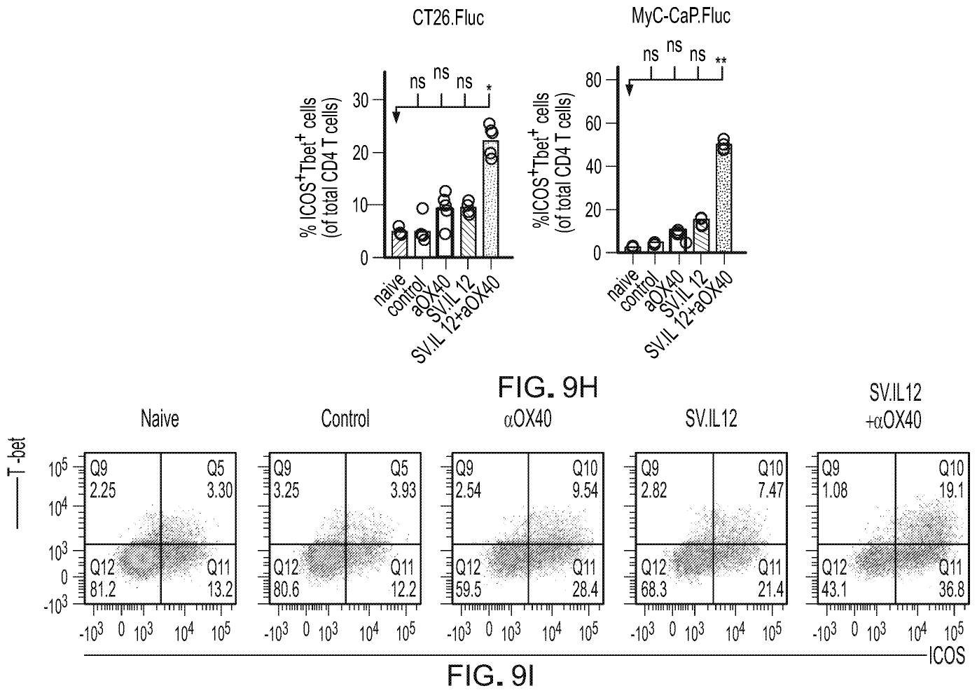

The requirement of T cells during SV.IL12 with anti-OX40 treatment was assessed. The presence of both CD4 and CD8 T cells was required for eliciting the observed therapeutic efficacy as mice treated with the corresponding depleting antibodies were unable to control tumor growth (FIG. 8). To better understand the impact of the combination therapy on T cells, RNA sequencing was performed on isolated T cells from spleens derived from naïve, control as well as anti-OX40 and SV with or without anti-OX40 treated mice on day 7. Principal component analysis (PCA) of normalized reads showed a distinct segregation between combined therapy and all other groups in both tumor models (FIGS. 9A and 9B). These data suggest that combined therapy induces a distinct T cell response in the periphery independently of tumor model and mouse strain suggesting an indirect and immunity driven role of SV vectors with a negligible direct effect of the vector in tumors (FIG. 9C). Indeed, gene expression profiles of control versus anti-OX40, SV or SV in combination with anti-OX40 were clearly distinct, with the most differentially expressed genes (DEG) in the latter one (503, 49 and 2100 DEG, respectively) (FIG. 9C). Of 2100 DEG in control versus combination therapy, more genes were downregulated than upregulated (1637 vs. 463)>two-fold. Similarly, in control versus anti-OX40 as well as control versus SV more DEG were downregulated than upregulated (393 versus 110 and 30 versus 19, respectively).

Unbiased pathway enrichment and network analyses of DEG from control versus combination therapy was performed to determine biological processes in T cells that are influenced by this treatment (FIG. 9D). Although both upregulated and downregulated DEG were included in the analysis, the vast majority of pathways were upregulated in T cells treated with combination therapy with the exception of four clusters (TGFbeta signaling, ribosomal biogenesis, translation and chromatin modification). The upregulated pathways were dominated by DNA replication, chromosomal organization and cell cycle regulation, but also included various metabolic and immunological processes, such as mitochondrial respiration, nucleotide metabolism, and adaptive immune responses.

Combination Therapy Enhances Systemic T Cell Responses, Favoring Th1 Like ICOS CD4 T Cells

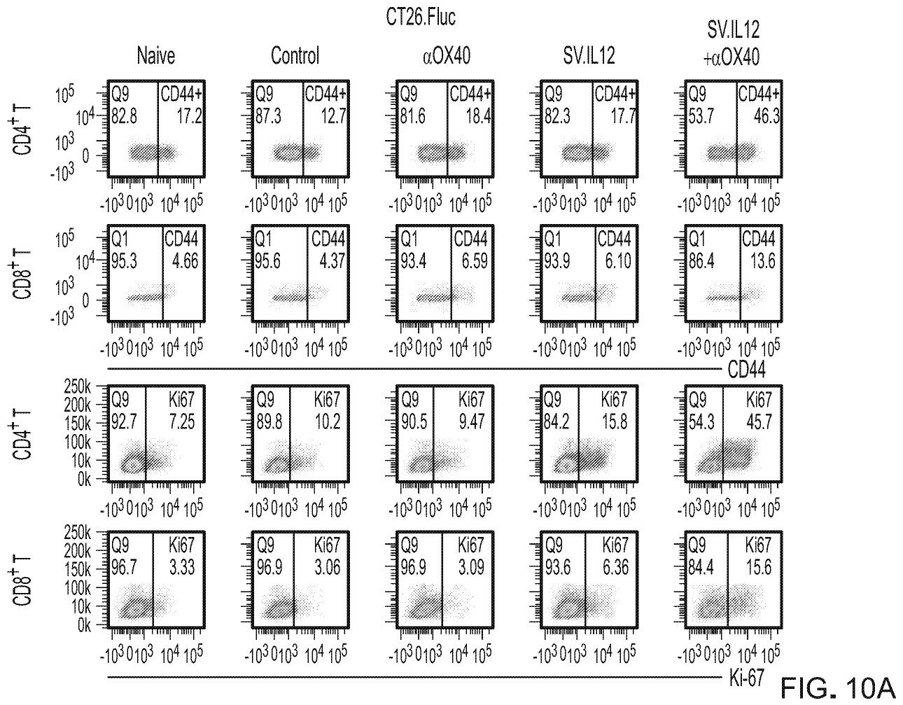

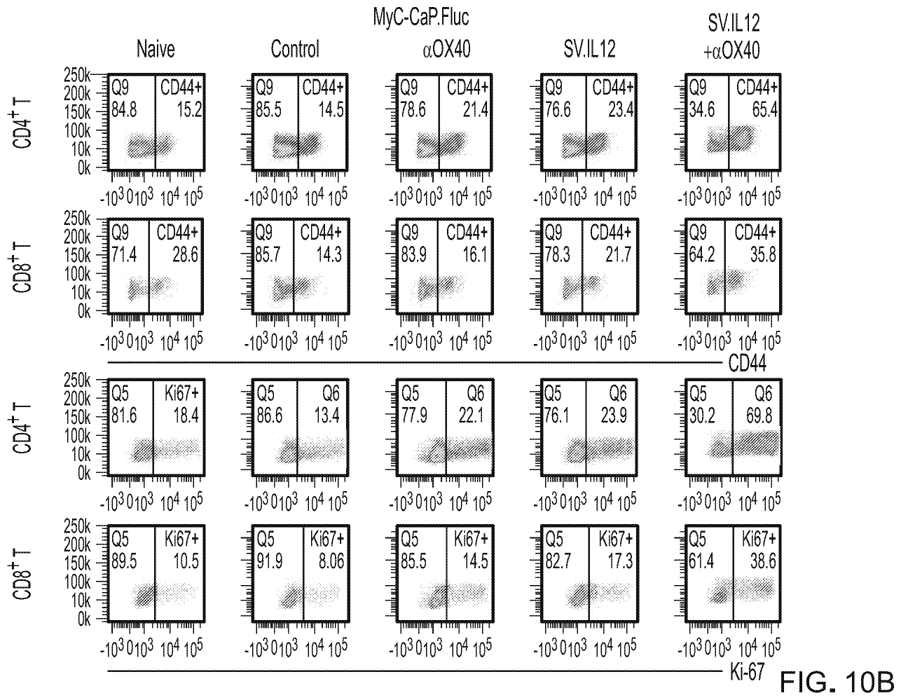

As T cells from combination therapy express a marked change in their transcriptome signature compared with all other groups, markers for T cell differentiation and activation (e.g., PD-1, ICOS, OX40, TIM3, KLRG1, IL7R) as well as T cell lineage transcription factors (e.g., EOMES, TBET, GATA3, BCL6, RORC, FOXP3) were analyzed (FIG. 9E). Only T cells from combined therapy expressed the gene signature of terminally differentiated effector T cells, which are characterized by high expression of the killer lectin-like receptor (KLRG1) and low expression of the interleukin 7 receptor (IL-7R) [Joshi N S et al., Immunity, 2007]. Furthermore, genes encoding products associated with the differentiation and function of effector cells, such as Batf, Id2, Tbet, Gzmb and Ifng, were also highly expressed in T cells isolated from mice treated with combined therapy compared with all other groups. The enhancement of effector T cells in combined therapy was confirmed by flow cytometry in both tumor models, as judged by the increased expression of the activation and proliferation markers CD44 and Ki-67, respectively (FIGS. 9F, 9G and 10). Interestingly, CD4 T cells also expressed a marked anti-tumor effector phenotype (ICOS⁺Tbet⁺) which was on average 2 to 3-fold higher during combined therapy compared with SV.IL12 or anti-OX40 treatment (FIGS. 9H and 9I). Previous studies reported a correlation between expansion of ICOS⁺Tbet⁺ CD4 T cells and clinical benefit in cancer patients who received anti-CTLA4 therapy [Wei S C et al., Cell, 2017; Ng Tang G et al., Cancer. Immunol. Res. 2003; Carthon B C et al., Clin. Cancer Res., 2010]. In summary, SV.IL12 in combination with anti-OX40 induces a marked systemic T cell response and favors the differentiation of terminal effector T cells. Furthermore, combined therapy induces a sustained increase in the frequency of ICOS⁺Tbet⁺ CD4 T cells which has also been reported to be elevated during successful anti-CTLA4 cancer therapy.

CD4 and CD8 T Cells are Metabolically Reprogrammed in Mice Treated with SV.IL12 and Anti-OX40

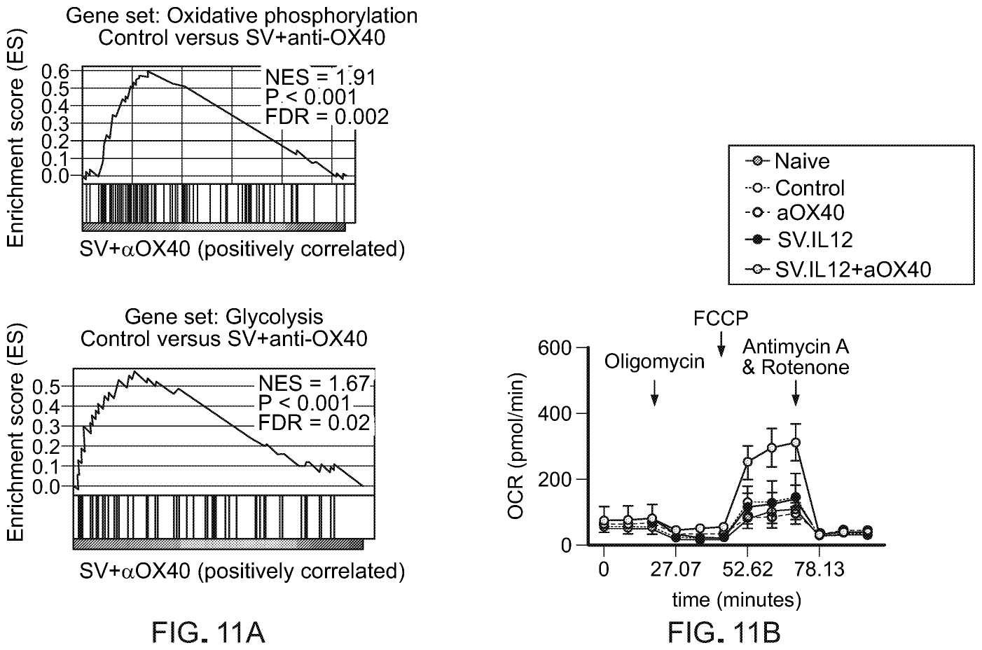

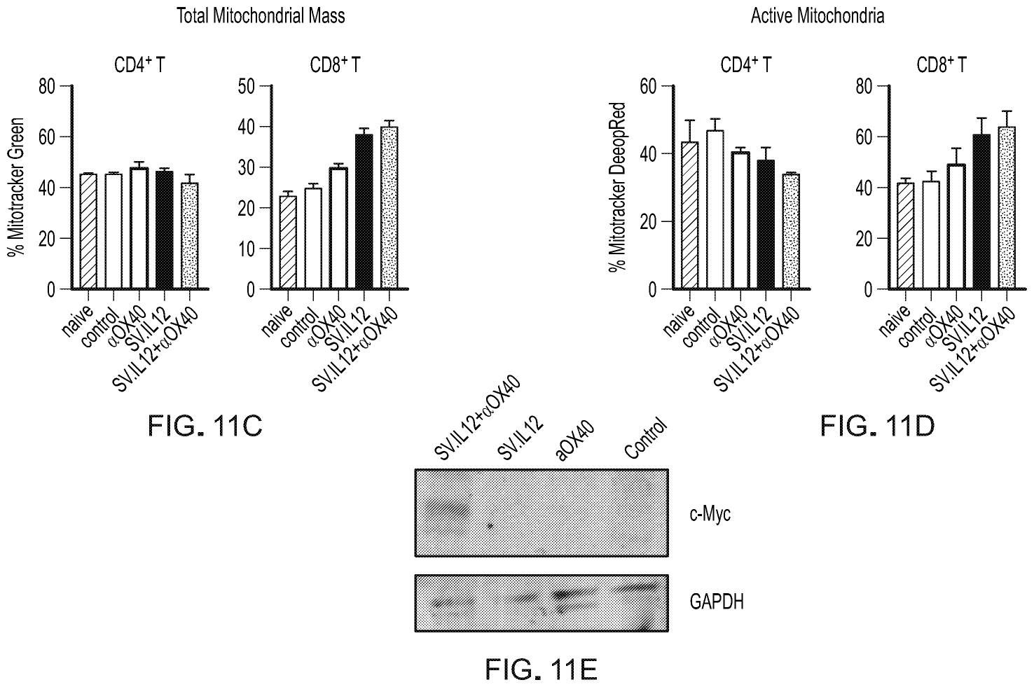

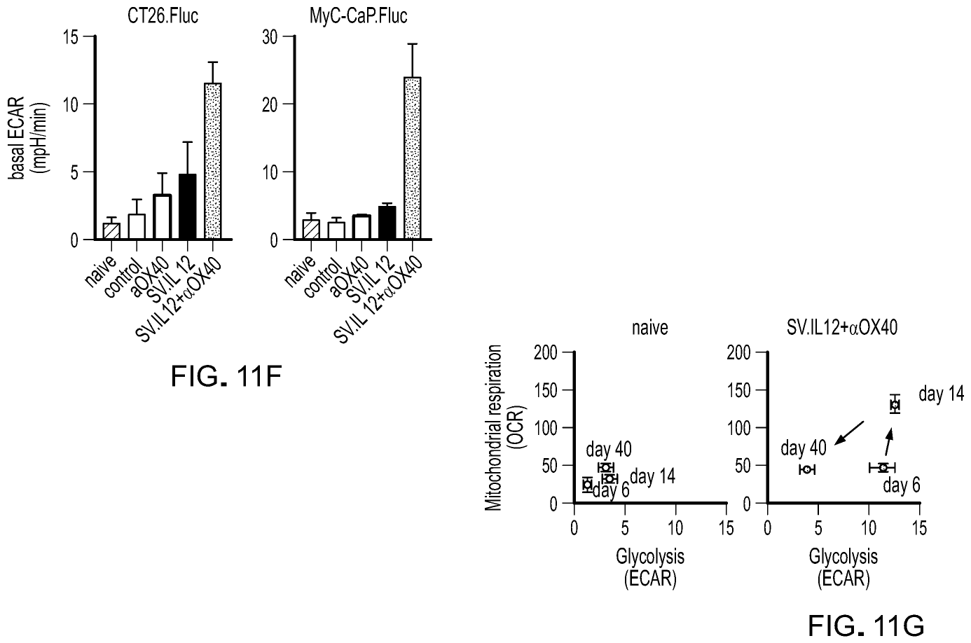

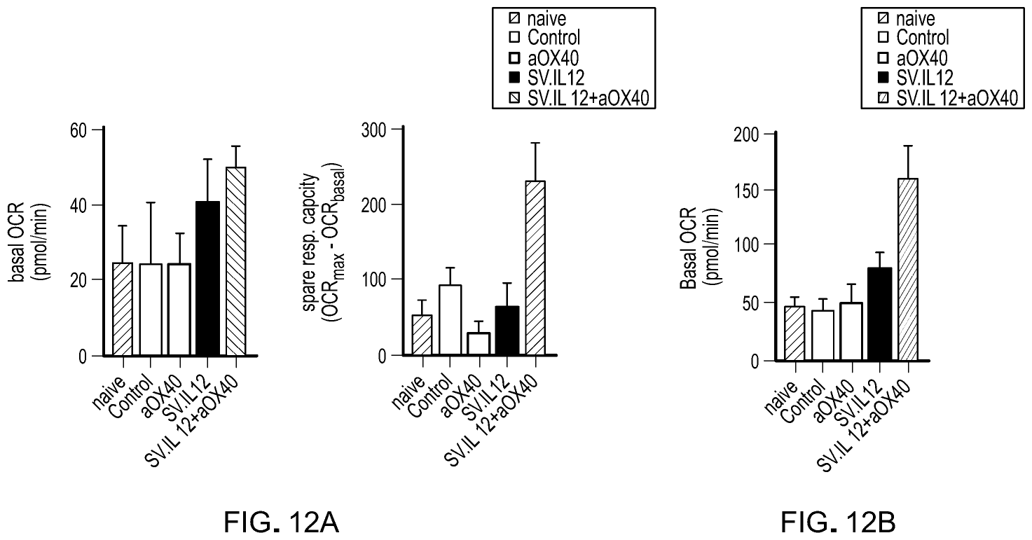

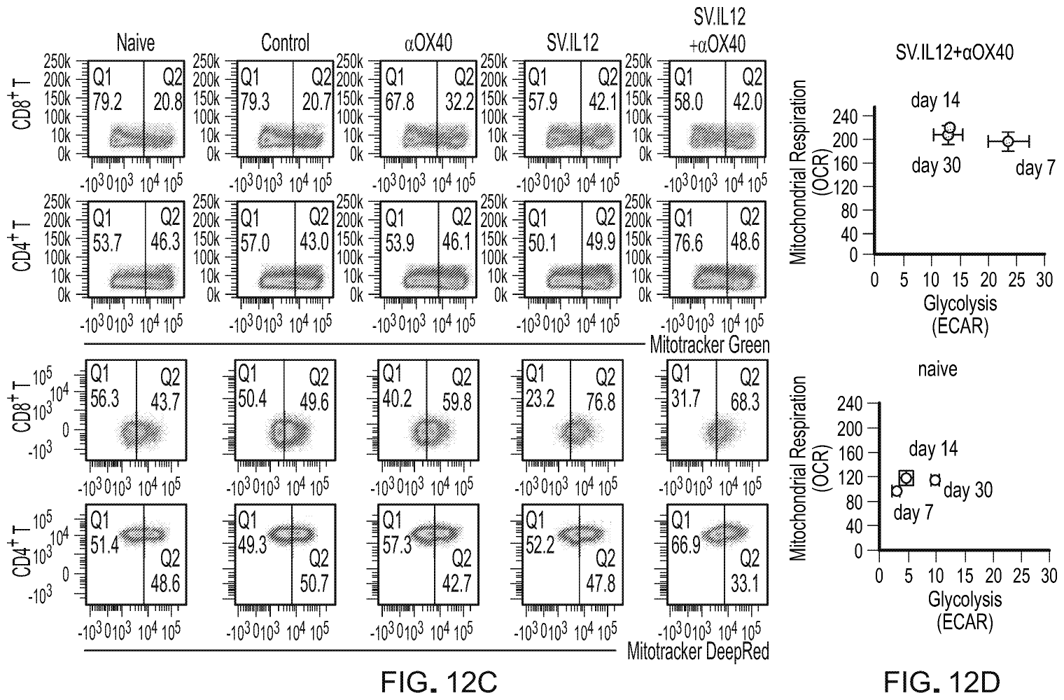

The tumor microenvironment can be a very challenging milieu for an effector T cell as it is characterized by hypoxia, acidosis and low levels of nutrient sources such as glucose and glutamine [Delgoffe, G M et al, Cancer Immunol. Res., 2016; Scharping, N. E, Vaccines, 2016; Chang, C H et al., Cell, 2015]. Even if T cell activation and initiation of effector function is allowed, T cells may be unable to generate the bioenergetic intermediates necessary to carry out effector function in the tumor microenvironment. Thus, providing a metabolic support for T cells is crucial for the success of cancer treatments as previously reported [Scharping, N. E. et al., Immunity, 2016; Ho, P C et al., Cell, 2015; Siska P J., et al, Trends immunol., 2015; Zhao et al., Nat. Immunol., 2016]. To test if SV.IL12 in combination with anti-OX40 influences the metabolic state of T cells, Gene Set Enrichment Analysis (GSEA) of the RNA sequencing data was performed between T cells from combined therapy and control. GSEA analysis showed significantly higher expression of genes involved in oxidative phosphorylation and glycolysis pathways during combination therapy (FIG. 11). To confirm GSEA analysis, peripheral T cells from both tumor models were metabolically profiled using Seahorse analysis on day 7 (FIGS. 111B, 12A and 12B). Oxidative phosphorylation and glycolytic profiles in T cells from naïve, control and mice treated with SV.IL12 and/or anti-OX40 were determined by measuring the rate of oxygen consumption (OCR) and the rate of extracellular acidification (ECAR), respectively. Basal OCR was enhanced in T cells from combined therapy and SV.IL12 treatment, but only the former harbored a dramatic increase in spare respiratory capacity in the CT26 model (FIGS. 11B and 12A). This was in contrast to T cells from combined therapy in the MyC-CaP.Fluc tumor model which expressed 3.75-fold higher basal OCR with no spare respiratory capacity (FIG. 12B). The reason for this discrepancy between the two models might be the differences in the number of treatments as MyC-CaP.Fluc bearing mice receive 3 and 4 times more injections of anti-OX40 and SV.IL12, respectively.

Analysis of mitochondrial mass (FIGS. 11C and 12C) and activity (FIG. 11D and FIG. 12C), using flow cytometry with the mitochondrial stain Mitotracker Green and DeepRed respectively, revealed that SV.IL12 with or without anti-OX40 induced higher mitochondrial mass and activity in CD8 T cells but not in CD4 T cells. These data suggest that the observed increase in basal OCR was mainly driven by CD8 T cells. Interestingly, a slight decrease of active mitochondria occurred in CD4 T cells from mice treated with combined therapy, which might explain the increase in spare respiratory capacity in this group. To test if the reduction of active mitochondria in CD4 T cells is associated with a switch towards glycolysis, the master regulator for glycolysis c-MYC and basal ECAR were measured in T cells from all groups. Indeed, the addition of anti-OX40 to SV.IL12 induced elevated protein expression of c-MYC as well as basal ECAR (FIGS. 11E and 11F). T cells from naïve and control as well as SV.IL12 or anti-OX40 treated mice showed no signs of elevations. Collectively, these findings reveal that SV.IL12 induces enhanced oxidative phosphorylation in CD8 T cells, whereas the addition of anti-OX40 to SV.IL12 is needed to push CD4 T cells towards glycolysis by increasing the protein expression of c-MYC.

To determine the kinetics of peripheral T cell metabolism over the course of treatment with SV.IL12 and anti-OX40, OCR and ECAR were measured on day 7, 14 and 40 in CT26.Fluc bearing mice (FIG. 11G). As shown above, T cells on day 7 shifted towards a glycolytic state which is associated with the initial effector phase. Two weeks into the treatment, T cells switched to a highly energetic state utilizing both metabolic pathways, oxidative phosphorylation and glycolysis, as reported for highly activated T cells [Buck M D et al., J. Exp. Med., 2015] Once tumors were fully rejected and mice were tumor-free for a month, T cells returned to a more quiescent state, such as naïve cells. Interestingly, T cells from MyC-CaP.Fluc bearing mice switched to a highly energetic state early on during treatment (day 7) and remained in this metabolic phenotype 2 weeks after treatment has stopped (FIG. 12D). The reason for this discrepancy might be the differences in the number of treatments applied in both tumor models as MyC-CaP.Fluc bearing mice receive 3 and 4 times more anti-OX40 and SV.IL12, respectively. T cells from control as well as anti-OX40 or SV.IL12 treated mice in both tumor models remained in a quiescent state over the course of treatment (FIG. 12E). In summary, SV.IL12 in combination with anti-OX40 metabolically rewires T cells to an energetic state using both metabolic pathways, oxidative phosphorylation and glycolysis. This phenotype does not occur in SV.IL12 or anti-OX40 treated mice, which succumb to cancer. Thus, the changed metabolic state of T cells correlate with an efficient anti-tumor response and better survival rate.

Metabolic Reprogrammed T Cells in SV.IL12 with Anti-OX40 Treated Mice Display Enhanced CD4 Mediated Cytokine Production and Anti-Tumor Activity

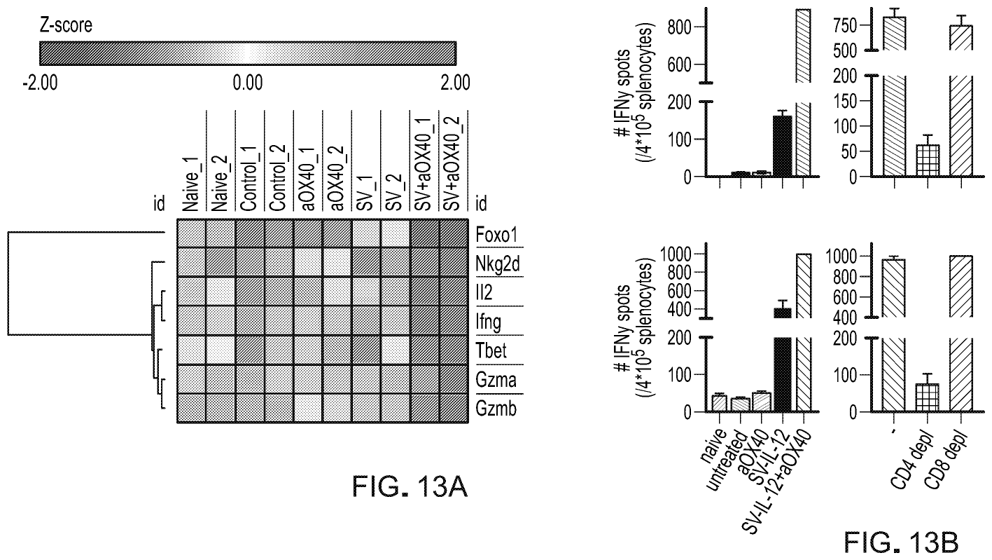

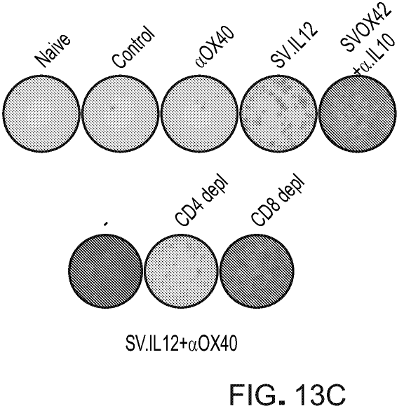

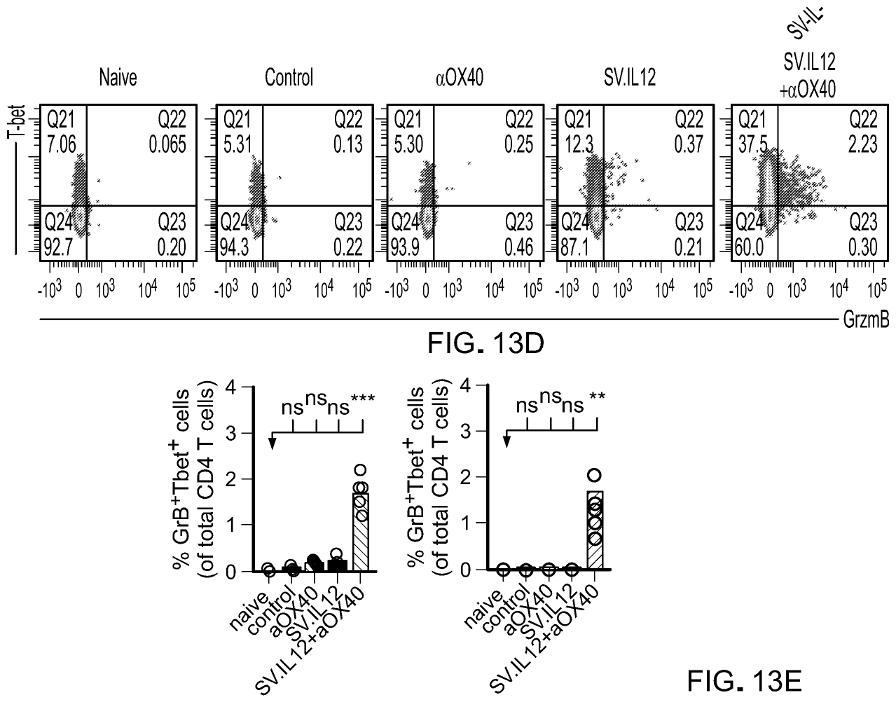

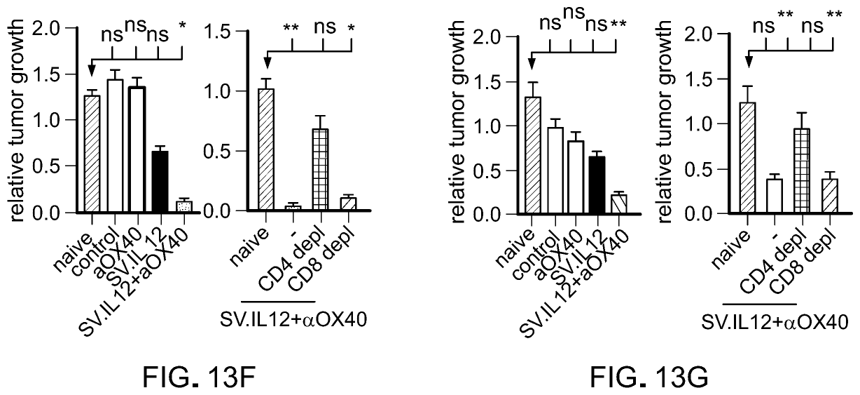

To test if metabolic reprogrammed T cells in combined therapy possess enhanced effector functions, cytokine production and cytotoxicity were analyzed in T cells isolated from spleens on day 7. Genes encoding pro-inflammatory cytokines ifng and il2 were upregulated in T cells from mice treated with SV in combination with anti-OX40 (FIG. 13A). ELISPOT analysis of interferon-γ (IFNγ) by splenocytes confirmed RNA sequencing data, showing the strongest IFNγ secretion in mice treated with combined therapy in both tumor models (FIGS. 13B and 13C). Splenocytes from SV.IL12 treated mice also produced IFNγ but to a lesser extent. Interestingly, the main producer of IFNγ were CD4 T cells as depletion of CD4 T cells but not CD8 T cells abolished IFNγ secretion in splenocytes from mice treated with combined therapy.

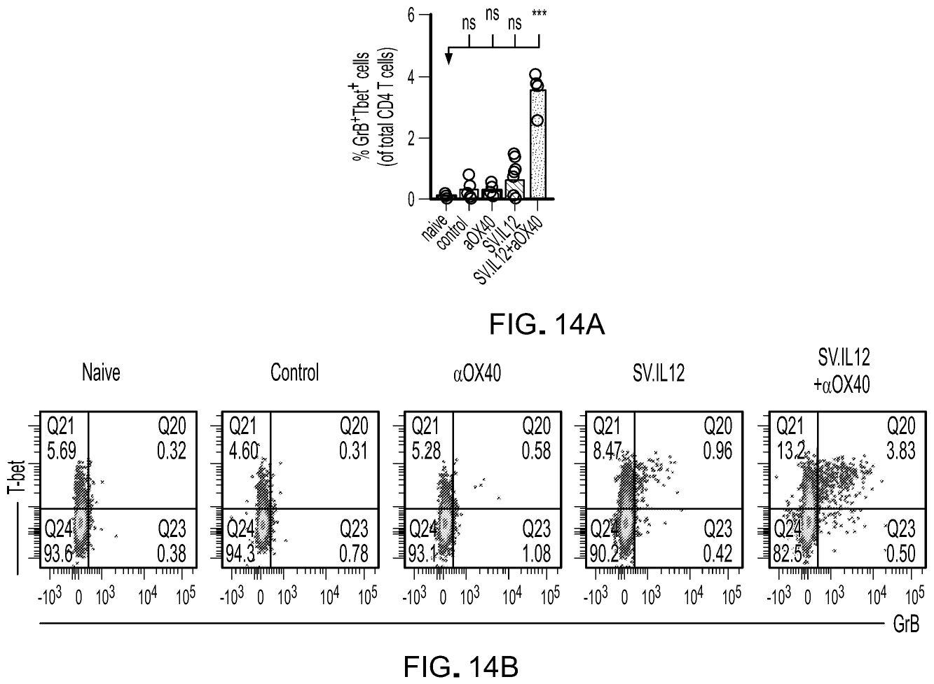

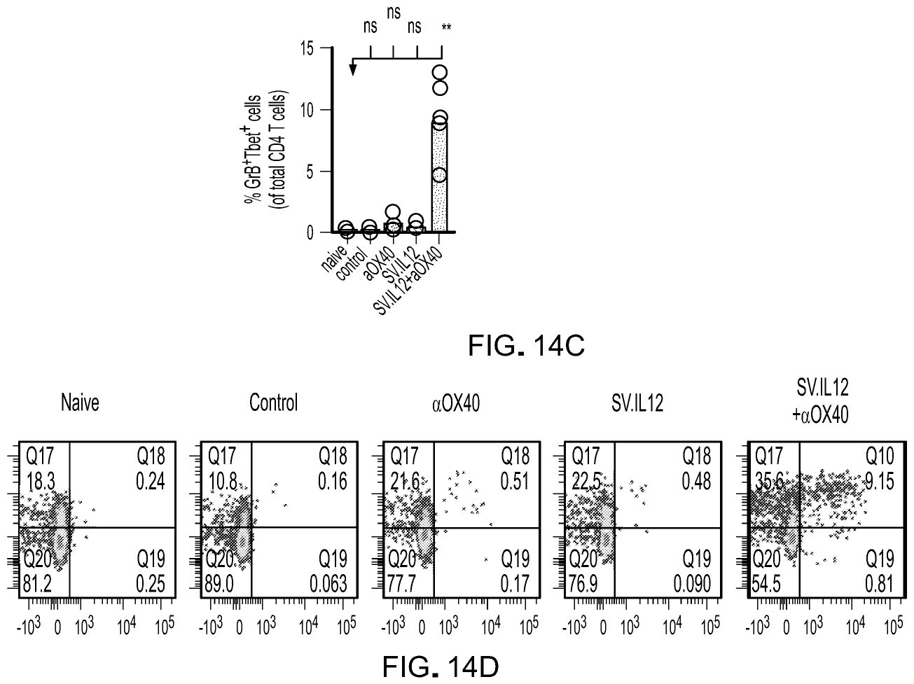

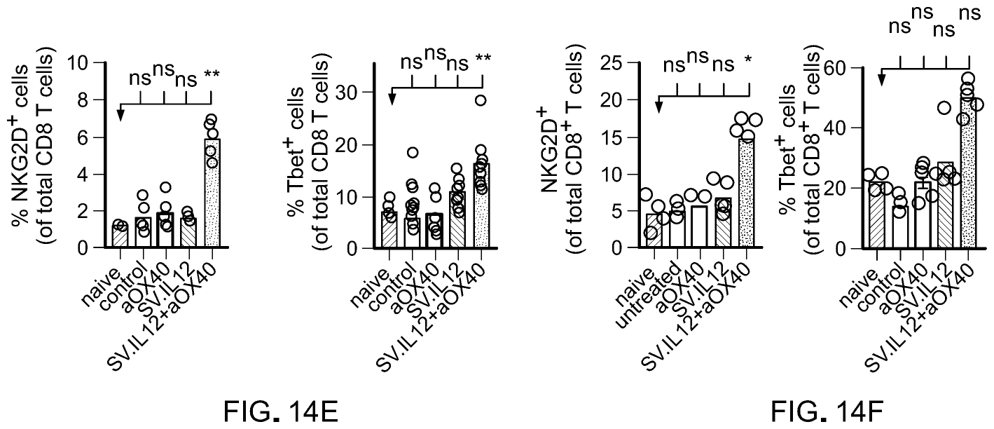

In addition, RNA levels of the cytotoxic proteases, granzyme A and B, were upregulated in mice treated with combination therapy compared with all other groups (FIG. 13A). Protein expression of granzyme B correlated with RNA levels as measured by flow cytometry in both tumor models (FIGS. 13D, 13E, and 14A-14D). Further, granzyme B positive cells were detected in CD8 as well as CD4 T cells, suggesting the presence of cytotoxic CD4 T cells in mice treated with combined therapy [Brown D M et al., Cell Immunol, 2010; Mucida D. et al., Nat Immunol, 2013; Reis B S et al., Nat Immunol 2013] Upregulation of granzyme B was associated with downregulation of the transcription factor Foxo1 which is known to control granzyme transcription through repression of the transcription factor T-bet (FIG. 13A) [Rao R R et al., Immunity, 2012]. Last, the enhanced cytotoxic potential of T cells from combined therapy was also supported by elevated expression of the NKG2D receptor which has been shown to trigger TCR-independent cytotoxicity in activated T cells (FIGS. 13A, 14E and 14F) [Verneris M R et al., Blood 2004].

Having observed upregulation of granzymes and cytotoxic receptors in combination therapy, the function of T cells was investigated using an ex vivo tumor growth assay. Splenocytes obtained from all groups were co-cultured at an effector-to-target cell ratio of 10:1 with either CT26.FLUC (FIG. 13F) or MyC-CaP.Fluc (FIG. 13G) tumor cell lines. The anti-tumor activity of splenocytes was determined by measuring the luciferase activity of cell lines, which correlates with tumor growth. Tumor growth was markedly reduced when co-cultured with splenocytes from mice receiving combined therapy compared with splenocytes from naïve, control and mice treated with anti-OX40 in both tumor models. The anti-tumor activity of splenocytes from mice treated with SV.IL12 alone was weaker than that from combined therapy. Surprisingly, tumor growth inhibition was mediated by CD4 T cells as depletion of CD4 T cells but not CD8 T cells abolished the inhibitory effect on tumor cells. Together, these results clearly show that T cells from combined therapy elicit enhanced anti-tumor and functional activity, such as granzyme B and IFNγ production driven by CD4 T cells.

Mice Treated with SV.IL12 in Combination with Anti-OX40 Display Enhanced T Cell Migration and Intratumoral T Cell Immunity

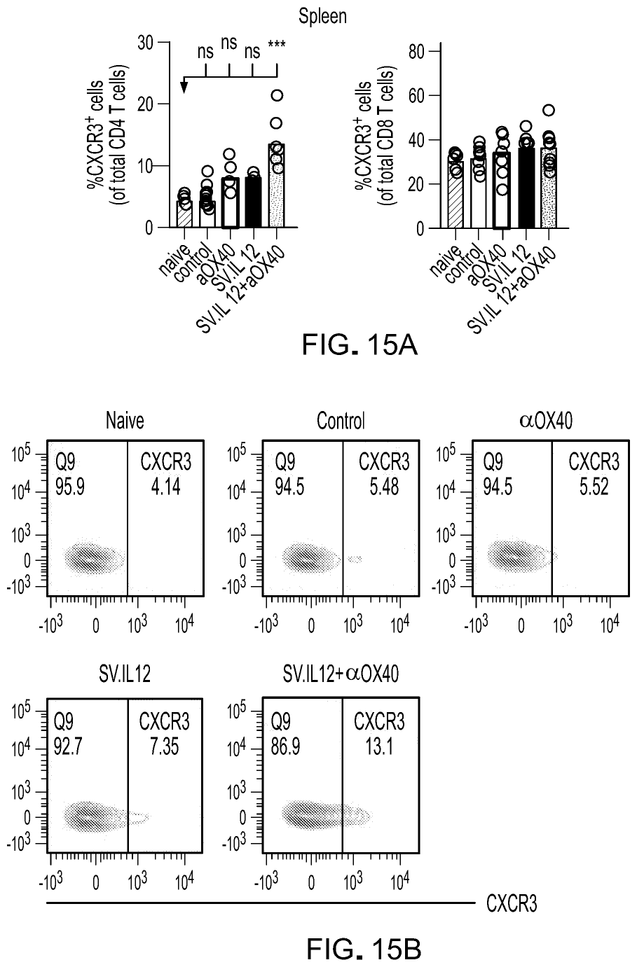

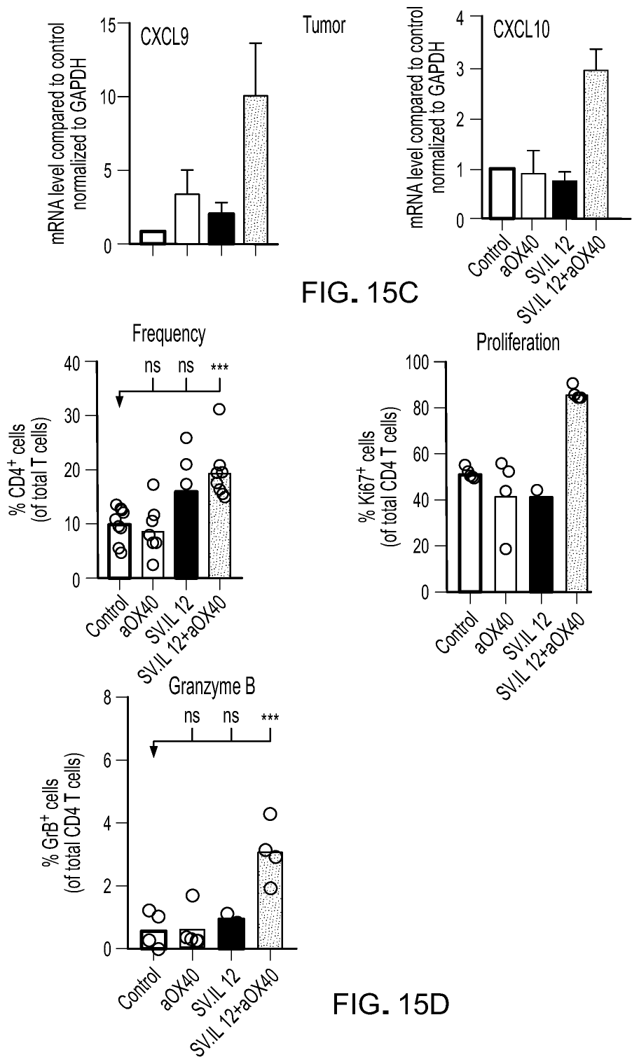

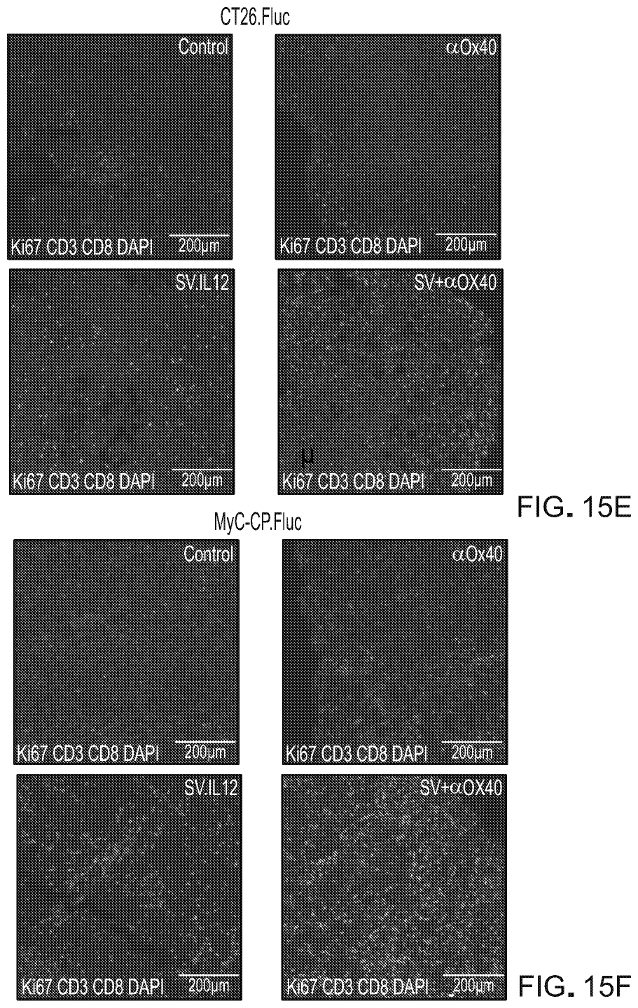

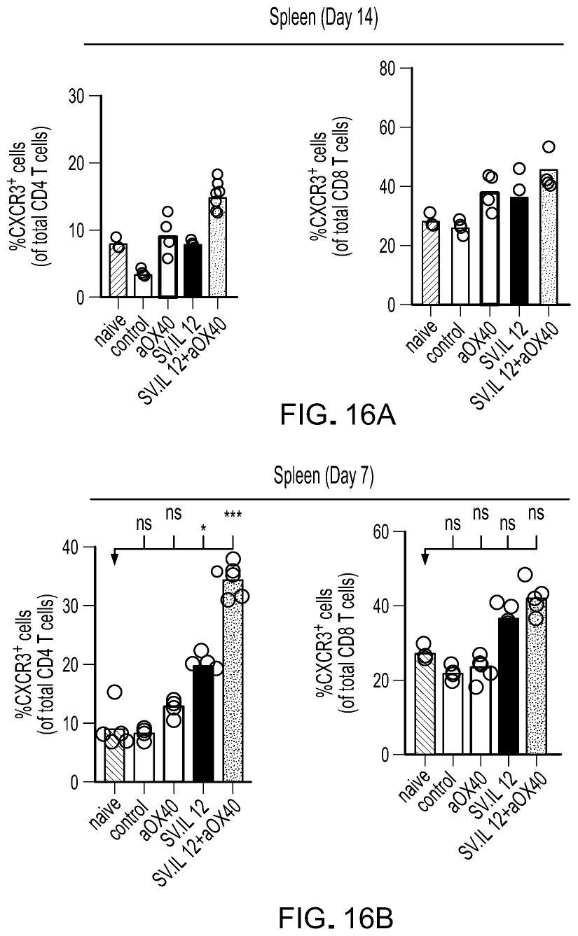

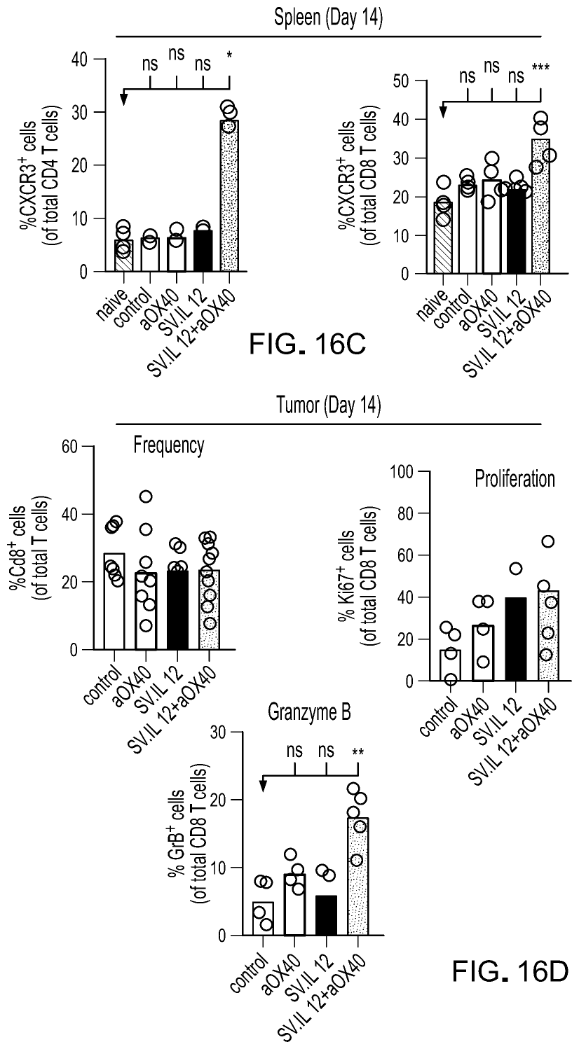

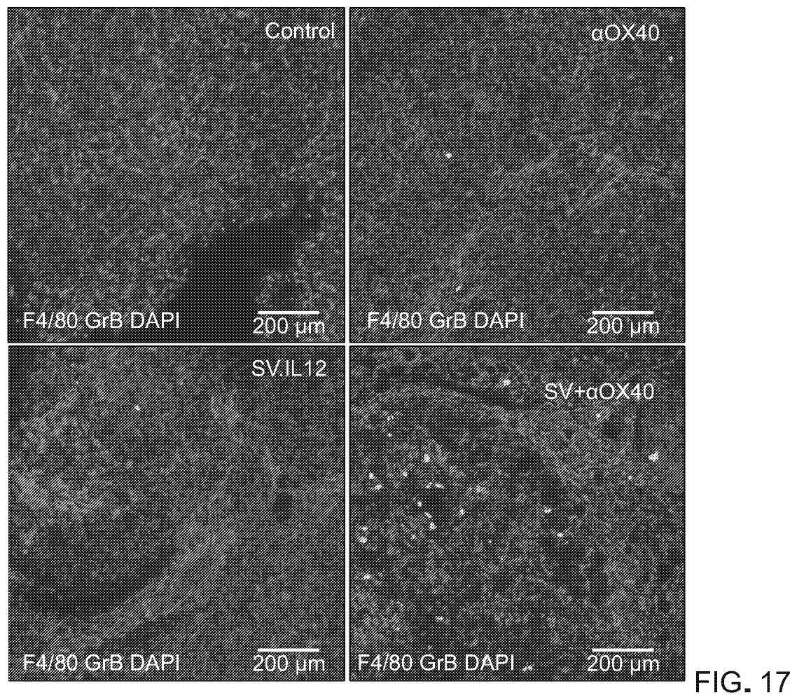

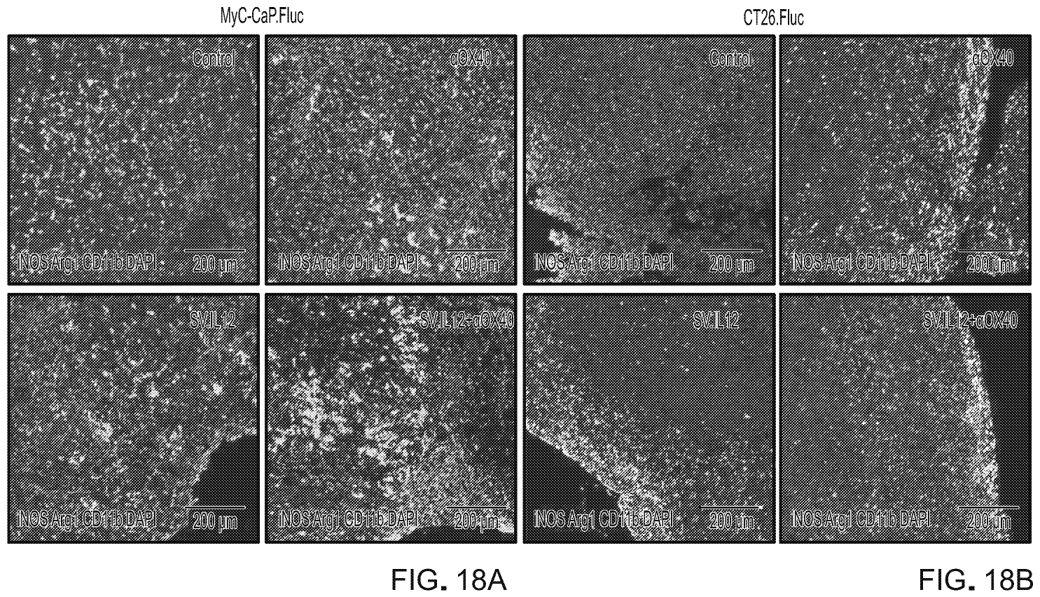

Only a minority of the total of treated patients respond to current immunotherapy and the presence of TILs has been shown to be one of the main factors that influence the responsiveness towards various therapies in multiple cancers [Galon, J et al., Science 2006; Hwang W T et al., Gynecol Oncol 2012]. Due to the fact that SV elicited anti-tumor responses do not necessarily require direct infection of the tumor or intratumoral injection, whether SV.IL12 therapy in combination with anti-OX40 could nevertheless alter the local tumor microenvironment and favor intratumoral immunity, was investigated. To assess whether SV.IL12 in combination with anti-OX40 induces T cell infiltration into the tumor, the chemokine receptor CXCR3 on peripheral T cells was analyzed after one week of treatment. In the CT26.Fluc model CXCR3 levels were significantly upregulated on CD4 T cells during combination therapy compared with all other groups and CXCR3 levels remained elevated over the course of treatment (FIGS. 15A, 15B and 16A). In contrast, CXCR3 expression on CD8 T cells only appeared later on in treatment, suggesting that CD4 T cells are first recruited to the inflamed site followed by CD8 T cells (FIG. 16A). MyC-CaP.Fluc tumor bearing mice showed elevated levels of CXCR3 on CD4 and CD8 T cells after one week of combination treatment (FIGS. 16B and 16C). Furthermore, SV.IL12 treatment also increased CXCR3 expression on T cells but to lesser extent. The reason for this discrepancy between the two models might be the differences in the number of treatments as MyC-CaP.Fluc bearing mice receive 3 and 4 times more injections of anti-OX40 and SV.IL12, respectively. Cells expressing CXCR3 follow the gradient of their ligands CXCL9, CXCL10 and CXCL11 [Groom, J. R. & Luster, A. D. Exp. Cell Res. 2011]. Indeed, combination therapy also enhanced the production of CXCL9 and CXCL10 in the tumor microenvironment, as judged by real-time PCR, suggesting that CXCR3 positive T cells migrate to the tumor site (FIG. 15C). Treatment of SV.IL12 or anti-OX40 alone did not alter the expression of these ligands. In line with these observations, an overall increase in T cells was observed in CT26.Fluc and MyC-CaP.Fluc peritoneally disseminated tumors from mice treated with combined therapy compared with control and anti-OX40 treated mice (FIGS. 15E and 15F). SV.IL12 treated mice also showed enhanced T cell infiltration but to a lesser extent. Furthermore, dissecting CD4 and CD8 T cells by flow cytometry revealed that combination therapy increases the proportion of CD4 T cells in CT26.Fluc tumors, which is consistent with the elevated CXCR3 expression on peripheral CD4 T cells (FIG. 15D). These results clearly show that SV.IL12 in combination with anti-OX40 alter the tumor microenvironment by facilitating T cell infiltration via modulation of the CXCR3/CXCL9-11 axis. Not only did combination therapy increase T cell infiltration in both tumor models but CD4 as well as CD8 T cells also demonstrated enhanced functional activity in the tumor, as judged by the Ki-67 and granzyme B expression (FIGS. 15D, 16D and 17). In line with these results, a decrease in proliferation, as judged by the expression of Ki-67 in tumor cells, was observed in CT26.Fluc and MyC-CaP.Fluc tumor cells when treated with combined therapy compared with all other treatments (FIGS. 15E and 15F). These results suggest that the presence of activated T cells in the tumor microenvironment exert anti-tumor activity which inhibits tumor growth. Besides from T cell activation, we also observed enhanced iNOS production in MyC-CaP.Fluc tumors treated with combination therapy compared with control or anti-OX40 treatment (FIG. 18). SV.IL12 treatment alone also induced iNOS production but to a lesser extent. Interestingly, the amount of iNOS inversely correlated with ariginase1 production, suggesting a repolarization of tumor associated macrophages from the M2-like (pro-tumor) into M1-like (anti-tumor) phenotype during combination therapy. These trends were only observed in MyC-CaP.Fluc and not in CT26.Fluc tumors, which might be a consequence of SV directly infecting MyC-CaP cells.

The study described herein provides a practical strategy for cancer immunotherapy using an OV and anti-OX40. This strategy takes advantage of the preexisting T cell immune repertoire in vivo, removing the need to know about present tumor antigens. The study described herein shows that the combination of replication-deficient SV.IL12 and anti-OX40 amplifies these antitumor T cells and induces their action throughout the body against two distinct solid tumors, reversing effectively local tumor-mediated immune suppression. This effect was specific for combination therapy and was not observed during SV.IL12 or anti-OX40 treatment alone.

The high metabolic activity of cancer cells together with the poor vasculature blood supply in the tumor microenvironment can induce nutrient deprivation [Delgoffe, G M et al., Cancer Immunol Res. 2016; Scharping, N E & Delgoffe, GM, Vaccines, 2016; Chang, C H et al., Cell, 2015]. These conditions can impair TCR signaling, glycolytic and mitochondrial metabolism, as well as decrease mitochondrial mass, all hallmarks of T effector cells, resulting in impaired anti-tumor effector functions of tumor-specific T cells. 39-42 Scharping, N. E. et al., Immunity 2016; Ho, P. C. et al., Cell 2015; Siska, P J & Rathmell, J C, Trends Immunol., 2015; Zhao, E. et al., Nat Immunol, 2016]. The data in two distinct models of cancer immunotherapy disclosed in the study described herein, shows that SV.IL12 in combination with OX40 signaling provides the necessary metabolic support to T cells to generate an efficient antitumor response. This metabolic support is characterized most prominently by elevated mitochondrial function and mass in CD8 T cells as well as a switch to aerobic glycolysis in CD4 T cells. T cells from mice treated with SV.IL12 in combination with anti-OX40 demonstrated enhanced protein expression of c-Myc compared with all other groups. Thus, the study described herein clearly shows that T cells are metabolically reprogrammed in the periphery during combination therapy.

The study described herein strongly shows that the therapeutic efficacy of SV.IL12 with anti-OX40 is driven by T cell modulation and reprogramming of its metabolic state, in order to enhance the anti-tumor response in the periphery and in the tumor microenvironment. Furthermore, the use of SV allows these metabolically reprogrammed T cells to better infiltrate the tumor microenvironment, which is crucial for an adequate immunotherapy. Anti-OX40 antibody is currently being studied in phase 1 and 2 clinical trials. SV will be tested as a single agent in its first clinical trial in the third quarter of 2020. The results from our current preclinical studies provide a strong rationale for combining SV.IL12 with agonistic anti-OX40 antibodies in a therapeutic format in patients with solid tumors. In summary, the studies described herein clearly show that even in absence of direct SV tumor targeting, SV.IL12 in combination with anti-OX40, or SV vector encoding IL-12 and anti-OX40, can alter the tumor microenvironment in distinct solid tumors through an indirect and immunity driven mechanism that enhances T cell infiltration and intratumoral T cell immunity.

### Example 2: Combination of IL-12 and Anti-OX40 Expressed by Sindbis Viral Vectors Synergistically Enhances Survival of Subjects with Established Tumors

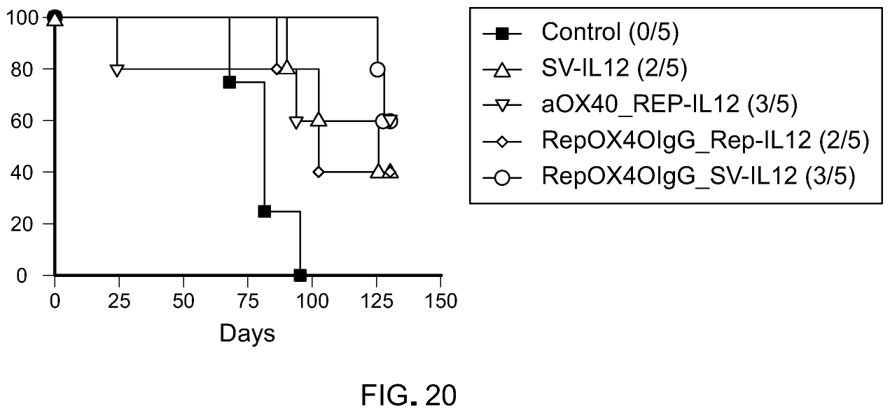

The study described herein investigates the effect of administering IL-12 and anti-OX40 antibody, both expressed by Sindbis viral vectors, on established tumors. This strategy is particularly advantageous for treatment of cancers like ovarian cancer, wherein the combination of SV.IL-12 and anti-OX40 antibody is not found to be as effective, as observed in colon and prostate cancers. The administration of SV/IL-12 and an anti-OX40 antibody enhanced clearance of established tumor of colon and prostate cancer cell lines, CT26 and MyC-Cap respectively. C57/B16 albino (female) mice re-injected with Alm5-2Fluc-17 ovarian cancer cells to establish a tumor (FIG. 19), and treated with: a) a SV vector expressing IL-12; b) a combination of SV vectors expressing IL-12 and an anti-OX-40 antibody (aOX40_REP-IL12), c) a 50% mixture of either a fragmented SV expressing OX-40 IgG plus a fragmented SV expressing IL-12 (RepOX4OIgG_Rep-IL12) or d) a 50% mix of a fragmented SV expressing OX-40 IgG plus a full length SV expressing IL-12 (RepOX4OIgG_SV-IL12). The percentage survival rate of the treatment groups RepOX4OIgG_SV-IL12 and aOX40_Rep-IL12 were comparable, and higher than the SV-IL-12 and RepOX4OIgG_Rep-IL12 treatment group. However, the results showed that, the RepOX4OIgG_SV-IL12 treatment group showed the highest enhancement of survival rate (FIG. 20).

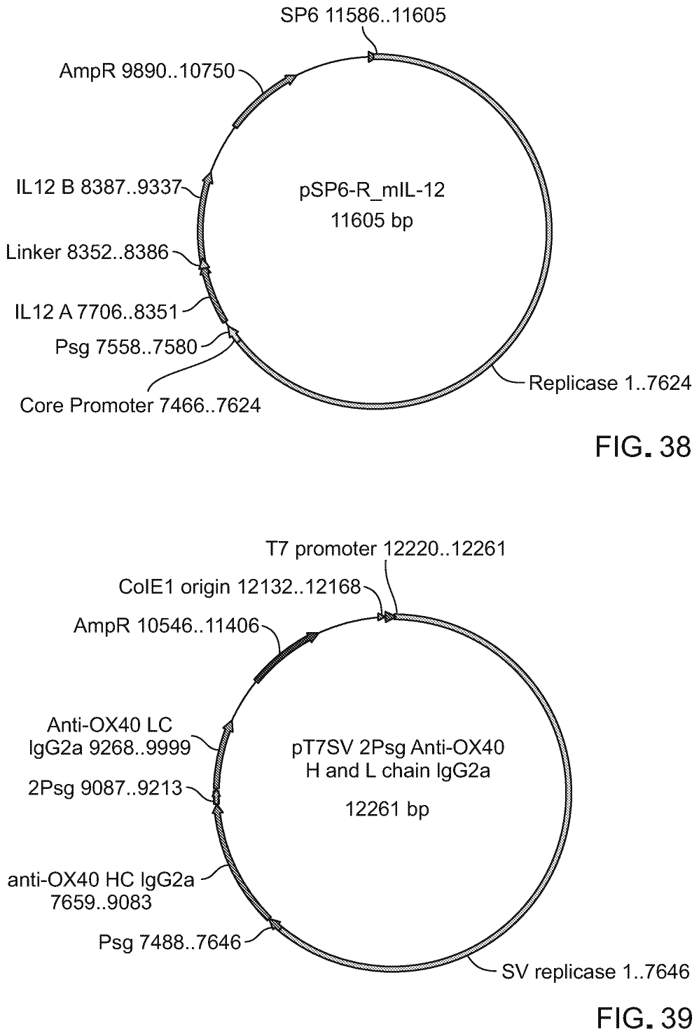

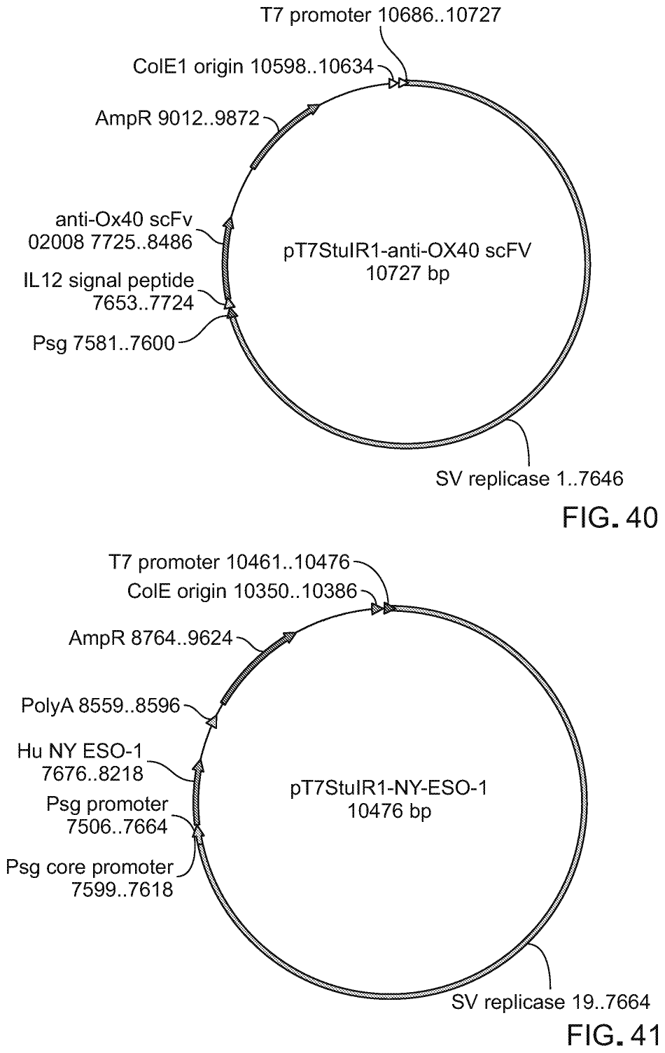

The study described herein, provides plasmid constructs for expressing IL-12, and anti-OX40 in a SV vector. The study described herein, provides plasmid constructs encoding IL-12 a and b subunits (FIG. 38), anti-OX40 IgG2a heavy and light chains (FIG. 39) and a single chain antibody to OX40 (FIG. 40).

SV.IL12 plasmid used in this study has been published in 2002 [Tseng J C et al., J Natl Cancer Inst. 2002]. To construct a Sindbis viral vector containing genes for interleukin 12 (IL-12), the Sindbis viral vector SinRep/2PSG was first constructed, which contains a secondary subgenomic promoter that is responsive to the Sindbis replicase. Two DNA oligonucleotide primers (sequence 5′ CGCGTAAAGCATCTCTACGGTGGTCCTAATAGTGCATG-3′; SEQ ID NO: 29) and its complementary strand 5′CACTATTAGGACCACCGTCGAGATGCTTTA-3′; SEQ ID NO: 30) containing the subgenomic promoter sequence were annealed and ligated into the MluI and SphI sites of the SinRep plasmid. The murine IL-12 α subunit gene (mp35; ATCC 87596) and the IL-12 β subunit gene (mp40; ATCC 87595) were subcloned into the MluI and the StuI sites of SinRep/2PSG, respectively, to produce the Sin-Rep/IL12 plasmid.

The H and L chains of the OX40 Ab are expressed from a single SV using two subgenomic promoters. The synthesized sequences were designed to encode an IL-12 secretory signal peptide upstream of both H and L polypeptide sequences preceded by a ribosome binding site and the start codon. The variable Ab binding sequences that functionally bind to activate the OX40 Receptor contain complementarity determining regions that are not unique. The variable chain is linked to the respective L (GenBank accession BAR42292) and H chain (GenBank accession CAC20702) constant region sequences of mouse IgG2a; the murine IgG2a isotype is comparable to the BioXcell OX40 Ab used in parallel in vivo experiments.

In summary results described herein clearly show the possibility of using a combination of SV vectors expressing IL-12 and anti-OX40 antibody or a SV vector expressing both expressing IL-12 and anti-OX40 antibody, for treatment of cancers that may be resistant to treatment with anti-OX40 antibody administered directly.

### Example 3. Molecular and Metabolic Pathways Mediating Curative Treatment of a Non-Hodgkin B Cell Lymphoma by Sindbis Viral Vectors and Anti-4-1BB Monoclonal Antibody

The studies described herein use an antibody directed at 4-1BB (CD137, TNFRSF9), a T cell costimulatory molecule. 4-1BB agonist stimulation greatly enhances NK and cytotoxic T cell activity. There are preclinical studies showing that α4-1BB effectively treats lymphoma and that depletion of Treg cells enhances the therapeutic effect of α4-1BB [Houot R et al., Blood, 2009]. The A20 tumor cells uses in the study described herein were derived from a spontaneously arising reticulum cell sarcoma (a non-Hodgkin lymphoma) in a BALB/c mouse.

Previously, SV carrying NYESO-1 was used, which encodes the cancer testis TAA, NYESO-1, to cure CT26 tumors expressing NYESO-1 [Scherwitzl I et al., Mol. Ther. Oncolytics, 2018]. The studies described herein show that systemically disseminated A20 lymphoma can be completely cured by SV plus α4-1BB mAb combination therapy without the need to produce a SV that encodes a TAA known to be present in the A20 lymphoma cells. Further, neither intratumoral injection of the SV vectors nor infection of the tumors is required as the A20 B lymphoma cells used in the current model are resistant to SV infection.

One difference in the current study, compared with those previously published, is the use of SV vector combination therapy that involves an agonistic mAb for a costimulatory receptor versus targeting checkpoint blockade molecules such as CTLA4 and PD-1. The studies described herein show that agonistic mAbs in combination with SV vectors trigger a cascade of events that results in curative results. The findings disclosed herein reveal the potential of SV combination therapy to cure tumors for which TAAs are completely unknown.

Materials and Methods

Firefly Luciferase (Fluc)-Expressing A20 Cells Generation

A20 cells were transfected with pGL4-neo_Fluc plasmid (Promega) by electroporation via Nucleofector™ kit V (Lonza). Fluc-A20 cell clones were selected and maintained in RPMI1640 (Cellgro)+10% FBS (Gibco)+250 μg/ml G418 (Gibco). One A20 clone stably expressed fLuc and was used for tumor inoculation and consecutive experiments.

SV Production

SV-LacZ production and titering were done the same as previously described [Scherwitzl I et al., Mol. Ther. Oncolytics, 2018].

SV-GFP Infection

A20 cells and control BHK cells were infected by SV carrying GFP for 1 h. The GFP expression was observed the next day by fluorescence microscopy.

A20 Tumor Inoculation and In Vivo Imaging System (IVIS) Imaging

3×10⁶ fLuc-A20 cells were inoculated to BALB/C mice by i.p injection. Tumor growth was monitored as previously described [Scherwitzl I et al., Mol. Ther. Oncolytics, 2018].

SV and α4-1BB Ab treatment

Treatment was started after successful tumor inoculation (4 days after tumor cell injection, confirmed by IVIS imaging). Tumor growth was measured every week by noninvasive bioluminescent imaging. SVLacZ was injected 4 times per week, for totally 3 weeks. The virus (10⁷-10⁸ TU/mL) in a total volume of 500 μL was i.p. injected. For 2 groups (4-1BB and SV plus 4-1BB), 350 μg/mouse 41BB Ab was injected 3 times/week for 2 weeks. InVivo MAb anti-mouse 4-1BB was ordered from BioXCell (Clone: LOB12.3, Cat. No. BE0169). In low dose treatment protocol, SVLacZ was injected i.p. 3 times per week, for totally 3 weeks. 41BB Ab (50 g/mouse) was injected once a week for 3 weeks.

Elispot

Mouse IFNγ ELISPOT was performed according to the manufacturer's protocol (BD Biosciences). 2×10⁵ splenocytes or 1×10⁵ T cells were plated per well O/N in RPMI supplemented with 10% FBS. For a positive control, splenocytes were stimulated with 5 ng/ml PMA+1 μg/ml Ionomycin.

Flow Cytometry

Fluorochrome-conjugated antibodies against mouse CD3, CD4, CD8, CD25, CD44, CD62L, ICOS, CD11 a, ICAM-1 were purchased from Biolegend (San Diego, Calif.). Fluorochrome-conjugated antibodies against mouse Foxp3, EOMES and CCR5 were purchased from Thermofisher. BUV395 conjugated antibody against mouse CD8a was purchased from BD Biosciences. For surface staining, cells were washed and stained with anti-mouse direct conjugated antibodies. Cells were analyzed using the LSRII flow cytometer (BD Biosciences) and data were analyzed using Flowjo software (Treestar, Ashland, Oreg.). For intracellular cytokines staining, stimulated cells were fixed with cytofix/cytoperm solution (BD Biosciences), permeablized with perm/wash buffer (BD Biosciences) and stained with anti-mouse IFNγ antibodies. For nuclear antigen, cells were fixed and permeabilized by Foxp3 fixation/permeabilization buffer (eBioscience) and stained with anti-Foxp3, T-bet, Ki67 and EOMES antibody.

RNA Isolation and Transcriptome Analysis

Total RNA was harvested by RNAeasy isolation kit (Qiagen, Valencia, Calif.). For each group, 3 BALB/C mice were used as biological repeats. RNA-seq was performed by NYUMC Genome Technology Center (GTC). To identify significant differences in expression between any pair of groups, differential expression analysis was performed using Deseq2 and an adjusted p value cutoff of 0.05 was applied [19, Love M I et al, Genome Biol. 2014] (q<0.05). To increase stringency, only genes with a Log 2 fold change ≥1 (upregulated) or ≤−1 (downregulated) were selected for further analysis. Gene cluster analysis was performed by DAVID analysis using the selected differentially expressed genes [Huang da W et al., Nucleic Acids Res 2009, Huang da W et al., Nat Protoc. 2009]. RNA-seq results (normalized counts) were used as input to perform with Gene Set Enrichment Analysis (GSEA) [Subramanian A, et al., Proc Natl Acad Sci USA. 2005]. Molecular Signatures Database (MSigDB)v4.0 were used as screening database. For each gene, the gene expression value is normalized by the relative log 2 fold change compared to the median value of this gene. Expression heatmap is drawn by Morpheus (https://software. broadinstitute.org/morpheus/). Cannonical pathway and disease and biological functional analysis were generated by ingenuity pathway analysis (IPA; Ingenuity Systems, Redwood City, Calif.) using the statistical differential expressed genes list. To increase the sample representativeness, for IPA, we choose nominal p<0.05 as cutoff value.

Tumor Infiltrating Lymphocyte (TIL) Harvest

To investigate the phenotype of TIL, all treatments were started 11 days after tumor inoculation, After 7 days treatment, tumor mass was harvested and the phenotype of TIL were analyzed as previously described [18, Scherwitzl I, Mol Ther Oncolytics. 2018].

T Cell Seahorse Assay

T cells were isolated from spleen by using pan T cell isolation kit (Stemcells). T cells were plated at 6×10⁵ cells/well in 24 well plate. Oxygen consumption rate (OCR) and excellular acidification rate (ECAR) were measured by Agilent Seahorse XFe24.

Statistical Analysis

For the two group comparison, statistical difference was determined by unpaired two tail Student t-test. The multiple sample comparison was analyzed by one way ANOVA. P<0.05 was determined to be significant for all experiments. All values were calculated with Excel (Microsoft) and Prism software (GraphPad).

SV and α4-1BB mAb Combination Completely Cured A20 Lymphoma

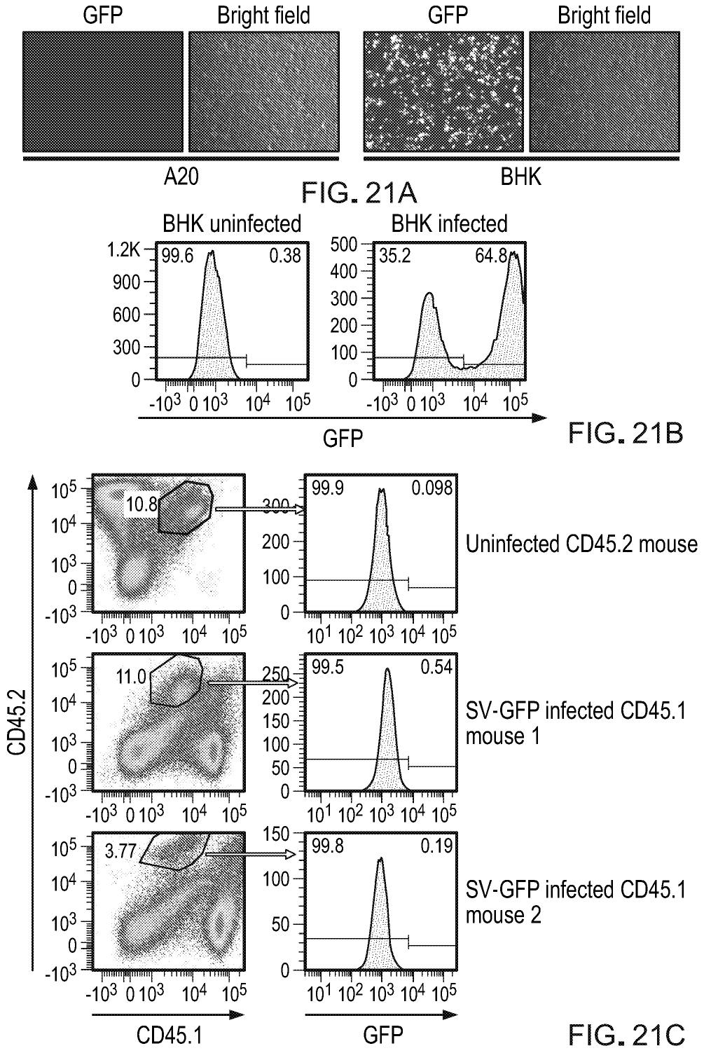

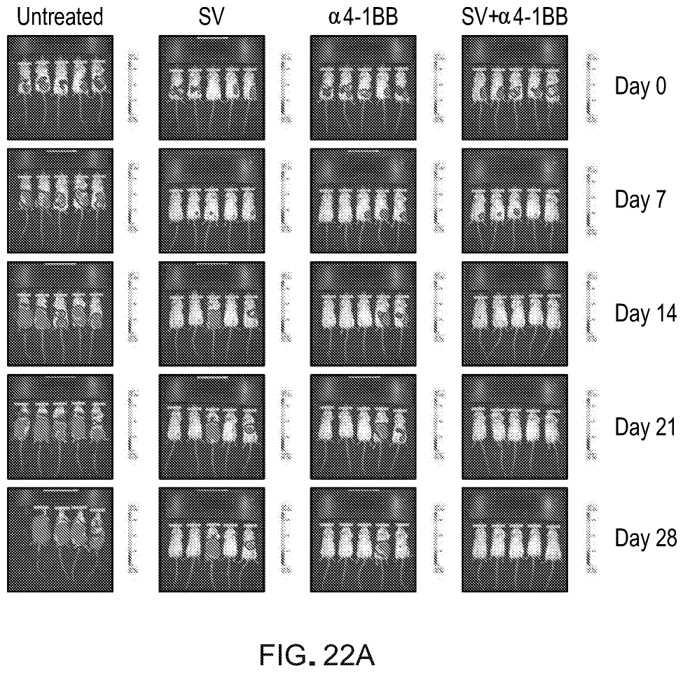

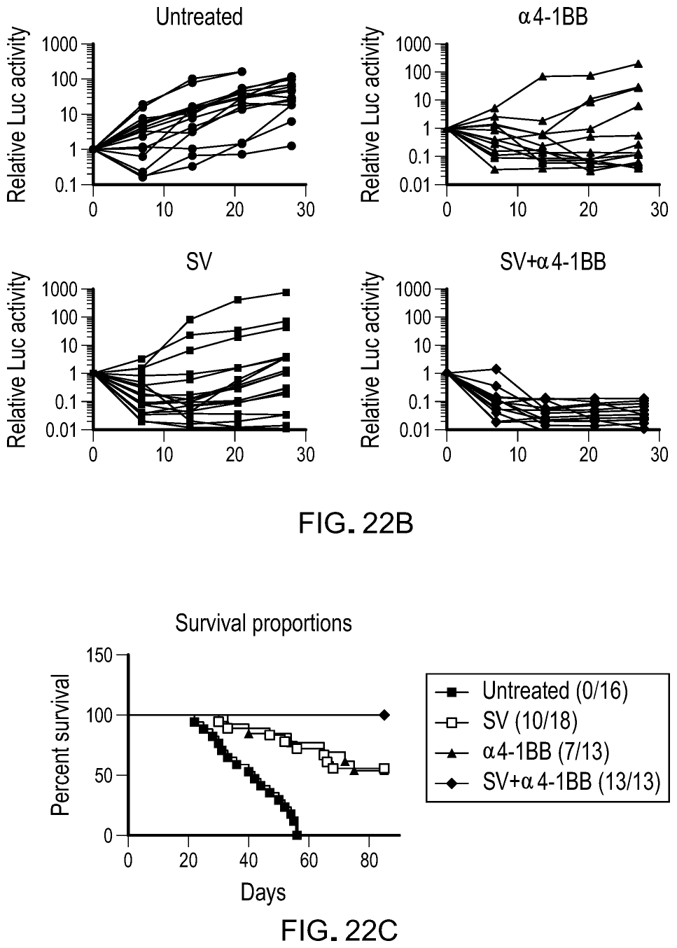

To explore if SV has therapeutic effect on tumors not targeted or infected by SV vectors, the A20 B cell lymphoma was used, which is highly resistant to SV infection (FIG. 21). To monitor tumor growth in vivo, a firefly luciferase (f-Luc) expression vector was transfected into the A20 lymphoma cell line by electroporation. A stable f-Luc expressing A20 clone was isolated through G418 selection. 3×10⁶/mouse f-Luc A20 tumor cells were inoculated by intraperitoneal (i.p.) injection. Tumor growth was monitored by IVIS imaging once per week. Tumors were successfully established after 4 days inoculation (FIG. 22A). After tumors were established, SV and α4-1BB mAb treatment started (designated as day 0). A therapeutic protocol similar to that previously described [Scherwitzl I et al., Mol Ther Oncolytics. 2018], was used. SV plus α4-1BB mAb combination achieved the best therapeutic effect (FIG. 22B). All mice in that group showed complete tumor regression in 2 weeks. Although, both SV or α4-1BB treatments alone achieved obvious therapeutic effects compared with untreated mice, they were not as effective as the combination and a fraction of mice in these two groups eventually succumbed to tumor (FIG. 22C).

SV Alone and SV Plus α4-1BB mAb Stimulated Cell Cycle Progression, Cytokine Production, and Activation

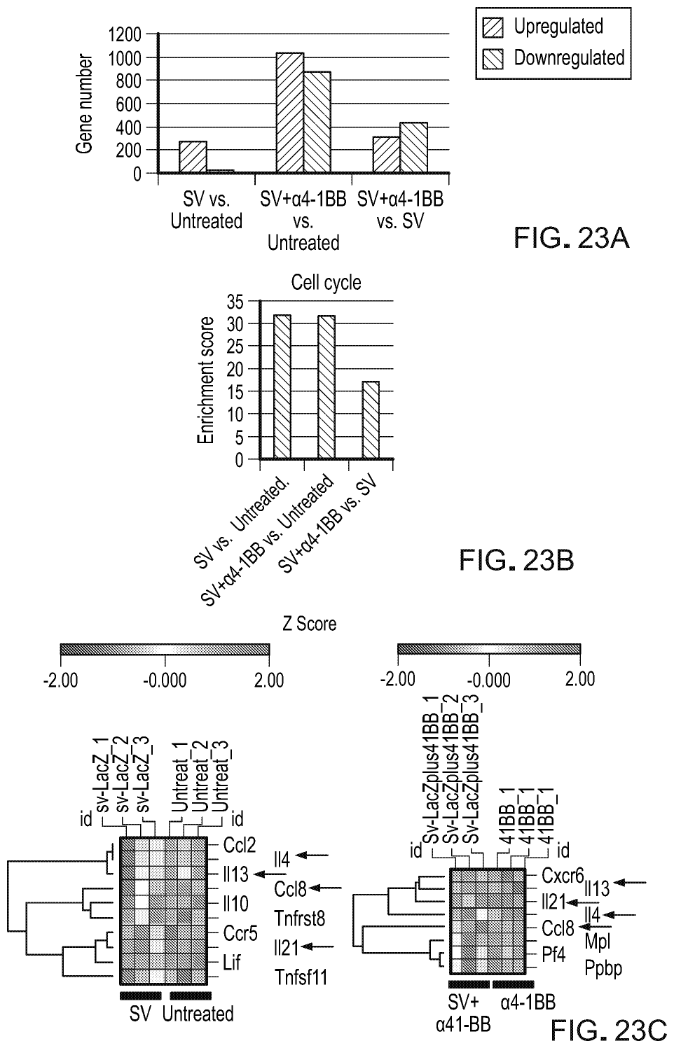

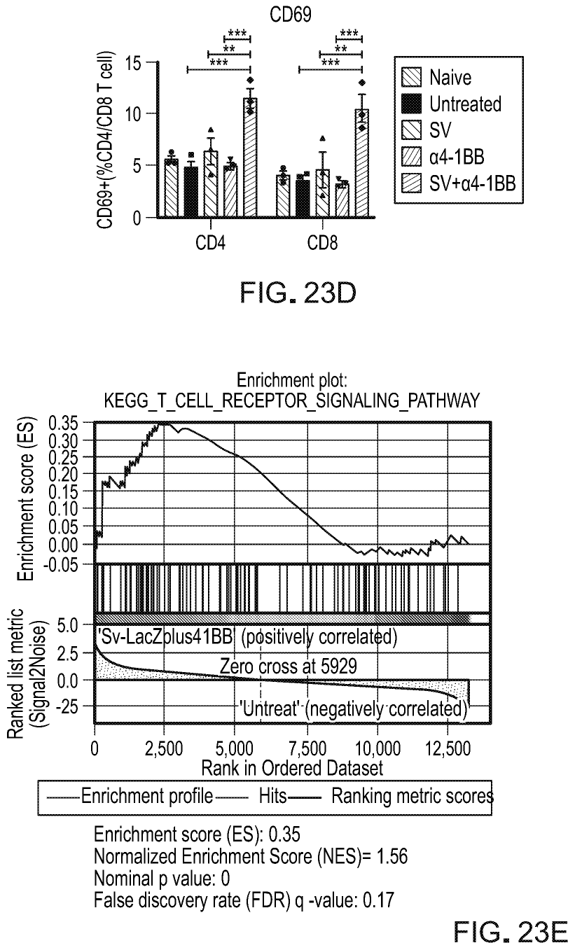

In the study described herein, SV significantly inhibited tumor growth by day 7 (FIG. 22A). T cells play a critical role in SV induced anti-tumor immunity. T cell response reaches a peaked on day 7 after infection [Scherwitzl I et al., Mol Ther Oncolytics. 2018]. To explore how SV induced T cell responses that help to eradicate A20 lymphoma, RNA-Seq was performed using purified splenic T cells from all groups on day 7. Compared with untreated samples, 271 genes upregulated (q<0.05 and Log 2 Fold Change≥1) and 28 genes downregulated (q<0.05 and Log 2 Fold Changes≤−1) were identified in the SV infected group through Deseq2 analysis (FIG. 23A, Table 1).

TABLE 1
The SD expressed genes list for SV vs. untreated group by RNA-Seq (q
< 0.05, Log2FC ≥ 1 and Log2FC ≤ −1).

| gene | baseMean | log2FC | log2FCunshrunk | pvalue | padj |
|---|---|---|---|---|---|
| Gm2762 | 38.5 | 3.15 | 4.04 | 0 | 0 |
| Il21 | 229 | 2.57 | 3.66 | 1.81E−13 | 2.63E−11 |
| Ascl2 | 24.4 | 2.55 | 3.83 | 2.83E−12 | 3.8E−10 |
| Akr1c18 | 25.3 | 2.4 | 4.32 | 6.37E−11 | 6.98E−09 |
| Angptl2 | 741.2 | 2.35 | 2.59 | 0 | 0 |
| E2f8 | 535.1 | 2.33 | 2.61 | 0 | 0 |
| Vat1l | 14.9 | 2.33 | 4.01 | 1.65E−10 | 1.68E−08 |
| Aurkb | 806.6 | 2.3 | 2.46 | 0 | 0 |
| Gzmb | 4113.9 | 2.26 | 2.51 | 0 | 0 |
| Cdc25c | 143.9 | 2.08 | 2.51 | 3E−15 | 5.19E−13 |
| Prr11 | 288 | 2.04 | 2.36 | 0 | 3.3E−14 |
| Ighg2c | 873 | 2.04 | 4.64 | 6.37E−08 | 4.29E−06 |
| Tpx2 | 1113.7 | 2.03 | 2.19 | 0 | 0 |
| Bub1 | 362.3 | 2.02 | 2.22 | 0 | 0 |
| Bub1b | 1344.3 | 2.01 | 2.14 | 0 | 0 |
| Nusap1 | 813.1 | 2 | 2.12 | 0 | 0 |
| Kntc1 | 402.4 | 2 | 2.22 | 0 | 0 |
| Kif18b | 606.6 | 1.99 | 2.14 | 0 | 0 |
| Gzmk | 1785.2 | 1.99 | 2.28 | 0 | 4E−15 |
| Nuf2 | 279.6 | 1.97 | 2.25 | 0 | 1.8E−14 |
| Mcm10 | 477.4 | 1.96 | 2.11 | 0 | 0 |
| Mki67 | 5899.1 | 1.96 | 2.12 | 0 | 0 |
| Ccnb2 | 1255.4 | 1.95 | 2.1 | 0 | 0 |
| Rad54l | 350.2 | 1.95 | 2.16 | 0 | 0 |
| Hmmr | 311.7 | 1.94 | 2.19 | 0 | 1E−15 |
| Chn1 | 24 | 1.94 | 2.52 | 2.4E−09 | 2.01E−07 |
| Ccna2 | 1680.2 | 1.93 | 2.04 | 0 | 0 |
| Pclaf | 522.9 | 1.93 | 2.17 | 0 | 5E−15 |
| Cenpf | 495.6 | 1.92 | 2.07 | 0 | 0 |
| Kif2c | 341.3 | 1.92 | 2.1 | 0 | 0 |
| Cdkn3 | 129.2 | 1.92 | 2.16 | 0 | 0 |
| Vdr | 250.7 | 1.92 | 2.2 | 1.02E−13 | 1.57E−11 |
| Fignl1 | 145.7 | 1.91 | 2.15 | 0 | 0 |
| Ttk | 211.5 | 1.9 | 2.14 | 0 | 5E−15 |
| Diaph3 | 356.3 | 1.89 | 2.03 | 0 | 0 |
| Oip5 | 50.6 | 1.88 | 2.49 | 1.92E−10 | 1.94E−08 |
| Kif4 | 650.2 | 1.87 | 1.98 | 0 | 0 |
| Rrm2 | 1718.2 | 1.87 | 2.01 | 0 | 0 |
| Sccpdh | 123.3 | 1.86 | 2.05 | 0 | 3.6E−14 |
| Clspn | 637.1 | 1.83 | 1.98 | 0 | 0 |
| Padi4 | 34.4 | 1.83 | 2.41 | 6.18E−08 | 4.18E−06 |
| Shcbp1 | 274.8 | 1.82 | 1.98 | 0 | 0 |
| Rad51 | 401.4 | 1.82 | 1.99 | 0 | 0 |
| Tox2 | 889.5 | 1.82 | 2.01 | 0 | 1.5E−14 |
| Spc24 | 506 | 1.81 | 1.92 | 0 | 0 |
| Sgo1 | 162.8 | 1.81 | 2.01 | 0 | 5E−15 |
| Depdc1a | 29.6 | 1.81 | 2.69 | 1.72E−08 | 1.26E−06 |
| Lrr1 | 59 | 1.8 | 2.37 | 9.15E−10 | 8.14E−08 |
| Cdca8 | 1251.5 | 1.79 | 1.91 | 0 | 0 |
| Kif20a | 898.5 | 1.78 | 1.9 | 0 | 0 |
| Espl1 | 809.6 | 1.78 | 1.9 | 0 | 0 |
| Cdk1 | 1228.8 | 1.78 | 1.92 | 0 | 0 |
| Sapcd2 | 275.5 | 1.78 | 1.98 | 0 | 5E−14 |
| Pbk | 107.5 | 1.78 | 2.08 | 1.08E−13 | 1.63E−11 |
| Gfra4 | 95 | 1.78 | 2.1 | 9.39E−11 | 9.94E−09 |
| Fcer1a | 69.7 | 1.77 | 2.87 | 1.43E−06 | 6.68E−05 |
| Lag3 | 2916.7 | 1.76 | 1.84 | 0 | 0 |
| Rad51ap1 | 205.6 | 1.76 | 1.92 | 0 | 0 |
| Ube2c | 1391 | 1.75 | 1.95 | 5E−15 | 9.54E−13 |
| Knl1 | 203.3 | 1.75 | 1.98 | 2.7E−14 | 4.39E−12 |
| Kif15 | 595.3 | 1.74 | 1.87 | 0 | 0 |
| Cep55 | 503.9 | 1.74 | 1.88 | 0 | 0 |
| Ticrr | 558.4 | 1.74 | 1.88 | 0 | 0 |
| Neil3 | 228.1 | 1.72 | 1.89 | 0 | 7E−15 |
| Spc25 | 211.3 | 1.72 | 1.94 | 1.08E−13 | 1.63E−11 |
| Lpar3 | 17.1 | 1.72 | 2.4 | 4.07E−07 | 2.23E−05 |
| Ncapg | 383.1 | 1.71 | 1.87 | 0 | 4E−15 |
| Iqgap3 | 284.2 | 1.71 | 1.86 | 0 | 4E−15 |
| Nek2 | 554.5 | 1.71 | 1.87 | 0 | 2.9E−14 |
| 1500009L16Rik | 143.2 | 1.69 | 2.2 | 2.79E−08 | 1.97E−06 |
| Ckap2l | 455.4 | 1.68 | 1.86 | 3E−15 | 6.37E−13 |
| Ncaph | 822.4 | 1.67 | 1.76 | 0 | 0 |
| Uhrf1 | 1603.6 | 1.67 | 1.77 | 0 | 0 |
| Pimreg | 230 | 1.67 | 1.88 | 1.32E−13 | 1.96E−11 |
| Top2a | 4726.3 | 1.66 | 1.73 | 0 | 0 |
| Spag5 | 1183.2 | 1.66 | 1.77 | 0 | 0 |
| Kif14 | 299.1 | 1.66 | 1.84 | 3E−15 | 6.22E−13 |
| Il10 | 1005.5 | 1.66 | 1.83 | 3.1E−14 | 4.98E−12 |
| Pif1 | 204 | 1.66 | 1.94 | 1.16E−11 | 1.41E−09 |
| Parpbp | 117.3 | 1.66 | 1.94 | 1.52E−11 | 1.79E−09 |
| Ly6a | 2208.2 | 1.65 | 2.56 | 2.41E−06 | 0.000106 |
| Melk | 329.8 | 1.64 | 1.75 | 0 | 0 |
| Ccnb1 | 160.8 | 1.63 | 1.88 | 1.91E−11 | 2.2E−09 |
| Cit | 784.3 | 1.62 | 1.74 | 0 | 4E−15 |
| Esco2 | 149.5 | 1.61 | 1.83 | 4.58E−12 | 5.99E−10 |
| CT030166.6 | 24.2 | 1.61 | 2.49 | 2.45E−06 | 0.000107 |
| Trip13 | 233.7 | 1.6 | 1.74 | 0 | 5E−14 |
| Cenpe | 685.5 | 1.59 | 1.71 | 0 | 1E−15 |
| Fads2 | 70.5 | 1.57 | 1.98 | 1.07E−07 | 6.9E−06 |
| Cdc45 | 628.7 | 1.56 | 1.62 | 0 | 0 |
| Cdca5 | 791.4 | 1.56 | 1.63 | 0 | 0 |
| Cdca3 | 1038.6 | 1.56 | 1.64 | 0 | 0 |
| Coro2b | 349.5 | 1.56 | 1.68 | 1.3E−14 | 2.31E−12 |
| Cenph | 201.9 | 1.55 | 1.73 | 2.02E−12 | 2.73E−10 |
| Troap | 236.4 | 1.54 | 1.73 | 6.69E−12 | 8.53E−10 |
| Birc5 | 1540.7 | 1.54 | 1.73 | 3.18E−11 | 3.56E−09 |
| Ifi27l2a | 8730.4 | 1.54 | 1.88 | 1.34E−07 | 8.38E−06 |
| Mcpt1 | 510.2 | 1.54 | 3.77 | 4.63E−05 | 0.001264 |
| Plk1 | 794.8 | 1.53 | 1.65 | 1E−15 | 1.43E−13 |
| Ankle1 | 238.9 | 1.53 | 1.72 | 1.64E−11 | 1.92E−09 |
| Cd109 | 30.7 | 1.53 | 2.47 | 3.8E−05 | 0.001099 |
| Cdca2 | 444.5 | 1.52 | 1.64 | 0 | 2.9E−14 |
| Siglec1 | 622.7 | 1.52 | 1.69 | 1.49E−11 | 1.77E−09 |
| Ska1 | 210.9 | 1.52 | 1.73 | 2.11E−10 | 2.12E−08 |
| Igkv14-111 | 309.6 | 1.51 | 2.66 | 7.06E−05 | 0.001789 |
| Alox5 | 104.2 | 1.5 | 2.08 | 6.41E−06 | 0.000241 |
| Stmn1 | 257.1 | 1.49 | 1.61 | 1.1E−14 | 2E−12 |
| Ect2 | 255.1 | 1.49 | 1.71 | 1.15E−09 | 9.97E−08 |
| Asf1b | 1276.4 | 1.48 | 1.55 | 0 | 0 |
| Ncapg2 | 526.2 | 1.48 | 1.58 | 0 | 1.5E−14 |
| Cenpm | 289.6 | 1.48 | 1.64 | 8.64E−12 | 1.07E−09 |
| Cenpp | 81.2 | 1.48 | 1.69 | 2.19E−10 | 2.19E−08 |
| F13a1 | 227.4 | 1.48 | 2.13 | 1.59E−05 | 0.000526 |
| Oas1a | 498.3 | 1.47 | 1.73 | 2.33E−08 | 1.66E−06 |
| Oas2 | 1573.5 | 1.47 | 2.24 | 3.32E−05 | 0.000987 |
| Cenpi | 136.8 | 1.46 | 1.61 | 1.49E−12 | 2.03E−10 |
| Tex15 | 58.4 | 1.46 | 1.88 | 2.46E−07 | 1.43E−05 |
| Lif | 439.6 | 1.45 | 1.5 | 0 | 0 |
| Tk1 | 1004.9 | 1.45 | 1.54 | 0 | 1E−15 |
| Kif22 | 1022.2 | 1.45 | 1.56 | 1.09E−13 | 1.63E−11 |
| BC030867 | 129.6 | 1.45 | 1.65 | 7E−10 | 6.44E−08 |
| Tnp2 | 17.6 | 1.45 | 2.43 | 6.42E−05 | 0.001655 |
| Tph1 | 43 | 1.45 | 2.97 | 0.000121 | 0.0028 |
| lghv1-39 | 28.6 | 1.45 | 2.67 | 0.000133 | 0.003011 |
| Tyms | 809.6 | 1.44 | 1.51 | 0 | 0 |
| Kif11 | 1109.9 | 1.44 | 1.51 | 0 | 0 |
| Ighv2-6 | 141.1 | 1.44 | 1.89 | 9.88E−06 | 0.000349 |
| Gng3 | 34.8 | 1.44 | 2.26 | 1.32E−05 | 0.000449 |
| Spdl1 | 204.5 | 1.43 | 1.54 | 1E−14 | 1.71E−12 |
| Cdh23 | 89.8 | 1.43 | 1.55 | 8.05E−12 | 1.01E−09 |
| Aspm | 319.4 | 1.43 | 1.59 | 9.94E−11 | 1.04E−08 |
| Dsp | 79.2 | 1.43 | 1.78 | 2.44E−06 | 0.000107 |
| Ighv1-19 | 10.2 | 1.43 | 2.88 | 0.000154 | 0.003401 |
| Depdc1b | 350.8 | 1.42 | 1.54 | 3.09E−13 | 4.45E−11 |
| Ctsg | 151.2 | 1.42 | 2.54 | 0.000128 | 0.002935 |
| Mxd3 | 280.1 | 1.41 | 1.5 | 2E−15 | 3.2E−13 |
| Chaf1a | 1027.2 | 1.4 | 1.49 | 2E−15 | 4.68E−13 |
| Aunip | 24.6 | 1.4 | 2.68 | 8.31E−05 | 0.00205 |
| Mt3 | 18.3 | 1.4 | 2.93 | 0.000162 | 0.003547 |
| Tcf19 | 1158.6 | 1.39 | 1.45 | 0 | 0 |
| Gpm6b | 315 | 1.39 | 1.48 | 1.5E−13 | 2.21E−11 |
| Cd5l | 2824.6 | 1.39 | 1.52 | 7.15E−11 | 7.78E−09 |
| Msr1 | 48.9 | 1.39 | 1.89 | 1.13E−05 | 0.000392 |
| Ighg1 | 2604.8 | 1.39 | 3.8 | 0.00023 | 0.004805 |
| Ncapd2 | 3741.3 | 1.38 | 1.42 | 0 | 0 |
| Oas3 | 1474.3 | 1.38 | 1.71 | 2.37E−06 | 0.000105 |
| Necab3 | 27.4 | 1.38 | 1.88 | 1.94E−05 | 0.000622 |
| Tpsab1 | 262.4 | 1.37 | 1.66 | 1.78E−06 | 8.17E−05 |
| Gm4951 | 44.3 | 1.37 | 1.8 | 3.61E−06 | 0.000148 |
| Exo1 | 187.1 | 1.36 | 1.54 | 1.17E−08 | 8.79E−07 |
| Adam33 | 60.3 | 1.36 | 1.62 | 1.37E−06 | 6.46E−05 |
| Klrb1a | 36.2 | 1.36 | 2 | 7.82E−05 | 0.001956 |
| Ndc80 | 345.5 | 1.35 | 1.44 | 2.6E−14 | 4.36E−12 |
| Slc16a2 | 105.6 | 1.35 | 1.56 | 1.32E−07 | 8.29E−06 |
| Klhl23 | 20.6 | 1.35 | 2.46 | 0.000156 | 0.003429 |
| Ckap2 | 394.5 | 1.32 | 1.39 | 0 | 2.3E−14 |
| Cdc6 | 570.6 | 1.32 | 1.4 | 2.3E−14 | 3.85E−12 |
| E2f7 | 275.3 | 1.32 | 1.48 | 3.98E−09 | 3.21E−07 |
| Mpo | 1003.3 | 1.32 | 2.2 | 0.000333 | 0.006459 |
| Mastl | 108.5 | 1.31 | 1.51 | 7.78E−08 | 5.19E−06 |
| Cenpn | 307 | 1.3 | 1.38 | 6.9E−14 | 1.09E−11 |
| Rad51b | 65.9 | 1.3 | 1.52 | 2.32E−07 | 1.37E−05 |
| Pgam2 | 13 | 1.3 | 2.75 | 0.000571 | 0.009954 |
| Smtn | 271.7 | 1.29 | 1.4 | 2.59E−10 | 2.57E−08 |
| Car5b | 136 | 1.29 | 1.45 | 6.02E−10 | 5.61E−08 |
| Wfdc17 | 99.9 | 1.29 | 1.47 | 2.18E−07 | 1.3E−05 |
| Xkr5 | 39.4 | 1.29 | 1.62 | 5.06E−06 | 0.000197 |
| Ifit1bl1 | 137.8 | 1.29 | 1.67 | 2.19E−05 | 0.000693 |
| Dhfr | 503.3 | 1.28 | 1.35 | 2.1E−14 | 3.58E−12 |
| Ccne1 | 503.1 | 1.28 | 1.37 | 8.53E−12 | 1.06E−09 |
| Il4 | 152.5 | 1.28 | 1.52 | 3.69E−06 | 0.000151 |
| Ms4a3 | 31.1 | 1.28 | 2.52 | 0.000653 | 0.011034 |
| Fancd2 | 392.9 | 1.27 | 1.33 | 0 | 3.4E−14 |
| Syce2 | 175.1 | 1.27 | 1.39 | 1.01E−09 | 8.79E−08 |
| Slc43a3 | 380.4 | 1.26 | 1.36 | 1.77E−11 | 2.05E−09 |
| Mcpt2 | 69.1 | 1.26 | 3.47 | 0.000769 | 0.012455 |
| Dna2 | 343.7 | 1.25 | 1.36 | 3.64E−10 | 3.52E−08 |
| Stil | 422.9 | 1.25 | 1.39 | 3.95E−08 | 2.72E−06 |
| Ms4a4a | 77.2 | 1.24 | 1.41 | 1.03E−07 | 6.69E−06 |
| Ifit3 | 421.9 | 1.24 | 2 | 0.000649 | 0.010983 |
| Ighv1-9 | 232.8 | 1.24 | 2.33 | 0.001087 | 0.016261 |
| Brca1 | 443.2 | 1.23 | 1.32 | 6.31E−12 | 8.12E−10 |
| Cks1b | 772.5 | 1.22 | 1.32 | 1.23E−09 | 1.05E−07 |
| Scin | 117.4 | 1.22 | 1.59 | 0.000102 | 0.002435 |
| Fanca | 375.8 | 1.21 | 1.29 | 3.34E−12 | 4.44E−10 |
| Sostdc1 | 55.7 | 1.2 | 1.42 | 2.05E−05 | 0.000654 |
| Tmprss4 | 19.7 | 1.2 | 1.67 | 0.000585 | 0.010122 |
| Pcdhgc4 | 19.5 | 1.2 | 1.98 | 0.000598 | 0.010298 |
| C3 | 1368.2 | 1.19 | 1.36 | 9.68E−07 | 4.74E−05 |
| Fbn2 | 27 | 1.19 | 1.43 | 6.33E−05 | 0.001639 |
| Igf2bp2 | 71.9 | 1.19 | 1.64 | 0.000461 | 0.008431 |
| Pask | 202.9 | 1.18 | 1.28 | 1.46E−09 | 1.24E−07 |
| Atp6v1g3 | 12.9 | 1.18 | 2.18 | 0.001663 | 0.022323 |
| Tmem121 | 19.5 | 1.17 | 1.86 | 0.001247 | 0.017975 |
| E2f1 | 876.4 | 1.16 | 1.19 | 0 | 0 |
| Zan | 929.3 | 1.16 | 1.27 | 3.35E−08 | 2.33E−06 |
| Plac8 | 2583 | 1.16 | 1.28 | 1.19E−07 | 7.61E−06 |
| Rad54b | 114.1 | 1.16 | 1.29 | 1.79E−07 | 1.09E−05 |
| Lig1 | 3384.1 | 1.15 | 1.2 | 7E−15 | 1.29E−12 |
| Gins2 | 413.5 | 1.15 | 1.22 | 1.04E−11 | 1.28E−09 |
| Gpsm2 | 436.7 | 1.15 | 1.23 | 2.7E−11 | 3.06E−09 |
| Arhgap11a | 847.4 | 1.15 | 1.22 | 7.3E−11 | 7.83E−09 |
| Gbp11 | 355.1 | 1.15 | 1.23 | 6.55E−10 | 6.06E−08 |
| Gm15987 | 44.4 | 1.15 | 1.51 | 0.000198 | 0.004191 |
| Efcab11 | 31.5 | 1.15 | 1.62 | 0.000442 | 0.008194 |
| Sncb | 15 | 1.15 | 2.01 | 0.001303 | 0.018592 |
| Il13 | 60.8 | 1.15 | 1.81 | 0.001306 | 0.01862 |
| Smpdl3b | 185.8 | 1.14 | 1.28 | 1.47E−07 | 9.11E−06 |
| Ispd | 31.4 | 1.14 | 1.42 | 0.000161 | 0.003543 |
| Igf2bp3 | 26.6 | 1.14 | 1.91 | 0.001053 | 0.015932 |
| Platr11 | 10.2 | 1.14 | 2.09 | 0.00249 | 0.029873 |
| Cpa3 | 467.2 | 1.14 | 2.12 | 0.002534 | 0.03018 |
| Tacc3 | 1922.3 | 1.13 | 1.16 | 0 | 1E−15 |
| Rmi2 | 184.3 | 1.13 | 1.21 | 5.58E−10 | 5.23E−08 |
| Nsl1 | 195.3 | 1.13 | 1.21 | 1.45E−09 | 1.23E−07 |
| AC151730.1 | 71.6 | 1.13 | 1.55 | 0.000357 | 0.006861 |
| Myh3 | 15.8 | 1.13 | 1.64 | 0.001352 | 0.019086 |
| Ighg2b | 522.3 | 1.13 | 2.65 | 0.002343 | 0.028708 |
| Klrg1 | 746.6 | 1.12 | 1.21 | 3.27E−10 | 3.18E−08 |
| Ociad2 | 184.7 | 1.12 | 1.27 | 1.87E−08 | 1.35E−06 |
| Mis18bp1 | 180.6 | 1.12 | 1.24 | 1.95E−07 | 1.17E−05 |
| Ccl1 | 31 | 1.12 | 1.77 | 0.001407 | 0.019614 |
| Prtn3 | 290.9 | 1.12 | 2.13 | 0.002845 | 0.032835 |
| Cdc20 | 1268.9 | 1.1 | 1.14 | 7.2E−14 | 1.12E−11 |
| Tfec | 71.7 | 1.1 | 1.32 | 6.43E−05 | 0.001657 |
| Isg15 | 787.9 | 1.1 | 1.38 | 0.000249 | 0.005119 |
| Klra7 | 38.4 | 1.1 | 1.58 | 0.00111 | 0.016512 |
| Hist1h2bj | 17.7 | 1.1 | 1.75 | 0.001687 | 0.022479 |
| Kbtbd6 | 12 | 1.1 | 2.04 | 0.002527 | 0.030129 |
| Stx11 | 1153.5 | 1.09 | 1.14 | 3.55E−12 | 4.68E−10 |
| Lgals1 | 11014.9 | 1.09 | 1.15 | 9.3E−10 | 8.22E−08 |
| Ccl8 | 29.5 | 1.09 | 4.6 | 0.003929 | 0.041586 |
| Nt5dc2 | 188.1 | 1.08 | 1.22 | 4.38E−06 | 0.000174 |
| Dnph1 | 136.4 | 1.08 | 1.29 | 6.66E−05 | 0.001706 |
| Bcat1 | 363.8 | 1.07 | 1.14 | 8.94E−10 | 8.07E−08 |
| Kifc1 | 310.5 | 1.07 | 1.17 | 8.33E−08 | 5.52E−06 |
| Mybl2 | 481.2 | 1.07 | 1.17 | 5.93E−07 | 3.09E−05 |
| Mx1 | 719.3 | 1.07 | 1.66 | 0.002705 | 0.031699 |
| Mcm5 | 5776.8 | 1.06 | 1.1 | 0 | 9.6E−14 |
| Cdkn2c | 421.3 | 1.06 | 1.11 | 1.25E−11 | 1.49E−09 |
| Ccl2 | 77.5 | 1.06 | 1.47 | 0.001637 | 0.022077 |
| Ifit1 | 298.3 | 1.06 | 1.61 | 0.002709 | 0.031699 |
| Ighv5-2 | 17.8 | 1.06 | 2.05 | 0.004935 | 0.049159 |
| Ccr5 | 1338.8 | 1.05 | 1.1 | 1.43E−12 | 1.97E−10 |
| Serpinb6b | 1199 | 1.05 | 1.12 | 3.96E−09 | 3.2E−07 |
| Smc2 | 1176.2 | 1.05 | 1.12 | 4.67E−09 | 3.71E−07 |
| Isg20 | 861.4 | 1.05 | 1.15 | 1.91E−06 | 8.67E−05 |
| Klrc3 | 41.9 | 1.05 | 1.37 | 0.000456 | 0.008347 |
| Rtp4 | 1189.3 | 1.05 | 1.43 | 0.001656 | 0.022272 |
| Nrg1 | 17.4 | 1.05 | 1.91 | 0.00423 | 0.043995 |
| Gins1 | 191.5 | 1.04 | 1.17 | 3.75E−06 | 0.000152 |
| Knstrn | 855.1 | 1.03 | 1.06 | 5.4E−14 | 8.61E−12 |
| Phf19 | 475.4 | 1.03 | 1.08 | 7.44E−11 | 7.92E−09 |
| Art2a-ps | 407.6 | 1.03 | 1.17 | 4.28E−05 | 0.001207 |
| Rfc4 | 533.3 | 1.02 | 1.04 | 0 | 0 |
| Tnfsf11 | 468.5 | 1.02 | 1.11 | 4.92E−07 | 2.61E−05 |
| Fn1 | 453.7 | 1.02 | 1.33 | 0.001188 | 0.01738 |
| Gna14 | 24.8 | 1.02 | 1.42 | 0.002372 | 0.028943 |
| Bard1 | 316.8 | 1.01 | 1.07 | 2.26E−09 | 1.9E−07 |
| Pole | 1124.1 | 1.01 | 1.07 | 2.85E−08 | 2.01E−06 |
| Pdcd1 | 880.8 | 1.01 | 1.08 | 6.75E−08 | 4.53E−06 |
| Prc1 | 830.9 | 1.01 | 1.1 | 1.43E−06 | 6.68E−05 |
| Art2b | 542.6 | 1.01 | 1.19 | 0.000104 | 0.002467 |
| Mcm8 | 150.3 | 1 | 1.09 | 8.42E−07 | 4.25E−05 |
| Xaf1 | 1257.6 | 1 | 1.1 | 1.6E−06 | 7.42E−05 |
| Tnfrsf8 | 167.1 | 1 | 1.1 | 4.36E−06 | 0.000173 |
| Apitd1 | 305.6 | 1 | 1.14 | 2.51E−05 | 0.000779 |
| Gstt3 | 58.6 | 1 | 1.15 | 5.96E−05 | 0.001564 |
| Hist1h1b | 56.5 | 1 | 1.32 | 0.001234 | 0.01782 |
| Cfap77 | 26 | 1 | 1.31 | 0.001439 | 0.019968 |
| Myo1d | 68 | 1 | 1.42 | 0.002852 | 0.032892 |
| 4930438A08Rik | 145.8 | −1.01 | −1.17 | 0.000312 | 0.006134 |
| Gm7860 | 120.6 | −1.02 | −1.09 | 1.76E−07 | 1.08E−05 |
| Gm38405 | 61.2 | −1.03 | −1.22 | 0.000285 | 0.005674 |
| Gm5608 | 44.9 | −1.03 | −1.27 | 0.000717 | 0.011861 |
| Fjx1 | 45.8 | −1.03 | −1.38 | 0.002866 | 0.033002 |
| Gm37510 | 31.1 | −1.03 | −1.48 | 0.004083 | 0.042776 |
| 9230114K14Rik | 64.2 | −1.04 | −1.19 | 6.16E−05 | 0.001606 |
| Cd164l2 | 60 | −1.04 | −1.24 | 0.000281 | 0.005618 |
| Podn | 23.2 | −1.04 | −1.32 | 0.001135 | 0.016833 |
| Mfrp | 55.6 | −1.04 | −1.33 | 0.0016 | 0.021746 |
| Timm8a2 | 27.2 | −1.05 | −1.28 | 0.000484 | 0.00875 |
| Gm11210 | 24.9 | −1.06 | −1.38 | 0.000895 | 0.014062 |
| Trim72 | 99 | −1.08 | −1.33 | 0.000468 | 0.008514 |
| Rbm44 | 28.3 | −1.09 | −1.5 | 0.001468 | 0.020306 |
| Pygm | 1170.9 | −1.12 | −1.19 | 1.17E−10 | 1.2E−08 |
| Prr15 | 32.2 | −1.12 | −1.31 | 5.32E−05 | 0.001422 |
| Prdm14 | 18.6 | −1.13 | −1.53 | 0.001494 | 0.020587 |
| Tm4sf1 | 20.6 | −1.14 | −1.56 | 0.001205 | 0.017549 |
| Rpl31-ps6 | 17.6 | −1.15 | −1.6 | 0.000885 | 0.013952 |
| Six4 | 17.7 | −1.17 | −1.57 | 0.000705 | 0.011726 |
| Tex45 | 736.4 | −1.19 | −1.29 | 6.92E−09 | 5.36E−07 |
| Apol8 | 1161.9 | −1.2 | −1.25 | 3E−15 | 4.88E−13 |
| AC158622.5 | 86.8 | −1.23 | −1.44 | 9.3E−06 | 0.00033 |
| Fgf17 | 23 | −1.27 | −1.72 | 0.000249 | 0.005119 |
| AC166361.2 | 101 | −1.3 | −1.42 | 2.71E−09 | 2.24E−07 |
| Gsdmc4 | 19.2 | −1.33 | −1.63 | 1.68E−05 | 0.00055 |
| Ap3s1-ps2 | 15.1 | −1.34 | −1.96 | 0.000176 | 0.003785 |
| Pmel | 62.3 | −1.73 | −4.35 | 1.15E−06 | 5.52E−05 |

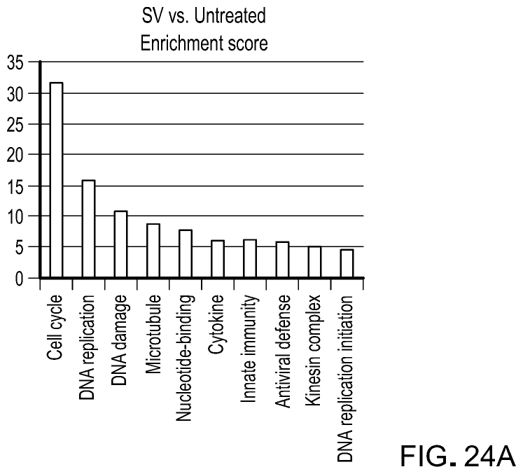

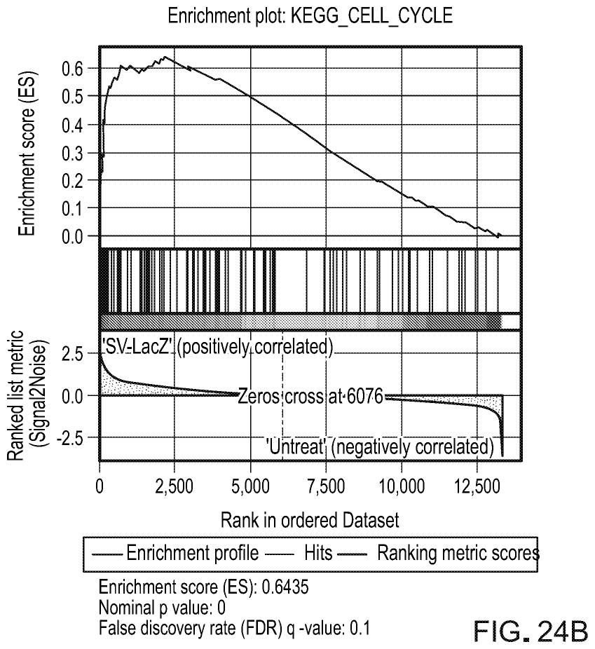

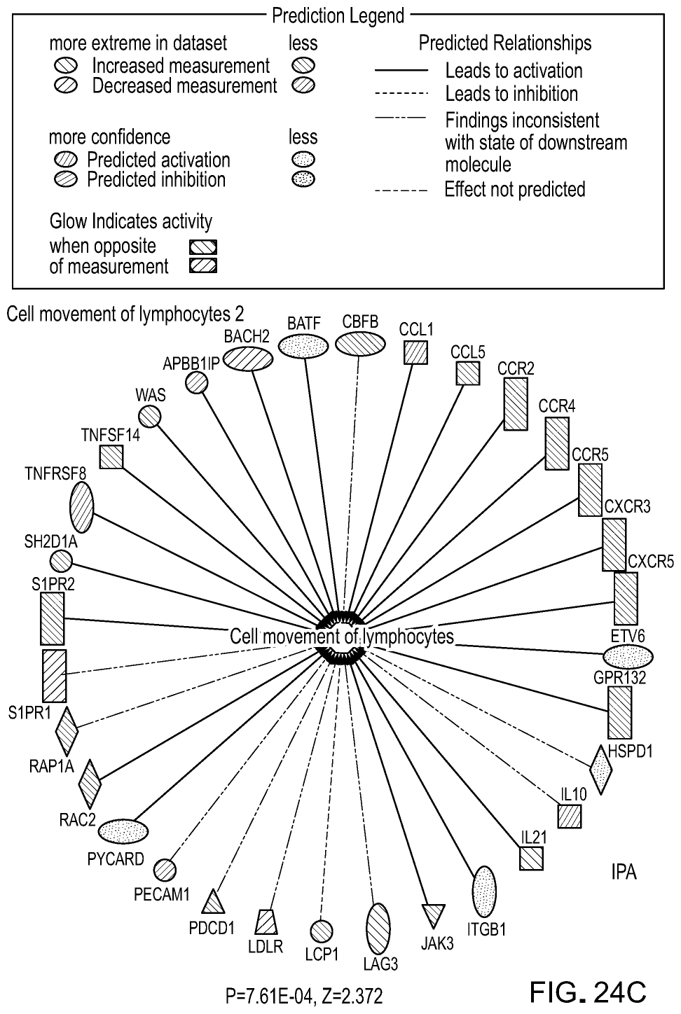

NIH DAVID cluster analysis was performed using the upregulated gene list. Enriched clusters were ranked based on enrichment score. Cell cycle gene cluster achieved the highest enrichment score (FIGS. 23B and 24A). This result was confirmed by KEGG gene set enrichment analysis (GSEA) (FIG. 24B). Cell cycle gene set ranks as the highest (enrichment score=0.64, FDR q value=0.1, nominal p value=0). These results indicate that SV infection enhances T cell cycle progression. SV induced upregulation of a series of cytokine and chemokine/chemokine receptors (FIG. 23C, left). To identify cytokines/chemokines that are upregulated by the administration of SV vectors, we compared SV plus α4-1BB mAb versus α4-1BB mAb (FIG. 23C, right). CCL8, IL-4, IL-13 and IL-21 were among those RNAs whose expression was upregulated by SV treatment. IL-21 anti-tumor effect is dependent on the activation of T, B and NK cells [Leonard W J et al., F1000Res. 2016]. IL-4, IL-10, IL-21 upregulation is consistent with previous reports [Rowell J F et al., J Immunol. 1999, Metcalf T U et al., J Virol. 2013]. In addition, Ingenuity Pathway Analysis (IPA) indicates that SV treatment enhances T cell movement by altering the expression of a number of molecules involved migration (Table 2, FIG. 24C), including a number of chemokines and chemokine receptors.

TABLE 2
The upregulated cell movement pathway for SV vs. untreated
group by IPA. SV induced SD upregulated gene sets are
clustered by DAVID analysis (SV vs. Untreated). Gene
clusters are ranked by enrichment score.

| | | Prediction | | |
|---|---|---|---|---|
| | | (based on | | |
| | Genes in | measurement | Expr Log | |
| ID | dataset | direction) | Ratio | Findings |
| Il21 | IL21 | Increased | 2.57 | Increases |
| | | | | (2) |
| Lag3 | LAG3 | Decreased | 1.76 | Decreases |
| | | | | (3) |
| Il10 | IL10 | Decreased | 1.66 | Decreases |
| | | | | (2) |
| Ccl1 | CCL1 | Increased | 1.12 | Increases |
| | | | | (1) |
| Ccr5 | CCR5 | Increased | 1.05 | Increases |
| | | | | (3) |
| Pdcd1 | PDCD1 | Decreased | 1.01 | Decreases |
| | | | | (3) |
| Tnfrsf8 | TNFRSF8 | Increased | 1 | Increases |
| | | | | (1) |
| Cxcr5 | CXCR5 | Increased | 0.98 | Increases |
| | | | | (1) |
| Ccr2 | CCR2 | Increased | 0.9 | Increases |
| | | | | (9) |
| Cxcr3 | CXCR3 | Increased | 0.77 | Increases |
| | | | | (1) |
| Sh2d1a | SH2D1A | Increased | 0.68 | Increases |
| | | | | (1) |
| Batf | BATF | Increased | 0.67 | Increases |
| | | | | (7) |
| Pycard | PYCARD | Increased | 0.64 | Increases |
| | | | | (3) |
| Ccr4 | CCR4 | Increased | 0.62 | Increases |
| | | | | (4) |
| Ccl5 | CCL5 | Increased | 0.62 | Increases |
| | | | | (2) |
| Itgb1 | ITGB1 | Increased | 0.51 | Increases |
| | | | | (1) |
| S1pr2 | S1PR2 | Increased | 0.5 | Increases |
| | | | | (1) |
| Lcp1 | LCP1 | Affected | 0.5 | Affects |
| | | | | (1) |
| Tnfsf14 | TNFSF14 | Increased | 0.5 | Increases |
| | | | | (1) |
| Hspd1 | HSPD1 | Decreased | 0.41 | Decreases |
| | | | | (1) |
| Cbfb | CBFB | Decreased | 0.4 | Decreases |
| | | | | (1) |
| Jak3 | JAK3 | Increased | 0.36 | Increases |
| | | | | (1) |
| Rap1a | RAP1A | Decreased | 0.31 | Decreases |
| | | | | (3) |
| Was | WAS | Increased | 0.29 | Increases |
| | | | | (2) |
| Etv6 | ETV6 | Increased | 0.26 | Increases |
| | | | | (3) |
| Rac2 | RAC2 | Increased | 0.25 | Increases |
| | | | | (36) |
| Apbb1ip | APBB1IP | Increased | −0.21 | Decreases |
| | | | | (2) |
| Pecam1 | PECAM1 | Decreased | −0.34 | Increases |
| | | | | (10) |
| Ldlr | LDLR | Decreased | −0.41 | Increases |
| | | | | (1) |
| Bach2 | BACH2 | Increased | −0.46 | Decreases |
| | | | | (1) |
| S1pr1 | S1PR1 | Decreased | −0.58 | Increases |
| | | | | (6) |
| Gpr132 | GPR132 | Increased | −0.6 | Decreases |
| | | | | (1) |

To understand why SV plus α4-1BB mAb achieves the best therapeutic effect, Deseq2 analysis was run for SV plus α4-1BB mAb vs. untreated samples. 1046 upregulated genes (q<0.05 and Log 2 Fold Change≥1) and 877 downregulated genes (q<0.05 and Log 2 Fold Change≤−1) in the SV plus α4-1BB mAb group were identified (FIG. 23A, Table 3). T cells from animals treated with SV+α4-1BB mAb vs. treated were also compared with SV only and 316 upregulated genes (p<0.05 and Log 2 Fold Change≥1) and 439 downregulated genes (p<0.05 and Log 2 Fold Change≤−1) in the SV+α4-1BB mAb treated group were found (FIG. 23A, Table 4).

TABLE 3
The SD expressed genes list for SV + α4-1BB vs. untreated group by RNA-
Seq (q < 0.05, Log2FC ≥ 1 and Log2FC ≤ −1).

| gene | baseMean | log2FC | log2FCunshrunk | pvalue | padj |
|---|---|---|---|---|---|
| Gzmk | 1785.2 | 4.75 | 5.32 | 0 | 0 |
| Gzmb | 4113.9 | 4.37 | 4.81 | 0 | 0 |
| Ccl8 | 29.5 | 3.64 | 8.24 | 0 | 0 |
| Ociad2 | 184.7 | 3.53 | 3.76 | 0 | 0 |
| Aurkb | 806.6 | 3.51 | 3.73 | 0 | 0 |
| Ret | 794.7 | 3.51 | 4.41 | 0 | 0 |
| E2f8 | 535.1 | 3.5 | 3.88 | 0 | 0 |
| Tpx2 | 1113.7 | 3.34 | 3.58 | 0 | 0 |
| Kif2c | 341.3 | 3.33 | 3.58 | 0 | 0 |
| Kif18b | 606.6 | 3.29 | 3.51 | 0 | 0 |
| Hmmr | 311.7 | 3.26 | 3.61 | 0 | 0 |
| Cdc25c | 143.9 | 3.25 | 3.81 | 0 | 0 |
| Pbk | 107.5 | 3.24 | 3.63 | 0 | 0 |
| Cenpf | 495.6 | 3.23 | 3.46 | 0 | 0 |
| Prr11 | 288 | 3.23 | 3.68 | 0 | 0 |
| Ccna2 | 1680.2 | 3.18 | 3.34 | 0 | 0 |
| Ccnb2 | 1255.4 | 3.16 | 3.38 | 0 | 0 |
| Bub1 | 362.3 | 3.16 | 3.44 | 0 | 0 |
| Pif1 | 204 | 3.16 | 3.57 | 0 | 0 |
| Klrg1 | 746.6 | 3.15 | 3.34 | 0 | 0 |
| Nusap1 | 813.1 | 3.14 | 3.31 | 0 | 0 |
| Bub1b | 1344.3 | 3.13 | 3.32 | 0 | 0 |
| Rad54l | 350.2 | 3.13 | 3.43 | 0 | 0 |
| Cdk1 | 1228.8 | 3.11 | 3.32 | 0 | 0 |
| Mki67 | 5899.1 | 3.11 | 3.36 | 0 | 0 |
| Rad51 | 401.4 | 3.08 | 3.34 | 0 | 0 |
| Ttk | 211.5 | 3.08 | 3.4 | 0 | 0 |
| Nuf2 | 279.6 | 3.08 | 3.46 | 0 | 0 |
| Fignl1 | 145.7 | 3.07 | 3.37 | 0 | 0 |
| Shcbp1 | 274.8 | 3.05 | 3.26 | 0 | 0 |
| Cdkn3 | 129.2 | 3.05 | 3.35 | 0 | 0 |
| Rrm2 | 1718.2 | 3.04 | 3.26 | 0 | 0 |
| Ube2c | 1391 | 3.03 | 3.35 | 0 | 0 |
| Pclaf | 522.9 | 3.03 | 3.36 | 0 | 0 |
| Lrr1 | 59 | 3.03 | 3.74 | 0 | 0 |
| Cdca8 | 1251.5 | 3.02 | 3.21 | 0 | 0 |
| Cep55 | 503.9 | 3.02 | 3.24 | 0 | 0 |
| Mem10 | 477.4 | 3.01 | 3.22 | 0 | 0 |
| Sapcd2 | 275.5 | 2.99 | 3.28 | 0 | 0 |
| Pimreg | 230 | 2.99 | 3.3 | 0 | 0 |
| Kif4 | 650.2 | 2.97 | 3.13 | 0 | 0 |
| Kntc1 | 402.4 | 2.96 | 3.25 | 0 | 0 |
| Ccr5 | 1338.8 | 2.95 | 3.07 | 0 | 0 |
| Kif14 | 299.1 | 2.94 | 3.2 | 0 | 0 |
| Sgo1 | 162.8 | 2.94 | 3.21 | 0 | 0 |
| Spc24 | 506 | 2.93 | 3.08 | 0 | 0 |
| Espl1 | 809.6 | 2.93 | 3.12 | 0 | 0 |
| Clspn | 637.1 | 2.93 | 3.14 | 0 | 0 |
| Wdr95 | 226 | 2.88 | 3.09 | 0 | 0 |
| Nek2 | 554.5 | 2.88 | 3.13 | 0 | 0 |
| Ccnb1 | 160.8 | 2.88 | 3.25 | 0 | 0 |
| Ncapg | 383.1 | 2.86 | 3.09 | 0 | 0 |
| Top2a | 4726.3 | 2.85 | 2.96 | 0 | 0 |
| Ncaph | 822.4 | 2.85 | 3 | 0 | 0 |
| Kif20a | 898.5 | 2.85 | 3.03 | 0 | 0 |
| Diaph3 | 356.3 | 2.85 | 3.04 | 0 | 0 |
| Akr1c18 | 25.3 | 2.85 | 4.88 | 6E−15 | 1.84E−13 |
| Ckap2l | 455.4 | 2.83 | 3.1 | 0 | 0 |
| Oip5 | 50.6 | 2.83 | 3.55 | 0 | 0 |
| Ermn | 17.7 | 2.82 | 5.41 | 3.2E−14 | 8.82E−13 |
| Spag5 | 1183.2 | 2.81 | 2.97 | 0 | 0 |
| Birc5 | 1540.7 | 2.81 | 3.13 | 0 | 0 |
| Dpysl5 | 94.9 | 2.78 | 3.57 | 0 | 0 |
| Cdca5 | 791.4 | 2.77 | 2.88 | 0 | 0 |
| Iqgap3 | 284.2 | 2.77 | 2.98 | 0 | 0 |
| Kif15 | 595.3 | 2.76 | 2.95 | 0 | 0 |
| Plk1 | 794.8 | 2.75 | 2.94 | 0 | 0 |
| Troap | 236.4 | 2.75 | 3.04 | 0 | 0 |
| Cenpe | 685.5 | 2.73 | 2.91 | 0 | 0 |
| Esco2 | 149.5 | 2.73 | 3.04 | 0 | 0 |
| Gng3 | 34.8 | 2.72 | 3.74 | 0 | 1E−15 |
| Cit | 784.3 | 2.71 | 2.9 | 0 | 0 |
| Kif22 | 1022.2 | 2.71 | 2.91 | 0 | 0 |
| Car5b | 136 | 2.71 | 2.94 | 0 | 0 |
| Ncald | 297.2 | 2.71 | 2.95 | 0 | 0 |
| Cdca2 | 444.5 | 2.7 | 2.87 | 0 | 0 |
| Knl1 | 203.3 | 2.7 | 3.01 | 0 | 0 |
| Hist1h1b | 56.5 | 2.7 | 3.28 | 0 | 0 |
| Aunip | 24.6 | 2.69 | 4.29 | 1.5E−14 | 4.26E−13 |
| 1500009L16Rik | 143.2 | 2.68 | 3.37 | 0 | 0 |
| Osr2 | 54.7 | 2.68 | 4.06 | 8E−15 | 2.43E−13 |
| Depdc1b | 350.8 | 2.67 | 2.86 | 0 | 0 |
| Ticrr | 558.4 | 2.67 | 2.87 | 0 | 0 |
| Melk | 329.8 | 2.66 | 2.82 | 0 | 0 |
| Tpsab1 | 262.4 | 2.66 | 3.16 | 0 | 0 |
| Smpdl3b | 185.8 | 2.65 | 2.9 | 0 | 0 |
| Spc25 | 211.3 | 2.65 | 2.95 | 0 | 0 |
| Cdc45 | 628.7 | 2.64 | 2.73 | 0 | 0 |
| Ccr2 | 1142.1 | 2.64 | 2.83 | 0 | 0 |
| Il21 | 229 | 2.64 | 3.76 | 3.3E−14 | 9.01E−13 |
| BC030867 | 129.6 | 2.63 | 2.93 | 0 | 0 |
| Uhrfl | 1603.6 | 2.61 | 2.76 | 0 | 0 |
| Mxd3 | 280.1 | 2.61 | 2.76 | 0 | 0 |
| Tyms | 809.6 | 2.6 | 2.7 | 0 | 0 |
| Neil3 | 228.1 | 2.6 | 2.82 | 0 | 0 |
| Trip13 | 233.7 | 2.59 | 2.78 | 0 | 0 |
| Rad51ap1 | 205.6 | 2.58 | 2.78 | 0 | 0 |
| Asf1b | 1276.4 | 2.57 | 2.69 | 0 | 0 |
| Kif11 | 1109.9 | 2.55 | 2.67 | 0 | 0 |
| Ska1 | 210.9 | 2.55 | 2.86 | 0 | 0 |
| Stmn1 | 257.1 | 2.53 | 2.71 | 0 | 0 |
| Ccl5 | 22142.8 | 2.53 | 2.75 | 0 | 0 |
| Ly6a | 2208.2 | 2.53 | 3.76 | 5.03E−13 | 1.2E−11 |
| Igf2bp3 | 26.6 | 2.52 | 3.61 | 1.47E−13 | 3.72E−12 |
| Aspm | 319.4 | 2.51 | 2.77 | 0 | 0 |
| Parpbp | 117.3 | 2.51 | 2.86 | 0 | 0 |
| Ect2 | 255.1 | 2.49 | 2.82 | 0 | 0 |
| Cdca3 | 1038.6 | 2.48 | 2.6 | 0 | 0 |
| E2f7 | 275.3 | 2.48 | 2.74 | 0 | 0 |
| Depdc1a | 29.6 | 2.48 | 3.46 | 2E−15 | 7.6E−14 |
| Gm33460 | 48.9 | 2.47 | 2.96 | 0 | 0 |
| Tex15 | 58.4 | 2.47 | 3 | 0 | 0 |
| Tk1 | 1004.9 | 2.46 | 2.59 | 0 | 0 |
| Lag3 | 2916.7 | 2.45 | 2.56 | 0 | 0 |
| Spdl1 | 204.5 | 2.45 | 2.61 | 0 | 0 |
| Gm15056 | 28.9 | 2.45 | 4.48 | 4.74E−11 | 8.5E−10 |
| Ckap2 | 394.5 | 2.44 | 2.55 | 0 | 0 |
| Ankle1 | 238.9 | 2.44 | 2.71 | 0 | 0 |
| Mt3 | 18.3 | 2.42 | 4.28 | 3.9E−11 | 7.15E−10 |
| Cenph | 201.9 | 2.41 | 2.66 | 0 | 0 |
| Arsb | 1795.9 | 2.4 | 2.55 | 0 | 0 |
| Cenpi | 136.8 | 2.4 | 2.6 | 0 | 0 |
| Gm6637 | 196.3 | 2.4 | 2.69 | 0 | 0 |
| Ndc80 | 345.5 | 2.39 | 2.53 | 0 | 0 |
| Chaf1a | 1027.2 | 2.39 | 2.53 | 0 | 0 |
| Cenpm | 289.6 | 2.39 | 2.61 | 0 | 0 |
| CT030166.6 | 24.2 | 2.37 | 3.39 | 1.66E−12 | 3.74E−11 |
| Ms4a3 | 31.1 | 2.37 | 4.07 | 2E−10 | 3.29E−09 |
| Il13 | 60.8 | 2.36 | 3.42 | 3.84E−11 | 7.06E−10 |
| Ctsg | 151.2 | 2.36 | 3.9 | 2.01E−10 | 3.3E−09 |
| Col6a5 | 17.7 | 2.36 | 6.13 | 4.48E−10 | 6.83E−09 |
| Ncapd2 | 3741.3 | 2.33 | 2.39 | 0 | 0 |
| Tcf19 | 1158.6 | 2.32 | 2.41 | 0 | 0 |
| Gbp11 | 355.1 | 2.31 | 2.46 | 0 | 0 |
| Fhl2 | 217.6 | 2.3 | 2.44 | 0 | 0 |
| Ncapg2 | 526.2 | 2.29 | 2.43 | 0 | 0 |
| Cks1b | 772.5 | 2.28 | 2.46 | 0 | 0 |
| Gzma | 3261.2 | 2.28 | 3.76 | 8.07E−10 | 1.18E−08 |
| Nkg7 | 8456.1 | 2.27 | 2.34 | 0 | 0 |
| Pask | 202.9 | 2.27 | 2.43 | 0 | 0 |
| Il10 | 1005.5 | 2.27 | 2.5 | 0 | 0 |
| Prc1 | 830.9 | 2.26 | 2.46 | 0 | 0 |
| Wipf3 | 37.6 | 2.26 | 3.33 | 3.67E−12 | 7.88E−11 |
| Mpo | 1003.3 | 2.26 | 3.54 | 7.46E−10 | 1.09E−08 |
| Osbpl3 | 1358.5 | 2.25 | 2.33 | 0 | 0 |
| Mastl | 108.5 | 2.25 | 2.54 | 0 | 0 |
| Serpinb6b | 1199 | 2.24 | 2.38 | 0 | 0 |
| Adgrg1 | 449.6 | 2.24 | 2.4 | 0 | 0 |
| Slc16a2 | 105.6 | 2.24 | 2.53 | 0 | 0 |
| Cel1 | 31 | 2.24 | 3.2 | 7.42E−11 | 1.29E−09 |
| Cdc6 | 570.6 | 2.22 | 2.34 | 0 | 0 |
| Bspry | 174.2 | 2.22 | 2.64 | 1E−15 | 3.6E−14 |
| Ccne1 | 503.1 | 2.21 | 2.36 | 0 | 0 |
| Cenpp | 81.2 | 2.21 | 2.47 | 0 | 0 |
| Lxn | 79 | 2.21 | 2.45 | 0 | 0 |
| Lgals1 | 11014.9 | 2.2 | 2.34 | 0 | 0 |
| Adap1 | 1257.2 | 2.18 | 2.3 | 0 | 0 |
| F13a1 | 227.4 | 2.17 | 3.04 | 1.97E−10 | 3.24E−09 |
| Cdc20 | 1268.9 | 2.16 | 2.24 | 0 | 0 |
| Tigit | 2942.9 | 2.16 | 2.38 | 0 | 0 |
| Cdkn2a | 59.4 | 2.15 | 2.56 | 3.4E−14 | 9.34E−13 |
| Exo1 | 187.1 | 2.14 | 2.4 | 0 | 0 |
| Clip4 | 39.8 | 2.14 | 2.75 | 7.69E−12 | 1.58E−10 |
| F2rl2 | 42.8 | 2.13 | 2.58 | 1.57E−13 | 3.95E−12 |
| Hist1h2bj | 17.7 | 2.13 | 2.99 | 4.05E−10 | 6.22E−09 |
| Gldc | 15.3 | 2.13 | 3.09 | 1.37E−09 | 1.92E−08 |
| Dhfr | 503.3 | 2.12 | 2.24 | 0 | 0 |
| Lig1 | 3384.1 | 2.11 | 2.19 | 0 | 0 |
| Cenpn | 307 | 2.11 | 2.23 | 0 | 0 |
| Tox2 | 889.5 | 2.11 | 2.32 | 0 | 0 |
| Cdkn2c | 421.3 | 2.1 | 2.2 | 0 | 0 |
| Kifc1 | 310.5 | 2.1 | 2.26 | 0 | 0 |
| Slc22a3 | 85.3 | 2.1 | 2.57 | 1.47E−12 | 3.33E−11 |
| Msr1 | 48.9 | 2.1 | 2.73 | 1.56E−11 | 3.04E−10 |
| Trp73 | 22 | 2.1 | 3.54 | 2.07E−08 | 2.37E−07 |
| Prtn3 | 290.9 | 2.1 | 3.62 | 2.37E−08 | 2.68E−07 |
| Csf2 | 72.9 | 2.09 | 2.47 | 8E−14 | 2.1E−12 |
| Fancd2 | 392.9 | 2.08 | 2.16 | 0 | 0 |
| Gm2788 | 29.7 | 2.08 | 2.77 | 5.16E−11 | 9.2E−10 |
| Nfe2 | 91.3 | 2.08 | 2.85 | 9.6E−10 | 1.38E−08 |
| Rnase2a | 5.3 | 2.08 | 6.48 | 1.58E−08 | 1.84E−07 |
| Mis18bp1 | 180.6 | 2.07 | 2.26 | 0 | 0 |
| Samd14 | 203.2 | 2.07 | 2.39 | 3E−15 | 8.5E−14 |
| Oas1a | 498.3 | 2.07 | 2.42 | 4E−15 | 1.1E−13 |
| Hist1h3c | 11.8 | 2.07 | 3.72 | 2.61E−08 | 2.93E−07 |
| Tacc3 | 1922.3 | 2.06 | 2.12 | 0 | 0 |
| Wdr31 | 15.1 | 2.06 | 3.44 | 1.23E−08 | 1.46E−07 |
| Knstrn | 855.1 | 2.05 | 2.12 | 0 | 0 |
| Anxa2 | 5777.8 | 2.05 | 2.21 | 0 | 0 |
| Tpbg | 19.3 | 2.04 | 3.63 | 2.53E−08 | 2.85E−07 |
| Cst7 | 1893.9 | 2.02 | 2.09 | 0 | 0 |
| Slc43a3 | 380.4 | 2.02 | 2.16 | 0 | 0 |
| Muc13 | 227 | 2.02 | 2.24 | 0 | 0 |
| Rad51b | 65.9 | 2.02 | 2.3 | 0 | 5E−15 |
| Xkr5 | 39.4 | 2.02 | 2.42 | 2.9E−13 | 7.07E−12 |
| Chil3 | 42.4 | 2.02 | 3.21 | 4.25E−08 | 4.61E−07 |
| Bard1 | 316.8 | 2.01 | 2.11 | 0 | 0 |
| Acot7 | 2318.6 | 2.01 | 2.12 | 0 | 0 |
| Arhgap11a | 847.4 | 2.01 | 2.13 | 0 | 0 |
| Rad54b | 114.1 | 2.01 | 2.21 | 0 | 0 |
| Pcdhgc4 | 19.5 | 2.01 | 2.97 | 5.51E−09 | 6.96E−08 |
| Mcm5 | 5776.8 | 2 | 2.06 | 0 | 0 |
| Rmi2 | 184.3 | 2 | 2.12 | 0 | 0 |
| Dna2 | 343.7 | 2 | 2.16 | 0 | 0 |
| S100a6 | 3227.2 | 2 | 2.17 | 0 | 0 |
| Mybl2 | 481.2 | 2 | 2.19 | 0 | 0 |
| Brca1 | 443.2 | 1.99 | 2.11 | 0 | 0 |
| Esm1 | 680.1 | 1.99 | 2.16 | 0 | 0 |
| E2fl | 876.4 | 1.98 | 2.04 | 0 | 0 |
| F10 | 33.6 | 1.98 | 3.1 | 4.08E−08 | 4.44E−07 |
| Lif | 439.6 | 1.97 | 2.05 | 0 | 0 |
| Pglyrp1 | 1046.5 | 1.97 | 2.15 | 0 | 0 |
| Fam19a3 | 168.7 | 1.97 | 2.22 | 1E−15 | 1.9E−14 |
| Spp1 | 51 | 1.97 | 2.41 | 2.47E−11 | 4.68E−10 |
| Faxc | 12.4 | 1.97 | 3.44 | 1.3E−07 | 1.3E−06 |
| Stil | 422.9 | 1.96 | 2.17 | 0 | 0 |
| Dapk2 | 315.5 | 1.95 | 2.04 | 0 | 0 |
| Serpinb9 | 1224.7 | 1.95 | 2.06 | 0 | 0 |
| Fanca | 375.8 | 1.95 | 2.06 | 0 | 0 |
| Bcat1 | 363.8 | 1.95 | 2.06 | 0 | 0 |
| Neurl1b | 49.9 | 1.95 | 2.46 | 2.74E−10 | 4.38E−09 |
| Gins1 | 191.5 | 1.94 | 2.14 | 0 | 0 |
| Tg | 98.4 | 1.94 | 2.15 | 0 | 1E−15 |
| Dnph1 | 136.4 | 1.94 | 2.27 | 5.77E−13 | 1.36E−11 |
| Tmem40 | 47.5 | 1.94 | 2.52 | 2.2E−09 | 2.98E−08 |
| Hist1h3g | 9.3 | 1.94 | 3.19 | 1.43E−07 | 1.41E−06 |
| Ighg1 | 2604.8 | 1.94 | 4.76 | 2.48E−07 | 2.33E−06 |
| Psrc1 | 122.1 | 1.93 | 2.07 | 0 | 0 |
| Serpina3f | 452 | 1.93 | 2.12 | 0 | 0 |
| Robo3 | 27.3 | 1.93 | 2.44 | 7.7E−10 | 1.13E−08 |
| Gcg | 62.2 | 1.93 | 5.91 | 2.19E−07 | 2.08E−06 |
| Smc2 | 1176.2 | 1.92 | 2.03 | 0 | 0 |
| Sgo2a | 143.4 | 1.92 | 2.11 | 0 | 0 |
| Nt5dc2 | 188.1 | 1.92 | 2.15 | 0 | 5E−15 |
| Klrc2 | 53.2 | 1.92 | 2.27 | 5.7E−13 | 1.35E−11 |
| Prss57 | 9.8 | 1.92 | 3.88 | 2.9E−07 | 2.7E−06 |
| Gpsm2 | 436.7 | 1.91 | 2.02 | 0 | 0 |
| Nsl1 | 195.3 | 1.9 | 2.03 | 0 | 0 |
| C3 | 1368.2 | 1.9 | 2.15 | 6E−15 | 1.81E−13 |
| Angpt1 | 63.2 | 1.9 | 2.28 | 1.89E−11 | 3.65E−10 |
| Popdc2 | 54.7 | 1.89 | 2.36 | 3.65E−10 | 5.66E−09 |
| Kbtbd6 | 12 | 1.89 | 3.03 | 1.35E−07 | 1.34E−06 |
| Stk32c | 341 | 1.87 | 1.99 | 0 | 0 |
| Syce2 | 175.1 | 1.87 | 2.03 | 0 | 0 |
| Gp9 | 24.8 | 1.87 | 2.53 | 3.19E−08 | 3.56E−07 |
| Poc1a | 388.3 | 1.86 | 1.95 | 0 | 0 |
| 2610318N02Rik | 193.2 | 1.86 | 2.02 | 0 | 0 |
| Ifng | 1413.7 | 1.86 | 2.11 | 5.7E−14 | 1.51E−12 |
| Hcn2 | 36.3 | 1.86 | 2.42 | 6.4E−09 | 7.97E−08 |
| Klhl23 | 20.6 | 1.86 | 3.13 | 1.4E−07 | 1.38E−06 |
| Nrg1 | 17.4 | 1.86 | 3 | 3.31E−07 | 3.06E−06 |
| Insrr | 64.7 | 1.85 | 2.05 | 3E−15 | 1.03E−13 |
| Cldnd2 | 53.5 | 1.85 | 2.12 | 3.45E−13 | 8.31E−12 |
| Ifit1bl1 | 137.8 | 1.85 | 2.34 | 1.18E−09 | 1.67E−08 |
| Scn11a | 5.9 | 1.85 | 5.36 | 7.48E−07 | 6.42E−06 |
| Pdzph1 | 10.7 | 1.85 | 4.11 | 9.95E−07 | 8.34E−06 |
| Gins2 | 413.5 | 1.84 | 1.94 | 0 | 0 |
| Gm12250 | 177.3 | 1.84 | 2.07 | 6E−15 | 1.73E−13 |
| Dach1 | 18.3 | 1.84 | 2.8 | 3.05E−07 | 2.83E−06 |
| Rasgef1a | 270.7 | 1.83 | 1.94 | 0 | 0 |
| Pcyt1b | 106.8 | 1.83 | 2.01 | 0 | 0 |
| Oas3 | 1474.3 | 1.83 | 2.25 | 3.83E−10 | 5.9E−09 |
| Cd70 | 12.5 | 1.83 | 2.73 | 3.29E−07 | 3.05E−06 |
| Tuba8 | 42.2 | 1.83 | 2.76 | 4.67E−07 | 4.21E−06 |
| Tph1 | 43 | 1.83 | 3.53 | 1.35E−06 | 1.11E−05 |
| Zbtb32 | 667.1 | 1.82 | 1.9 | 0 | 0 |
| Tfr2 | 139.5 | 1.82 | 2.41 | 2.31E−08 | 2.62E−07 |
| Mmrn1 | 13.3 | 1.82 | 2.99 | 7.75E−07 | 6.64E−06 |
| Gm24289 | 6.9 | 1.82 | 5.21 | 1.22E−06 | 1.01E−05 |
| Dlgap5 | 705.3 | 1.81 | 1.9 | 0 | 0 |
| Ska3 | 252.1 | 1.81 | 1.91 | 0 | 0 |
| Fam81a | 55.4 | 1.81 | 2.16 | 1.92E−10 | 3.16E−09 |
| Plppr3 | 42.4 | 1.81 | 2.74 | 7.18E−07 | 6.2E−06 |
| Mpl | 45.7 | 1.81 | 2.69 | 7.96E−07 | 6.8E−06 |
| Hist1h2ag | 6.4 | 1.81 | 3.59 | 1.52E−06 | 1.23E−05 |
| Fbxo41 | 15.3 | 1.8 | 2.59 | 1.51E−07 | 1.48E−06 |
| Mcm3 | 5809.4 | 1.79 | 1.84 | 0 | 0 |
| Ska2 | 254.2 | 1.79 | 1.91 | 0 | 0 |
| Art2b | 542.6 | 1.79 | 2.08 | 6.27E−12 | 1.3E−10 |
| Necab3 | 27.4 | 1.79 | 2.36 | 1.87E−08 | 2.16E−07 |
| Lilr4b | 1553.9 | 1.78 | 1.91 | 0 | 0 |
| AA467197 | 30.3 | 1.78 | 2.21 | 9E−09 | 1.09E−07 |
| Maats1 | 7.8 | 1.78 | 3.55 | 2.36E−06 | 1.84E−05 |
| Col6a2 | 16.9 | 1.78 | 3.82 | 2.57E−06 | 1.99E−05 |
| Cdca7 | 631.9 | 1.77 | 1.89 | 0 | 0 |
| Serpina3g | 2916.5 | 1.77 | 1.92 | 0 | 0 |
| Dyrk3 | 249.1 | 1.77 | 1.95 | 2E−15 | 5.8E−14 |
| Apitd1 | 305.6 | 1.77 | 1.98 | 9.1E−14 | 2.37E−12 |
| AC153938.2 | 40.5 | 1.77 | 2.19 | 2.58E−09 | 3.47E−08 |
| Eomes | 4109.7 | 1.75 | 1.8 | 0 | 0 |
| Pola1 | 686.1 | 1.75 | 1.83 | 0 | 0 |
| Pole | 1124.1 | 1.75 | 1.86 | 0 | 0 |
| Eme1 | 219.4 | 1.75 | 1.88 | 0 | 0 |
| Map6 | 151 | 1.75 | 1.88 | 0 | 0 |
| Gm4951 | 44.3 | 1.75 | 2.24 | 1.88E−09 | 2.57E−08 |
| Tff3 | 16.7 | 1.75 | 2.36 | 1.47E−07 | 1.45E−06 |
| Nmral1 | 561.7 | 1.74 | 1.86 | 0 | 0 |
| Plac8 | 2583 | 1.74 | 1.92 | 2E−15 | 6.3E−14 |
| Klrc1 | 236.6 | 1.74 | 1.95 | 5E−14 | 1.34E−12 |
| S100a4 | 1170.3 | 1.74 | 1.96 | 7.04E−13 | 1.66E−11 |
| Mns1 | 129.3 | 1.74 | 2.05 | 2.42E−10 | 3.91E−09 |
| Miat | 31.2 | 1.74 | 2.07 | 5.13E−10 | 7.74E−09 |
| Efcab11 | 31.5 | 1.74 | 2.33 | 6.62E−08 | 6.98E−07 |
| Erfe | 15.4 | 1.74 | 2.77 | 9.89E−07 | 8.3E−06 |
| Kcnk5 | 399.5 | 1.73 | 1.81 | 0 | 0 |
| Stx11 | 1153.5 | 1.73 | 1.81 | 0 | 0 |
| Smtn | 271.7 | 1.73 | 1.87 | 0 | 0 |
| Anln | 217.2 | 1.73 | 1.89 | 1E−15 | 3.1E−14 |
| Gm17745 | 84 | 1.73 | 1.92 | 1.9E−14 | 5.44E−13 |
| Krt18 | 26.4 | 1.73 | 2.17 | 1.75E−08 | 2.03E−07 |
| 2900011O08Rik | 14.8 | 1.73 | 2.39 | 9.4E−07 | 7.92E−06 |
| Tpi1 | 2445.6 | 1.72 | 1.78 | 0 | 0 |
| Chsy1 | 2251.6 | 1.72 | 1.8 | 0 | 0 |
| Gtse1 | 554 | 1.72 | 1.81 | 0 | 0 |
| Pdcd1 | 880.8 | 1.72 | 1.84 | 0 | 0 |
| Vdr | 250.7 | 1.72 | 1.97 | 2.66E−11 | 5E−10 |
| Ltb4r1 | 44.5 | 1.72 | 2.06 | 1.19E−09 | 1.68E−08 |
| 2010110K18Rik | 30.1 | 1.72 | 2.11 | 9.25E−09 | 1.12E−07 |
| Alox5 | 104.2 | 1.72 | 2.35 | 2.33E−07 | 2.2E−06 |
| Pde10a | 17 | 1.72 | 2.48 | 7.24E−07 | 6.24E−06 |
| Dscc1 | 150.3 | 1.71 | 1.83 | 0 | 0 |
| Mcm6 | 5226.1 | 1.7 | 1.74 | 0 | 0 |
| Tfdp1 | 1161.4 | 1.7 | 1.76 | 0 | 0 |
| E2f2 | 2088.1 | 1.7 | 1.78 | 0 | 0 |
| 1700011L03Rik | 6.7 | 1.7 | 5.77 | 5.28E−06 | 3.81E−05 |
| Col6a1 | 15.6 | 1.7 | 4.62 | 6.57E−06 | 4.64E−05 |
| Chst11 | 1091.1 | 1.69 | 1.75 | 0 | 0 |
| Il12rb1 | 1086.8 | 1.69 | 1.76 | 0 | 0 |
| Gm4841 | 29.9 | 1.69 | 2.35 | 3.46E−07 | 3.19E−06 |
| Treml1 | 31.4 | 1.69 | 2.36 | 1.75E−06 | 1.4E−05 |
| Fcer1a | 69.7 | 1.69 | 2.77 | 3.83E−06 | 2.85E−05 |
| Rfc4 | 533.3 | 1.68 | 1.72 | 0 | 0 |
| Rgs16 | 7742.7 | 1.68 | 1.75 | 0 | 0 |
| Ezh2 | 1820.5 | 1.68 | 1.76 | 0 | 0 |
| Lockd | 222.2 | 1.68 | 1.85 | 3.1E−14 | 8.5E−13 |
| Fam57b | 23.3 | 1.68 | 5.08 | 4.18E−07 | 3.8E−06 |
| Dixdc1 | 18.6 | 1.68 | 2.66 | 2.93E−06 | 2.25E−05 |
| Trpc6 | 7.4 | 1.68 | 4.05 | 5.76E−06 | 4.12E−05 |
| Adra1b | 14.9 | 1.68 | 2.83 | 6.8E−06 | 4.78E−05 |
| Ttc39c | 656.7 | 1.67 | 1.72 | 0 | 0 |
| Wee1 | 206.8 | 1.67 | 1.75 | 0 | 0 |
| Neb | 354.6 | 1.67 | 1.9 | 2.03E−11 | 3.9E−10 |
| Prdm1 | 912.1 | 1.66 | 1.74 | 0 | 0 |
| Gimap7 | 2437.5 | 1.66 | 1.76 | 0 | 0 |
| Mcm8 | 150.3 | 1.66 | 1.79 | 0 | 4E−15 |
| Tnfrsf8 | 167.1 | 1.66 | 1.82 | 1E−14 | 3.02E−13 |
| Lgalsl | 189.5 | 1.66 | 1.82 | 2.5E−14 | 7.06E−13 |
| Ptgr1 | 201.9 | 1.66 | 1.84 | 1.25E−13 | 3.19E−12 |
| Fads2 | 70.5 | 1.66 | 2.07 | 1.89E−08 | 2.18E−07 |
| Usp18 | 491.6 | 1.66 | 2.34 | 1.26E−06 | 1.03E−05 |
| Serpinb9b | 63.4 | 1.66 | 2.45 | 1.55E−06 | 1.26E−05 |
| Vwa2 | 4.2 | 1.66 | 5.89 | 3.9E−06 | 2.9E−05 |
| Saa3 | 14.8 | 1.66 | 4.96 | 6.94E−06 | 4.87E−05 |
| Mad2l1 | 857.6 | 1.65 | 1.69 | 0 | 0 |
| Hells | 609.3 | 1.65 | 1.71 | 0 | 0 |
| Cisd1 | 468.5 | 1.65 | 1.73 | 0 | 0 |
| Slc35d3 | 110.5 | 1.65 | 1.77 | 0 | 5E−15 |
| Sncb | 15 | 1.65 | 2.65 | 2.82E−06 | 2.17E−05 |
| Ppbp | 30.6 | 1.65 | 2.38 | 3.36E−06 | 2.54E−05 |
| Tff1 | 8.6 | 1.65 | 3.22 | 1.24E−05 | 8.24E−05 |
| Nsd2 | 2242.8 | 1.64 | 1.68 | 0 | 0 |
| Phf19 | 475.4 | 1.64 | 1.71 | 0 | 0 |
| Ccdc34 | 308.1 | 1.64 | 1.73 | 0 | 0 |
| 1700001O22Rik | 140.3 | 1.64 | 1.78 | 4E−15 | 1.36E−13 |
| Gm20667 | 5.2 | 1.64 | 4.98 | 1.13E−05 | 7.61E−05 |
| Ppp1r3g | 7.3 | 1.64 | 3.91 | 1.3E−05 | 8.58E−05 |
| 4930519L02Rik | 9.9 | 1.64 | 4.33 | 1.41E−05 | 9.23E−05 |
| Racgap1 | 3392.2 | 1.63 | 1.67 | 0 | 0 |
| Lmnb1 | 6788.3 | 1.63 | 1.69 | 0 | 0 |
| Kif23 | 1414.3 | 1.63 | 1.69 | 0 | 0 |
| Ryk | 207.2 | 1.63 | 1.76 | 1E−15 | 2.6E−14 |
| Heatr9 | 204.6 | 1.63 | 1.85 | 1.58E−11 | 3.07E−10 |
| Ifi27l2a | 8730.4 | 1.63 | 1.99 | 2.36E−08 | 2.67E−07 |
| Tnip3 | 36.1 | 1.63 | 2.1 | 2.07E−07 | 1.97E−06 |
| Vat1l | 14.9 | 1.62 | 3.1 | 9.48E−06 | 6.47E−05 |
| Tpsb2 | 486.9 | 1.62 | 2.93 | 2.12E−05 | 0.000133 |
| AW112010 | 5981.3 | 1.61 | 1.69 | 0 | 0 |
| 9630013D21Rik | 122.8 | 1.61 | 1.8 | 4.6E−12 | 9.71E−11 |
| Mtfp1 | 42.1 | 1.61 | 1.98 | 4.08E−08 | 4.44E−07 |
| Col1a2 | 95 | 1.61 | 2.11 | 6.19E−07 | 5.41E−06 |
| Ascl2 | 24.4 | 1.61 | 2.62 | 1.12E−05 | 7.53E−05 |
| Ccdc18 | 45.6 | 1.6 | 1.91 | 1.15E−08 | 1.37E−07 |
| Myct1 | 34.7 | 1.6 | 2.16 | 1.88E−06 | 1.49E−05 |
| Hist1h2bm | 8.5 | 1.6 | 3.51 | 2.45E−05 | 0.000151 |
| Elane | 132 | 1.6 | 3.14 | 2.69E−05 | 0.000165 |
| Serping1 | 49.6 | 1.59 | 1.9 | 6.03E−09 | 7.54E−08 |
| Rab44 | 136.4 | 1.59 | 2.08 | 8.37E−07 | 7.11E−06 |
| Dcn | 49 | 1.59 | 2.48 | 3.24E−06 | 2.45E−05 |
| 6530402F18Rik | 553.7 | 1.58 | 1.63 | 0 | 0 |
| Klrk1 | 793.9 | 1.58 | 1.68 | 0 | 0 |
| Nup37 | 207.1 | 1.58 | 1.74 | 3.05E−13 | 7.43E−12 |
| Bst1 | 74.2 | 1.58 | 1.75 | 1.64E−12 | 3.71E−11 |
| Il1rl1 | 328.6 | 1.58 | 1.74 | 1.73E−12 | 3.88E−11 |
| Klrc3 | 41.9 | 1.58 | 1.99 | 7.66E−08 | 7.98E−07 |
| Adgrg7 | 5.3 | 1.58 | 5.46 | 9.43E−06 | 6.44E−05 |
| Gp1bb | 21.6 | 1.58 | 2.44 | 2.04E−05 | 0.000128 |
| Sh2d1a | 1356.7 | 1.57 | 1.64 | 0 | 0 |
| Areg | 192.8 | 1.57 | 1.78 | 1.04E−10 | 1.78E−09 |
| Mlkl | 114.8 | 1.57 | 1.78 | 1.33E−10 | 2.24E−09 |
| D630039A03Rik | 66.5 | 1.57 | 1.86 | 1.8E−08 | 2.09E−07 |
| Sdsl | 60 | 1.57 | 2.27 | 7.96E−06 | 5.53E−05 |
| Oas2 | 1573.5 | 1.57 | 2.38 | 8.81E−06 | 6.05E−05 |
| Tjp2 | 411.7 | 1.56 | 1.67 | 1E−15 | 3E−14 |
| Fut7 | 69.8 | 1.56 | 1.8 | 1.61E−09 | 2.23E−08 |
| Ces2g | 190 | 1.56 | 2.29 | 8.88E−06 | 6.1E−05 |
| Tmem59l | 15.2 | 1.56 | 2.3 | 1.24E−05 | 8.24E−05 |
| Kcnj5 | 5.3 | 1.56 | 5.41 | 1.25E−05 | 8.31E−05 |
| Maob | 9.3 | 1.56 | 3.57 | 3.89E−05 | 0.000229 |
| Dut | 2331.7 | 1.55 | 1.6 | 0 | 0 |
| 4933404O12Rik | 326.5 | 1.55 | 1.66 | 0 | 1.4E−14 |
| Alox8 | 57.9 | 1.55 | 1.79 | 1.05E−09 | 1.5E−08 |
| Runx2os1 | 41.2 | 1.55 | 2.01 | 1.56E−06 | 1.26E−05 |
| Etv4 | 18.3 | 1.55 | 2.25 | 1.04E−05 | 7E−05 |
| Ccnf | 676.9 | 1.54 | 1.62 | 0 | 0 |
| Spns2 | 183.2 | 1.54 | 1.86 | 1.1E−07 | 1.11E−06 |
| H1fx | 65.9 | 1.54 | 1.97 | 1.34E−06 | 1.1E−05 |
| Chtf18 | 707.9 | 1.53 | 1.58 | 0 | 0 |
| Gbp2b | 1682.8 | 1.53 | 1.59 | 0 | 0 |
| Cmtm7 | 2372.8 | 1.53 | 1.59 | 0 | 0 |
| Gstt3 | 58.6 | 1.53 | 1.73 | 3.64E−10 | 5.65E−09 |
| Chek2 | 113.8 | 1.53 | 1.77 | 3.66E−09 | 4.79E−08 |
| Rab39b | 35.1 | 1.53 | 1.86 | 7.94E−08 | 8.25E−07 |
| Vash1 | 14.6 | 1.53 | 2.36 | 2.63E−05 | 0.000162 |
| Incenp | 3803.5 | 1.52 | 1.56 | 0 | 0 |
| Zwilch | 309.4 | 1.52 | 1.59 | 0 | 0 |
| Brip1 | 231.5 | 1.52 | 1.6 | 0 | 0 |
| Gm14005 | 181.4 | 1.52 | 1.61 | 0 | 2E−15 |
| Foxm1 | 1814.7 | 1.52 | 1.62 | 0 | 5E−15 |
| 1700020L24Rik | 90.2 | 1.52 | 1.72 | 8.22E−10 | 1.2E−08 |
| Il4 | 152.5 | 1.52 | 1.8 | 3.53E−08 | 3.9E−07 |
| Mx1 | 719.3 | 1.52 | 2.3 | 2.01E−05 | 0.000127 |
| Gna15 | 580.1 | 1.51 | 1.6 | 0 | 1E−15 |
| Dctpp1 | 771.7 | 1.51 | 1.64 | 2.43E−13 | 5.96E−12 |
| Angptl2 | 741.2 | 1.51 | 1.67 | 1.1E−11 | 2.2E−10 |
| Mgarp | 30.5 | 1.51 | 1.87 | 4.92E−07 | 4.41E−06 |
| Gm19585 | 364.2 | 1.5 | 1.56 | 0 | 0 |
| Zfp367 | 650.8 | 1.5 | 1.58 | 0 | 0 |
| S100a10 | 7676.1 | 1.5 | 1.58 | 0 | 0 |
| C330027C09Rik | 421.9 | 1.5 | 1.63 | 1.44E−13 | 3.66E−12 |
| Cenpk | 43.5 | 1.5 | 1.73 | 9.19E−09 | 1.11E−07 |
| Ly6e | 13759 | 1.5 | 1.83 | 2.5E−07 | 2.35E−06 |
| Ccl4 | 2257.9 | 1.5 | 1.9 | 1.59E−06 | 1.28E−05 |
| Casp7 | 625.6 | 1.49 | 1.53 | 0 | 0 |
| Zfand4 | 84.9 | 1.49 | 1.61 | 1.84E−13 | 4.58E−12 |
| Tstd3 | 269.1 | 1.49 | 1.65 | 1.75E−11 | 3.39E−10 |
| Lmtk3 | 107.1 | 1.49 | 1.75 | 4.88E−08 | 5.24E−07 |
| AC153498.1 | 62.1 | 1.49 | 1.77 | 7.06E−08 | 7.4E−07 |
| Grtp1 | 36.2 | 1.49 | 1.96 | 3.97E−06 | 2.94E−05 |
| Gm2762 | 38.5 | 1.49 | 2.09 | 6.91E−06 | 4.85E−05 |
| D130058E05Rik | 5.1 | 1.49 | 5.39 | 2.27E−05 | 0.000142 |
| Npy | 11.8 | 1.49 | 2.45 | 6.48E−05 | 0.00036 |
| Serpina3h | 8.8 | 1.49 | 2.77 | 7.52E−05 | 0.000411 |
| Ighg2c | 873 | 1.49 | 3.73 | 7.66E−05 | 0.000418 |
| Nkain1 | 8 | 1.49 | 3.23 | 7.74E−05 | 0.000421 |
| Podnl1 | 2382.6 | 1.48 | 1.53 | 0 | 0 |
| Gzmm | 496.8 | 1.48 | 1.56 | 0 | 0 |
| Haspin | 214.2 | 1.48 | 1.6 | 1.76E−13 | 4.4E−12 |
| Csfl | 1178.9 | 1.48 | 1.64 | 5.94E−11 | 1.05E−09 |
| Lamc2 | 28.8 | 1.48 | 1.89 | 2.97E−06 | 2.28E−05 |
| Gm11454 | 22.4 | 1.48 | 1.93 | 3.25E−06 | 2.46E−05 |
| Tal1 | 90.9 | 1.48 | 2.14 | 2.23E−05 | 0.000139 |
| Ybx3 | 2394.9 | 1.47 | 1.56 | 0 | 0 |
| Rln3 | 181.3 | 1.47 | 1.61 | 1.29E−11 | 2.56E−10 |
| Rai14 | 77.8 | 1.47 | 1.68 | 9.24E−09 | 1.11E−07 |
| Gmpr | 24.8 | 1.47 | 2.3 | 5.94E−05 | 0.000334 |
| Drp2 | 31.1 | 1.47 | 2.94 | 9.79E−05 | 0.000518 |
| Cma1 | 339 | 1.47 | 2.71 | 0.00011 | 0.000572 |
| Gbp5 | 1110.5 | 1.46 | 1.49 | 0 | 0 |
| Alad | 1397.6 | 1.46 | 1.63 | 2.48E−10 | 4E−09 |
| Tnfsf4 | 81.6 | 1.46 | 1.73 | 2.63E−07 | 2.47E−06 |
| Rtkn2 | 30.4 | 1.46 | 1.85 | 1.64E−06 | 1.32E−05 |
| Klra7 | 38.4 | 1.46 | 2.03 | 1.43E−05 | 9.35E−05 |
| Pf4 | 162.2 | 1.46 | 2.25 | 9.08E−05 | 0.000485 |
| Gbp2 | 3001.3 | 1.45 | 1.5 | 0 | 0 |
| Ercc6l | 189.4 | 1.45 | 1.55 | 2.2E−14 | 6.19E−13 |
| Iigp1 | 872.5 | 1.45 | 1.55 | 2.8E−14 | 7.75E−13 |
| Gemin6 | 89.9 | 1.45 | 1.55 | 8.5E−14 | 2.2E−12 |
| Paqr4 | 188.3 | 1.45 | 1.58 | 4.17E−12 | 8.88E−11 |
| Cd160 | 483.3 | 1.45 | 1.63 | 1.06E−09 | 1.51E−08 |
| Ttc16 | 66.2 | 1.45 | 1.72 | 1.54E−07 | 1.51E−06 |
| Ulk4 | 24.5 | 1.45 | 1.83 | 2.23E−06 | 1.75E−05 |
| Ccnb1ip1 | 40.8 | 1.45 | 1.87 | 6.02E−06 | 4.29E−05 |
| Tubb1 | 17.5 | 1.45 | 2.25 | 5.51E−05 | 0.000312 |
| Vax2 | 12.7 | 1.45 | 2.38 | 0.000106 | 0.000558 |
| Slc16a11 | 9.1 | 1.45 | 2.57 | 0.000108 | 0.000563 |
| Col3a1 | 82.1 | 1.45 | 2.78 | 0.000124 | 0.000638 |
| Sdf2l1 | 994.9 | 1.44 | 1.51 | 0 | 0 |
| Psmc3ip | 149.8 | 1.44 | 1.53 | 1E−15 | 3.8E−14 |
| Tmem107 | 70.8 | 1.44 | 1.63 | 4.82E−09 | 6.16E−08 |
| Tmem121 | 19.5 | 1.44 | 2.22 | 6.17E−05 | 0.000344 |
| Platr11 | 10.2 | 1.44 | 2.5 | 0.00012 | 0.000617 |
| 9230102O04Rik | 5.8 | 1.44 | 3.53 | 0.000127 | 0.000649 |
| Ms4a4b | 9942.9 | 1.43 | 1.46 | 0 | 0 |
| Gmnn | 974.1 | 1.43 | 1.47 | 0 | 0 |
| Bak1 | 2591.4 | 1.43 | 1.48 | 0 | 0 |
| Casp3 | 2922.8 | 1.43 | 1.48 | 0 | 0 |
| Sass6 | 197.1 | 1.43 | 1.51 | 0 | 4E−15 |
| 2310031A07Rik | 64.6 | 1.43 | 1.81 | 4.21E−06 | 3.1E−05 |
| Gda | 44.3 | 1.43 | 2.06 | 3.9E−05 | 0.00023 |
| Ckm | 8 | 1.43 | 3.17 | 0.000143 | 0.000725 |
| Mfsd13a | 222.3 | 1.42 | 1.53 | 1.9E−13 | 4.74E−12 |
| Gna14 | 24.8 | 1.42 | 1.92 | 1.63E−05 | 0.000106 |
| Retnla | 7.6 | 1.42 | 5.6 | 8.17E−05 | 0.000441 |
| 4930579G24Rik | 292.8 | 1.41 | 1.44 | 0 | 0 |
| Alcam | 430 | 1.41 | 1.5 | 0 | 1.7E−14 |
| Ak6 | 275.7 | 1.41 | 1.56 | 3.31E−10 | 5.2E−09 |
| Ica1 | 85.1 | 1.41 | 1.64 | 1.66E−07 | 1.62E−06 |
| Smo | 59 | 1.41 | 1.66 | 3.83E−07 | 3.5E−06 |
| A930002I21Rik | 49.6 | 1.41 | 1.91 | 2.49E−05 | 0.000154 |
| Col1a1 | 89.2 | 1.41 | 1.94 | 2.5E−05 | 0.000154 |
| Cd34 | 96.8 | 1.41 | 2.01 | 5.84E−05 | 0.000329 |
| Lhx2 | 15.9 | 1.41 | 2.11 | 6.05E−05 | 0.000339 |
| Ptger3 | 20.8 | 1.41 | 2.29 | 0.000156 | 0.000779 |
| Xdh | 1588.9 | 1.4 | 1.45 | 0 | 0 |
| Ttn | 292.1 | 1.4 | 1.52 | 3.22E−11 | 5.97E−10 |
| Asns | 221.7 | 1.4 | 1.57 | 2.68E−09 | 3.59E−08 |
| Cmc2 | 422.7 | 1.4 | 1.6 | 2.68E−08 | 3.01E−07 |
| Cks2 | 105.1 | 1.4 | 1.64 | 3.29E−07 | 3.05E−06 |
| Gzmc | 56.6 | 1.4 | 1.74 | 4.29E−06 | 3.16E−05 |
| Hist1h2bb | 17.7 | 1.4 | 2 | 7.72E−05 | 0.00042 |
| Epx | 8 | 1.4 | 6.63 | 8.75E−05 | 0.00047 |
| Gata2 | 341.6 | 1.4 | 2.14 | 0.000102 | 0.000536 |
| Hp | 143.1 | 1.4 | 2.47 | 0.000179 | 0.000879 |
| Gm11843 | 7.5 | 1.4 | 4.37 | 0.000181 | 0.000888 |
| Batf | 1910.4 | 1.39 | 1.44 | 0 | 0 |
| Hip1 | 1867.7 | 1.39 | 1.46 | 0 | 0 |
| Havcr2 | 486.2 | 1.39 | 1.5 | 1.88E−12 | 4.18E−11 |
| Acod1 | 232.1 | 1.39 | 1.69 | 1.35E−06 | 1.11E−05 |
| Grb10 | 61 | 1.39 | 1.77 | 5.98E−06 | 4.26E−05 |
| Reg2 | 5.6 | 1.39 | 5.03 | 7.66E−06 | 5.33E−05 |
| N4bp1 | 3266.2 | 1.38 | 1.4 | 0 | 0 |
| Prim1 | 1043.4 | 1.38 | 1.41 | 0 | 0 |
| Mtfr2 | 115.7 | 1.38 | 1.47 | 2.56E−13 | 6.28E−12 |
| Hmgb2 | 1572.1 | 1.38 | 1.49 | 1.93E−12 | 4.28E−11 |
| Isg20 | 861.4 | 1.38 | 1.52 | 2.64E−10 | 4.24E−09 |
| Cmpk2 | 311.4 | 1.38 | 1.66 | 1E−06 | 8.41E−06 |
| Isg15 | 787.9 | 1.38 | 1.72 | 4.32E−06 | 3.17E−05 |
| Il1rn | 44.9 | 1.38 | 1.79 | 1.05E−05 | 7.06E−05 |
| Fkbp1b | 22.4 | 1.38 | 1.9 | 3.16E−05 | 0.00019 |
| Nwd1 | 24.3 | 1.38 | 2.01 | 7.94E−05 | 0.000431 |
| Tnp2 | 17.6 | 1.38 | 2.34 | 0.000128 | 0.000654 |
| Ifit3 | 421.9 | 1.38 | 2.21 | 0.000147 | 0.00074 |
| Hist1h2ai | 9.8 | 1.38 | 2.17 | 0.000184 | 0.000901 |
| Cdc20b | 14.4 | 1.38 | 2.55 | 0.00019 | 0.000927 |
| a | 12.6 | 1.38 | 2.36 | 0.000204 | 0.000988 |
| Tubb5 | 27245.9 | 1.37 | 1.4 | 0 | 0 |
| Myo1f | 6082 | 1.37 | 1.41 | 0 | 0 |
| Cdc25b | 5230.3 | 1.37 | 1.42 | 0 | 0 |
| E2f3 | 421.5 | 1.37 | 1.42 | 0 | 0 |
| Impa1 | 900.5 | 1.37 | 1.42 | 0 | 0 |
| Kif24 | 76.5 | 1.37 | 1.56 | 2.19E−08 | 2.49E−07 |
| Art2a-ps | 407.6 | 1.37 | 1.56 | 4.59E−08 | 4.94E−07 |
| Gp5 | 24.7 | 1.37 | 1.84 | 5.7E−05 | 0.000321 |
| Dio2 | 18.9 | 1.37 | 2.13 | 0.000105 | 0.000551 |
| Cdk6 | 2038 | 1.36 | 1.38 | 0 | 0 |
| Ppil1 | 932 | 1.36 | 1.4 | 0 | 0 |
| Shmt1 | 546.5 | 1.36 | 1.41 | 0 | 0 |
| Plxdc1 | 231.9 | 1.36 | 1.46 | 6.89E−12 | 1.42E−10 |
| Hopx | 270.1 | 1.36 | 1.48 | 2.27E−11 | 4.33E−10 |
| Serp2 | 160.9 | 1.36 | 1.5 | 5.14E−10 | 7.76E−09 |
| Dusp14 | 437.4 | 1.36 | 1.5 | 1.35E−09 | 1.89E−08 |
| Lgals7 | 154 | 1.36 | 1.59 | 1.04E−06 | 8.72E−06 |
| Nqo1 | 35.8 | 1.36 | 1.7 | 5.6E−06 | 4.01E−05 |
| Aldh1a1 | 40.1 | 1.36 | 1.98 | 0.000105 | 0.000552 |
| Ifit1 | 298.3 | 1.36 | 2.02 | 0.000125 | 0.000642 |
| Psat1 | 1715.9 | 1.35 | 1.37 | 0 | 0 |
| Gins3 | 181.3 | 1.35 | 1.46 | 6.05E−11 | 1.07E−09 |
| Slc16a3 | 161.5 | 1.35 | 1.49 | 3.5E−10 | 5.45E−09 |
| Gbp10 | 432.2 | 1.35 | 1.51 | 4.13E−09 | 5.34E−08 |
| Ube2l6 | 515.6 | 1.35 | 1.54 | 4.68E−08 | 5.03E−07 |
| Slfn3 | 67.7 | 1.35 | 1.6 | 6.85E−07 | 5.93E−06 |
| Nccrp1 | 65 | 1.35 | 1.64 | 4.48E−06 | 3.27E−05 |
| Rgs8 | 18.7 | 1.35 | 1.85 | 9.21E−05 | 0.000492 |
| Trim58 | 10.1 | 1.35 | 2.9 | 0.000362 | 0.001642 |
| Cpa3 | 467.2 | 1.35 | 2.45 | 0.000376 | 0.001695 |
| Fkbp5 | 2388.9 | 1.34 | 1.37 | 0 | 0 |
| Tmpo | 4623.5 | 1.34 | 1.39 | 0 | 0 |
| Cxcr3 | 2032 | 1.34 | 1.39 | 0 | 0 |
| Mrps25 | 464.1 | 1.34 | 1.41 | 0 | 7E−15 |
| Zdhhc2 | 533.9 | 1.34 | 1.42 | 3E−15 | 8.8E−14 |
| Slc25a13 | 161.1 | 1.34 | 1.42 | 1.21E−13 | 3.1E−12 |
| Nudcd1 | 238.9 | 1.34 | 1.43 | 1.69E−13 | 4.24E−12 |
| Phf11b | 840.6 | 1.34 | 1.45 | 4.58E−12 | 9.71E−11 |
| Galr3 | 5.7 | 1.34 | 4.53 | 0.000359 | 0.001631 |
| Kctd17 | 526.7 | 1.33 | 1.39 | 0 | 0 |
| Klre1 | 117.7 | 1.33 | 1.66 | 8.94E−06 | 6.13E−05 |
| Gm14130 | 18.9 | 1.33 | 1.81 | 4.17E−05 | 0.000244 |
| Myo1d | 68 | 1.33 | 1.84 | 7.41E−05 | 0.000405 |
| Ica1l | 24.1 | 1.33 | 1.82 | 0.000114 | 0.00059 |
| Coa6 | 201.6 | 1.32 | 1.4 | 5.2E−14 | 1.39E−12 |
| Mettl7a1 | 240.2 | 1.32 | 1.42 | 4.43E−12 | 9.41E−11 |
| Sytl3 | 850.3 | 1.32 | 1.44 | 5.64E−10 | 8.46E−09 |
| Lrrc75a | 59.6 | 1.32 | 1.6 | 5.23E−06 | 3.78E−05 |
| Hspa1b | 148.2 | 1.32 | 1.66 | 1.9E−05 | 0.000121 |
| Serpina3i | 24.8 | 1.32 | 2.37 | 0.000411 | 0.00183 |
| Mmp8 | 16.8 | 1.32 | 2.57 | 0.000494 | 0.002149 |
| Mcm2 | 4604.3 | 1.31 | 1.34 | 0 | 0 |
| Wdr62 | 861.5 | 1.31 | 1.35 | 0 | 0 |
| Cdk5r1 | 454.4 | 1.31 | 1.35 | 0 | 0 |
| Cenpu | 141.6 | 1.31 | 1.39 | 1.83E−13 | 4.57E−12 |
| Myl6b | 55.6 | 1.31 | 1.55 | 2.08E−06 | 1.64E−05 |
| Nmur1 | 2.7 | 1.31 | 4.59 | 9.87E−05 | 0.000522 |
| Prim2 | 843.4 | 1.3 | 1.34 | 0 | 0 |
| Rrm1 | 4168.2 | 1.3 | 1.34 | 0 | 0 |
| Dnajc15 | 778.1 | 1.3 | 1.35 | 0 | 0 |
| Brip1os | 583.1 | 1.3 | 1.37 | 1E−15 | 3.8E−14 |
| Perp | 113.8 | 1.3 | 1.41 | 4.33E−10 | 6.63E−09 |
| Msantd3 | 9.2 | 1.3 | 2.05 | 0.000408 | 0.00182 |
| Col6a6 | 6.3 | 1.3 | 4.16 | 0.000432 | 0.001913 |
| Gnaz | 9.7 | 1.3 | 2.35 | 0.000531 | 0.002295 |
| Syt11 | 1025 | 1.29 | 1.31 | 0 | 0 |
| Rab27a | 1564.8 | 1.29 | 1.32 | 0 | 0 |
| Stip1 | 4349.6 | 1.29 | 1.32 | 0 | 0 |
| Cep19 | 280.3 | 1.29 | 1.34 | 0 | 0 |
| Selenoh | 1560.4 | 1.29 | 1.34 | 0 | 1E−15 |
| Clec1b | 45.4 | 1.29 | 1.59 | 1.54E−05 | 0.0001 |
| Il5 | 3.6 | 1.29 | 5.79 | 0.000125 | 0.000643 |
| Klrb1a | 36.2 | 1.29 | 1.9 | 0.000175 | 0.000861 |
| Serpina3n | 16.7 | 1.29 | 2.04 | 0.000428 | 0.001901 |
| Ms4a2 | 35.1 | 1.29 | 2.29 | 0.000563 | 0.002414 |
| Tmem171 | 12.4 | 1.29 | 2.26 | 0.000567 | 0.002428 |
| Snai3 | 164.9 | 1.28 | 1.39 | 2.53E−10 | 4.06E−09 |
| CT030173.1 | 47.8 | 1.28 | 1.54 | 5.74E−06 | 4.11E−05 |
| Iqcg | 28.8 | 1.28 | 1.6 | 1.76E−05 | 0.000113 |
| Mst1r | 38.6 | 1.28 | 1.61 | 2.34E−05 | 0.000146 |
| Serpine2 | 31.1 | 1.28 | 1.66 | 0.000104 | 0.000547 |
| Pawr | 19.1 | 1.28 | 1.82 | 0.00015 | 0.000752 |
| D630045J12Rik | 11.4 | 1.28 | 2.12 | 0.000411 | 0.001831 |
| Oasl2 | 98.6 | 1.28 | 3 | 0.000737 | 0.003051 |
| Rab19 | 1074.2 | 1.27 | 1.31 | 0 | 0 |
| Rap2a | 566 | 1.27 | 1.31 | 0 | 0 |
| Tm6sf1 | 1262.4 | 1.27 | 1.32 | 0 | 0 |
| Caskin2 | 253.4 | 1.27 | 1.33 | 6E−15 | 1.65E−13 |
| Gm7901 | 153.5 | 1.27 | 1.38 | 7.96E−10 | 1.16E−08 |
| Ly6c1 | 2390.4 | 1.27 | 1.41 | 2.71E−08 | 3.04E−07 |
| Pxmp2 | 56.9 | 1.27 | 1.44 | 4.38E−07 | 3.98E−06 |
| Il3 | 4 | 1.27 | 5.13 | 0.000123 | 0.000633 |
| C1ql1 | 3.8 | 1.27 | 5.38 | 0.000393 | 0.001762 |
| Car1 | 559.7 | 1.27 | 1.97 | 0.000423 | 0.001879 |
| Sla | 9431 | 1.26 | 1.29 | 0 | 0 |
| Runx2 | 2056.2 | 1.26 | 1.3 | 0 | 0 |
| Impa2 | 575 | 1.26 | 1.3 | 0 | 0 |
| Atp2b4 | 1723.8 | 1.26 | 1.3 | 0 | 0 |
| Cpsf2 | 1192.5 | 1.26 | 1.32 | 2.6E−14 | 7.37E−13 |
| Bcl2l1 | 1866.8 | 1.26 | 1.33 | 3.2E−14 | 8.76E−13 |
| 2810006K23Rik | 128.8 | 1.26 | 1.41 | 3.26E−08 | 3.63E−07 |
| Sostdc1 | 55.7 | 1.26 | 1.49 | 6.65E−06 | 4.69E−05 |
| Gm15987 | 44.4 | 1.26 | 1.64 | 4.28E−05 | 0.00025 |
| Mest | 21.1 | 1.26 | 1.73 | 0.000157 | 0.000783 |
| Fkbp10 | 29.4 | 1.26 | 1.74 | 0.000252 | 0.001191 |
| Hnf4a | 4.7 | 1.26 | 3.99 | 0.000517 | 0.002238 |
| Gp1ba | 18.9 | 1.26 | 2.02 | 0.000659 | 0.002769 |
| AC153369.2 | 8.4 | 1.26 | 2.51 | 0.000813 | 0.003321 |
| Prss34 | 356.8 | 1.26 | 2.96 | 0.000841 | 0.003422 |
| Tarm1 | 9.2 | 1.26 | 3.21 | 0.000847 | 0.003442 |
| Nudt5 | 931.4 | 1.25 | 1.28 | 0 | 0 |
| Aurka | 633.5 | 1.25 | 1.29 | 0 | 0 |
| C1qtnf6 | 340.9 | 1.25 | 1.3 | 0 | 0 |
| Nrm | 1120.2 | 1.25 | 1.3 | 0 | 0 |
| Kifc5b | 272.7 | 1.25 | 1.31 | 1E−15 | 3.3E−14 |
| H2afx | 3402.7 | 1.25 | 1.31 | 2E−15 | 5.8E−14 |
| Gyg | 764.1 | 1.25 | 1.31 | 8E−15 | 2.44E−13 |
| Lsm2 | 779.8 | 1.25 | 1.34 | 8.52E−12 | 1.74E−10 |
| Xaf1 | 1257.6 | 1.25 | 1.36 | 2.65E−09 | 3.56E−08 |
| Kndc1 | 53.5 | 1.25 | 1.48 | 2.11E−06 | 1.66E−05 |
| Xkr8 | 51.5 | 1.25 | 1.48 | 3.91E−06 | 2.91E−05 |
| Gm37004 | 26 | 1.25 | 1.56 | 3.67E−05 | 0.000218 |
| Scin | 117.4 | 1.25 | 1.62 | 6.9E−05 | 0.000381 |
| Sgms2 | 21.4 | 1.25 | 1.8 | 0.00038 | 0.00171 |
| Vcan | 11.1 | 1.25 | 2.23 | 0.000894 | 0.003604 |
| Lrp3 | 7.7 | 1.25 | 2.52 | 0.000956 | 0.003818 |
| Nde1 | 1235.2 | 1.24 | 1.26 | 0 | 0 |
| Hn1l | 449.3 | 1.24 | 1.3 | 6E−15 | 1.74E−13 |
| Lamc1 | 923.9 | 1.24 | 1.31 | 1.71E−13 | 4.3E−12 |
| Alg6 | 94.2 | 1.24 | 1.39 | 1.32E−07 | 1.31E−06 |
| AC126459.2 | 179 | 1.24 | 1.4 | 3.61E−07 | 3.32E−06 |
| Dsp | 79.2 | 1.24 | 1.55 | 4.45E−05 | 0.000258 |
| Gm14148 | 21.4 | 1.24 | 1.54 | 5.86E−05 | 0.00033 |
| AC151730.1 | 71.6 | 1.24 | 1.69 | 8.59E−05 | 0.000463 |
| Gm13461 | 16.5 | 1.24 | 1.82 | 0.000465 | 0.002039 |
| Fhl1 | 19.9 | 1.24 | 1.74 | 0.000492 | 0.002143 |
| Smpx | 6.2 | 1.24 | 4.22 | 0.000628 | 0.002658 |
| Tom1l1 | 20.3 | 1.24 | 1.95 | 0.00073 | 0.003028 |
| Hist1h2ab | 3.6 | 1.24 | 3.05 | 0.000791 | 0.003245 |
| Il33 | 4.5 | 1.24 | 3.09 | 0.000871 | 0.003531 |
| Epha3 | 8.5 | 1.24 | 2.62 | 0.000892 | 0.003598 |
| Hist1h2ae | 10.9 | 1.24 | 2.06 | 0.000918 | 0.003688 |
| Gm13031 | 8 | 1.24 | 2.51 | 0.000941 | 0.003764 |
| Gm14569 | 11.7 | 1.24 | 3.38 | 0.000999 | 0.003964 |
| Slamf7 | 804.9 | 1.23 | 1.27 | 0 | 0 |
| Cxcr6 | 818 | 1.23 | 1.27 | 0 | 0 |
| Cpox | 574.5 | 1.23 | 1.28 | 0 | 8E−15 |
| Adam19 | 5999.2 | 1.23 | 1.29 | 2E−15 | 5.2E−14 |
| Ms4a4c | 692.6 | 1.23 | 1.3 | 4.5E−14 | 1.22E−12 |
| Penk | 1018 | 1.23 | 1.3 | 2.51E−12 | 5.5E−11 |
| Klrb1f | 190 | 1.23 | 1.32 | 3.65E−10 | 5.66E−09 |
| Gm6166 | 141.1 | 1.23 | 1.32 | 1.18E−09 | 1.67E−08 |
| Fam184a | 33.5 | 1.23 | 1.5 | 1.8E−05 | 0.000116 |
| Meis1 | 68.8 | 1.23 | 1.55 | 8.54E−05 | 0.00046 |
| Erg | 37.2 | 1.23 | 1.55 | 0.000101 | 0.000531 |
| Enpp6 | 21.8 | 1.23 | 1.82 | 0.00032 | 0.001474 |
| Gm6982 | 3.3 | 1.23 | 4.32 | 0.000669 | 0.002804 |
| Gm7456 | 10.4 | 1.23 | 2.12 | 0.000809 | 0.003311 |
| Spon1 | 28.9 | 1.23 | 2.16 | 0.000894 | 0.003604 |
| Unc80 | 11 | 1.23 | 2.08 | 0.001129 | 0.004421 |
| Ran | 2657.7 | 1.22 | 1.24 | 0 | 0 |
| Zranb3 | 259.4 | 1.22 | 1.27 | 0 | 0 |
| Cbx5 | 1657.3 | 1.22 | 1.27 | 0 | 1E−15 |
| Ms4a6d | 734.6 | 1.22 | 1.28 | 3E−15 | 1.07E−13 |
| Fbxo5 | 492.7 | 1.22 | 1.28 | 1.3E−14 | 3.61E−13 |
| Hsph1 | 3580.6 | 1.22 | 1.28 | 4.8E−14 | 1.29E−12 |
| Pttg1 | 1600.8 | 1.22 | 1.28 | 1.21E−13 | 3.11E−12 |
| Cysltr2 | 169.1 | 1.22 | 1.37 | 3.3E−07 | 3.06E−06 |
| Phf11a | 221.4 | 1.22 | 1.4 | 9.86E−07 | 8.28E−06 |
| Rgs18 | 52.3 | 1.22 | 1.54 | 9.22E−05 | 0.000492 |
| Wdr54 | 19.6 | 1.22 | 1.61 | 0.000186 | 0.000909 |
| Clstn3 | 34.6 | 1.22 | 1.6 | 0.000219 | 0.001054 |
| Aqp1 | 400.8 | 1.22 | 1.93 | 0.00081 | 0.003312 |
| Il20ra | 9.2 | 1.22 | 1.97 | 0.001035 | 0.00409 |
| Hist1h2bk | 8.4 | 1.22 | 2.66 | 0.001176 | 0.004581 |
| Chaf1b | 1135 | 1.21 | 1.23 | 0 | 0 |
| Rnaseh2b | 648.9 | 1.21 | 1.24 | 0 | 0 |
| Tnfrsf9 | 1547.1 | 1.21 | 1.29 | 1.28E−10 | 2.17E−09 |
| Smoc2 | 90.6 | 1.21 | 1.34 | 6.72E−08 | 7.07E−07 |
| Fancf | 96.5 | 1.21 | 1.34 | 9.78E−08 | 1E−06 |
| Tex13c2 | 4.1 | 1.21 | 3.28 | 0.000884 | 0.003573 |
| Mcpt1 | 510.2 | 1.21 | 3.19 | 0.001329 | 0.005105 |
| Chit1 | 6.9 | 1.21 | 2.51 | 0.00139 | 0.005311 |
| Psma1 | 2415.5 | 1.2 | 1.22 | 0 | 0 |
| Zcchc18 | 377.7 | 1.2 | 1.23 | 0 | 0 |
| Ppa1 | 1390.3 | 1.2 | 1.25 | 3E−15 | 9.8E−14 |
| Alms1 | 195.5 | 1.2 | 1.33 | 1.17E−07 | 1.18E−06 |
| Ms4a4a | 77.2 | 1.2 | 1.36 | 2.06E−07 | 1.97E−06 |
| Farp1 | 144.7 | 1.2 | 1.34 | 4.58E−07 | 4.13E−06 |
| Lrrc49 | 50.8 | 1.2 | 1.38 | 1.78E−06 | 1.42E−05 |
| Tfec | 71.7 | 1.2 | 1.43 | 1.18E−05 | 7.88E−05 |
| Gm15232 | 43.8 | 1.2 | 1.46 | 1.89E−05 | 0.00012 |
| Fgd1 | 24 | 1.2 | 1.52 | 0.000114 | 0.00059 |
| C78197 | 19.1 | 1.2 | 1.74 | 0.000569 | 0.002437 |
| Slc18a2 | 46.4 | 1.2 | 1.9 | 0.000856 | 0.003474 |
| Clgn | 5.1 | 1.2 | 3.51 | 0.001392 | 0.005316 |
| Calm3 | 5962.7 | 1.19 | 1.21 | 0 | 0 |
| Dtl | 1077.7 | 1.19 | 1.26 | 4.67E−11 | 8.43E−10 |
| Ptpn13 | 1107.8 | 1.19 | 1.3 | 6.78E−09 | 8.4E−08 |
| Mrpl34 | 516.5 | 1.19 | 1.33 | 2.57E−07 | 2.41E−06 |
| Nphp4 | 99.7 | 1.19 | 1.33 | 3.18E−07 | 2.95E−06 |
| Trim46 | 87.2 | 1.19 | 1.34 | 6.83E−07 | 5.91E−06 |
| Fn1 | 453.7 | 1.19 | 1.54 | 0.000169 | 0.000836 |
| I830127L07Rik | 6.6 | 1.19 | 5.81 | 0.00103 | 0.004071 |
| Reps2 | 11 | 1.19 | 2.16 | 0.001383 | 0.005292 |
| Mcm7 | 3981.2 | 1.18 | 1.21 | 0 | 0 |
| Trim37 | 677.9 | 1.18 | 1.21 | 0 | 0 |
| BC055324 | 200.3 | 1.18 | 1.22 | 0 | 7E−15 |
| Gm4737 | 198.1 | 1.18 | 1.25 | 3.91E−11 | 7.17E−10 |
| Pycard | 1522.9 | 1.18 | 1.25 | 8.74E−11 | 1.51E−09 |
| Fam185a | 192.8 | 1.18 | 1.28 | 2.12E−09 | 2.88E−08 |
| Bag2 | 63 | 1.18 | 1.31 | 4.71E−07 | 4.24E−06 |
| Ifitm1 | 214.1 | 1.18 | 1.44 | 6.52E−05 | 0.000362 |
| Alox12 | 39.7 | 1.18 | 1.46 | 0.00013 | 0.000665 |
| Cebpe | 5.7 | 1.18 | 3.45 | 0.001328 | 0.005104 |
| Ppp2r2c | 6.5 | 1.18 | 4.48 | 0.00155 | 0.005841 |
| Mcpt2 | 69.1 | 1.18 | 3.32 | 0.001613 | 0.006049 |
| Fam46c | 5669.2 | 1.17 | 1.2 | 0 | 0 |
| Plscr1 | 472.1 | 1.17 | 1.22 | 7.7E−14 | 2.01E−12 |
| Grb7 | 380.9 | 1.17 | 1.3 | 7.86E−07 | 6.72E−06 |
| Phlda3 | 37.7 | 1.17 | 1.38 | 2.49E−05 | 0.000154 |
| Rtkn | 29.5 | 1.17 | 1.44 | 0.000126 | 0.000646 |
| Cym | 46.2 | 1.17 | 1.51 | 0.000165 | 0.000821 |
| Cfap77 | 26 | 1.17 | 1.51 | 0.000167 | 0.000826 |
| Gm10286 | 2.8 | 1.17 | 4.98 | 0.000868 | 0.00352 |
| AC158990.2 | 7.6 | 1.17 | 2.88 | 0.001943 | 0.007104 |
| Entpd1 | 1241.1 | 1.16 | 1.2 | 0 | 0 |
| Dbi | 1291.8 | 1.16 | 1.21 | 2.3E−14 | 6.44E−13 |
| Hmga1-rs1 | 312.3 | 1.16 | 1.21 | 1.05E−12 | 2.43E−11 |
| Tmem237 | 165.9 | 1.16 | 1.22 | 1.29E−12 | 2.96E−11 |
| Raph1 | 355.4 | 1.16 | 1.22 | 5.71E−12 | 1.19E−10 |
| Echdc1 | 172.2 | 1.16 | 1.23 | 3.63E−11 | 6.71E−10 |
| Tuba1b | 1989.9 | 1.16 | 1.26 | 1.8E−08 | 2.09E−07 |
| Ifi214 | 383.7 | 1.16 | 1.31 | 9.86E−07 | 8.28E−06 |
| Pvrig | 126.3 | 1.16 | 1.31 | 1.56E−06 | 1.26E−05 |
| Steap3 | 134.6 | 1.16 | 1.4 | 5.47E−05 | 0.00031 |
| Fam89a | 34.7 | 1.16 | 1.45 | 5.54E−05 | 0.000313 |
| Gm8719 | 29.4 | 1.16 | 1.47 | 0.000149 | 0.000748 |
| Epas1 | 1633 | 1.16 | 1.5 | 0.000353 | 0.001608 |
| Adamts3 | 19.6 | 1.16 | 1.69 | 0.000885 | 0.003576 |
| Opn3 | 13.4 | 1.16 | 1.74 | 0.000965 | 0.003851 |
| Syngr4 | 9.5 | 1.16 | 2.04 | 0.001733 | 0.006439 |
| Tmem98 | 10.9 | 1.16 | 1.94 | 0.002001 | 0.007282 |
| Rfc5 | 1166.1 | 1.15 | 1.17 | 0 | 0 |
| Pdk3 | 769.1 | 1.15 | 1.17 | 0 | 0 |
| Samd3 | 769 | 1.15 | 1.2 | 5E−15 | 1.56E−13 |
| Psmb9 | 7187.6 | 1.15 | 1.22 | 2.5E−11 | 4.73E−10 |
| Slc9a5 | 265.3 | 1.15 | 1.22 | 7.94E−11 | 1.38E−09 |
| Fbxl8 | 274.2 | 1.15 | 1.23 | 6.27E−10 | 9.33E−09 |
| Myh10 | 131.2 | 1.15 | 1.37 | 3.82E−05 | 0.000226 |
| Ighv1-81 | 14 | 1.15 | 3.56 | 0.00155 | 0.005841 |
| Palm3 | 10.2 | 1.15 | 1.86 | 0.001827 | 0.006731 |
| Ifit3b | 107.9 | 1.15 | 2.04 | 0.002247 | 0.008038 |
| Pla2g16 | 2185.4 | 1.14 | 1.15 | 0 | 0 |
| Ptprcap | 9906.9 | 1.14 | 1.18 | 0 | 1E−15 |
| Zbp1 | 5544.8 | 1.14 | 1.22 | 1.03E−09 | 1.47E−08 |
| Eif2ak2 | 397.5 | 1.14 | 1.23 | 5.43E−09 | 6.87E−08 |
| Ifi209 | 837.9 | 1.14 | 1.24 | 2.48E−08 | 2.79E−07 |
| Prkar2b | 166.3 | 1.14 | 1.28 | 1.79E−06 | 1.43E−05 |
| Gm5620 | 33.2 | 1.14 | 1.38 | 0.000109 | 0.00057 |
| Tspo2 | 25.6 | 1.14 | 2.05 | 0.002092 | 0.007567 |
| Gm12641 | 4.5 | 1.14 | 2.63 | 0.002216 | 0.007947 |
| Gm5391 | 5.3 | 1.14 | 2.44 | 0.002442 | 0.008648 |
| Chga | 5.7 | 1.14 | 2.51 | 0.002652 | 0.00928 |
| Bhlhe40 | 29564.9 | 1.13 | 1.15 | 0 | 0 |
| Scd2 | 2011 | 1.13 | 1.16 | 0 | 0 |
| Arl6ip1 | 6248 | 1.13 | 1.17 | 0 | 1E−15 |
| Pycrl | 578 | 1.13 | 1.17 | 1E−15 | 1.8E−14 |
| Erg28 | 683.6 | 1.13 | 1.18 | 3E−15 | 8.3E−14 |
| Pgk1 | 407 | 1.13 | 1.18 | 3.15E−13 | 7.63E−12 |
| Rom1 | 500.9 | 1.13 | 1.2 | 4.67E−11 | 8.43E−10 |
| Eef1akmt1 | 350.7 | 1.13 | 1.22 | 1.39E−08 | 1.64E−07 |
| Cenpv | 293.7 | 1.13 | 1.23 | 6.57E−08 | 6.93E−07 |
| Xk | 84.8 | 1.13 | 1.25 | 2.68E−07 | 2.51E−06 |
| Clec7a | 192.7 | 1.13 | 1.26 | 6.18E−07 | 5.4E−06 |
| Nudt1 | 390.3 | 1.13 | 1.26 | 6.42E−07 | 5.59E−06 |
| Ltbp1 | 63.3 | 1.13 | 1.33 | 3.43E−05 | 0.000205 |
| 9430037O13Rik | 48.3 | 1.13 | 1.37 | 0.000118 | 0.000609 |
| Dgkg | 28.8 | 1.13 | 1.49 | 0.000398 | 0.001781 |
| Bex4 | 6.5 | 1.13 | 2.52 | 0.002771 | 0.009627 |
| Prf1 | 1704.5 | 1.12 | 1.14 | 0 | 0 |
| Dpagt1 | 515.2 | 1.12 | 1.18 | 7.25E−12 | 1.49E−10 |
| Enkd1 | 215 | 1.12 | 1.2 | 5.04E−09 | 6.42E−08 |
| Klra3 | 69.3 | 1.12 | 1.33 | 3.42E−05 | 0.000204 |
| G0s2 | 77.4 | 1.12 | 1.38 | 0.000194 | 0.000946 |
| 4930520O04Rik | 19.9 | 1.12 | 1.53 | 0.00072 | 0.002993 |
| Gm24507 | 10.1 | 1.12 | 1.9 | 0.002546 | 0.008969 |
| Irx3 | 8.6 | 1.12 | 1.79 | 0.002652 | 0.00928 |
| Zfp575 | 7.4 | 1.12 | 2.11 | 0.002982 | 0.010267 |
| 4930427A07Rik | 1017.7 | 1.11 | 1.13 | 0 | 0 |
| Fen1 | 1518.2 | 1.11 | 1.13 | 0 | 0 |
| Gng2 | 1586.5 | 1.11 | 1.15 | 0 | 0 |
| Polq | 483.2 | 1.11 | 1.17 | 4.68E−11 | 8.45E−10 |
| Aplf | 178.2 | 1.11 | 1.19 | 2.84E−09 | 3.78E−08 |
| Optn | 199.7 | 1.11 | 1.22 | 3.7E−07 | 3.4E−06 |
| Dhx58 | 1168.4 | 1.11 | 1.24 | 8.05E−07 | 6.86E−06 |
| Nudt7 | 57.3 | 1.11 | 1.34 | 6.92E−05 | 0.000382 |
| Prr18 | 25.2 | 1.11 | 1.42 | 0.000467 | 0.002048 |
| Fam109b | 49.4 | 1.11 | 1.49 | 0.000642 | 0.002708 |
| Thbs1 | 150.8 | 1.11 | 1.48 | 0.00069 | 0.002882 |
| 2210011C24Rik | 23 | 1.11 | 1.46 | 0.000846 | 0.003439 |
| Rtp4 | 1189.3 | 1.11 | 1.5 | 0.000884 | 0.003572 |
| Myom2 | 3.3 | 1.11 | 2.84 | 0.002323 | 0.00828 |
| Ncr1 | 264.6 | 1.11 | 1.76 | 0.002392 | 0.008499 |
| Adgrl4 | 27.6 | 1.11 | 1.74 | 0.002603 | 0.009132 |
| Gm15941 | 4.3 | 1.11 | 2.54 | 0.003237 | 0.011024 |
| Hoxa9 | 9 | 1.11 | 2.09 | 0.003272 | 0.011122 |
| Myrf | 11.2 | 1.11 | 2.56 | 0.00334 | 0.011303 |
| Endod1 | 867.3 | 1.1 | 1.12 | 0 | 0 |
| Ruvbl2 | 1746.2 | 1.1 | 1.14 | 0 | 2E−15 |
| Mrps6 | 469.4 | 1.1 | 1.16 | 1.21E−11 | 2.4E−10 |
| Naa38 | 642.5 | 1.1 | 1.18 | 1.39E−08 | 1.64E−07 |
| Gcat | 256.4 | 1.1 | 1.23 | 2.8E−06 | 2.16E−05 |
| Tmem163 | 137 | 1.1 | 1.32 | 0.000126 | 0.000646 |
| Ccdc80 | 29.4 | 1.1 | 1.48 | 0.000747 | 0.003085 |
| Epdr1 | 29.9 | 1.1 | 1.56 | 0.001471 | 0.005584 |
| Naaladl1 | 17.4 | 1.1 | 1.55 | 0.001554 | 0.005857 |
| Gm5541 | 13.5 | 1.1 | 1.97 | 0.003169 | 0.010832 |
| Cfb | 9.7 | 1.1 | 2.3 | 0.003841 | 0.012715 |
| Tipin | 1045.9 | 1.09 | 1.13 | 1E−15 | 4.3E−14 |
| Hikeshi | 408 | 1.09 | 1.15 | 6.66E−12 | 1.38E−10 |
| Epsti1 | 2970.1 | 1.09 | 1.14 | 2.87E−11 | 5.39E−10 |
| Mfsd2b | 366.8 | 1.09 | 1.19 | 1.24E−07 | 1.24E−06 |
| Zan | 929.3 | 1.09 | 1.19 | 2.42E−07 | 2.29E−06 |
| 1190007I07Rik | 77.8 | 1.09 | 1.22 | 2.98E−06 | 2.28E−05 |
| Kcnip3 | 48.9 | 1.09 | 1.29 | 3.51E−05 | 0.000209 |
| Ryr1 | 67.2 | 1.09 | 1.33 | 0.000206 | 0.000995 |
| Rsph1 | 29 | 1.09 | 1.35 | 0.000451 | 0.001985 |
| Slfn4 | 78.7 | 1.09 | 1.51 | 0.000918 | 0.003688 |
| Gm42517 | 3 | 1.09 | 4.44 | 0.00121 | 0.004704 |
| Gm31597 | 30.9 | 1.09 | 1.54 | 0.001558 | 0.005869 |
| Col8a2 | 11.9 | 1.09 | 2.08 | 0.003591 | 0.012023 |
| Gm5787 | 4.5 | 1.09 | 2.84 | 0.003715 | 0.012368 |
| Glp1r | 11.2 | 1.09 | 2.1 | 0.004071 | 0.013338 |
| Taf6 | 1182.3 | 1.08 | 1.1 | 0 | 0 |
| Plp2 | 432.8 | 1.08 | 1.12 | 1E−15 | 2.3E−14 |
| Atcay | 258.4 | 1.08 | 1.12 | 3E−15 | 1.06E−13 |
| Casp4 | 449.3 | 1.08 | 1.13 | 8.76E−12 | 1.79E−10 |
| Sytl2 | 1359.2 | 1.08 | 1.14 | 6.48E−11 | 1.14E−09 |
| Txn1 | 4856.9 | 1.08 | 1.14 | 1.86E−09 | 2.55E−08 |
| Banf1 | 2759.2 | 1.08 | 1.17 | 1.7E−07 | 1.65E−06 |
| Tubd1 | 123.6 | 1.08 | 1.19 | 5.64E−07 | 4.98E−06 |
| Apol10b | 57.6 | 1.08 | 1.37 | 0.000331 | 0.001516 |
| 9130604C24Rik | 12.8 | 1.08 | 1.68 | 0.002537 | 0.008944 |
| AC133083.4 | 8.9 | 1.08 | 2.13 | 0.003676 | 0.012261 |
| Mcpt8 | 355.9 | 1.08 | 2.25 | 0.004527 | 0.01461 |
| Bub3 | 2635.7 | 1.07 | 1.09 | 0 | 0 |
| Sept11 | 2112.3 | 1.07 | 1.11 | 5.1E−14 | 1.36E−12 |
| 5830432E09Rik | 221.9 | 1.07 | 1.12 | 1.77E−12 | 3.97E−11 |
| Tbc1d31 | 347.8 | 1.07 | 1.11 | 2.85E−12 | 6.2E−11 |
| Sema7a | 543.5 | 1.07 | 1.12 | 7.39E−12 | 1.52E−10 |
| PgP | 482.7 | 1.07 | 1.12 | 9.9E−11 | 1.69E−09 |
| Mrpl51 | 433.6 | 1.07 | 1.13 | 1.05E−09 | 1.5E−08 |
| AC123720.1 | 301.8 | 1.07 | 1.14 | 5.56E−09 | 7.01E−08 |
| Slc29a4 | 63.5 | 1.07 | 1.17 | 9.44E−07 | 7.95E−06 |
| Dctd | 147.9 | 1.07 | 1.19 | 2.49E−06 | 1.93E−05 |
| Csrp2 | 91.9 | 1.07 | 1.19 | 3.25E−06 | 2.46E−05 |
| Ptrh1 | 154.5 | 1.07 | 1.2 | 6.71E−06 | 4.72E−05 |
| Unc5b | 14.9 | 1.07 | 1.45 | 0.001958 | 0.00715 |
| A430018G15Rik | 17 | 1.07 | 1.55 | 0.001969 | 0.007187 |
| Upk1b | 3.1 | 1.07 | 3.96 | 0.003029 | 0.010405 |
| Gm11223 | 13 | 1.07 | 1.72 | 0.003491 | 0.011736 |
| Scrn1 | 9.5 | 1.07 | 1.96 | 0.004421 | 0.014317 |
| G6b | 5.8 | 1.07 | 2.42 | 0.004604 | 0.014825 |
| Rangap1 | 5185.4 | 1.06 | 1.07 | 0 | 0 |
| Cd8b1 | 8616.8 | 1.06 | 1.08 | 0 | 0 |
| Mms22l | 883 | 1.06 | 1.08 | 0 | 0 |
| Cchcr1 | 724.1 | 1.06 | 1.09 | 0 | 0 |
| Rnpep | 2406 | 1.06 | 1.1 | 0 | 1E−15 |
| Cntln | 217.1 | 1.06 | 1.09 | 0 | 7E−15 |
| Clic1 | 10203.8 | 1.06 | 1.09 | 1E−15 | 2.1E−14 |
| Pkmyt1 | 1872.8 | 1.06 | 1.1 | 6E−15 | 1.73E−13 |
| Phgdh | 591.6 | 1.06 | 1.1 | 6.8E−14 | 1.79E−12 |
| Lcp1 | 29534.7 | 1.06 | 1.1 | 9.5E−14 | 2.46E−12 |
| Trbv3 | 327 | 1.06 | 1.1 | 1.27E−13 | 3.23E−12 |
| Gm13394 | 2531.6 | 1.06 | 1.11 | 1.77E−11 | 3.42E−10 |
| Gm5855 | 163.2 | 1.06 | 1.12 | 3.97E−10 | 6.11E−09 |
| Rad51c | 174.3 | 1.06 | 1.13 | 6.37E−09 | 7.94E−08 |
| Fgl2 | 387.2 | 1.06 | 1.15 | 4.93E−07 | 4.42E−06 |
| Fam69b | 58.7 | 1.06 | 1.17 | 1.34E−06 | 1.1E−05 |
| Ddah2 | 90 | 1.06 | 1.21 | 3.01E−05 | 0.000182 |
| Mid2 | 43.1 | 1.06 | 1.37 | 0.000679 | 0.002842 |
| C430042M11Rik | 20.4 | 1.06 | 1.44 | 0.001415 | 0.00539 |
| 1700006J14Rik | 34.9 | 1.06 | 1.52 | 0.002226 | 0.007977 |
| Kifc5c-ps | 3.2 | 1.06 | 5.01 | 0.002273 | 0.008113 |
| Gm4316 | 14.1 | 1.06 | 1.62 | 0.002956 | 0.010189 |
| Ighv1-77 | 5.6 | 1.06 | 2.91 | 0.004291 | 0.013953 |
| Syngr3 | 14.2 | 1.06 | 1.89 | 0.004613 | 0.014847 |
| Garem1 | 10.4 | 1.06 | 2.16 | 0.004725 | 0.015161 |
| Gm5732 | 8.2 | 1.06 | 2.1 | 0.004985 | 0.01591 |
| 9230110C19Rik | 6.9 | 1.06 | 2.22 | 0.005156 | 0.016393 |
| Cdk4 | 5123.3 | 1.05 | 1.07 | 0 | 0 |
| Psmb8 | 13142.6 | 1.05 | 1.09 | 5.3E−14 | 1.42E−12 |
| Cdc25a | 933.6 | 1.05 | 1.09 | 5.4E−14 | 1.43E−12 |
| Pmf1 | 1789.9 | 1.05 | 1.09 | 1.87E−12 | 4.17E−11 |
| Galm | 344.1 | 1.05 | 1.1 | 1.09E−11 | 2.19E−10 |
| Tpgs1 | 515.9 | 1.05 | 1.11 | 2.78E−09 | 3.71E−08 |
| 2610524H06Rik | 134.2 | 1.05 | 1.12 | 3.58E−08 | 3.95E−07 |
| St14 | 207.6 | 1.05 | 1.16 | 2.31E−06 | 1.8E−05 |
| Ifi206 | 467.7 | 1.05 | 1.18 | 6.08E−06 | 4.32E−05 |
| F2rl3 | 91.5 | 1.05 | 1.18 | 1.08E−05 | 7.26E−05 |
| Ctnnbip1 | 164 | 1.05 | 1.18 | 1.81E−05 | 0.000116 |
| Ccl12 | 4.4 | 1.05 | 3.63 | 0.003485 | 0.011718 |
| Slc17a6 | 4.8 | 1.05 | 3.66 | 0.003716 | 0.012368 |
| Gm867 | 11.8 | 1.05 | 1.74 | 0.00416 | 0.013584 |
| Srd5a1 | 9.7 | 1.05 | 1.76 | 0.004432 | 0.014342 |
| Bad | 457.8 | 1.04 | 1.08 | 1.55E−11 | 3.03E−10 |
| Pafah2 | 209.2 | 1.04 | 1.11 | 2.07E−08 | 2.37E−07 |
| Dzip3 | 213.7 | 1.04 | 1.12 | 4.28E−08 | 4.63E−07 |
| Casp1 | 695.8 | 1.04 | 1.13 | 1.06E−07 | 1.08E−06 |
| Nrgn | 587.4 | 1.04 | 1.2 | 5.55E−05 | 0.000314 |
| Gm28942 | 25.8 | 1.04 | 1.35 | 0.000795 | 0.003259 |
| Gm13361 | 26.6 | 1.04 | 1.39 | 0.00157 | 0.005905 |
| AC151730.3 | 14.5 | 1.04 | 1.64 | 0.003469 | 0.011674 |
| Cyp4f39 | 4 | 1.04 | 3.04 | 0.003903 | 0.012858 |
| Ranbp17 | 12.9 | 1.04 | 1.93 | 0.005338 | 0.01689 |
| Gm6419 | 7.9 | 1.04 | 2.1 | 0.006041 | 0.0188 |
| Unc119b | 2622.3 | 1.03 | 1.05 | 0 | 0 |
| Ak3 | 1405.9 | 1.03 | 1.05 | 0 | 0 |
| Brca2 | 378 | 1.03 | 1.06 | 0 | 0 |
| Prelid2 | 433.7 | 1.03 | 1.06 | 0 | 1E−15 |
| Hmbs | 853.4 | 1.03 | 1.07 | 2.67E−12 | 5.82E−11 |
| Cobll1 | 564 | 1.03 | 1.07 | 1.31E−11 | 2.6E−10 |
| Ttf2 | 773.8 | 1.03 | 1.08 | 1.77E−10 | 2.94E−09 |
| Ap1s2 | 357.9 | 1.03 | 1.09 | 6.21E−10 | 9.25E−09 |
| Arhgap33 | 461.4 | 1.03 | 1.1 | 1.1E−07 | 1.11E−06 |
| Cxcr5 | 1400.5 | 1.03 | 1.11 | 1.62E−07 | 1.58E−06 |
| Zfp324 | 80.8 | 1.03 | 1.16 | 1.23E−05 | 8.15E−05 |
| 1700008J07Rik | 46 | 1.03 | 1.2 | 8.13E−05 | 0.00044 |
| Pla2g1b | 3.1 | 1.03 | 5.68 | 0.000671 | 0.002812 |
| Rasd2 | 26.4 | 1.03 | 1.31 | 0.001055 | 0.004161 |
| Gm19412 | 24.7 | 1.03 | 1.45 | 0.002222 | 0.007968 |
| Gm21850 | 5.8 | 1.03 | 5.15 | 0.003849 | 0.012735 |
| Ninj2 | 22.5 | 1.03 | 1.57 | 0.003881 | 0.012815 |
| Cacna1b | 12.9 | 1.03 | 1.59 | 0.003981 | 0.013088 |
| Hspd1 | 2211.8 | 1.02 | 1.03 | 0 | 0 |
| Gins4 | 994.2 | 1.02 | 1.03 | 0 | 0 |
| Hirip3 | 1284.6 | 1.02 | 1.04 | 0 | 1E−15 |
| Dtx3l | 2072.2 | 1.02 | 1.05 | 1E−15 | 3.7E−14 |
| Atad5 | 649 | 1.02 | 1.05 | 4E−15 | 1.19E−13 |
| Slc22a15 | 789 | 1.02 | 1.05 | 2.1E−13 | 5.2E−12 |
| Nefh | 428.6 | 1.02 | 1.08 | 2.24E−09 | 3.03E−08 |
| Gemin8 | 131.1 | 1.02 | 1.14 | 1.99E−05 | 0.000126 |
| Coa3 | 751.4 | 1.02 | 1.17 | 3.92E−05 | 0.000231 |
| AL591582.1 | 67.5 | 1.02 | 1.2 | 0.000137 | 0.000694 |
| Rabl2 | 66.4 | 1.02 | 1.21 | 0.000142 | 0.000719 |
| BC147527 | 314.7 | 1.02 | 1.21 | 0.000232 | 0.001111 |
| F5 | 26.3 | 1.02 | 1.32 | 0.001751 | 0.006497 |
| Cysltr1 | 12.2 | 1.02 | 1.53 | 0.003789 | 0.012571 |
| Ankrd29 | 17.2 | 1.02 | 1.59 | 0.003942 | 0.01298 |
| Serpinb1b | 5.6 | 1.02 | 3.25 | 0.00527 | 0.016699 |
| Cxcl11 | 12.9 | 1.02 | 1.71 | 0.005596 | 0.017603 |
| A730089K16Rik | 8.9 | 1.02 | 3.51 | 0.006351 | 0.019658 |
| Gm12669 | 5.9 | 1.02 | 2.72 | 0.006491 | 0.020036 |
| Draxin | 7.5 | 1.02 | 2.51 | 0.006903 | 0.021116 |
| Lrrn4 | 10.2 | 1.02 | 2.39 | 0.00739 | 0.022374 |
| Fbxw8 | 1446.7 | 1.01 | 1.03 | 0 | 0 |
| Mis18a | 835 | 1.01 | 1.03 | 0 | 0 |
| Adprh | 1898 | 1.01 | 1.03 | 0 | 1E−15 |
| Gmds | 345.1 | 1.01 | 1.05 | 2.03E−12 | 4.49E−11 |
| Hat1 | 1129 | 1.01 | 1.06 | 7.79E−10 | 1.14E−08 |
| Psmb10 | 4675.3 | 1.01 | 1.07 | 1.49E−09 | 2.07E−08 |
| F730043M19Rik | 151.6 | 1.01 | 1.1 | 5.92E−07 | 5.2E−06 |
| Map1a | 43.3 | 1.01 | 1.14 | 5.25E−05 | 0.000299 |
| Gata1 | 221 | 1.01 | 1.18 | 0.000138 | 0.0007 |
| 4930524J08Rik | 30.3 | 1.01 | 1.2 | 0.00043 | 0.001909 |
| Ly6c2 | 4431.1 | 1.01 | 1.23 | 0.000513 | 0.002224 |
| Gm30211 | 96.4 | 1.01 | 1.34 | 0.001432 | 0.005448 |
| Slc30a2 | 29 | 1.01 | 1.33 | 0.002911 | 0.010056 |
| Lpar3 | 17.1 | 1.01 | 1.53 | 0.003294 | 0.011174 |
| Gm35037 | 11.6 | 1.01 | 1.65 | 0.005347 | 0.016913 |
| Pcdh7 | 8.7 | 1.01 | 1.64 | 0.006542 | 0.020151 |
| Ntf5 | 8.2 | 1.01 | 1.67 | 0.006819 | 0.020886 |
| Olfr414 | 5.8 | 1.01 | 3.46 | 0.007252 | 0.022 |
| AC153562.2 | 4.6 | 1.01 | 2.14 | 0.007428 | 0.022477 |
| Cd248 | 7.6 | 1.01 | 2.32 | 0.007647 | 0.023068 |
| Ccdc120 | 9.6 | 1.01 | 1.83 | 0.007745 | 0.023309 |
| Ahsa1 | 2898.5 | 1 | 1.02 | 0 | 0 |
| Cdca4 | 1330.7 | 1 | 1.03 | 0 | 0 |
| Ebp | 1270.8 | 1 | 1.03 | 8E−15 | 2.22E−13 |
| Thop1 | 736 | 1 | 1.03 | 6.1E−14 | 1.6E−12 |
| Jdp2 | 335.6 | 1 | 1.03 | 2.76E−13 | 6.73E−12 |
| Rpa3 | 304.5 | 1 | 1.04 | 3E−10 | 4.76E−09 |
| Ece2 | 225.8 | 1 | 1.06 | 1.04E−08 | 1.25E−07 |
| Cttn | 156.2 | 1 | 1.07 | 2.39E−08 | 2.7E−07 |
| Ydjc | 187.1 | 1 | 1.07 | 1.09E−07 | 1.1E−06 |
| 5033430I15Rik | 174.1 | 1 | 1.07 | 4.67E−07 | 4.21E−06 |
| Orc1 | 394.8 | 1 | 1.09 | 8.9E−07 | 7.52E−06 |
| Cx3cr1 | 141.8 | 1 | 1.09 | 3.2E−06 | 2.43E−05 |
| Ltbp3 | 64 | 1 | 1.12 | 2.83E−05 | 0.000173 |
| Zfp239 | 60.8 | 1 | 1.15 | 5.9E−05 | 0.000332 |
| Mansc1 | 76.8 | 1 | 1.17 | 0.000128 | 0.000654 |
| Bahcc1 | 65.2 | 1 | 1.17 | 0.000156 | 0.00078 |
| Rpl39-ps | 163.8 | 1 | 1.2 | 0.000445 | 0.001964 |
| Ccl2 | 77.5 | 1 | 1.39 | 0.002955 | 0.010188 |
| Col5a1 | 37.5 | 1 | 1.48 | 0.004515 | 0.014573 |
| Wisp1 | 171.4 | 1 | 1.46 | 0.004818 | 0.015407 |
| Hist1h2bl | 3.2 | 1 | 2.02 | 0.00672 | 0.020634 |
| Gm11658 | 7.3 | 1 | 1.65 | 0.007621 | 0.023001 |
| Gm44101 | 5.1 | 1 | 2.58 | 0.007779 | 0.02339 |
| Atp6v1g3 | 12.9 | 1 | 1.89 | 0.007957 | 0.023852 |
| Bbs2 | 593.4 | −1 | −1.02 | 0 | 0 |
| Cdc42bpb | 487.3 | −1 | −1.06 | 4.36E−09 | 5.61E−08 |
| Dapk1 | 459.6 | −1 | −1.06 | 1.22E−08 | 1.45E−07 |
| Lonrf1 | 133.9 | −1 | −1.06 | 2.72E−08 | 3.04E−07 |
| Vps37b | 99356.7 | −1 | −1.07 | 1.11E−07 | 1.12E−06 |
| C130050O18Rik | 95.9 | −1 | −1.14 | 1.81E−05 | 0.000116 |
| Thrb | 56.4 | −1 | −1.13 | 3.27E−05 | 0.000196 |
| Gm45743 | 60.6 | −1 | −1.14 | 4.4E−05 | 0.000256 |
| Gm42743 | 41.9 | −1 | −1.13 | 4.43E−05 | 0.000257 |
| Nectin1 | 128.6 | −1 | −1.15 | 7.02E−05 | 0.000387 |
| Proscos | 49.9 | −1 | −1.15 | 0.000156 | 0.00078 |
| Snx24 | 100.2 | −1 | −1.2 | 0.000273 | 0.00128 |
| Gm43919 | 34.9 | −1 | −1.19 | 0.000325 | 0.001493 |
| Fbxl12os | 26.5 | −1 | −1.25 | 0.001077 | 0.004236 |
| Robo4 | 55.1 | −1 | −1.28 | 0.001574 | 0.00592 |
| Fam149a | 48.8 | −1 | −1.32 | 0.001849 | 0.006801 |
| Gm17021 | 42.4 | −1 | −1.35 | 0.00255 | 0.008981 |
| Gm15918 | 28.4 | −1 | −1.36 | 0.003178 | 0.010856 |
| Dcst2 | 12.5 | −1 | −1.43 | 0.004038 | 0.013242 |
| Traj23 | 11.2 | −1 | −1.45 | 0.004281 | 0.013929 |
| Cyp1a1 | 14.7 | −1 | −1.53 | 0.005398 | 0.017057 |
| Siglecg | 751.8 | −1 | −1.64 | 0.005784 | 0.018124 |
| Gm5538 | 4.9 | −1 | −3.78 | 0.006528 | 0.020119 |
| 2310026I22Rik | 9.2 | −1 | −1.66 | 0.006733 | 0.020671 |
| Zfp536 | 9.7 | −1 | −1.65 | 0.006926 | 0.02116 |
| Slc8a2 | 9.9 | −1 | −1.82 | 0.007393 | 0.022379 |
| C130074G19Rik | 7.9 | −1 | −1.78 | 0.008117 | 0.024268 |
| Gm37078 | 5.3 | −1 | −2.53 | 0.008127 | 0.024294 |
| Enthd1 | 11.5 | −1 | −1.83 | 0.008208 | 0.024502 |
| Oprl1 | 7.9 | −1 | −1.99 | 0.008234 | 0.024563 |
| Gm16069 | 513.8 | −1.01 | −1.03 | 0 | 1.4E−14 |
| Slc12a7 | 12768.1 | −1.01 | −1.05 | 2.7E−14 | 7.4E−13 |
| Gm1043 | 603.2 | −1.01 | −1.06 | 2.48E−10 | 4E−09 |
| Gm16638 | 160.1 | −1.01 | −1.07 | 7.79E−10 | 1.14E−08 |
| Skil | 15256 | −1.01 | −1.08 | 3.99E−08 | 4.35E−07 |
| Gm6085 | 80.3 | −1.01 | −1.16 | 0.000113 | 0.000586 |
| Arhgap20 | 94.6 | −1.01 | −1.18 | 0.000163 | 0.00081 |
| AC158605.3 | 38.2 | −1.01 | −1.27 | 0.000516 | 0.002237 |
| She | 55.5 | −1.01 | −1.25 | 0.000684 | 0.002858 |
| Abca9 | 130.3 | −1.01 | −1.29 | 0.000993 | 0.003946 |
| Chrna9 | 16 | −1.01 | −1.35 | 0.002576 | 0.009052 |
| Cplx1 | 15.4 | −1.01 | −1.49 | 0.004189 | 0.013661 |
| Gm2694 | 5.2 | −1.01 | −3.48 | 0.006165 | 0.019142 |
| Fxyd1 | 9.9 | −1.01 | −1.64 | 0.006183 | 0.01919 |
| Gm31479 | 6.5 | −1.01 | −2.85 | 0.007441 | 0.022507 |
| Clec2h | 5.3 | −1.01 | −2.58 | 0.007558 | 0.022832 |
| Hba-a2 | 368.3 | −1.01 | −2.17 | 0.007639 | 0.023049 |
| G730013B05Rik | 7.5 | −1.01 | −1.88 | 0.00769 | 0.02318 |
| A430072C10Rik | 4.4 | −1.01 | −2.03 | 0.007723 | 0.023258 |
| Slc6a19os | 5.5 | −1.01 | −1.86 | 0.008023 | 0.024027 |
| Sema3b | 11.3 | −1.01 | −1.97 | 0.008087 | 0.024202 |
| Ldhal6b | 9.7 | −1.01 | −1.95 | 0.008111 | 0.024255 |
| Dip2c | 434.7 | −1.02 | −1.08 | 1.85E−09 | 2.54E−08 |
| Trio | 1619.6 | −1.02 | −1.08 | 2.11E−08 | 2.41E−07 |
| Il13ra1 | 83.3 | −1.02 | −1.13 | 3.14E−06 | 2.39E−05 |
| Gm45059 | 77.9 | −1.02 | −1.12 | 5.67E−06 | 4.06E−05 |
| Lrrc25 | 483.1 | −1.02 | −1.15 | 9.03E−06 | 6.18E−05 |
| Ptpro | 88.1 | −1.02 | −1.16 | 1.63E−05 | 0.000106 |
| Susd4 | 89.1 | −1.02 | −1.16 | 2.64E−05 | 0.000162 |
| AC152827.1 | 55.1 | −1.02 | −1.19 | 0.000137 | 0.000695 |
| Epb41l1 | 134.6 | −1.02 | −1.21 | 0.000176 | 0.000867 |
| Cd177 | 45.2 | −1.02 | −1.33 | 0.001186 | 0.004619 |
| AC104880.1 | 22.4 | −1.02 | −1.36 | 0.00212 | 0.007654 |
| Gm16060 | 18 | −1.02 | −1.37 | 0.002489 | 0.008799 |
| Pmepa1os | 12.1 | −1.02 | −1.46 | 0.003899 | 0.012852 |
| Hoga1 | 13.2 | −1.02 | −1.61 | 0.004419 | 0.014313 |
| Gm37109 | 3.1 | −1.02 | −2.45 | 0.005212 | 0.016548 |
| Plxna2 | 19.9 | −1.02 | −1.61 | 0.005262 | 0.016685 |
| A430027C01Rik | 13.4 | −1.02 | −1.85 | 0.006086 | 0.018924 |
| Gm21984 | 9.4 | −1.02 | −1.85 | 0.006525 | 0.020112 |
| Gm44860 | 11.3 | −1.02 | −2.21 | 0.006672 | 0.020505 |
| Gramd2 | 8.5 | −1.02 | −2.62 | 0.006793 | 0.020823 |
| Mcf2 | 7.3 | −1.02 | −3.16 | 0.006935 | 0.02118 |
| Ablim3 | 13.3 | −1.02 | −2.32 | 0.006988 | 0.021312 |
| 4922502D21Rik | 7.8 | −1.02 | −1.93 | 0.007227 | 0.021936 |
| Cyp4f13 | 1429.7 | −1.03 | −1.04 | 0 | 0 |
| Gm37494 | 567.7 | −1.03 | −1.07 | 1.06E−11 | 2.12E−10 |
| Maff | 11634.4 | −1.03 | −1.08 | 1.68E−11 | 3.25E−10 |
| Tdrp | 4170.6 | −1.03 | −1.08 | 1.84E−10 | 3.04E−09 |
| Dennd2c | 139.5 | −1.03 | −1.1 | 2.13E−08 | 2.43E−07 |
| Pla2g7 | 1374.2 | −1.03 | −1.11 | 9.79E−08 | 1E−06 |
| Apoe | 8629.4 | −1.03 | −1.11 | 1.62E−07 | 1.58E−06 |
| AC140264.2 | 106 | −1.03 | −1.13 | 1.08E−06 | 8.95E−06 |
| Alpk1 | 263.7 | −1.03 | −1.12 | 1.08E−06 | 8.96E−06 |
| Jmy | 4073.6 | −1.03 | −1.13 | 2.17E−06 | 1.7E−05 |
| Fdx1l | 80.3 | −1.03 | −1.13 | 2.58E−06 | 1.99E−05 |
| Pnck | 68.8 | −1.03 | −1.15 | 9.22E−06 | 6.3E−05 |
| Ttc12 | 74.5 | −1.03 | −1.25 | 0.000243 | 0.001157 |
| Gm12966 | 32.5 | −1.03 | −1.23 | 0.000274 | 0.001283 |
| AC117769.3 | 40.9 | −1.03 | −1.27 | 0.000618 | 0.002621 |
| Foxq1 | 65.3 | −1.03 | −1.28 | 0.0007 | 0.002921 |
| Gm12000 | 21.3 | −1.03 | −1.42 | 0.002794 | 0.009693 |
| Mir7046 | 10.4 | −1.03 | −1.64 | 0.005264 | 0.016687 |
| Gm31532 | 8.9 | −1.03 | −1.73 | 0.006325 | 0.019582 |
| L3mbtl1 | 7.3 | −1.03 | −2.07 | 0.006502 | 0.020058 |
| Bnip3l-ps | 6.6 | −1.03 | −1.96 | 0.006735 | 0.020672 |
| Sfmbt2 | 429.2 | −1.04 | −1.09 | 2.21E−11 | 4.22E−10 |
| Cd86 | 1049.2 | −1.04 | −1.09 | 3.35E−10 | 5.26E−09 |
| Cdc14b | 1062 | −1.04 | −1.1 | 2.04E−09 | 2.79E−08 |
| Gm42595 | 333.4 | −1.04 | −1.1 | 2.24E−09 | 3.02E−08 |
| Tlr13 | 195.6 | −1.04 | −1.13 | 5.29E−08 | 5.62E−07 |
| Egf | 186.8 | −1.04 | −1.12 | 1.95E−07 | 1.87E−06 |
| Fgfr1 | 240.7 | −1.04 | −1.13 | 6.63E−07 | 5.75E−06 |
| Cap2 | 83.3 | −1.04 | −1.14 | 8.33E−07 | 7.09E−06 |
| C1qb | 6167.6 | −1.04 | −1.15 | 4.45E−06 | 3.26E−05 |
| Fam167a | 155 | −1.04 | −1.16 | 5.75E−06 | 4.11E−05 |
| Cspg5 | 62.6 | −1.04 | −1.17 | 2.27E−05 | 0.000142 |
| Tnnt2 | 65.1 | −1.04 | −1.2 | 2.53E−05 | 0.000156 |
| Cxcl16 | 314.6 | −1.04 | −1.2 | 5.52E−05 | 0.000313 |
| A430057M04Rik | 48.4 | −1.04 | −1.27 | 0.000387 | 0.001739 |
| Oscp1 | 44.4 | −1.04 | −1.29 | 0.000555 | 0.002385 |
| H2-Ea-ps | 7790.9 | −1.04 | −1.31 | 0.000565 | 0.002422 |
| Il12b | 54.2 | −1.04 | −1.45 | 0.001388 | 0.005304 |
| Pde8b | 30.7 | −1.04 | −1.64 | 0.003238 | 0.011026 |
| Ptch2 | 15.2 | −1.04 | −1.56 | 0.003758 | 0.01249 |
| Tmtc1 | 26.5 | −1.04 | −1.66 | 0.004404 | 0.01428 |
| 1810011O10Rik | 24.2 | −1.04 | −1.65 | 0.004405 | 0.014281 |
| Gm12167 | 10.2 | −1.04 | −1.7 | 0.005323 | 0.016848 |
| Gm12089 | 3.8 | −1.04 | −2.79 | 0.005526 | 0.017413 |
| Gm28035 | 6 | −1.04 | −2.57 | 0.006042 | 0.0188 |
| C230037L18Rik | 243.6 | −1.05 | −1.1 | 7.77E−13 | 1.82E−11 |
| Dyx1c1 | 106 | −1.05 | −1.13 | 3.09E−07 | 2.87E−06 |
| Hebp1 | 934.9 | −1.05 | −1.18 | 5.55E−06 | 3.99E−05 |
| Tmem51 | 428.5 | −1.05 | −1.17 | 6E−06 | 4.27E−05 |
| Naip5 | 181.7 | −1.05 | −1.19 | 1.01E−05 | 6.87E−05 |
| Cd300c2 | 673.6 | −1.05 | −1.2 | 2.32E−05 | 0.000144 |
| Card10 | 54.5 | −1.05 | −1.19 | 2.47E−05 | 0.000153 |
| Coro6 | 40.8 | −1.05 | −1.22 | 8.79E−05 | 0.000471 |
| Bhlhe41 | 99.6 | −1.05 | −1.24 | 9.66E−05 | 0.000512 |
| Fhit | 63.9 | −1.05 | −1.24 | 0.000136 | 0.000692 |
| Gm18194 | 29.5 | −1.05 | −1.28 | 0.000246 | 0.001168 |
| Lin7b | 27.7 | −1.05 | −1.28 | 0.000301 | 0.001393 |
| Gm6012 | 40.5 | −1.05 | −1.29 | 0.000404 | 0.001806 |
| Sox5 | 27.1 | −1.05 | −1.31 | 0.000448 | 0.001973 |
| P2ry2 | 61.4 | −1.05 | −1.36 | 0.000804 | 0.003293 |
| Gm44214 | 21.7 | −1.05 | −1.35 | 0.000952 | 0.003805 |
| Blnk | 622 | −1.05 | −1.41 | 0.001157 | 0.004518 |
| Acot4 | 15.6 | −1.05 | −1.67 | 0.003129 | 0.010702 |
| AC160562.1 | 14.3 | −1.05 | −1.6 | 0.003251 | 0.011059 |
| Myo15 | 15.2 | −1.05 | −1.66 | 0.003332 | 0.011284 |
| Extl1 | 17.9 | −1.05 | −1.78 | 0.005054 | 0.0161 |
| Gm15929 | 8.8 | −1.05 | −1.92 | 0.005178 | 0.016449 |
| Gm43777 | 8.3 | −1.05 | −1.79 | 0.005291 | 0.016755 |
| Gm2814 | 9.6 | −1.05 | −2.68 | 0.005673 | 0.017801 |
| Rassf3 | 6479.1 | −1.06 | −1.09 | 0 | 8E−15 |
| 2810021J22Rik | 1264.6 | −1.06 | −1.12 | 9.26E−11 | 1.59E−09 |
| Ovgp1 | 326.4 | −1.06 | −1.12 | 1.35E−10 | 2.27E−09 |
| Mapk12 | 152.1 | −1.06 | −1.15 | 5.43E−08 | 5.76E−07 |
| AC125071.1 | 106 | −1.06 | −1.17 | 6.39E−07 | 5.57E−06 |
| Bbs1 | 97.2 | −1.06 | −1.17 | 1.57E−06 | 1.27E−05 |
| Gm10167 | 58.6 | −1.06 | −1.22 | 2.61E−05 | 0.00016 |
| Rassf8 | 83.1 | −1.06 | −1.23 | 4.05E−05 | 0.000238 |
| Mapk15 | 40.7 | −1.06 | −1.27 | 0.000162 | 0.000807 |
| Ghrl | 55 | −1.06 | −1.33 | 0.000426 | 0.001892 |
| Slc4a11 | 40.7 | −1.06 | −1.33 | 0.000485 | 0.002114 |
| Cybrd1 | 29.8 | −1.06 | −1.35 | 0.000586 | 0.002499 |
| Gm8251 | 24.9 | −1.06 | −1.4 | 0.000924 | 0.003706 |
| Tceal1 | 18.7 | −1.06 | −1.52 | 0.002435 | 0.00863 |
| Pifo | 13.3 | −1.06 | −1.59 | 0.003076 | 0.010546 |
| Bicdl2 | 10.3 | −1.06 | −1.77 | 0.003577 | 0.011987 |
| CT009757.4 | 3.8 | −1.06 | −3.2 | 0.004176 | 0.013625 |
| Tmem30b | 16 | −1.06 | −1.86 | 0.004968 | 0.015862 |
| Gm826 | 7.6 | −1.06 | −2.41 | 0.0051 | 0.01623 |
| Tmem236 | 8.3 | −1.06 | −1.94 | 0.005165 | 0.016415 |
| Gm13201 | 8.2 | −1.06 | −2.49 | 0.00522 | 0.01657 |
| Zbtb20 | 1429.4 | −1.07 | −1.1 | 0 | 1E−15 |
| Dstyk | 697.7 | −1.07 | −1.1 | 0 | 8E−15 |
| 1830077J02Rik | 399 | −1.07 | −1.15 | 9.22E−09 | 1.11E−07 |
| Stac3 | 119.4 | −1.07 | −1.15 | 1.12E−07 | 1.13E−06 |
| Xkrx | 235.4 | −1.07 | −1.18 | 5.59E−07 | 4.94E−06 |
| Aph1c | 92.6 | −1.07 | −1.18 | 1.71E−06 | 1.37E−05 |
| Ly86 | 762.3 | −1.07 | −1.21 | 8.49E−06 | 5.85E−05 |
| Stab2 | 1255.1 | −1.07 | −1.21 | 8.58E−06 | 5.91E−05 |
| 4921507P07Rik | 57.2 | −1.07 | −1.21 | 8.97E−06 | 6.15E−05 |
| Lst1 | 1097.1 | −1.07 | −1.21 | 1.14E−05 | 7.63E−05 |
| Erbb2 | 135 | −1.07 | −1.26 | 4.73E−05 | 0.000273 |
| Syde2 | 248.2 | −1.07 | −1.24 | 4.75E−05 | 0.000274 |
| Hspg2 | 41.3 | −1.07 | −1.3 | 0.00025 | 0.001187 |
| Cfap74 | 36.6 | −1.07 | −1.35 | 0.000347 | 0.001581 |
| Rusc2 | 49.2 | −1.07 | −1.34 | 0.000354 | 0.001609 |
| Cpe | 36.2 | −1.07 | −1.35 | 0.000616 | 0.002616 |
| 1700061G19Rik | 22.8 | −1.07 | −1.36 | 0.000645 | 0.002719 |
| Rab30 | 116 | −1.07 | −1.57 | 0.001884 | 0.006917 |
| Gm15537 | 21 | −1.07 | −1.58 | 0.002688 | 0.009387 |
| Dlk1 | 20.9 | −1.07 | −1.54 | 0.002932 | 0.010115 |
| Akp-ps1 | 12.6 | −1.07 | −1.63 | 0.003213 | 0.010955 |
| Gm23346 | 11.3 | −1.07 | −2.42 | 0.003589 | 0.012019 |
| Col2a1 | 13.8 | −1.07 | −1.69 | 0.003605 | 0.012063 |
| Muc3 | 12.6 | −1.07 | −1.82 | 0.004403 | 0.014278 |
| Rgsl1 | 4.5 | −1.07 | −2.66 | 0.004427 | 0.014331 |
| Gm42655 | 7.7 | −1.07 | −2.37 | 0.004556 | 0.014693 |
| Hbb-bs | 1829.7 | −1.07 | −2.23 | 0.004758 | 0.015252 |
| Crip3 | 8.6 | −1.07 | −2.83 | 0.004816 | 0.015403 |
| 4732440D04Rik | 113 | −1.08 | −1.14 | 2.24E−10 | 3.65E−09 |
| Mmp15 | 73.3 | −1.08 | −1.16 | 2.28E−08 | 2.6E−07 |
| Rgl1 | 1317.4 | −1.08 | −1.18 | 1.34E−07 | 1.33E−06 |
| Gm15706 | 118.9 | −1.08 | −1.18 | 1.66E−07 | 1.62E−06 |
| Cdk14 | 85 | −1.08 | −1.28 | 5.71E−05 | 0.000322 |
| Gm18310 | 30.9 | −1.08 | −1.37 | 0.000572 | 0.002447 |
| 4933433G15Rik | 17.6 | −1.08 | −1.52 | 0.001341 | 0.005145 |
| Blk | 523.7 | −1.08 | −1.89 | 0.0037 | 0.012329 |
| Hrh1 | 9.5 | −1.08 | −1.97 | 0.003762 | 0.012499 |
| Dusp10 | 33817.9 | −1.09 | −1.12 | 0 | 1E−15 |
| Gm42829 | 278.5 | −1.09 | −1.12 | 0 | 1E−15 |
| Klf7 | 2672.7 | −1.09 | −1.12 | 0 | 1E−15 |
| Gpr157 | 197.5 | −1.09 | −1.12 | 7E−15 | 2.04E−13 |
| Sez6l2 | 335 | −1.09 | −1.13 | 1.4E−14 | 3.99E−13 |
| 1700109H08Rik | 571.4 | −1.09 | −1.15 | 4.74E−11 | 8.5E−10 |
| Arsg | 171.4 | −1.09 | −1.17 | 4.69E−09 | 6E−08 |
| Lypd6b | 490.3 | −1.09 | −1.17 | 1.28E−08 | 1.51E−07 |
| Gm14168 | 317.9 | −1.09 | −1.17 | 3.99E−08 | 4.35E−07 |
| Rnd2 | 50.2 | −1.09 | −1.21 | 3.16E−06 | 2.4E−05 |
| 1700003F12Rik | 31.2 | −1.09 | −1.35 | 0.000266 | 0.001254 |
| Cd300c | 47.9 | −1.09 | −1.42 | 0.000366 | 0.001658 |
| 1700028N14Rik | 48.1 | −1.09 | −1.37 | 0.000451 | 0.001986 |
| Gm11210 | 24.9 | −1.09 | −1.41 | 0.000578 | 0.002472 |
| Stac2 | 76.6 | −1.09 | −1.44 | 0.000652 | 0.002746 |
| Gm13199 | 23 | −1.09 | −1.44 | 0.000847 | 0.003442 |
| Wfdc3 | 16.1 | −1.09 | −1.49 | 0.00138 | 0.005282 |
| D630033O11Rik | 38.5 | −1.09 | −1.71 | 0.002251 | 0.00805 |
| Pcdhga12 | 13.8 | −1.09 | −1.85 | 0.003166 | 0.010824 |
| Mcmdc2 | 8.5 | −1.09 | −2.47 | 0.004266 | 0.013892 |
| AtG | 5537.4 | −1.1 | −1.17 | 2.92E−10 | 4.66E−09 |
| Cage1 | 267.2 | −1.1 | −1.17 | 4.95E−09 | 6.32E−08 |
| Plce1 | 62.8 | −1.1 | −1.22 | 1.85E−06 | 1.47E−05 |
| Wdfy3 | 451.6 | −1.1 | −1.27 | 1.85E−05 | 0.000118 |
| Gm13054 | 37.4 | −1.1 | −1.34 | 0.000134 | 0.000681 |
| Snord72 | 35.8 | −1.1 | −1.34 | 0.000207 | 0.000998 |
| Tdrd9 | 21.8 | −1.1 | −1.44 | 0.00065 | 0.00274 |
| Fam229a | 25.2 | −1.1 | −1.44 | 0.000757 | 0.003124 |
| Gm38192 | 22.6 | −1.1 | −1.57 | 0.001304 | 0.005024 |
| A930038B10Rik | 14.3 | −1.1 | −1.62 | 0.002057 | 0.007455 |
| AL607131.1 | 38.2 | −1.1 | −1.7 | 0.002202 | 0.007906 |
| Prox1 | 12.4 | −1.1 | −1.72 | 0.002422 | 0.008591 |
| Alas2 | 140.4 | −1.1 | −1.81 | 0.002845 | 0.009851 |
| Gm15696 | 19.1 | −1.1 | −1.81 | 0.00296 | 0.010201 |
| Arhgap42 | 15.2 | −1.1 | −1.96 | 0.002998 | 0.010315 |
| Fam13a | 8.3 | −1.1 | −2.15 | 0.003567 | 0.011962 |
| Rgcc | 877.6 | −1.11 | −1.14 | 0 | 0 |
| Thbs3 | 181.1 | −1.11 | −1.16 | 2.09E−12 | 4.62E−11 |
| Tnfrsf12a | 1193.9 | −1.11 | −1.18 | 2.33E−10 | 3.77E−09 |
| Wnt5b | 363.9 | −1.11 | −1.17 | 2.97E−10 | 4.72E−09 |
| D7Bwg0826e | 309.2 | −1.11 | −1.18 | 6.4E−10 | 9.5E−09 |
| Cdo1 | 154.9 | −1.11 | −1.18 | 1.84E−09 | 2.52E−08 |
| Ing4 | 286.6 | −1.11 | −1.2 | 1.16E−08 | 1.38E−07 |
| Dock4 | 301.1 | −1.11 | −1.21 | 3.54E−08 | 3.91E−07 |
| Sh2d4b | 213.3 | −1.11 | −1.21 | 1.23E−07 | 1.23E−06 |
| Nr4a3 | 16228.2 | −1.11 | −1.22 | 6.69E−07 | 5.8E−06 |
| Fam213b | 477.2 | −1.11 | −1.23 | 6.75E−07 | 5.85E−06 |
| Gk5 | 113.2 | −1.11 | −1.23 | 8.68E−07 | 7.36E−06 |
| Hey1 | 1015.7 | −1.11 | −1.23 | 1.04E−06 | 8.68E−06 |
| Bach2it1 | 141.1 | −1.11 | −1.23 | 1.16E−06 | 9.58E−06 |
| Sorcs2 | 70.7 | −1.11 | −1.26 | 5.81E−06 | 4.15E−05 |
| Bmp2 | 169.3 | −1.11 | −1.27 | 7.01E−06 | 4.92E−05 |
| Snta1 | 208.6 | −1.11 | −1.27 | 1.06E−05 | 7.16E−05 |
| Gm13710 | 186.2 | −1.11 | −1.29 | 1.71E−05 | 0.00011 |
| Cascl | 50.4 | −1.11 | −1.3 | 2.08E−05 | 0.000131 |
| Nr3c2 | 42.8 | −1.11 | −1.35 | 7.47E−05 | 0.000408 |
| Myo5c | 12.5 | −1.11 | −1.65 | 0.001671 | 0.006233 |
| Gm45140 | 14.1 | −1.11 | −1.7 | 0.001727 | 0.006423 |
| Scx | 11.1 | −1.11 | −1.65 | 0.002003 | 0.007288 |
| AC156952.1 | 12.6 | −1.11 | −1.89 | 0.002764 | 0.009612 |
| D6Ertd474e | 4.7 | −1.11 | −3.38 | 0.003004 | 0.010332 |
| Gm9873 | 11.3 | −1.11 | −1.97 | 0.003018 | 0.010375 |
| Smim6 | 7.7 | −1.11 | −2.44 | 0.003338 | 0.011301 |
| Gm20513 | 6.4 | −1.11 | −2.77 | 0.003468 | 0.011673 |
| Hap1 | 446.1 | −1.12 | −1.18 | 2.38E−10 | 3.85E−09 |
| Epb4113 | 649.5 | −1.12 | −1.22 | 1.19E−07 | 1.19E−06 |
| Gm9403 | 86.5 | −1.12 | −1.24 | 2.13E−07 | 2.03E−06 |
| Ccpg1 | 685.5 | −1.12 | −1.24 | 7.55E−07 | 6.47E−06 |
| Clec4a1 | 389.4 | −1.12 | −1.28 | 6.12E−06 | 4.35E−05 |
| CAAA01194877.1 | 40.7 | −1.12 | −1.37 | 0.000103 | 0.000544 |
| Padi1 | 28.6 | −1.12 | −1.39 | 0.000201 | 0.000973 |
| Cd36 | 136.3 | −1.12 | −1.45 | 0.000339 | 0.001549 |
| Bcar1 | 37.6 | −1.12 | −1.43 | 0.00035 | 0.001594 |
| Snx22 | 65.3 | −1.12 | −1.61 | 0.001167 | 0.004552 |
| Trim7 | 268.2 | −1.12 | −1.78 | 0.001673 | 0.006239 |
| Actl7b | 11.5 | −1.12 | −1.66 | 0.001764 | 0.006538 |
| Cacna1f | 12.7 | −1.12 | −1.62 | 0.001813 | 0.006692 |
| Gm10425 | 5.3 | −1.12 | −3.54 | 0.002191 | 0.007874 |
| Prph | 18.6 | −1.12 | −1.79 | 0.002429 | 0.00861 |
| Mfap2 | 4.7 | −1.12 | −2.58 | 0.00275 | 0.009577 |
| Gm16083 | 11.2 | −1.12 | −2.24 | 0.002964 | 0.010212 |
| Peg13 | 3375.2 | −1.13 | −1.17 | 0 | 0 |
| Basp1 | 514.9 | −1.13 | −1.21 | 5.81E−10 | 8.69E−09 |
| Gm42659 | 103.8 | −1.13 | −1.21 | 4.02E−09 | 5.22E−08 |
| Tagln | 142.5 | −1.13 | −1.21 | 1.06E−08 | 1.27E−07 |
| Syk | 3325.1 | −1.13 | −1.23 | 5.23E−08 | 5.56E−07 |
| Gm28731 | 120.2 | −1.13 | −1.23 | 6.72E−08 | 7.06E−07 |
| Trim36 | 327.4 | −1.13 | −1.23 | 7.28E−08 | 7.61E−07 |
| Smagp | 295.1 | −1.13 | −1.24 | 1.66E−07 | 1.62E−06 |
| Wdfy4 | 2179.7 | −1.13 | −1.39 | 9.1E−05 | 0.000486 |
| Tenm3 | 7.8 | −1.13 | −3.31 | 0.000878 | 0.003554 |
| Srms | 29.4 | −1.13 | −1.59 | 0.000938 | 0.003755 |
| Ccr10 | 27.6 | −1.13 | −1.67 | 0.001242 | 0.00481 |
| Scn3a | 17.9 | −1.13 | −1.76 | 0.001259 | 0.00487 |
| Six1 | 18.8 | −1.13 | −1.61 | 0.00127 | 0.004905 |
| Mab21l3 | 31.1 | −1.13 | −1.89 | 0.001644 | 0.006154 |
| Tcf21 | 11.3 | −1.13 | −1.75 | 0.002034 | 0.007381 |
| Gm20506 | 6.3 | −1.13 | −3.45 | 0.00239 | 0.008493 |
| Hbb-bt | 232 | −1.13 | −2.38 | 0.002847 | 0.009853 |
| Chka | 3724.8 | −1.14 | −1.18 | 0 | 7E−15 |
| Ttc28 | 2577.9 | −1.14 | −1.22 | 6.08E−10 | 9.07E−09 |
| C330013E15Rik | 164.4 | −1.14 | −1.25 | 2.81E−07 | 2.62E−06 |
| E230029C05Rik | 167.6 | −1.14 | −1.28 | 7.77E−07 | 6.65E−06 |
| Gm22596 | 38.9 | −1.14 | −1.32 | 1.85E−05 | 0.000118 |
| Gm37856 | 32.5 | −1.14 | −1.38 | 5.67E−05 | 0.00032 |
| Acot1 | 60.2 | −1.14 | −1.39 | 6.79E−05 | 0.000375 |
| Zfp811 | 50.1 | −1.14 | −1.41 | 0.000133 | 0.000678 |
| Ctnnd2 | 131.9 | −1.14 | −1.44 | 0.000139 | 0.000703 |
| 5430402O13Rik | 22.1 | −1.14 | −1.55 | 0.000666 | 0.002793 |
| Btnl4 | 51.4 | −1.14 | −1.72 | 0.000896 | 0.00361 |
| Tex26 | 22.2 | −1.14 | −1.65 | 0.000907 | 0.003649 |
| Cecr2 | 73.9 | −1.14 | −1.95 | 0.001666 | 0.00622 |
| Cd209c | 9.8 | −1.14 | −2.49 | 0.002577 | 0.009055 |
| Msi1 | 220.6 | −1.15 | −1.2 | 8.41E−13 | 1.97E−11 |
| P2ry13 | 230.8 | −1.15 | −1.26 | 1.71E−08 | 1.99E−07 |
| Sgip1 | 455.7 | −1.15 | −1.28 | 3.33E−07 | 3.08E−06 |
| C1qc | 6828 | −1.15 | −1.29 | 6.45E−07 | 5.61E−06 |
| 1700047K16Rik | 77 | −1.15 | −1.31 | 6.01E−06 | 4.28E−05 |
| Fam83e | 25.7 | −1.15 | −1.41 | 8.3E−05 | 0.000448 |
| Mxra7 | 39.3 | −1.15 | −1.43 | 0.00011 | 0.000574 |
| Cspg4 | 3.3 | −1.15 | −5.62 | 0.000117 | 0.000603 |
| Car3 | 34.9 | −1.15 | −1.84 | 0.001092 | 0.00429 |
| Ephx3 | 23.3 | −1.15 | −1.75 | 0.001523 | 0.005749 |
| Des | 17.8 | −1.15 | −1.87 | 0.00184 | 0.006771 |
| Neurl1a | 7.4 | −1.15 | −2.41 | 0.002443 | 0.008651 |
| Stox2 | 6.7 | −1.15 | −2.3 | 0.00249 | 0.008799 |
| Lepr | 5.9 | −1.15 | −2.45 | 0.002517 | 0.008882 |
| Gm45212 | 5.8 | −1.15 | −2.56 | 0.002531 | 0.008927 |
| Creg1 | 2831 | −1.16 | −1.22 | 4.72E−12 | 9.94E−11 |
| Txnrd3 | 195.6 | −1.16 | −1.25 | 7.32E−10 | 1.08E−08 |
| Cd163 | 1248.1 | −1.16 | −1.31 | 6.08E−07 | 5.33E−06 |
| Rab34 | 100.4 | −1.16 | −1.31 | 1.51E−06 | 1.23E−05 |
| Fgf17 | 23 | −1.16 | −1.55 | 0.000756 | 0.00312 |
| Prlr | 10.5 | −1.16 | −2.27 | 0.001924 | 0.007048 |
| Il1bos | 10.4 | −1.16 | −2.22 | 0.002031 | 0.007373 |
| Gm38299 | 5.4 | −1.16 | −3.44 | 0.002059 | 0.007461 |
| Gm35584 | 9 | −1.16 | −2.58 | 0.002101 | 0.007598 |
| Rasl10b | 6.3 | −1.16 | −2.53 | 0.002111 | 0.007625 |
| Il22ra2 | 7.5 | −1.16 | −2.84 | 0.002262 | 0.008085 |
| Tgfbr3 | 2708.7 | −1.17 | −1.2 | 0 | 0 |
| Bach2 | 5671.9 | −1.17 | −1.21 | 0 | 6E−15 |
| Gm43352 | 344.3 | −1.17 | −1.24 | 3.79E−11 | 6.97E−10 |
| Tnfrsf21 | 641.6 | −1.17 | −1.25 | 8.28E−11 | 1.43E−09 |
| Mpeg1 | 6302.5 | −1.17 | −1.25 | 7.72E−10 | 1.13E−08 |
| Tbxas1 | 561.4 | −1.17 | −1.27 | 7.87E−09 | 9.61E−08 |
| Thsd1 | 89.3 | −1.17 | −1.3 | 2.62E−07 | 2.46E−06 |
| Il1b | 670.8 | −1.17 | −1.31 | 7.38E−07 | 6.36E−06 |
| Tbx2 | 144.3 | −1.17 | −1.34 | 3.67E−06 | 2.74E−05 |
| March1 | 226 | −1.17 | −1.41 | 1.83E−05 | 0.000117 |
| Serpine1 | 218.8 | −1.17 | −1.42 | 5.48E−05 | 0.00031 |
| Gm37509 | 25.6 | −1.17 | −1.44 | 6.32E−05 | 0.000352 |
| Ptgis | 82.5 | −1.17 | −1.47 | 9.03E−05 | 0.000483 |
| Crnde | 21.9 | −1.17 | −1.47 | 0.000116 | 0.000599 |
| Gm15448 | 29.6 | −1.17 | −1.71 | 0.000537 | 0.002315 |
| Mir5107 | 156.7 | −1.17 | −1.83 | 0.001041 | 0.004111 |
| 1700001J03Rik | 13.9 | −1.17 | −2.01 | 0.001786 | 0.006611 |
| Gm17999 | 9.8 | −1.17 | −2.54 | 0.0018 | 0.006652 |
| Gm37621 | 8.8 | −1.17 | −2.16 | 0.001873 | 0.006882 |
| Myzap | 9.4 | −1.17 | −2.33 | 0.001875 | 0.006886 |
| Gm15156 | 8.3 | −1.17 | −2.23 | 0.001925 | 0.00705 |
| 4930455G09Rik | 13.9 | −1.17 | −2.58 | 0.002094 | 0.007572 |
| Mtss1 | 2502.5 | −1.18 | −1.19 | 0 | 0 |
| 1700056E22Rik | 810.3 | −1.18 | −1.24 | 3E−14 | 8.31E−13 |
| Plekha6 | 574.9 | −1.18 | −1.24 | 3.19E−13 | 7.72E−12 |
| Gnal | 314.2 | −1.18 | −1.26 | 4.93E−10 | 7.47E−09 |
| Wnk4 | 72.2 | −1.18 | −1.29 | 1.22E−08 | 1.45E−07 |
| Clec12a | 552.3 | −1.18 | −1.35 | 2.93E−06 | 2.25E−05 |
| 9330102E08Rik | 20.4 | −1.18 | −1.59 | 0.000312 | 0.001441 |
| Adgrl3 | 33.2 | −1.18 | −1.74 | 0.00049 | 0.002135 |
| AC152065.1 | 24.8 | −1.18 | −1.71 | 0.000632 | 0.002673 |
| Gm26685 | 15.5 | −1.18 | −1.8 | 0.000915 | 0.003677 |
| Gm5466 | 17.5 | −1.18 | −1.99 | 0.001304 | 0.005023 |
| Reln | 18.1 | −1.18 | −2.28 | 0.001614 | 0.006051 |
| Gucy2c | 7.2 | −1.18 | −2.88 | 0.001714 | 0.006382 |
| Gm45572 | 4.9 | −1.18 | −3.42 | 0.001752 | 0.0065 |
| Gm2058 | 5.4 | −1.18 | −2.64 | 0.001753 | 0.006503 |
| Fcnaos | 7.6 | −1.18 | −2.37 | 0.001762 | 0.006532 |
| Gm13868 | 10 | −1.18 | −2.61 | 0.001817 | 0.006701 |
| Ldlr | 1329.5 | −1.19 | −1.23 | 0 | 1E−15 |
| S1pr1 | 21169 | −1.19 | −1.24 | 3.9E−14 | 1.05E−12 |
| Ksr2 | 333.8 | −1.19 | −1.27 | 7.47E−11 | 1.3E−09 |
| Lrrc75b | 342.7 | −1.19 | −1.3 | 5.18E−09 | 6.59E−08 |
| Dnah17 | 303 | −1.19 | −1.3 | 4.01E−08 | 4.37E−07 |
| Tmem141 | 255.2 | −1.19 | −1.37 | 1.75E−06 | 1.4E−05 |
| Gpat3 | 224.7 | −1.19 | −1.35 | 1.96E−06 | 1.55E−05 |
| Adhfe1 | 58 | −1.19 | −1.4 | 5.68E−06 | 4.06E−05 |
| Poln | 40.7 | −1.19 | −1.45 | 5.15E−05 | 0.000294 |
| Klra17 | 46.4 | −1.19 | −1.57 | 0.000193 | 0.000938 |
| Syce1l | 12.2 | −1.19 | −1.87 | 0.000875 | 0.003544 |
| Gm15523 | 17.3 | −1.19 | −1.93 | 0.001035 | 0.00409 |
| 0610040J01Rik | 15.3 | −1.19 | −2.08 | 0.001054 | 0.004156 |
| Gm11525 | 16.1 | −1.19 | −2.03 | 0.001213 | 0.004715 |
| 6330403L08Rik | 20.6 | −1.19 | −2.07 | 0.001227 | 0.00476 |
| Gm15848 | 16.2 | −1.19 | −3.07 | 0.001697 | 0.006322 |
| Gm26799 | 792.8 | −1.2 | −1.26 | 7.8E−14 | 2.04E−12 |
| Nr1d2 | 2875.9 | −1.2 | −1.26 | 3.15E−13 | 7.65E−12 |
| Tcf4 | 708.9 | −1.2 | −1.3 | 6.96E−09 | 8.59E−08 |
| Cd300a | 1109.4 | −1.2 | −1.33 | 5.7E−08 | 6.03E−07 |
| Ccnd1 | 258 | −1.2 | −1.35 | 1.29E−07 | 1.29E−06 |
| Gm12474 | 77.6 | −1.2 | −1.34 | 2.3E−07 | 2.18E−06 |
| Kcnj9 | 35.1 | −1.2 | −1.78 | 0.000455 | 0.002001 |
| Baiap2l1 | 10.8 | −1.2 | −2.01 | 0.001135 | 0.00444 |
| Slc6a1 | 17.6 | −1.2 | −2.23 | 0.001191 | 0.004634 |
| Gpr152 | 13.5 | −1.2 | −2.12 | 0.001386 | 0.0053 |
| Gm44735 | 8.1 | −1.2 | −2.18 | 0.001412 | 0.005383 |
| Gm15675 | 565.2 | −1.21 | −1.28 | 2.06E−13 | 5.1E−12 |
| Plxna1 | 900.2 | −1.21 | −1.3 | 3.45E−10 | 5.38E−09 |
| Slc7a7 | 700.4 | −1.21 | −1.31 | 1.07E−09 | 1.52E−08 |
| P4ha2 | 71.8 | −1.21 | −1.35 | 9.41E−08 | 9.65E−07 |
| Dbndd2 | 61.2 | −1.21 | −1.35 | 1.45E−07 | 1.43E−06 |
| Angptl7 | 118.5 | −1.21 | −1.38 | 5.17E−07 | 4.61E−06 |
| Sirpb1a | 105.6 | −1.21 | −1.39 | 5.82E−07 | 5.13E−06 |
| Kcnk13 | 70 | −1.21 | −1.41 | 2.14E−06 | 1.68E−05 |
| Gm2238 | 53.2 | −1.21 | −1.43 | 9.18E−06 | 6.28E−05 |
| AC139941.2 | 39.3 | −1.21 | −1.54 | 8.74E−05 | 0.000469 |
| Plekhg1 | 29 | −1.21 | −1.66 | 0.000292 | 0.001357 |
| AC154548.2 | 22 | −1.21 | −1.72 | 0.000515 | 0.002231 |
| Gm36937 | 12.9 | −1.21 | −2.31 | 0.001265 | 0.004889 |
| Gm36159 | 8.5 | −1.21 | −2.36 | 0.001348 | 0.005169 |
| Rgs7bp | 8.5 | −1.21 | −2.61 | 0.001437 | 0.005464 |
| Amigo2 | 1152.3 | −1.22 | −1.27 | 1E−15 | 3.2E−14 |
| Pde5a | 641 | −1.22 | −1.29 | 4E−14 | 1.08E−12 |
| Myo9a | 1226.4 | −1.22 | −1.29 | 1.92E−13 | 4.77E−12 |
| Ptpdc1 | 185.8 | −1.22 | −1.3 | 3.71E−12 | 7.94E−11 |
| Rab7b | 324.8 | −1.22 | −1.32 | 6.47E−10 | 9.59E−09 |
| Ptgs1 | 1227.5 | −1.22 | −1.31 | 6.72E−10 | 9.94E−09 |
| Siglech | 840.4 | −1.22 | −1.36 | 9.21E−08 | 9.46E−07 |
| Gm13340 | 40.7 | −1.22 | −1.42 | 3.06E−06 | 2.34E−05 |
| Slpi | 1443.3 | −1.22 | −1.51 | 3.7E−05 | 0.00022 |
| Crabp2 | 41.6 | −1.22 | −1.55 | 0.000115 | 0.000594 |
| Cpne9 | 20.6 | −1.22 | −1.59 | 0.000175 | 0.000861 |
| Wtip | 28.7 | −1.22 | −1.67 | 0.00019 | 0.000927 |
| Nox1 | 20 | −1.22 | −1.77 | 0.000288 | 0.00134 |
| Kcnj2 | 25 | −1.22 | −2.13 | 0.000891 | 0.003595 |
| Gm38160 | 15.5 | −1.22 | −2.33 | 0.001201 | 0.004671 |
| Tulp1 | 4.4 | −1.22 | −2.59 | 0.001242 | 0.00481 |
| Tbx4 | 5.5 | −1.22 | −2.3 | 0.001274 | 0.004917 |
| Rapgef4 | 2629.2 | −1.23 | −1.28 | 0 | 1E−15 |
| Zfyve28 | 1185 | −1.23 | −1.32 | 2.46E−11 | 4.68E−10 |
| Tns3 | 884 | −1.23 | −1.34 | 1.44E−09 | 2.01E−08 |
| Alpk3 | 97.8 | −1.23 | −1.35 | 3.57E−09 | 4.69E−08 |
| Ucp3 | 43.4 | −1.23 | −1.42 | 1.96E−06 | 1.55E−05 |
| Slc26a8 | 47 | −1.23 | −1.43 | 3.29E−06 | 2.48E−05 |
| Slc16a7 | 76.5 | −1.23 | −1.46 | 6.11E−06 | 4.34E−05 |
| Ssc5d | 41.7 | −1.23 | −1.5 | 2.14E−05 | 0.000134 |
| AC159649.1 | 24.5 | −1.23 | −1.54 | 6.02E−05 | 0.000337 |
| Clec9a | 155.9 | −1.23 | −1.61 | 8.74E−05 | 0.00047 |
| Has3 | 31.1 | −1.23 | −1.68 | 0.000212 | 0.001023 |
| Muc2 | 15.7 | −1.23 | −1.78 | 0.000371 | 0.001676 |
| Lsamp | 11.3 | −1.23 | −1.86 | 0.000524 | 0.002268 |
| Gm44185 | 16.5 | −1.23 | −1.85 | 0.000526 | 0.002273 |
| Gm12263 | 11.8 | −1.23 | −2.06 | 0.000838 | 0.003411 |
| Adam11 | 386.6 | −1.24 | −1.3 | 0 | 1.4E−14 |
| 9230114K14Rik | 64.2 | −1.24 | −1.43 | 1.77E−06 | 1.42E−05 |
| AC158605.2 | 23.8 | −1.24 | −1.72 | 0.000182 | 0.000892 |
| Ccser1 | 25.6 | −1.24 | −1.92 | 0.000376 | 0.001694 |
| Kncn | 8.8 | −1.24 | −1.98 | 0.000829 | 0.00338 |
| 5930403N24Rik | 16 | −1.24 | −2.23 | 0.000843 | 0.003427 |
| Sbsn | 107.9 | −1.25 | −1.36 | 1.8E−09 | 2.47E−08 |
| Adgrl2 | 148.4 | −1.25 | −1.38 | 1.1E−08 | 1.31E−07 |
| Slc16a9 | 181.6 | −1.25 | −1.43 | 4.53E−07 | 4.09E−06 |
| Gm17116 | 29.3 | −1.25 | −1.58 | 3.4E−05 | 0.000203 |
| Cxcl2 | 1196 | −1.25 | −1.58 | 4.79E−05 | 0.000276 |
| Tpm2 | 26.7 | −1.25 | −1.65 | 9.83E−05 | 0.00052 |
| AC158622.3 | 22.2 | −1.25 | −1.68 | 0.00016 | 0.0008 |
| Gm37755 | 11.4 | −1.25 | −2.1 | 0.000797 | 0.003266 |
| Tmem132b | 9.3 | −1.25 | −3.04 | 0.001003 | 0.003981 |
| Gtf2ird1 | 693.3 | −1.26 | −1.37 | 1.66E−10 | 2.76E−09 |
| Rhbdf1 | 115.9 | −1.26 | −1.4 | 2.97E−08 | 3.32E−07 |
| Slc23a3 | 71.8 | −1.26 | −1.41 | 1.12E−07 | 1.13E−06 |
| Spon2 | 49.3 | −1.26 | −1.42 | 1.54E−07 | 1.51E−06 |
| Gm11342 | 46.6 | −1.26 | −1.46 | 9.11E−07 | 7.69E−06 |
| Gm15728 | 46.1 | −1.26 | −1.49 | 4.42E−06 | 3.24E−05 |
| Skor1 | 38.4 | −1.26 | −1.48 | 5.95E−06 | 4.25E−05 |
| Olfr1033 | 26.2 | −1.26 | −1.71 | 0.000174 | 0.000858 |
| Gm15327 | 25.1 | −1.26 | −1.81 | 0.000356 | 0.001619 |
| Gm32999 | 14.5 | −1.26 | −2.13 | 0.000663 | 0.002785 |
| 4833411C07Rik | 12.7 | −1.26 | −2.47 | 0.000791 | 0.003247 |
| Gm12290 | 7.9 | −1.26 | −2.93 | 0.000865 | 0.003511 |
| Slc28a1 | 7.4 | −1.26 | −2.34 | 0.000891 | 0.003595 |
| Cd55 | 2669.5 | −1.27 | −1.32 | 0 | 0 |
| Adra2a | 136.7 | −1.27 | −1.34 | 4.7E−14 | 1.26E−12 |
| Dnhd1 | 272.9 | −1.27 | −1.35 | 6.78E−13 | 1.6E−11 |
| Pirb | 1339.8 | −1.27 | −1.41 | 1.54E−08 | 1.8E−07 |
| Cd300lf | 581.8 | −1.27 | −1.41 | 1.64E−08 | 1.92E−07 |
| Zfhx3 | 113.3 | −1.27 | −1.44 | 1.52E−07 | 1.49E−06 |
| Amn | 69.8 | −1.27 | −1.5 | 2.81E−06 | 2.16E−05 |
| Gm44401 | 34.9 | −1.27 | −1.51 | 3.5E−06 | 2.62E−05 |
| Cecr6 | 48.6 | −1.27 | −1.52 | 4.28E−06 | 3.15E−05 |
| Sirpb1b | 32.1 | −1.27 | −1.69 | 2.66E−05 | 0.000163 |
| Zfp618 | 38 | −1.27 | −1.64 | 4.75E−05 | 0.000274 |
| Apoc1 | 69.6 | −1.27 | −1.69 | 7.23E−05 | 0.000397 |
| Ltbp2 | 21 | −1.27 | −1.83 | 0.000197 | 0.000955 |
| Gm17455 | 30.9 | −1.27 | −1.88 | 0.000295 | 0.001371 |
| St18 | 3.6 | −1.27 | −4.89 | 0.000421 | 0.001874 |
| Gpbar1 | 9.8 | −1.27 | −2.27 | 0.000836 | 0.003403 |
| Ptk2 | 617.3 | −1.28 | −1.32 | 0 | 0 |
| Sbf2 | 450.1 | −1.28 | −1.33 | 0 | 0 |
| AC135019.1 | 300.9 | −1.28 | −1.34 | 0 | 1E−15 |
| Ctsf | 383.8 | −1.28 | −1.37 | 4.71E−12 | 9.94E−11 |
| Npm2 | 80.8 | −1.28 | −1.37 | 9.51E−11 | 1.63E−09 |
| Rin1 | 193.4 | −1.28 | −1.38 | 5.16E−10 | 7.79E−09 |
| Adgre1 | 1926.6 | −1.28 | −1.4 | 8.52E−10 | 1.24E−08 |
| Hpgd | 485.4 | −1.28 | −1.4 | 2.16E−09 | 2.93E−08 |
| Gm18407 | 52.8 | −1.28 | −1.47 | 3.97E−07 | 3.62E−06 |
| AC164573.1 | 54.6 | −1.28 | −1.47 | 7.45E−07 | 6.4E−06 |
| Arg2 | 120.5 | −1.28 | −1.49 | 7.92E−07 | 6.76E−06 |
| Flt3 | 441.7 | −1.28 | −1.5 | 8.85E−07 | 7.48E−06 |
| Gm26615 | 44.6 | −1.28 | −1.65 | 2.71E−05 | 0.000166 |
| A530064D06Rik | 30 | −1.28 | −1.72 | 3.42E−05 | 0.000204 |
| Gm33280 | 43.4 | −1.28 | −1.76 | 9.08E−05 | 0.000485 |
| Gm15930 | 22 | −1.28 | −1.93 | 0.000219 | 0.001054 |
| Hoxaas3 | 15.8 | −1.28 | −1.9 | 0.000251 | 0.001189 |
| Gm11980 | 17.1 | −1.28 | −1.92 | 0.000307 | 0.001421 |
| Gm43857 | 14.2 | −1.28 | −2.08 | 0.000361 | 0.001637 |
| Gm37510 | 31.1 | −1.28 | −1.87 | 0.000376 | 0.001697 |
| Gm12972 | 20.1 | −1.28 | −2.06 | 0.000381 | 0.001713 |
| Cyp26b1 | 13.2 | −1.28 | −1.96 | 0.000381 | 0.001714 |
| P4ha3 | 10.9 | −1.28 | −1.95 | 0.000471 | 0.002065 |
| Prex2 | 8.7 | −1.28 | −2.59 | 0.000692 | 0.00289 |
| Usp2 | 832.7 | −1.29 | −1.39 | 4.34E−11 | 7.86E−10 |
| Tlr2 | 527.6 | −1.29 | −1.46 | 7.25E−08 | 7.59E−07 |
| Matn2 | 73.1 | −1.29 | −1.57 | 2.24E−06 | 1.75E−05 |
| AC159196.3 | 73.6 | −1.29 | −1.53 | 2.47E−06 | 1.92E−05 |
| Fam135a | 30.1 | −1.29 | −1.79 | 7.1E−05 | 0.000391 |
| Gm4247 | 22.2 | −1.29 | −1.72 | 7.22E−05 | 0.000397 |
| Fam83c | 5.2 | −1.29 | −3.81 | 0.000414 | 0.001843 |
| Otud1 | 1927.6 | −1.3 | −1.33 | 0 | 0 |
| Fam209 | 161.5 | −1.3 | −1.41 | 5.91E−10 | 8.83E−09 |
| Spred3 | 56.8 | −1.3 | −1.44 | 8.54E−09 | 1.04E−07 |
| Lman1l | 148.2 | −1.3 | −1.46 | 2.39E−08 | 2.7E−07 |
| C1qa | 7016.9 | −1.3 | −1.46 | 2.44E−08 | 2.75E−07 |
| Msantd1 | 67.1 | −1.3 | −1.47 | 1.68E−07 | 1.64E−06 |
| Nlgn3 | 46.3 | −1.3 | −1.5 | 3.68E−07 | 3.38E−06 |
| Samd4 | 57.2 | −1.3 | −1.64 | 1.07E−05 | 7.19E−05 |
| Cyp2d40 | 3.9 | −1.3 | −3.38 | 0.000438 | 0.001938 |
| Usp28 | 3778 | −1.31 | −1.34 | 0 | 0 |
| Ntn4 | 238.3 | −1.31 | −1.42 | 1.89E−10 | 3.13E−09 |
| Gm19325 | 31.3 | −1.31 | −1.59 | 6.63E−06 | 4.67E−05 |
| Ccdc148 | 86.4 | −1.31 | −1.62 | 6.67E−06 | 4.7E−05 |
| Matn1 | 29.8 | −1.31 | −1.6 | 7.41E−06 | 5.18E−05 |
| Art5 | 20.4 | −1.31 | −1.74 | 4.51E−05 | 0.000261 |
| Slc6a7 | 16 | −1.31 | −1.9 | 0.000175 | 0.000862 |
| Paqr6 | 11.7 | −1.31 | −2.35 | 0.000444 | 0.001958 |
| 6430548M08Rik | 386.7 | −1.32 | −1.41 | 4.35E−13 | 1.04E−11 |
| Jup | 1184.5 | −1.32 | −1.41 | 4.36E−13 | 1.04E−11 |
| Lima1 | 405.9 | −1.32 | −1.43 | 4.11E−11 | 7.48E−10 |
| Tgm2 | 2908.8 | −1.32 | −1.44 | 1.38E−10 | 2.32E−09 |
| Cfp | 5197 | −1.32 | −1.44 | 4.73E−10 | 7.18E−09 |
| Igf1 | 580.6 | −1.32 | −1.5 | 3.7E−08 | 4.07E−07 |
| Gm44053 | 47.1 | −1.32 | −1.6 | 1.61E−06 | 1.3E−05 |
| Gpr4 | 64.4 | −1.32 | −1.62 | 4.44E−06 | 3.25E−05 |
| Mc1r | 8.3 | −1.32 | −2.63 | 0.000507 | 0.002201 |
| Ampd1 | 414.5 | −1.33 | −1.39 | 0 | 0 |
| C6 | 1221.3 | −1.33 | −1.46 | 3.07E−10 | 4.85E−09 |
| Gfra2 | 1060.6 | −1.33 | −1.46 | 5.81E−10 | 8.69E−09 |
| Clec4n | 902.8 | −1.33 | −1.49 | 4.25E−09 | 5.48E−08 |
| Krt83 | 243.6 | −1.33 | −1.49 | 2.68E−08 | 3.01E−07 |
| Clec4b1 | 78.8 | −1.33 | −1.72 | 1.43E−05 | 9.36E−05 |
| Clec4a4 | 10.7 | −1.33 | −2.9 | 0.000393 | 0.001763 |
| Nr2e3 | 6.8 | −1.33 | −4.34 | 0.000443 | 0.001955 |
| Irs2 | 11401.3 | −1.34 | −1.4 | 0 | 1E−15 |
| Fbxl22 | 362.3 | −1.34 | −1.41 | 6E−15 | 1.88E−13 |
| Csf1r | 9890.8 | −1.34 | −1.45 | 2.94E−12 | 6.38E−11 |
| Zfp608 | 304 | −1.34 | −1.47 | 7.59E−10 | 1.11E−08 |
| Gm42636 | 106.9 | −1.34 | −1.48 | 1.27E−09 | 1.79E−08 |
| Fgd4 | 96.4 | −1.34 | −1.66 | 3.01E−06 | 2.3E−05 |
| Gm9530 | 13.7 | −1.34 | −2.18 | 0.00018 | 0.000884 |
| Gli1 | 13 | −1.34 | −2.06 | 0.00019 | 0.000928 |
| Epha4 | 19.9 | −1.34 | −2.3 | 0.000222 | 0.001066 |
| Tmem132c | 5.6 | −1.34 | −3.55 | 0.000311 | 0.001435 |
| Vipr1 | 6553.5 | −1.35 | −1.4 | 0 | 0 |
| Adamdec1 | 659.5 | −1.35 | −1.47 | 7.08E−11 | 1.24E−09 |
| Fmnl2 | 329.1 | −1.35 | −1.5 | 1.5E−09 | 2.08E−08 |
| Guca2a | 39.9 | −1.35 | −1.58 | 4.21E−07 | 3.82E−06 |
| Tgm4 | 40.9 | −1.35 | −1.6 | 7.48E−07 | 6.42E−06 |
| Gm12259 | 65.1 | −1.35 | −1.67 | 4.31E−06 | 3.17E−05 |
| Trf | 681.7 | −1.35 | −1.77 | 1.94E−05 | 0.000123 |
| Podn | 23.2 | −1.35 | −1.75 | 2.38E−05 | 0.000148 |
| Aldh6a1 | 522.8 | −1.36 | −1.47 | 2.47E−11 | 4.68E−10 |
| Adh1 | 149.8 | −1.36 | −1.49 | 4.27E−10 | 6.55E−09 |
| Gm20544 | 121 | −1.36 | −1.51 | 9.16E−10 | 1.32E−08 |
| Lpcat2 | 297.8 | −1.36 | −1.51 | 1.63E−09 | 2.25E−08 |
| Rufy4 | 34.6 | −1.36 | −1.67 | 2.98E−06 | 2.28E−05 |
| Syt1 | 3.7 | −1.36 | −5.79 | 0.000188 | 0.000918 |
| Ankrd63 | 174.2 | −1.37 | −1.48 | 8.29E−12 | 1.7E−10 |
| Slco2b1 | 818.9 | −1.37 | −1.49 | 9.09E−12 | 1.84E−10 |
| Gm44907 | 211 | −1.37 | −1.48 | 2.4E−11 | 4.58E−10 |
| Gm26888 | 163 | −1.37 | −1.5 | 8.25E−11 | 1.43E−09 |
| Bcl2a1a | 59.4 | −1.37 | −1.77 | 7.74E−06 | 5.39E−05 |
| AC153955.5 | 18.5 | −1.37 | −2.1 | 9.26E−05 | 0.000494 |
| Lrrc3 | 23.5 | −1.37 | −2.17 | 0.000121 | 0.000625 |
| Armcx4 | 774.4 | −1.37 | −2.13 | 0.000162 | 0.000806 |
| Gm12689 | 10.9 | −1.37 | −2.37 | 0.000236 | 0.001124 |
| Slc40a1 | 4625.9 | −1.38 | −1.49 | 3.67E−12 | 7.88E−11 |
| Tmem8b | 139.4 | −1.38 | −1.5 | 8.99E−12 | 1.83E−10 |
| Gm14027 | 93.7 | −1.38 | −1.5 | 3.48E−11 | 6.44E−10 |
| Gm15503 | 167.6 | −1.38 | −1.51 | 8.32E−11 | 1.44E−09 |
| Cd302 | 387 | −1.38 | −1.53 | 1.75E−10 | 2.9E−09 |
| Mafb | 1846.3 | −1.38 | −1.52 | 2.27E−10 | 3.69E−09 |
| Gm15931 | 220.2 | −1.38 | −1.57 | 6.29E−09 | 7.84E−08 |
| Apol9b | 51.1 | −1.38 | −1.56 | 1.78E−08 | 2.06E−07 |
| Pid1 | 185.8 | −1.38 | −1.68 | 8.53E−07 | 7.24E−06 |
| Hes1 | 309.1 | −1.38 | −1.68 | 1.9E−06 | 1.51E−05 |
| Jag1 | 135.9 | −1.38 | −1.7 | 4.46E−06 | 3.26E−05 |
| A530099J19Rik | 38 | −1.38 | −2.21 | 9.87E−05 | 0.000522 |
| 4930471C04Rik | 10.8 | −1.38 | −2.24 | 0.000147 | 0.000741 |
| Slc13a2 | 13.1 | −1.38 | −2.43 | 0.000153 | 0.000766 |
| Gm15880 | 16.2 | −1.38 | −2.53 | 0.000207 | 0.000999 |
| H2-Ob | 1767 | −1.39 | −1.47 | 2E−15 | 6.2E−14 |
| St8sia6 | 1584.6 | −1.39 | −1.47 | 4E−15 | 1.36E−13 |
| Kcnip2 | 299.8 | −1.39 | −1.49 | 9.54E−13 | 2.22E−11 |
| Agap1 | 495.9 | −1.39 | −1.54 | 3.02E−10 | 4.8E−09 |
| Cyp27a1 | 464.9 | −1.39 | −1.57 | 5.08E−09 | 6.47E−08 |
| Glis3 | 183.7 | −1.39 | −1.6 | 3.44E−08 | 3.81E−07 |
| Ttc36 | 55 | −1.39 | −1.62 | 1.95E−07 | 1.88E−06 |
| S1pr3 | 58.4 | −1.39 | −1.66 | 2.67E−07 | 2.5E−06 |
| Shisa4 | 42.4 | −1.39 | −1.69 | 1.93E−06 | 1.53E−05 |
| Notch4 | 137.5 | −1.39 | −1.8 | 8.56E−06 | 5.9E−05 |
| Unc5a | 20.3 | −1.39 | −2.82 | 0.000189 | 0.000922 |
| Gm43364 | 7 | −1.39 | −2.54 | 0.000253 | 0.001196 |
| Myo10 | 1090.4 | −1.4 | −1.49 | 8E−15 | 2.44E−13 |
| Ifi207 | 440.3 | −1.4 | −1.55 | 2.58E−10 | 4.15E−09 |
| Ppfibp2 | 358.3 | −1.4 | −1.57 | 9.23E−10 | 1.33E−08 |
| Kcnh4 | 54 | −1.4 | −1.8 | 8.38E−06 | 5.79E−05 |
| Ppp1r14a | 48.4 | −1.4 | −1.89 | 1.95E−05 | 0.000124 |
| AC160403.2 | 22.2 | −1.4 | −1.93 | 2.84E−05 | 0.000173 |
| Gm37900 | 17.4 | −1.4 | −1.95 | 3.9E−05 | 0.00023 |
| Tal2 | 7.8 | −1.4 | −3.32 | 0.000204 | 0.000985 |
| Gper1 | 10.3 | −1.4 | −2.81 | 0.000226 | 0.001083 |
| Gm45053 | 497.4 | −1.41 | −1.46 | 0 | 0 |
| Erp27 | 298.4 | −1.41 | −1.48 | 0 | 0 |
| Rims3 | 1029.1 | −1.41 | −1.5 | 2E−15 | 5.8E−14 |
| Epha2 | 215.5 | −1.41 | −1.55 | 4.1E−11 | 7.48E−10 |
| Dll4 | 78 | −1.41 | −1.6 | 4.69E−09 | 6E−08 |
| Cacna1e | 284.9 | −1.41 | −1.69 | 2.23E−07 | 2.12E−06 |
| AC159502.1 | 24.7 | −1.41 | −1.84 | 1.34E−05 | 8.79E−05 |
| Gm14221 | 27.8 | −1.41 | −2.18 | 5.98E−05 | 0.000335 |
| Gm24371 | 8.6 | −1.41 | −2.69 | 0.000193 | 0.000938 |
| Haglr | 8.2 | −1.41 | −2.62 | 0.000212 | 0.001022 |
| Tnfrsf10b | 1091.3 | −1.42 | −1.48 | 0 | 0 |
| Lhfpl3 | 194 | −1.42 | −1.5 | 0 | 1E−15 |
| Ppp1r32 | 136.8 | −1.42 | −1.54 | 1E−12 | 2.32E−11 |
| Cystm1 | 143.7 | −1.42 | −1.62 | 6.84E−09 | 8.46E−08 |
| Bank1 | 715.4 | −1.42 | −1.65 | 3.75E−08 | 4.12E−07 |
| Trim47 | 215.2 | −1.42 | −1.64 | 3.98E−08 | 4.35E−07 |
| Gm14548 | 229.3 | −1.42 | −1.68 | 5.07E−08 | 5.42E−07 |
| A930036K24Rik | 102.7 | −1.42 | −1.69 | 4.41E−07 | 4E−06 |
| Bmf | 141.4 | −1.42 | −1.87 | 8.04E−06 | 5.59E−05 |
| Tnnt3 | 267.5 | −1.43 | −1.49 | 0 | 0 |
| Abcg3 | 1117.1 | −1.43 | −1.52 | 0 | 3E−15 |
| Lilra6 | 252.1 | −1.43 | −1.59 | 5.27E−11 | 9.37E−10 |
| Kirrel3 | 96.4 | −1.43 | −1.63 | 2.29E−09 | 3.09E−08 |
| Nlrp3 | 1148.6 | −1.43 | −1.64 | 6.8E−09 | 8.42E−08 |
| Tspan15 | 134 | −1.43 | −1.63 | 6.99E−09 | 8.63E−08 |
| Pdzd2 | 59.3 | −1.43 | −1.68 | 4.54E−08 | 4.89E−07 |
| Adap2 | 260.1 | −1.43 | −1.7 | 7.96E−08 | 8.26E−07 |
| E230013L22Rik | 56 | −1.43 | −1.81 | 1.9E−06 | 1.5E−05 |
| Kcnj10 | 1374.3 | −1.44 | −1.55 | 1.4E−14 | 4.01E−13 |
| Plbd1 | 1881.3 | −1.44 | −1.58 | 7.79E−12 | 1.6E−10 |
| Nphp3 | 103.3 | −1.44 | −1.58 | 3.52E−11 | 6.5E−10 |
| Trim72 | 99 | −1.44 | −1.79 | 3.19E−06 | 2.42E−05 |
| 9330179D12Rik | 27.2 | −1.44 | −1.85 | 4E−06 | 2.96E−05 |
| Gm44889 | 45.5 | −1.44 | −1.82 | 4.43E−06 | 3.25E−05 |
| Mcc | 21.2 | −1.44 | −2.36 | 4.81E−05 | 0.000277 |
| Gm14137 | 13.8 | −1.44 | −3.59 | 0.000153 | 0.000766 |
| Ifngr2 | 7439.3 | −1.45 | −1.52 | 0 | 0 |
| Ripor3 | 228.4 | −1.45 | −1.53 | 0 | 0 |
| Atp2a1 | 251.9 | −1.45 | −1.54 | 0 | 1.7E−14 |
| Lrrc23 | 180.5 | −1.45 | −1.54 | 1E−15 | 2.6E−14 |
| Map3k9 | 162.8 | −1.45 | −1.56 | 9E−15 | 2.57E−13 |
| Ccr3 | 902.9 | −1.45 | −1.57 | 1.22E−12 | 2.78E−11 |
| Gm13919 | 14.4 | −1.45 | −2.29 | 6.69E−05 | 0.00037 |
| Gm43263 | 4.5 | −1.45 | −4.23 | 0.000109 | 0.00057 |
| Gm16378 | 7.4 | −1.45 | −3.04 | 0.00012 | 0.000616 |
| Hpgds | 565.3 | −1.46 | −1.58 | 3.5E−14 | 9.57E−13 |
| Grk3 | 1230.7 | −1.46 | −1.58 | 6.3E−14 | 1.66E−12 |
| Itgb5 | 955.2 | −1.46 | −1.58 | 2.04E−13 | 5.07E−12 |
| Slc45a3 | 1350.4 | −1.46 | −1.61 | 1.37E−11 | 2.69E−10 |
| St6galnac2 | 280.3 | −1.46 | −1.66 | 1.57E−09 | 2.17E−08 |
| B230398E01Rik | 42.2 | −1.46 | −1.79 | 5.23E−07 | 4.65E−06 |
| Tm4sf1 | 20.6 | −1.46 | −2.07 | 3.08E−05 | 0.000186 |
| Elfn2 | 10.1 | −1.46 | −2.85 | 0.000117 | 0.000607 |
| 4930426D05Rik | 77.3 | −1.46 | −3.31 | 0.000118 | 0.000608 |
| Neurl3 | 9998.4 | −1.47 | −1.51 | 0 | 0 |
| Gas6 | 210.8 | −1.47 | −1.69 | 7.28E−09 | 8.96E−08 |
| Arhgap32 | 163.9 | −1.47 | −1.72 | 1.72E−08 | 2E−07 |
| Slc8a1 | 117.6 | −1.47 | −1.87 | 8.73E−07 | 7.39E−06 |
| Riiad1 | 6.9 | −1.47 | −3.36 | 0.000112 | 0.000581 |
| Gm5150 | 271.8 | −1.48 | −1.61 | 1.69E−13 | 4.24E−12 |
| Ctsk | 47.3 | −1.48 | −1.67 | 1.41E−09 | 1.96E−08 |
| Vwa3b | 95.8 | −1.48 | −1.69 | 2.72E−09 | 3.64E−08 |
| Gm42778 | 66.3 | −1.48 | −1.69 | 2.76E−09 | 3.68E−08 |
| Apol9a | 29.5 | −1.48 | −1.72 | 3.39E−08 | 3.76E−07 |
| Capn9 | 80.5 | −1.48 | −1.77 | 3.53E−08 | 3.9E−07 |
| Cd164l2 | 60 | −1.48 | −1.78 | 2.67E−07 | 2.5E−06 |
| Jhy | 47.7 | −1.48 | −2.71 | 6.28E−05 | 0.00035 |
| Gm26583 | 155.4 | −1.49 | −1.61 | 1E−13 | 2.59E−12 |
| Axl | 7411.5 | −1.5 | −1.62 | 4.7E−14 | 1.27E−12 |
| Cd209b | 9.6 | −1.5 | −3.41 | 7.28E−05 | 0.0004 |
| Mpzl1 | 469.8 | −1.51 | −1.65 | 1.12E−12 | 2.58E−11 |
| Stra6l | 77.8 | −1.51 | −1.78 | 3.26E−09 | 4.3E−08 |
| Slc1a3 | 115.6 | −1.51 | −1.87 | 2.05E−07 | 1.96E−06 |
| Plaur | 2412.8 | −1.53 | −1.62 | 0 | 0 |
| Tmem26 | 319.6 | −1.53 | −1.69 | 2.21E−12 | 4.88E−11 |
| Islr2 | 51.9 | −1.53 | −1.85 | 1.27E−07 | 1.27E−06 |
| Serinc4 | 39.2 | −1.53 | −1.89 | 2.14E−07 | 2.04E−06 |
| Gm28496 | 23.9 | −1.53 | −1.91 | 7E−07 | 6.05E−06 |
| Adam22 | 142.7 | −1.53 | −2.01 | 7.91E−07 | 6.76E−06 |
| Gm39059 | 17 | −1.53 | −2.24 | 8.57E−06 | 5.9E−05 |
| Rbm44 | 28.3 | −1.53 | −2.17 | 8.64E−06 | 5.94E−05 |
| Gm45767 | 59.3 | −1.54 | −1.71 | 2.82E−11 | 5.29E−10 |
| Rgl3 | 91.8 | −1.54 | −1.8 | 1.32E−09 | 1.86E−08 |
| Rasal2 | 84.5 | −1.54 | −1.79 | 2.62E−09 | 3.51E−08 |
| Gpc4 | 55.1 | −1.54 | −1.88 | 6.39E−08 | 6.74E−07 |
| Mybpc3 | 45.4 | −1.54 | −1.95 | 3.3E−07 | 3.06E−06 |
| Timm8a2 | 27.2 | −1.54 | −1.94 | 3.96E−07 | 3.61E−06 |
| Cryaa | 25.4 | −1.54 | −2.02 | 1.86E−06 | 1.48E−05 |
| Gm32914 | 19 | −1.54 | −2.22 | 9.67E−06 | 6.58E−05 |
| Cd207 | 65.2 | −1.54 | −3.46 | 4.96E−05 | 0.000285 |
| Kcnj16 | 221.3 | −1.55 | −1.75 | 2.52E−11 | 4.76E−10 |
| Sult6b2 | 52.2 | −1.55 | −1.74 | 3.35E−10 | 5.26E−09 |
| 9430038I01Rik | 518.8 | −1.55 | −1.75 | 3.56E−10 | 5.54E−09 |
| Dmpk | 201.4 | −1.55 | −1.77 | 3.83E−10 | 5.9E−09 |
| Ace | 93.4 | −1.55 | −2.34 | 4.87E−06 | 3.54E−05 |
| Maml3 | 448.5 | −1.56 | −1.64 | 0 | 0 |
| Vcam1 | 9147.6 | −1.56 | −1.73 | 1.93E−12 | 4.28E−11 |
| AC160405.2 | 68.7 | −1.56 | −1.74 | 2.56E−11 | 4.83E−10 |
| Marcks | 880 | −1.56 | −1.77 | 9.63E−11 | 1.65E−09 |
| Gm16486 | 59.7 | −1.56 | −1.75 | 1.02E−10 | 1.74E−09 |
| Myo18b | 126.8 | −1.56 | −1.88 | 6.78E−09 | 8.4E−08 |
| Adgrg6 | 43.3 | −1.56 | −2.1 | 9.01E−07 | 7.61E−06 |
| Kif26a | 36.7 | −1.56 | −2.4 | 9E−06 | 6.17E−05 |
| Carns1 | 4046.3 | −1.57 | −1.6 | 0 | 0 |
| Hcar2 | 808.2 | −1.57 | −1.79 | 2.26E−10 | 3.67E−09 |
| Pyroxd2 | 242.8 | −1.58 | −1.75 | 1.52E−12 | 3.44E−11 |
| Lhfp | 173.9 | −1.58 | −1.82 | 4.67E−10 | 7.1E−09 |
| Sned1 | 230.5 | −1.58 | −1.89 | 5.36E−09 | 6.79E−08 |
| Adam30 | 27.5 | −1.59 | −2.32 | 5.57E−06 | 4E−05 |
| Rasgef1b | 1868 | −1.61 | −1.68 | 0 | 0 |
| Shd | 168 | −1.61 | −1.78 | 9.04E−13 | 2.11E−11 |
| Ear2 | 200.6 | −1.61 | −1.83 | 1.1E−11 | 2.2E−10 |
| Dgki | 117.3 | −1.61 | −1.86 | 5.63E−11 | 9.99E−10 |
| Mrc1 | 3441.3 | −1.62 | −1.79 | 4.62E−13 | 1.1E−11 |
| Plpp3 | 349.2 | −1.62 | −1.82 | 3.16E−12 | 6.83E−11 |
| Armcx1 | 41.6 | −1.62 | −2.1 | 4.7E−07 | 4.23E−06 |
| AC152979.5 | 38.7 | −1.62 | −2.27 | 1.36E−06 | 1.11E−05 |
| Cfap61 | 26 | −1.62 | −2.46 | 3.27E−06 | 2.48E−05 |
| Gm44577 | 14.8 | −1.62 | −2.61 | 5.02E−06 | 3.64E−05 |
| Gm17040 | 9.2 | −1.62 | −2.56 | 9.58E−06 | 6.53E−05 |
| Paqr9 | 427.9 | −1.63 | −1.84 | 1.58E−12 | 3.57E−11 |
| Gm20692 | 32.4 | −1.63 | −2.15 | 3.32E−07 | 3.07E−06 |
| Treml4 | 1601.2 | −1.64 | −1.75 | 0 | 0 |
| Pcolce2 | 67.5 | −1.64 | −2.02 | 1.22E−08 | 1.45E−07 |
| Slc11a1 | 1813.6 | −1.65 | −1.79 | 0 | 1.3E−14 |
| 1700029H14Rik | 34.9 | −1.65 | −2 | 1.69E−08 | 1.97E−07 |
| Prrt3 | 12.9 | −1.65 | −2.43 | 3.28E−06 | 2.48E−05 |
| Hepacam2 | 34.3 | −1.65 | −3 | 7.92E−06 | 5.51E−05 |
| Ucp1 | 7.3 | −1.65 | −4.17 | 1.21E−05 | 8.04E−05 |
| Lrp4 | 160.7 | −1.66 | −1.93 | 6.9E−11 | 1.2E−09 |
| Etv1 | 13.7 | −1.66 | −2.72 | 2.68E−06 | 2.07E−05 |
| Prig | 9.9 | −1.66 | −2.49 | 5.13E−06 | 3.72E−05 |
| Zbtb10 | 1450.8 | −1.67 | −1.75 | 0 | 0 |
| Nr1d1 | 927.5 | −1.67 | −1.77 | 0 | 0 |
| Prkcg | 96.9 | −1.67 | −1.9 | 5.06E−12 | 1.06E−10 |
| Gpr137b | 186 | −1.67 | −1.92 | 8.97E−12 | 1.82E−10 |
| Tnnc2 | 16.7 | −1.67 | −2.35 | 6.15E−07 | 5.38E−06 |
| Fstl4 | 14.3 | −1.68 | −3.16 | 3.97E−06 | 2.95E−05 |
| Hfe | 584 | −1.69 | −1.89 | 1.61E−13 | 4.06E−12 |
| Prkar1b | 244.4 | −1.7 | −1.87 | 2E−15 | 6.5E−14 |
| Scnn1a | 79.5 | −1.7 | −2.03 | 3.02E−10 | 4.79E−09 |
| Fndc7 | 27.5 | −1.7 | −2.39 | 2.09E−07 | 1.99E−06 |
| Mfrp | 55.6 | −1.7 | −2.23 | 2.88E−07 | 2.68E−06 |
| Cd209d | 96.9 | −1.7 | −2.46 | 7.68E−07 | 6.58E−06 |
| Cd14 | 1110.8 | −1.71 | −1.92 | 9.3E−14 | 2.42E−12 |
| Slc15a2 | 279.3 | −1.71 | −1.97 | 5.11E−12 | 1.07E−10 |
| Gm43063 | 40.1 | −1.71 | −2.17 | 8.64E−09 | 1.05E−07 |
| Gm26575 | 11.1 | −1.71 | −3.24 | 5.02E−06 | 3.64E−05 |
| Gm8463 | 8.7 | −1.71 | −4.85 | 6.5E−06 | 4.6E−05 |
| Gm20219 | 486.3 | −1.72 | −1.84 | 0 | 0 |
| Aatk | 130.6 | −1.72 | −1.94 | 9.9E−14 | 2.57E−12 |
| Postn | 510.9 | −1.72 | −1.93 | 1.24E−13 | 3.17E−12 |
| Gm43786 | 17.6 | −1.72 | −2.36 | 5.5E−07 | 4.88E−06 |
| Amz1 | 508.4 | −1.73 | −1.88 | 0 | 1E−15 |
| Nuak1 | 190.3 | −1.73 | −1.88 | 0 | 1E−15 |
| Pmel | 62.3 | −1.73 | −4.31 | 1.27E−06 | 1.04E−05 |
| Itga9 | 882.5 | −1.74 | −1.91 | 0 | 1.1E−14 |
| Prr15 | 32.2 | −1.74 | −2.1 | 8.59E−10 | 1.25E−08 |
| AC125351.1 | 47.4 | −1.74 | −2.21 | 3.88E−09 | 5.05E−08 |
| Mmp19 | 385.3 | −1.74 | −2.31 | 5.08E−08 | 5.43E−07 |
| Prdm14 | 18.6 | −1.74 | −2.51 | 1.15E−06 | 9.57E−06 |
| Gm4258 | 54.5 | −1.75 | −1.99 | 2.33E−12 | 5.13E−11 |
| Nav1 | 825.1 | −1.76 | −1.96 | 4E−15 | 1.36E−13 |
| Nptxr | 82 | −1.76 | −2.03 | 2.98E−12 | 6.45E−11 |
| AC158622.5 | 86.8 | −1.76 | −2.1 | 2.83E−10 | 4.52E−09 |
| Gsdmc4 | 19.2 | −1.76 | −2.22 | 1.78E−08 | 2.07E−07 |
| AC153955.2 | 13.5 | −1.76 | −3.93 | 1.96E−06 | 1.55E−05 |
| Ppp1r9a | 128.9 | −1.77 | −2.16 | 2.22E−10 | 3.61E−09 |
| Gm28050 | 11.2 | −1.77 | −2.76 | 8.49E−07 | 7.21E−06 |
| Pilra | 854.5 | −1.78 | −1.94 | 0 | 0 |
| Trpm2 | 1260.6 | −1.78 | −1.96 | 0 | 6E−15 |
| Lilra5 | 286.1 | −1.78 | −2.02 | 7.9E−14 | 2.05E−12 |
| Ap3s1-ps2 | 15.1 | −1.78 | −2.77 | 7.4E−07 | 6.37E−06 |
| Slc22a23 | 202.4 | −1.79 | −1.98 | 0 | 2E−15 |
| 4930438A08Rik | 145.8 | −1.79 | −2.11 | 2.09E−10 | 3.43E−09 |
| A930030B08Rik | 34.4 | −1.79 | −2.41 | 3.65E−08 | 4.02E−07 |
| Ptgds | 5.4 | −1.79 | −5.45 | 1.85E−07 | 1.79E−06 |
| Vstm4 | 221 | −1.8 | −2.08 | 3.94E−13 | 9.46E−12 |
| Gm9889 | 639.6 | −1.81 | −1.87 | 0 | 0 |
| Pira2 | 53.5 | −1.81 | −2.39 | 3.64E−09 | 4.77E−08 |
| Six4 | 17.7 | −1.81 | −2.59 | 2.33E−07 | 2.2E−06 |
| Gm7860 | 120.6 | −1.82 | −1.97 | 0 | 0 |
| B230303O12Rik | 93.4 | −1.82 | −1.99 | 0 | 0 |
| Cadm1 | 1067.9 | −1.82 | −1.99 | 0 | 0 |
| Mertk | 1388.9 | −1.82 | −2.24 | 2.24E−10 | 3.64E−09 |
| Mmp12 | 54 | −1.82 | −2.56 | 4.76E−08 | 5.11E−07 |
| Npc1l1 | 18.3 | −1.82 | −2.72 | 2E−07 | 1.92E−06 |
| Sema6a | 113.4 | −1.83 | −2.2 | 1.34E−11 | 2.65E−10 |
| Trpv4 | 50.5 | −1.83 | −2.26 | 1.61E−10 | 2.68E−09 |
| Snx31 | 36.9 | −1.85 | −2.5 | 4.1E−09 | 5.31E−08 |
| Plch2 | 20.7 | −1.85 | −2.61 | 3.52E−08 | 3.9E−07 |
| Jag2 | 544.2 | −1.86 | −1.97 | 0 | 0 |
| Fst | 29.8 | −1.86 | −2.48 | 1.16E−08 | 1.38E−07 |
| Apol8 | 1161.9 | −1.87 | −1.95 | 0 | 0 |
| Siglece | 886.5 | −1.88 | −2.03 | 0 | 0 |
| Tbc1d9 | 512.6 | −1.88 | −2.05 | 0 | 0 |
| Tcf712 | 320.1 | −1.88 | −2.17 | 7.7E−14 | 2.03E−12 |
| Pygm | 1170.9 | −1.89 | −2 | 0 | 0 |
| Gm5608 | 44.9 | −1.89 | −2.41 | 1.16E−09 | 1.65E−08 |
| Gm38405 | 61.2 | −1.9 | −2.33 | 5.15E−11 | 9.19E−10 |
| Gm13830 | 33 | −1.9 | −2.36 | 3.07E−10 | 4.85E−09 |
| Sash1 | 878.3 | −1.91 | −2.13 | 0 | 0 |
| Dnah2 | 262.4 | −1.91 | −2.26 | 3.83E−13 | 9.2E−12 |
| Gm6377 | 147.2 | −1.91 | −2.41 | 6.81E−11 | 1.19E−09 |
| Cbln1 | 33.6 | −1.92 | −2.75 | 2.21E−09 | 3E−08 |
| Zfp819 | 24.1 | −1.93 | −2.62 | 5.98E−09 | 7.49E−08 |
| AC160028.2 | 11.6 | −1.94 | −3.91 | 2.08E−07 | 1.99E−06 |
| Tenm4 | 307.5 | −1.95 | −2.18 | 0 | 1E−15 |
| Dlc1 | 173.9 | −1.95 | −2.23 | 1E−15 | 4.2E−14 |
| Cd209a | 127.2 | −1.95 | −2.5 | 5.42E−11 | 9.63E−10 |
| Pilrb1 | 346.4 | −1.96 | −2.22 | 0 | 3E−15 |
| Aspa | 38.6 | −1.97 | −2.81 | 9.1E−10 | 1.31E−08 |
| Abcc3 | 2372 | −1.98 | −2.14 | 0 | 0 |
| Tex45 | 736.4 | −1.98 | −2.15 | 0 | 0 |
| Rnf150 | 123.1 | −1.98 | −2.22 | 0 | 0 |
| Cd300e | 272.8 | −1.98 | −2.43 | 1.21E−12 | 2.77E−11 |
| 2900052N01Rik | 111.1 | −2.01 | −2.36 | 7E−15 | 1.96E−13 |
| Lrp1 | 4560.8 | −2.04 | −2.28 | 0 | 0 |
| Gpd1 | 933.9 | −2.05 | −2.29 | 0 | 0 |
| Fam71a | 57.8 | −2.05 | −2.6 | 1.94E−11 | 3.73E−10 |
| Mybpc2 | 29.1 | −2.06 | −3.63 | 9.03E−09 | 1.09E−07 |
| Itgad | 5960.5 | −2.07 | −2.28 | 0 | 0 |
| Cxcl1 | 159.6 | −2.08 | −2.59 | 1.52E−12 | 3.44E−11 |
| Prss29 | 62.2 | −2.09 | −2.5 | 4E−15 | 1.24E−13 |
| Pilrb2 | 349.2 | −2.1 | −2.38 | 0 | 0 |
| Gm10605 | 75.3 | −2.1 | −2.44 | 1E−15 | 4.2E−14 |
| Tecta | 37.6 | −2.1 | −2.63 | 7.07E−12 | 1.46E−10 |
| Adam23 | 301.5 | −2.13 | −2.37 | 0 | 0 |
| Cbr2 | 76.5 | −2.15 | −3.07 | 5.98E−11 | 1.06E−09 |
| Spic | 2050.5 | −2.16 | −2.37 | 0 | 0 |
| Sema6d | 939.7 | −2.17 | −2.48 | 0 | 0 |
| Hmox1 | 16492.8 | −2.19 | −2.65 | 4E−15 | 1.36E−13 |
| Tgfb2 | 26.2 | −2.19 | −2.92 | 4.6E−12 | 9.71E−11 |
| Fcna | 5828.3 | −2.21 | −2.4 | 0 | 0 |
| Col14a1 | 699.7 | −2.37 | −2.68 | 0 | 0 |
| Wisp2 | 41.1 | −2.37 | −3.69 | 2.46E−12 | 5.41E−11 |
| Ptprm | 389.7 | −2.42 | −2.7 | 0 | 0 |
| Hs3st2 | 297.9 | −2.45 | −2.9 | 0 | 0 |
| Adgre4 | 851.8 | −2.47 | −2.72 | 0 | 0 |
| AC166361.2 | 101 | −2.48 | −2.78 | 0 | 0 |
| Akr1b7 | 148.5 | −2.51 | −2.99 | 0 | 0 |
| Gdf15 | 168.7 | −2.59 | −3.42 | 0 | 5E−15 |
| Nfasc | 204.4 | −2.61 | −3.25 | 0 | 0 |
| Il1a | 348.3 | −2.63 | −3.24 | 0 | 0 |
| Fjx1 | 45.8 | −2.75 | −4.7 | 7E−15 | 2.03E−13 |

TABLE 4
The SD expressed genes list for SV + α4-1BB vs. SV group
by RNA-Seq (q < 0.05, Log2FC ≥ 1 and Log2FC ≤ −1).

| gene | baseMean | log2FC | log2FCunshrunk | pvalue | padj |
|---|---|---|---|---|---|
| Ret | 794.7 | 3.23 | 4.01 | 0 | 0 |
| Gzmk | 1785.2 | 2.77 | 3.04 | 0 | 0 |
| Ermn | 17.7 | 2.73 | 4.67 | 1.64E−13 | 2.4E−11 |
| Ccl8 | 29.5 | 2.55 | 3.64 | 1.07E−11 | 9.75E−10 |
| Wdr95 | 226 | 2.44 | 2.58 | 0 | 0 |
| Ociad2 | 184.7 | 2.4 | 2.5 | 0 | 0 |
| Col6a5 | 17.7 | 2.39 | 5.83 | 3.12E−10 | 1.88E−08 |
| Trp73 | 22 | 2.15 | 3.6 | 9.67E−09 | 4.02E−07 |
| Ncald | 297.2 | 2.11 | 2.28 | 0 | 0 |
| Gzmb | 4113.9 | 2.11 | 2.3 | 0 | 0 |
| Wipf3 | 37.6 | 2.1 | 2.84 | 2.82E−11 | 2.25E−09 |
| 2900011O08Rik | 14.8 | 2.04 | 2.95 | 8.03E−09 | 3.4E−07 |
| Klrg1 | 746.6 | 2.03 | 2.13 | 0 | 0 |
| Clip4 | 39.8 | 1.98 | 2.46 | 1.07E−10 | 7.2E−09 |
| Cd70 | 12.5 | 1.98 | 3.01 | 2.87E−08 | 1.05E−06 |
| Adgrg1 | 449.6 | 1.93 | 2.06 | 0 | 0 |
| Ccl5 | 22142.8 | 1.91 | 2.08 | 0 | 0 |
| Ccr5 | 1338.8 | 1.89 | 1.96 | 0 | 0 |
| Faxc | 12.4 | 1.88 | 3.08 | 3.97E−07 | 1.08E−05 |
| Dpysl5 | 94.9 | 1.84 | 2.16 | 2.13E−11 | 1.76E−09 |
| Gm6637 | 196.3 | 1.83 | 2.02 | 3E−15 | 7.75E−13 |
| Mpl | 45.7 | 1.8 | 2.67 | 8.7E−07 | 2.14E−05 |
| Cdkn2a | 59.4 | 1.79 | 2.09 | 6.97E−11 | 5.07E−09 |
| 1700011L03Rik | 6.7 | 1.77 | 6.04 | 2.28E−06 | 4.92E−05 |
| Treml1 | 31.4 | 1.75 | 2.44 | 6.78E−07 | 1.73E−05 |
| Ccr2 | 1142.1 | 1.74 | 1.86 | 0 | 0 |
| Saa3 | 14.8 | 1.71 | 5.22 | 3.99E−06 | 7.95E−05 |
| Hist1h1b | 56.5 | 1.7 | 1.96 | 2.2E−09 | 1.07E−07 |
| Osr2 | 54.7 | 1.69 | 2.19 | 3.53E−07 | 9.87E−06 |
| Col6a1 | 15.6 | 1.69 | 4.32 | 7.89E−06 | 0.000144 |
| Tff1 | 8.6 | 1.69 | 3.25 | 8.36E−06 | 0.000151 |
| Fam57b | 23.3 | 1.68 | 5.02 | 4.51E−07 | 1.21E−05 |
| Col3a1 | 82.1 | 1.68 | 3.39 | 9.31E−06 | 0.000166 |
| Cldnd2 | 53.5 | 1.67 | 1.88 | 1.05E−11 | 9.69E−10 |
| Col6a2 | 16.9 | 1.67 | 3.3 | 1.11E−05 | 0.000192 |
| Insrr | 64.7 | 1.62 | 1.77 | 7.83E−13 | 9.25E−11 |
| Dixdc1 | 18.6 | 1.62 | 2.45 | 5.33E−06 | 0.000102 |
| Gm33460 | 48.9 | 1.61 | 1.8 | 6.88E−11 | 5.04E−09 |
| Csf2 | 72.9 | 1.61 | 1.86 | 3.27E−09 | 1.52E−07 |
| Gcg | 62.2 | 1.61 | 3.73 | 1.52E−05 | 0.000252 |
| Nkg7 | 8456.1 | 1.57 | 1.62 | 0 | 0 |
| Tmem40 | 47.5 | 1.57 | 1.98 | 8.68E−07 | 2.14E−05 |
| Stk32c | 341 | 1.55 | 1.64 | 0 | 1E−15 |
| Serpine2 | 31.1 | 1.55 | 2.06 | 2.38E−06 | 5.09E−05 |
| Wdr31 | 15.1 | 1.55 | 2.2 | 1.3E−05 | 0.00022 |
| S100a4 | 1170.3 | 1.54 | 1.73 | 2.18E−10 | 1.37E−08 |
| AA467197 | 30.3 | 1.54 | 1.86 | 3.68E−07 | 1.02E−05 |
| Dcn | 49 | 1.54 | 2.29 | 5.15E−06 | 9.93E−05 |
| Gp1bb | 21.6 | 1.54 | 2.35 | 3.11E−05 | 0.000458 |
| Osbpl3 | 1358.5 | 1.52 | 1.57 | 0 | 0 |
| Smpdl3b | 185.8 | 1.52 | 1.62 | 6E−15 | 1.27E−12 |
| Gldc | 15.3 | 1.51 | 1.94 | 9.21E−06 | 0.000165 |
| Arsb | 1795.9 | 1.5 | 1.59 | 0 | 2E−15 |
| Bspry | 174.2 | 1.5 | 1.74 | 3.88E−08 | 1.38E−06 |
| Gm15056 | 28.9 | 1.5 | 2.19 | 4.69E−05 | 0.000651 |
| Esm1 | 680.1 | 1.49 | 1.62 | 2.19E−13 | 3.05E−11 |
| Pif1 | 204 | 1.49 | 1.64 | 3.87E−11 | 2.98E−09 |
| 2310031A07Rik | 64.6 | 1.49 | 1.87 | 1.65E−06 | 3.7E−05 |
| Pglyrp1 | 1046.5 | 1.48 | 1.61 | 9.35E−13 | 1.08E−10 |
| Slc35d3 | 110.5 | 1.46 | 1.56 | 3.8E−14 | 6.51E−12 |
| Pbk | 107.5 | 1.46 | 1.55 | 8.9E−14 | 1.4E−11 |
| Gm2788 | 29.7 | 1.46 | 1.78 | 1.1E−06 | 2.61E−05 |
| Slc16a11 | 9.1 | 1.46 | 2.52 | 9.04E−05 | 0.001144 |
| Miat | 31.2 | 1.45 | 1.67 | 5.48E−08 | 1.88E−06 |
| Samd14 | 203.2 | 1.44 | 1.65 | 2.06E−08 | 8.04E−07 |
| Tnfsf4 | 81.6 | 1.44 | 1.71 | 2.52E−07 | 7.28E−06 |
| Gp5 | 24.7 | 1.44 | 1.94 | 1.94E−05 | 0.000307 |
| Kcnj5 | 5.3 | 1.44 | 3.82 | 6.73E−05 | 0.000888 |
| Vax2 | 12.7 | 1.43 | 2.3 | 0.00012 | 0.001443 |
| Car5b | 136 | 1.42 | 1.49 | 0 | 7.2E−14 |
| Reg2 | 5.6 | 1.42 | 5.34 | 5.5E−06 | 0.000105 |
| Tpbg | 19.3 | 1.42 | 2.07 | 7.65E−05 | 0.00099 |
| Pf4 | 162.2 | 1.42 | 2.19 | 0.000132 | 0.001566 |
| Trpc6 | 7.4 | 1.42 | 2.85 | 0.000145 | 0.001696 |
| Ppbp | 30.6 | 1.41 | 1.97 | 6.29E−05 | 0.000839 |
| Adap1 | 1257.2 | 1.4 | 1.48 | 1E−15 | 2.09E−13 |
| Kif2c | 341.3 | 1.4 | 1.48 | 4E−15 | 8.96E−13 |
| Tff3 | 16.7 | 1.4 | 1.76 | 1.49E−05 | 0.000248 |
| Muc13 | 227 | 1.39 | 1.53 | 8.75E−10 | 4.68E−08 |
| Serpina3h | 8.8 | 1.39 | 2.39 | 0.000211 | 0.002313 |
| Rgs16 | 7742.7 | 1.38 | 1.44 | 0 | 0 |
| Fhl2 | 217.6 | 1.38 | 1.45 | 0 | 2.7E−14 |
| Serping1 | 49.6 | 1.38 | 1.61 | 2.11E−07 | 6.25E−06 |
| Slc22a3 | 85.3 | 1.38 | 1.65 | 1.66E−06 | 3.71E−05 |
| Nfe2 | 91.3 | 1.38 | 1.81 | 4.07E−05 | 0.000577 |
| Igf2bp3 | 26.6 | 1.37 | 1.7 | 2.05E−05 | 0.000323 |
| Ppp1r3g | 7.3 | 1.37 | 2.61 | 0.000309 | 0.003167 |
| Map6 | 151 | 1.36 | 1.45 | 4.41E−13 | 5.67E−11 |
| Fkbp10 | 29.4 | 1.36 | 1.89 | 6.66E−05 | 0.00088 |
| Tuba8 | 42.2 | 1.36 | 1.95 | 0.000162 | 0.00185 |
| Mmrn1 | 13.3 | 1.36 | 1.98 | 0.000187 | 0.002084 |
| Hist1h2ag | 6.4 | 1.36 | 2.16 | 0.000327 | 0.003318 |
| Rasgef1a | 270.7 | 1.34 | 1.42 | 4E−15 | 8.29E−13 |
| Fam81a | 55.4 | 1.34 | 1.55 | 1.08E−06 | 2.55E−05 |
| Col1a2 | 95 | 1.34 | 1.73 | 2.79E−05 | 0.000418 |
| Fhl1 | 19.9 | 1.34 | 1.9 | 0.000148 | 0.001721 |
| Fam19a3 | 168.7 | 1.33 | 1.48 | 2.06E−08 | 8.04E−07 |
| Cdk1 | 1228.8 | 1.32 | 1.41 | 5.17E−13 | 6.5E−11 |
| 1700001O22Rik | 140.3 | 1.32 | 1.42 | 7.29E−11 | 5.24E−09 |
| Pimreg | 230 | 1.32 | 1.42 | 1.77E−10 | 1.14E−08 |
| F2rl2 | 42.8 | 1.32 | 1.52 | 1.19E−06 | 2.78E−05 |
| Dach1 | 18.3 | 1.32 | 1.82 | 0.000178 | 0.002004 |
| Cenpf | 495.6 | 1.31 | 1.39 | 6.5E−14 | 1.05E−11 |
| Tpx2 | 1113.7 | 1.31 | 1.39 | 1.56E−12 | 1.7E−10 |
| Hmmr | 311.7 | 1.31 | 1.42 | 2.97E−10 | 1.8E−08 |
| Tubb1 | 17.5 | 1.31 | 1.94 | 0.000239 | 0.002557 |
| Upk3b | 15.3 | 1.31 | 2.92 | 0.000536 | 0.005053 |
| Kif18b | 606.6 | 1.3 | 1.37 | 1.37E−13 | 2.06E−11 |
| Adgrg7 | 5.3 | 1.3 | 3.04 | 0.00033 | 0.003346 |
| Cep55 | 503.9 | 1.29 | 1.36 | 1.3E−12 | 1.42E−10 |
| Tpsab1 | 262.4 | 1.29 | 1.5 | 4E−06 | 7.97E−05 |
| Unc5b | 14.9 | 1.29 | 1.8 | 0.000177 | 0.001988 |
| Sytl2 | 1359.2 | 1.28 | 1.34 | 1.2E−14 | 2.26E−12 |
| Kif14 | 299.1 | 1.28 | 1.36 | 6.35E−11 | 4.68E−09 |
| Tg | 98.4 | 1.28 | 1.39 | 2.03E−09 | 9.93E−08 |
| Ube2c | 1391 | 1.28 | 1.4 | 6.71E−09 | 2.91E−07 |
| Ifng | 1413.7 | 1.28 | 1.45 | 2.03E−07 | 6.03E−06 |
| Angpt1 | 63.2 | 1.28 | 1.48 | 3.02E−06 | 6.25E−05 |
| Aunip | 24.6 | 1.28 | 1.61 | 9.97E−05 | 0.00124 |
| Birc5 | 1540.7 | 1.27 | 1.41 | 2.84E−08 | 1.05E−06 |
| AC153938.2 | 40.5 | 1.27 | 1.51 | 9.04E−06 | 0.000162 |
| Gng3 | 34.8 | 1.27 | 1.49 | 1.41E−05 | 0.000237 |
| Robo3 | 27.3 | 1.27 | 1.51 | 2.28E−05 | 0.000357 |
| Myct1 | 34.7 | 1.27 | 1.65 | 0.000137 | 0.001615 |
| Kif22 | 1022.2 | 1.26 | 1.35 | 3.03E−11 | 2.39E−09 |
| Rad51 | 401.4 | 1.26 | 1.34 | 5.95E−11 | 4.4E−09 |
| Havcr2 | 486.2 | 1.26 | 1.36 | 1.36E−10 | 9.01E−09 |
| S100a6 | 3227.2 | 1.26 | 1.37 | 1.24E−09 | 6.42E−08 |
| Neurl1b | 49.9 | 1.26 | 1.52 | 2.54E−05 | 0.000389 |
| Irx3 | 8.6 | 1.26 | 2.06 | 0.000738 | 0.006563 |
| Scrn1 | 9.5 | 1.26 | 2.43 | 0.000777 | 0.006836 |
| Depdc1b | 350.8 | 1.25 | 1.32 | 7.29E−12 | 6.94E−10 |
| Prc1 | 830.9 | 1.25 | 1.36 | 9.49E−10 | 5E−08 |
| Lxn | 79 | 1.25 | 1.35 | 3.32E−09 | 1.53E−07 |
| Ccnb1 | 160.8 | 1.25 | 1.37 | 1.44E−08 | 5.81E−07 |
| Ccna2 | 1680.2 | 1.24 | 1.3 | 7E−15 | 1.41E−12 |
| Anxa2 | 5777.8 | 1.24 | 1.34 | 3.39E−10 | 2.01E−08 |
| Gzma | 3261.2 | 1.24 | 1.86 | 0.000818 | 0.007121 |
| Shcbp1 | 274.8 | 1.23 | 1.29 | 2.78E−13 | 3.78E−11 |
| Cdca8 | 1251.5 | 1.23 | 1.3 | 2.54E−12 | 2.68E−10 |
| 2610318N02Rik | 193.2 | 1.23 | 1.32 | 8.3E−10 | 4.48E−08 |
| Bst1 | 74.2 | 1.23 | 1.33 | 7.18E−09 | 3.08E−07 |
| Sgo2a | 143.4 | 1.23 | 1.33 | 9.33E−09 | 3.88E−07 |
| Lrr1 | 59 | 1.23 | 1.37 | 8.05E−07 | 1.99E−05 |
| Epas1 | 1633 | 1.23 | 1.59 | 0.000164 | 0.001873 |
| Plppr3 | 42.4 | 1.23 | 1.76 | 0.000686 | 0.006205 |
| Dapk2 | 315.5 | 1.22 | 1.27 | 0 | 7.7E−14 |
| Aurkb | 806.6 | 1.22 | 1.28 | 4.93E−13 | 6.25E−11 |
| Plk1 | 794.8 | 1.22 | 1.3 | 3.01E−11 | 2.38E−09 |
| Heatr9 | 204.6 | 1.22 | 1.36 | 3.37E−07 | 9.5E−06 |
| Npy | 11.8 | 1.22 | 1.86 | 0.001046 | 0.008713 |
| Myrf | 11.2 | 1.22 | 2.86 | 0.001338 | 0.010572 |
| Chsy1 | 2251.6 | 1.21 | 1.27 | 3E−15 | 6.66E−13 |
| Mxd3 | 280.1 | 1.21 | 1.26 | 2.7E−14 | 4.71E−12 |
| Sapcd2 | 275.5 | 1.21 | 1.3 | 1.06E−09 | 5.54E−08 |
| Troap | 236.4 | 1.21 | 1.31 | 6.8E−09 | 2.94E−07 |
| Tigit | 2942.9 | 1.21 | 1.33 | 3.76E−08 | 1.35E−06 |
| Rtkn | 29.5 | 1.21 | 1.48 | 5.85E−05 | 0.000791 |
| Alox12 | 39.7 | 1.21 | 1.5 | 7.16E−05 | 0.000937 |
| Il13 | 60.8 | 1.21 | 1.61 | 0.000618 | 0.005699 |
| Hist1h3g | 9.3 | 1.21 | 1.63 | 0.000849 | 0.007357 |
| Cdca5 | 791.4 | 1.2 | 1.25 | 0 | 1.5E−14 |
| Gimap7 | 2437.5 | 1.2 | 1.27 | 1.58E−11 | 1.35E−09 |
| Ccnb2 | 1255.4 | 1.2 | 1.28 | 8.31E−11 | 5.8E−09 |
| Fam78b | 52.7 | 1.2 | 1.45 | 1.68E−05 | 0.000274 |
| Gm9531 | 16 | 1.2 | 1.66 | 0.000496 | 0.004738 |
| Tmem98 | 10.9 | 1.2 | 2.01 | 0.001307 | 0.010371 |
| Eomes | 4109.7 | 1.19 | 1.22 | 0 | 0 |
| Penk | 1018 | 1.19 | 1.26 | 1.11E−11 | 9.94E−10 |
| Serpinb6b | 1199 | 1.19 | 1.26 | 1.24E−11 | 1.08E−09 |
| Prr11 | 288 | 1.19 | 1.32 | 2.76E−07 | 7.89E−06 |
| Ica1 | 85.1 | 1.19 | 1.38 | 6.49E−06 | 0.000121 |
| Gm4841 | 29.9 | 1.19 | 1.54 | 0.000218 | 0.002367 |
| Fbxo41 | 15.3 | 1.19 | 1.51 | 0.00028 | 0.002912 |
| Col6a6 | 6.3 | 1.19 | 3.11 | 0.001409 | 0.011028 |
| Cst7 | 1893.9 | 1.18 | 1.22 | 0 | 1.5E−14 |
| Top2a | 4726.3 | 1.18 | 1.23 | 0 | 1.3E−13 |
| Ncaph | 822.4 | 1.18 | 1.24 | 3.2E−14 | 5.46E−12 |
| Cdca2 | 444.5 | 1.18 | 1.24 | 1.09E−11 | 9.85E−10 |
| Rrm2 | 1718.2 | 1.18 | 1.26 | 3.64E−10 | 2.15E−08 |
| Rad54l | 350.2 | 1.18 | 1.27 | 2.64E−09 | 1.27E−07 |
| Ttk | 211.5 | 1.18 | 1.27 | 4.53E−09 | 2.04E−07 |
| BC030867 | 129.6 | 1.18 | 1.27 | 2.3E−08 | 8.82E−07 |
| Cdc25c | 143.9 | 1.18 | 1.3 | 5.8E−07 | 1.52E−05 |
| Rai14 | 77.8 | 1.18 | 1.34 | 2.2E−06 | 4.79E−05 |
| Hist1h3c | 11.8 | 1.18 | 1.67 | 0.001203 | 0.009747 |
| Gbp11 | 355.1 | 1.17 | 1.23 | 2.77E−11 | 2.23E−09 |
| Foxd2os | 95 | 1.17 | 1.24 | 8.95E−11 | 6.2E−09 |
| Fignl1 | 145.7 | 1.17 | 1.23 | 1.17E−10 | 7.79E−09 |
| Nek2 | 554.5 | 1.17 | 1.26 | 3.39E−09 | 1.56E−07 |
| E2f8 | 535.1 | 1.17 | 1.27 | 3.38E−08 | 1.23E−06 |
| C78197 | 19.1 | 1.17 | 1.65 | 0.00074 | 0.006571 |
| Mki67 | 5899.1 | 1.16 | 1.24 | 4.42E−09 | 1.99E−07 |
| E2f7 | 275.3 | 1.16 | 1.26 | 5.55E−08 | 1.9E−06 |
| Spp1 | 51 | 1.16 | 1.36 | 4.36E−05 | 0.000612 |
| Gp9 | 24.8 | 1.16 | 1.46 | 0.000374 | 0.003703 |
| Vash1 | 14.6 | 1.16 | 1.66 | 0.001212 | 0.009798 |
| Gm867 | 11.8 | 1.16 | 1.91 | 0.001595 | 0.012127 |
| Nlrp6 | 5.5 | 1.16 | 3.11 | 0.002039 | 0.014755 |
| Tyms | 809.6 | 1.15 | 1.19 | 0 | 7.4E−14 |
| Espl1 | 809.6 | 1.15 | 1.22 | 2.11E−11 | 1.75E−09 |
| Ncapg | 383.1 | 1.15 | 1.22 | 8.97E−10 | 4.77E−08 |
| Ckap2l | 455.4 | 1.15 | 1.24 | 2.19E−08 | 8.5E−07 |
| Gm12250 | 177.3 | 1.15 | 1.28 | 5.22E−07 | 1.38E−05 |
| Spns2 | 183.2 | 1.15 | 1.38 | 6.6E−05 | 0.000874 |
| Il20ra | 9.2 | 1.15 | 1.8 | 0.001826 | 0.01357 |
| Nusap1 | 813.1 | 1.14 | 1.19 | 1.06E−12 | 1.21E−10 |
| Serpinb9 | 1224.7 | 1.14 | 1.21 | 1.43E−11 | 1.24E−09 |
| Spag5 | 1183.2 | 1.14 | 1.2 | 3.25E−11 | 2.54E−09 |
| Cenpe | 685.5 | 1.14 | 1.21 | 1.07E−10 | 7.2E−09 |
| Bub1 | 362.3 | 1.14 | 1.22 | 3.72E−09 | 1.7E−07 |
| Serpina3f | 452 | 1.14 | 1.24 | 7.57E−08 | 2.52E−06 |
| Ccl4 | 2257.9 | 1.14 | 1.43 | 0.000269 | 0.002817 |
| Ckap2 | 394.5 | 1.13 | 1.17 | 9E−15 | 1.82E−12 |
| Cdkn3 | 129.2 | 1.13 | 1.18 | 1.04E−10 | 7.09E−09 |
| Sgo1 | 162.8 | 1.13 | 1.19 | 6.38E−10 | 3.57E−08 |
| Serpina3g | 2916.5 | 1.13 | 1.23 | 3.48E−08 | 1.25E−06 |
| Ccl1 | 31 | 1.13 | 1.43 | 0.000689 | 0.006224 |
| Prf1 | 1704.5 | 1.12 | 1.14 | 0 | 0 |
| Spc24 | 506 | 1.12 | 1.16 | 1.96E−13 | 2.79E−11 |
| Kif11 | 1109.9 | 1.12 | 1.17 | 2.93E−13 | 3.94E−11 |
| Bub1b | 1344.3 | 1.12 | 1.18 | 7.17E−11 | 5.17E−09 |
| Lgals1 | 11014.9 | 1.12 | 1.18 | 2.68E−10 | 1.64E−08 |
| Esco2 | 149.5 | 1.12 | 1.21 | 1.11E−07 | 3.55E−06 |
| Tek | 24.8 | 1.12 | 1.43 | 0.000437 | 0.004239 |
| Msantd3 | 9.2 | 1.12 | 1.65 | 0.002189 | 0.015686 |
| Acot7 | 2318.6 | 1.11 | 1.17 | 2.16E−11 | 1.77E−09 |
| Ttn | 292.1 | 1.11 | 1.21 | 8E−08 | 2.66E−06 |
| Nuf2 | 279.6 | 1.11 | 1.21 | 5.67E−07 | 1.49E−05 |
| Rgs18 | 52.3 | 1.11 | 1.38 | 0.00036 | 0.003592 |
| Dio2 | 18.9 | 1.11 | 1.6 | 0.001465 | 0.011345 |
| Pcdh7 | 8.7 | 1.11 | 1.82 | 0.002625 | 0.018256 |
| Kif4 | 650.2 | 1.1 | 1.15 | 1.06E−11 | 9.7E−10 |
| Clspn | 637.1 | 1.1 | 1.17 | 1.4E−09 | 7.11E−08 |
| Dyrk3 | 249.1 | 1.1 | 1.21 | 4.14E−07 | 1.12E−05 |
| 1700020L24Rik | 90.2 | 1.1 | 1.23 | 4.95E−06 | 9.58E−05 |
| Lmtk3 | 107.1 | 1.1 | 1.28 | 4.1E−05 | 0.00058 |
| Ms4a3 | 31.1 | 1.1 | 1.55 | 0.002955 | 0.019997 |
| Asf1b | 1276.4 | 1.09 | 1.14 | 1.09E−12 | 1.23E−10 |
| Pask | 202.9 | 1.09 | 1.15 | 7.34E−10 | 4.02E−08 |
| Cit | 784.3 | 1.09 | 1.16 | 2.83E−09 | 1.35E−07 |
| Pclaf | 522.9 | 1.09 | 1.19 | 6.53E−07 | 1.68E−05 |
| Klrc1 | 236.6 | 1.09 | 1.2 | 1.62E−06 | 3.64E−05 |
| Rnase2a | 5.3 | 1.09 | 1.76 | 0.003732 | 0.024243 |
| 6530402F18Rik | 553.7 | 1.08 | 1.1 | 0 | 0 |
| Cdc45 | 628.7 | 1.08 | 1.11 | 0 | 2E−15 |
| Podnl1 | 2382.6 | 1.08 | 1.12 | 4.8E−14 | 7.96E−12 |
| Lilr4b | 1553.9 | 1.08 | 1.16 | 4.21E−08 | 1.49E−06 |
| Perp | 113.8 | 1.08 | 1.17 | 9.07E−08 | 2.95E−06 |
| Aspm | 319.4 | 1.08 | 1.17 | 2.91E−07 | 8.3E−06 |
| Gzmc | 56.6 | 1.08 | 1.32 | 0.000285 | 0.002956 |
| Clstn3 | 34.6 | 1.08 | 1.39 | 0.000965 | 0.008168 |
| Traj35 | 15.6 | 1.08 | 1.44 | 0.001132 | 0.009302 |
| Pcp4l1 | 21 | 1.08 | 1.56 | 0.002232 | 0.015944 |
| Serpina3n | 16.7 | 1.08 | 1.62 | 0.002935 | 0.01989 |
| Chil3 | 42.4 | 1.08 | 1.54 | 0.003165 | 0.021138 |
| AC131739.1 | 7.2 | 1.08 | 1.94 | 0.003928 | 0.025182 |
| Kif20a | 898.5 | 1.07 | 1.13 | 3.27E−10 | 1.95E−08 |
| Tjp2 | 411.7 | 1.07 | 1.14 | 2.03E−08 | 7.97E−07 |
| Gm5391 | 5.3 | 1.07 | 2.14 | 0.004733 | 0.02915 |
| Cdc20 | 1268.9 | 1.06 | 1.1 | 1.22E−13 | 1.87E−11 |
| Kcnk5 | 399.5 | 1.06 | 1.1 | 6.1E−13 | 7.51E−11 |
| Psrc1 | 122.1 | 1.06 | 1.12 | 2.48E−09 | 1.19E−07 |
| Iqgap3 | 284.2 | 1.06 | 1.13 | 7.9E−09 | 3.37E−07 |
| Cks1b | 772.5 | 1.06 | 1.14 | 6.32E−08 | 2.16E−06 |
| Grb10 | 61 | 1.06 | 1.32 | 0.000464 | 0.004477 |
| Ptger3 | 20.8 | 1.06 | 1.62 | 0.004232 | 0.026687 |
| Cdkn2c | 421.3 | 1.05 | 1.08 | 8.39E−13 | 9.78E−11 |
| Ndc80 | 345.5 | 1.05 | 1.1 | 1.74E−10 | 1.12E−08 |
| S100a10 | 7676.1 | 1.05 | 1.1 | 5.19E−10 | 2.98E−08 |
| Mcm10 | 477.4 | 1.05 | 1.11 | 1.26E−09 | 6.54E−08 |
| Fut7 | 69.8 | 1.05 | 1.18 | 2.47E−05 | 0.000382 |
| Tfr2 | 139.5 | 1.05 | 1.34 | 0.001188 | 0.009649 |
| Hist1h2bm | 8.5 | 1.05 | 1.8 | 0.005816 | 0.034129 |
| Stmn1 | 257.1 | 1.04 | 1.1 | 2.99E−09 | 1.41E−07 |
| Ska1 | 210.9 | 1.04 | 1.13 | 4.21E−06 | 8.29E−05 |
| Klrc2 | 53.2 | 1.04 | 1.17 | 2.62E−05 | 0.000399 |
| Cym | 46.2 | 1.04 | 1.31 | 0.000743 | 0.006595 |
| Hist1h2ai | 9.8 | 1.04 | 1.51 | 0.004383 | 0.027452 |
| Phlda3 | 37.7 | 1.03 | 1.2 | 0.000147 | 0.00171 |
| Gm14148 | 21.4 | 1.03 | 1.25 | 0.000572 | 0.005313 |
| Hist1h2bj | 17.7 | 1.03 | 1.24 | 0.001234 | 0.009915 |
| a | 12.6 | 1.03 | 1.58 | 0.004957 | 0.030315 |
| 4930519L02Rik | 9.9 | 1.03 | 1.87 | 0.006787 | 0.038499 |
| Knstrn | 855.1 | 1.02 | 1.05 | 5E−15 | 1.13E−12 |
| Chst11 | 1091.1 | 1.02 | 1.05 | 1.6E−14 | 2.94E−12 |
| Spdl1 | 204.5 | 1.02 | 1.07 | 3.91E−10 | 2.29E−08 |
| Melk | 329.8 | 1.02 | 1.07 | 7E−10 | 3.88E−08 |
| Kifc1 | 310.5 | 1.02 | 1.09 | 8.1E−08 | 2.69E−06 |
| Anln | 217.2 | 1.02 | 1.1 | 1.27E−06 | 2.94E−05 |
| Gm17745 | 84 | 1.02 | 1.11 | 1.34E−06 | 3.07E−05 |
| Runx2os1 | 41.2 | 1.02 | 1.28 | 0.001157 | 0.009439 |
| H1fx | 65.9 | 1.02 | 1.27 | 0.001224 | 0.00986 |
| Col1a1 | 89.2 | 1.02 | 1.37 | 0.002114 | 0.015218 |
| Slc30a2 | 29 | 1.02 | 1.34 | 0.002562 | 0.017915 |
| Mt3 | 18.3 | 1.02 | 1.35 | 0.003871 | 0.024928 |
| Cxcr6 | 818 | 1.01 | 1.04 | 7.2E−14 | 1.15E−11 |
| Kif15 | 595.3 | 1.01 | 1.07 | 1.32E−08 | 5.37E−07 |
| Ect2 | 255.1 | 1.01 | 1.12 | 1.58E−05 | 0.00026 |
| Tex15 | 58.4 | 1.01 | 1.12 | 4.2E−05 | 0.000591 |
| Ddah2 | 90 | 1.01 | 1.15 | 6.54E−05 | 0.000868 |
| G0s2 | 77.4 | 1.01 | 1.24 | 0.000666 | 0.006046 |
| Hist1h3a | 32.2 | 1.01 | 1.25 | 0.001415 | 0.011057 |
| Pde10a | 17 | 1.01 | 1.3 | 0.002501 | 0.017557 |
| Morc1 | 8.4 | 1.01 | 2.01 | 0.007372 | 0.040918 |
| Lmnb1 | 6788.3 | 1 | 1.03 | 5.3E−14 | 8.63E−12 |
| Bard1 | 316.8 | 1 | 1.05 | 1.61E−10 | 1.06E−08 |
| Ska3 | 252.1 | 1 | 1.05 | 4.68E−10 | 2.72E−08 |
| Tk1 | 1004.9 | 1 | 1.05 | 5.56E−10 | 3.14E−08 |
| Sytl3 | 850.3 | 1 | 1.09 | 2.44E−06 | 5.19E−05 |
| Bag2 | 63 | 1 | 1.1 | 1.06E−05 | 0.000185 |
| F10 | 33.6 | 1 | 1.38 | 0.004704 | 0.029055 |
| C1s1 | 21.8 | 1 | 1.52 | 0.005149 | 0.031153 |
| Usp50 | 16.7 | 1 | 1.55 | 0.006828 | 0.038667 |
| Dlk2 | 11.4 | 1 | 1.71 | 0.0082 | 0.044224 |
| Efna5 | 6.6 | 1 | 1.98 | 0.00842 | 0.045107 |
| Ptk2 | 617.3 | −1 | −1.03 | 0 | 1.3E−13 |
| Myo10 | 1090.4 | −1 | −1.06 | 3.72E−08 | 1.34E−06 |
| Tmem8b | 139.4 | −1 | −1.09 | 8.06E−07 | 1.99E−05 |
| Tgm2 | 2908.8 | −1 | −1.09 | 1.15E−06 | 2.7E−05 |
| Dysf | 302 | −1 | −1.1 | 3.86E−06 | 7.73E−05 |
| Mreg | 104.8 | −1 | −1.11 | 7.36E−06 | 0.000136 |
| Fam167a | 155 | −1 | −1.11 | 1.28E−05 | 0.000218 |
| Adam33 | 60.3 | −1 | −1.17 | 0.00027 | 0.002823 |
| Eps8 | 43.8 | −1 | −1.19 | 0.000346 | 0.003473 |
| Syt3 | 18.7 | −1 | −1.32 | 0.001772 | 0.013249 |
| Rubcnl | 142.1 | −1 | −1.34 | 0.001911 | 0.014015 |
| Napb | 20.4 | −1 | −1.57 | 0.004628 | 0.02868 |
| Sat2 | 9 | −1 | −1.91 | 0.008166 | 0.044102 |
| Tsix | 8.2 | −1 | −2.12 | 0.008513 | 0.045495 |
| Gm42870 | 22.1 | −1 | −2.22 | 0.008535 | 0.045552 |
| Myo9a | 1226.4 | −1.01 | −1.06 | 1.33E−09 | 6.81E−08 |
| Tns3 | 884 | −1.01 | −1.1 | 6.53E−07 | 1.68E−05 |
| Nat8l | 123.4 | −1.01 | −1.11 | 2.77E−06 | 5.81E−05 |
| Col17a1 | 97 | −1.01 | −1.13 | 1.15E−05 | 0.000198 |
| Adrb1 | 207 | −1.01 | −1.15 | 3.23E−05 | 0.00047 |
| Sh3tc1 | 174.9 | −1.01 | −1.16 | 6.02E−05 | 0.000809 |
| Abca9 | 130.3 | −1.01 | −1.29 | 0.000973 | 0.008226 |
| Igkv6-25 | 29.5 | −1.01 | −1.73 | 0.006776 | 0.038448 |
| Gm36159 | 8.5 | −1.01 | −2.04 | 0.007309 | 0.040646 |
| Gm8463 | 8.7 | −1.01 | −3.66 | 0.00768 | 0.042258 |
| Ebf1 | 216.1 | −1.01 | −2.27 | 0.007802 | 0.042823 |
| Tbc1d8 | 696.1 | −1.02 | −1.12 | 1.77E−06 | 3.92E−05 |
| Adgrl2 | 148.4 | −1.02 | −1.13 | 3.22E−06 | 6.58E−05 |
| Ppp1r14a | 48.4 | −1.02 | −1.4 | 0.00191 | 0.014015 |
| Padi4 | 34.4 | −1.02 | −1.28 | 0.002072 | 0.014949 |
| Gm30292 | 19.5 | −1.02 | −1.37 | 0.00212 | 0.01525 |
| Slc13a2 | 13.1 | −1.02 | −1.9 | 0.005131 | 0.031092 |
| Epha4 | 19.9 | −1.02 | −1.81 | 0.005323 | 0.031904 |
| Stfa3 | 14.7 | −1.02 | −1.71 | 0.005735 | 0.03378 |
| Gm25776 | 9.5 | −1.02 | −1.77 | 0.00584 | 0.034194 |
| Crisp3 | 7.4 | −1.02 | −4.01 | 0.00631 | 0.03631 |
| Igha | 3248.3 | −1.02 | −1.95 | 0.006417 | 0.036825 |
| Igkv1-135 | 261.8 | −1.02 | −1.85 | 0.006595 | 0.037626 |
| Pax5 | 1019.4 | −1.02 | −2.07 | 0.007319 | 0.040689 |
| Plxna1 | 900.2 | −1.03 | −1.11 | 8.86E−08 | 2.89E−06 |
| Cpq | 349.9 | −1.03 | −1.12 | 6.17E−07 | 1.61E−05 |
| Hck | 1125 | −1.03 | −1.12 | 6.33E−07 | 1.64E−05 |
| Pnck | 68.8 | −1.03 | −1.16 | 6.44E−06 | 0.000121 |
| Nectin4 | 97.5 | −1.03 | −1.18 | 5.48E−05 | 0.000746 |
| Cd180 | 364.5 | −1.03 | −1.2 | 6.2E−05 | 0.000829 |
| Btnl2 | 69.3 | −1.03 | −1.22 | 0.000154 | 0.001777 |
| Zfyve9 | 64.7 | −1.03 | −1.3 | 0.000587 | 0.00543 |
| Krt80 | 33 | −1.03 | −1.33 | 0.000948 | 0.00806 |
| Cmtm8 | 27.8 | −1.03 | −1.34 | 0.000991 | 0.008347 |
| Adgra2 | 49.3 | −1.03 | −1.44 | 0.001853 | 0.013683 |
| Tnnc2 | 16.7 | −1.03 | −1.54 | 0.002356 | 0.016673 |
| Klhl14 | 29.4 | −1.03 | −1.95 | 0.005716 | 0.033707 |
| Rasgrp3 | 260.5 | −1.03 | −1.87 | 0.006222 | 0.035888 |
| Tcn2 | 1368.6 | −1.04 | −1.08 | 1E−15 | 1.65E−13 |
| Ppp1r32 | 136.8 | −1.04 | −1.13 | 2.11E−07 | 6.25E−06 |
| Fcgrt | 2555.6 | −1.04 | −1.13 | 5.87E−07 | 1.54E−05 |
| Nphp3 | 103.3 | −1.04 | −1.16 | 1.69E−06 | 3.77E−05 |
| Cd300lf | 581.8 | −1.04 | −1.16 | 3.88E−06 | 7.77E−05 |
| Ly86 | 762.3 | −1.04 | −1.17 | 1.71E−05 | 0.000278 |
| Clec12a | 552.3 | −1.04 | −1.2 | 3.22E−05 | 0.00047 |
| Arhgap32 | 163.9 | −1.04 | −1.22 | 6.6E−05 | 0.000874 |
| Eva1a | 33 | −1.04 | −1.25 | 0.000199 | 0.002202 |
| Wtip | 28.7 | −1.04 | −1.44 | 0.00145 | 0.011259 |
| B3gnt7 | 121.7 | −1.04 | −1.43 | 0.001841 | 0.013654 |
| Npc1l1 | 18.3 | −1.04 | −1.68 | 0.003219 | 0.021411 |
| Prex2 | 8.7 | −1.04 | −2.21 | 0.005599 | 0.03317 |
| Cd209c | 9.8 | −1.04 | −2.31 | 0.005938 | 0.034665 |
| Zbtb10 | 1450.8 | −1.05 | −1.09 | 3.33E−12 | 3.42E−10 |
| Pla2g7 | 1374.2 | −1.05 | −1.13 | 6.38E−08 | 2.18E−06 |
| Fcgr1 | 319.2 | −1.05 | −1.15 | 3.57E−07 | 9.94E−06 |
| Pla2g15 | 1269.3 | −1.05 | −1.15 | 5.99E−07 | 1.57E−05 |
| B230303O12Rik | 93.4 | −1.05 | −1.16 | 7.3E−07 | 1.84E−05 |
| Nr3c2 | 42.8 | −1.05 | −1.28 | 0.000159 | 0.001828 |
| Gpr4 | 64.4 | −1.05 | −1.29 | 0.00028 | 0.002912 |
| Chn1 | 24 | −1.05 | −1.26 | 0.000645 | 0.005891 |
| Bmf | 141.4 | −1.05 | −1.4 | 0.000933 | 0.007945 |
| Gm44860 | 11.3 | −1.05 | −2.25 | 0.005162 | 0.031194 |
| Mcf2 | 7.3 | −1.05 | −3.2 | 0.005808 | 0.034089 |
| Hebp1 | 934.9 | −1.06 | −1.19 | 4.49E−06 | 8.77E−05 |
| Susd4 | 89.1 | −1.06 | −1.2 | 1.03E−05 | 0.000181 |
| Tspan15 | 134 | −1.06 | −1.22 | 1.65E−05 | 0.00027 |
| Fpr1 | 89.9 | −1.06 | −1.32 | 0.000293 | 0.003019 |
| Ighv2-3 | 64.5 | −1.06 | −1.36 | 0.000905 | 0.007792 |
| Alas2 | 140.4 | −1.06 | −1.75 | 0.004005 | 0.025599 |
| Paqr6 | 11.7 | −1.06 | −1.97 | 0.004373 | 0.02741 |
| Gm15929 | 8.8 | −1.06 | −1.92 | 0.004709 | 0.029072 |
| Gm37829 | 7 | −1.06 | −2.2 | 0.004866 | 0.02984 |
| Gm38160 | 15.5 | −1.06 | −2.07 | 0.004891 | 0.029962 |
| Gm26575 | 11.1 | −1.06 | −2.25 | 0.004985 | 0.03041 |
| Gm2814 | 9.6 | −1.06 | −2.68 | 0.005305 | 0.031827 |
| Sort1 | 1028.3 | −1.07 | −1.19 | 9.44E−07 | 2.29E−05 |
| Cdk14 | 85 | −1.07 | −1.26 | 6.08E−05 | 0.000815 |
| Shank1 | 125.8 | −1.07 | −1.41 | 0.000743 | 0.006595 |
| Ighv1-43 | 24.1 | −1.07 | −1.92 | 0.004221 | 0.026637 |
| Ctsf | 383.8 | −1.08 | −1.16 | 4.68E−09 | 2.1E−07 |
| Tbxas1 | 561.4 | −1.08 | −1.17 | 1.11E−07 | 3.55E−06 |
| Alpk1 | 263.7 | −1.08 | −1.18 | 2.53E−07 | 7.29E−06 |
| Sccpdh | 123.3 | −1.08 | −1.17 | 6.21E−07 | 1.61E−05 |
| Lpl | 1230.2 | −1.08 | −1.21 | 3.08E−06 | 6.34E−05 |
| Stab2 | 1255.1 | −1.08 | −1.22 | 7.89E−06 | 0.000144 |
| Apol9b | 51.1 | −1.08 | −1.23 | 9.87E−06 | 0.000174 |
| Gm4258 | 54.5 | −1.08 | −1.25 | 1.92E−05 | 0.000305 |
| H2-Ea-ps | 7790.9 | −1.08 | −1.36 | 0.000366 | 0.003633 |
| Ciita | 568.4 | −1.08 | −1.39 | 0.000641 | 0.005863 |
| Igkv4-70 | 65.4 | −1.08 | −1.47 | 0.001124 | 0.009265 |
| Ighv1-42 | 5.8 | −1.08 | −3.23 | 0.002259 | 0.016093 |
| Igkv4-50 | 54.1 | −1.08 | −1.76 | 0.003011 | 0.020314 |
| Cd86 | 1049.2 | −1.09 | −1.15 | 3.61E−11 | 2.81E−09 |
| Fam43a | 1414.6 | −1.09 | −1.18 | 3.94E−08 | 1.39E−06 |
| Smagp | 295.1 | −1.09 | −1.2 | 3.82E−07 | 1.05E−05 |
| Zfhx3 | 113.3 | −1.09 | −1.24 | 6.09E−06 | 0.000115 |
| Trim30b | 52.4 | −1.09 | −1.36 | 0.000154 | 0.001778 |
| Fst | 29.8 | −1.09 | −1.52 | 0.000952 | 0.008075 |
| Pigr | 17.1 | −1.09 | −1.7 | 0.002701 | 0.018704 |
| 2010007H06Rik | 20.7 | −1.09 | −1.99 | 0.003403 | 0.022397 |
| Rgl1 | 1317.4 | −1.1 | −1.2 | 8.26E−08 | 2.73E−06 |
| Clec4a2 | 262.2 | −1.1 | −1.26 | 1.09E−05 | 0.00019 |
| Adhfe1 | 58 | −1.1 | −1.3 | 2.42E−05 | 0.000374 |
| Iglv3 | 74.4 | −1.1 | −1.68 | 0.001978 | 0.014396 |
| C130050O18Rik | 95.9 | −1.11 | −1.26 | 1.45E−06 | 3.32E−05 |
| Slpi | 1443.3 | −1.11 | −1.38 | 0.00016 | 0.001838 |
| Mybpc3 | 45.4 | −1.11 | −1.44 | 0.000231 | 0.002478 |
| Adgrl3 | 33.2 | −1.11 | −1.64 | 0.001005 | 0.008438 |
| Gm9530 | 13.7 | −1.11 | −1.85 | 0.001935 | 0.014156 |
| Igkv17-127 | 57.8 | −1.11 | −2.86 | 0.003056 | 0.020527 |
| 4930426D05Rik | 77.3 | −1.11 | −2.7 | 0.003313 | 0.021946 |
| Fcer2a | 783 | −1.11 | −2.3 | 0.003321 | 0.021992 |
| Nfam1 | 713.9 | −1.12 | −1.19 | 1.76E−10 | 1.14E−08 |
| Epb41l3 | 649.5 | −1.12 | −1.23 | 9.17E−08 | 2.97E−06 |
| Naip5 | 181.7 | −1.12 | −1.27 | 2.06E−06 | 4.5E−05 |
| Gas6 | 210.8 | −1.12 | −1.29 | 1.06E−05 | 0.000185 |
| A530099J19Rik | 38 | −1.12 | −1.84 | 0.001581 | 0.012052 |
| 0610040J01Rik | 15.3 | −1.12 | −1.97 | 0.001998 | 0.014505 |
| Gm17999 | 9.8 | −1.12 | −2.45 | 0.00274 | 0.018921 |
| Gm14963 | 10.5 | −1.12 | −1.98 | 0.002748 | 0.018963 |
| Tal2 | 7.8 | −1.12 | −2.87 | 0.002908 | 0.019726 |
| Gm10605 | 75.3 | −1.13 | −1.35 | 2.32E−05 | 0.000361 |
| Abcd2 | 85.6 | −1.13 | −1.35 | 3.96E−05 | 0.000563 |
| Pcolce2 | 67.5 | −1.13 | −1.42 | 9.12E−05 | 0.001153 |
| Pigz | 58.4 | −1.13 | −1.4 | 0.000141 | 0.001652 |
| Trf | 681.7 | −1.13 | −1.49 | 0.000353 | 0.003532 |
| A930030B08Rik | 34.4 | −1.13 | −1.59 | 0.000525 | 0.004961 |
| Mir5107 | 156.7 | −1.13 | −1.77 | 0.001511 | 0.011598 |
| Kcnj2 | 25 | −1.13 | −2 | 0.001945 | 0.014221 |
| Tlr7 | 305.4 | −1.14 | −1.22 | 3.03E−09 | 1.42E−07 |
| Igsf6 | 661.8 | −1.14 | −1.24 | 3.88E−08 | 1.38E−06 |
| Lrrc25 | 483.1 | −1.14 | −1.28 | 6.57E−07 | 1.69E−05 |
| Ptpro | 88.1 | −1.14 | −1.29 | 1.06E−06 | 2.52E−05 |
| Tppp | 109.8 | −1.14 | −1.29 | 1.47E−06 | 3.35E−05 |
| Dll4 | 78 | −1.14 | −1.29 | 2.41E−06 | 5.14E−05 |
| Snn | 825.3 | −1.14 | −1.31 | 3.98E−06 | 7.95E−05 |
| Pdzd2 | 59.3 | −1.14 | −1.35 | 1.39E−05 | 0.000234 |
| Erbb2 | 135 | −1.14 | −1.33 | 1.55E−05 | 0.000255 |
| Ctnnd2 | 131.9 | −1.14 | −1.44 | 0.000131 | 0.001557 |
| Gm15930 | 22 | −1.14 | −1.73 | 0.001034 | 0.008627 |
| Igkv8-30 | 318.2 | −1.14 | −1.89 | 0.002044 | 0.014786 |
| Blk | 523.7 | −1.14 | −1.98 | 0.002156 | 0.015487 |
| Cyp4f37 | 6.6 | −1.14 | −2.97 | 0.002672 | 0.018535 |
| Tcf4 | 708.9 | −1.15 | −1.26 | 2.31E−08 | 8.84E−07 |
| C1qb | 6167.6 | −1.15 | −1.28 | 3.36E−07 | 9.48E−06 |
| Ccnd1 | 258 | −1.15 | −1.29 | 3.86E−07 | 1.05E−05 |
| Cfap61 | 26 | −1.15 | −1.82 | 0.001029 | 0.008606 |
| Hepacam2 | 34.3 | −1.15 | −2.23 | 0.00188 | 0.013838 |
| Gm15880 | 16.2 | −1.15 | −2.16 | 0.002139 | 0.015378 |
| Clec4a4 | 10.7 | −1.15 | −2.6 | 0.002248 | 0.01604 |
| Fmnl2 | 329.1 | −1.16 | −1.29 | 1.74E−07 | 5.28E−06 |
| Pdgfc | 56.2 | −1.16 | −1.45 | 8.78E−05 | 0.001115 |
| Pde8b | 30.7 | −1.16 | −1.81 | 0.000914 | 0.007828 |
| Gm5466 | 17.5 | −1.16 | −1.97 | 0.001427 | 0.011109 |
| Lima1 | 405.9 | −1.17 | −1.27 | 4.02E−09 | 1.82E−07 |
| Slc16a7 | 76.5 | −1.17 | −1.39 | 1.46E−05 | 0.000243 |
| Snx24 | 100.2 | −1.17 | −1.4 | 1.79E−05 | 0.000288 |
| Fcrl1 | 520.9 | −1.17 | −1.52 | 0.000191 | 0.002126 |
| Plxnb3 | 46.1 | −1.17 | −1.66 | 0.000571 | 0.00531 |
| Gm36937 | 12.9 | −1.17 | −2.24 | 0.001847 | 0.013668 |
| Igkv5-39 | 123.1 | −1.17 | −2.24 | 0.00205 | 0.014817 |
| Tnfrsf21 | 641.6 | −1.18 | −1.26 | 5.23E−11 | 3.91E−09 |
| Syk | 3325.1 | −1.18 | −1.29 | 1.05E−08 | 4.34E−07 |
| Cmbl | 316.4 | −1.18 | −1.31 | 3.55E−07 | 9.91E−06 |
| AC166361.2 | 101 | −1.18 | −1.36 | 5.35E−07 | 1.42E−05 |
| Stac2 | 76.6 | −1.18 | −1.55 | 0.000206 | 0.002258 |
| Ighv2-6 | 141.1 | −1.18 | −1.53 | 0.000266 | 0.002791 |
| Igkv4-63 | 73.9 | −1.18 | −1.74 | 0.000748 | 0.006628 |
| Igkv9-124 | 50.6 | −1.18 | −2.27 | 0.001681 | 0.012661 |
| Mpeg1 | 6302.5 | −1.19 | −1.28 | 3.51E−10 | 2.08E−08 |
| Siglech | 840.4 | −1.19 | −1.32 | 1.89E−07 | 5.64E−06 |
| Ccdc148 | 86.4 | −1.19 | −1.48 | 4.04E−05 | 0.000573 |
| Blnk | 622 | −1.19 | −1.59 | 0.000221 | 0.002396 |
| Ighv1-4 | 158 | −1.19 | −1.8 | 0.000906 | 0.007795 |
| Siglecg | 751.8 | −1.19 | −1.93 | 0.001034 | 0.008627 |
| Slc6a1 | 17.6 | −1.19 | −2.21 | 0.001218 | 0.009819 |
| Ighv9-2 | 13.7 | −1.19 | −2.05 | 0.001368 | 0.010761 |
| Igkv4-58 | 47 | −1.19 | −2.02 | 0.001407 | 0.011025 |
| Fam213b | 477.2 | −1.2 | −1.33 | 6.6E−08 | 2.24E−06 |
| Gm13710 | 186.2 | −1.2 | −1.39 | 3.19E−06 | 6.52E−05 |
| S1pr3 | 58.4 | −1.2 | −1.43 | 8.88E−06 | 0.00016 |
| Tecta | 37.6 | −1.2 | −1.56 | 0.000104 | 0.001281 |
| Fam135a | 30.1 | −1.2 | −1.66 | 0.000222 | 0.002413 |
| Gbgt1 | 24 | −1.2 | −1.71 | 0.000334 | 0.003374 |
| AC153955.5 | 18.5 | −1.2 | −1.86 | 0.000622 | 0.005722 |
| Trim7 | 268.2 | −1.2 | −1.88 | 0.000812 | 0.007083 |
| Igkv6-23 | 42.9 | −1.2 | −1.99 | 0.001136 | 0.009317 |
| Map3k9 | 162.8 | −1.21 | −1.3 | 9.73E−11 | 6.66E−09 |
| Fndc7 | 27.5 | −1.21 | −1.78 | 0.00023 | 0.002473 |
| Apoe | 8629.4 | −1.22 | −1.32 | 4.19E−10 | 2.45E−08 |
| Sirpa | 4992.1 | −1.22 | −1.35 | 2.63E−08 | 9.78E−07 |
| Cd300a | 1109.4 | −1.22 | −1.35 | 3.42E−08 | 1.24E−06 |
| Cystm1 | 143.7 | −1.22 | −1.4 | 5.65E−07 | 1.49E−05 |
| Snta1 | 208.6 | −1.22 | −1.4 | 1.01E−06 | 2.43E−05 |
| Trpv4 | 50.5 | −1.22 | −1.54 | 2.56E−05 | 0.000392 |
| Lifr | 153.4 | −1.22 | −1.49 | 2.67E−05 | 0.000404 |
| Pcdhgb4 | 14.2 | −1.22 | −1.79 | 0.00053 | 0.005005 |
| Sdc3 | 7230.4 | −1.23 | −1.34 | 1.71E−09 | 8.52E−08 |
| Marcks | 880 | −1.23 | −1.39 | 3.81E−07 | 1.04E−05 |
| Flt3 | 441.7 | −1.23 | −1.43 | 2.6E−06 | 5.48E−05 |
| Wdfy4 | 2179.7 | −1.23 | −1.51 | 1.94E−05 | 0.000307 |
| Igkv5-45 | 19.6 | −1.23 | −2.23 | 0.001131 | 0.009302 |
| H2-Ob | 1767 | −1.24 | −1.32 | 1.21E−12 | 1.33E−10 |
| Fgd2 | 1084.5 | −1.24 | −1.37 | 3.13E−08 | 1.14E−06 |
| Dmpk | 201.4 | −1.24 | −1.43 | 5.01E−07 | 1.34E−05 |
| Ttc12 | 74.5 | −1.24 | −1.49 | 8.57E−06 | 0.000154 |
| AC153955.2 | 13.5 | −1.24 | −3.15 | 0.000817 | 0.00712 |
| Dennd5b | 158.4 | −1.24 | −2.45 | 0.001058 | 0.008786 |
| Ppfibp2 | 358.3 | −1.25 | −1.4 | 4.67E−08 | 1.62E−06 |
| Gm15922 | 138.1 | −1.25 | −1.47 | 2E−06 | 4.38E−05 |
| Slc1a3 | 115.6 | −1.25 | −1.56 | 1.64E−05 | 0.000269 |
| Fam71a | 57.8 | −1.25 | −1.63 | 5E−05 | 0.000691 |
| Btnl4 | 51.4 | −1.25 | −1.87 | 0.000251 | 0.002666 |
| Plxnb2 | 2017.4 | −1.26 | −1.36 | 2.1E−10 | 1.33E−08 |
| Mafb | 1846.3 | −1.26 | −1.39 | 6.8E−09 | 2.94E−07 |
| Scamp5 | 313.9 | −1.26 | −1.38 | 8.04E−09 | 3.4E−07 |
| Lpcat2 | 297.8 | −1.26 | −1.41 | 1.94E−08 | 7.66E−07 |
| Prkcg | 96.9 | −1.26 | −1.45 | 1.88E−07 | 5.63E−06 |
| Cd300c2 | 673.6 | −1.26 | −1.44 | 3.62E−07 | 1E−05 |
| Cecr6 | 48.6 | −1.26 | −1.51 | 3.93E−06 | 7.85E−05 |
| Scn3a | 17.9 | −1.26 | −1.92 | 0.000287 | 0.002968 |
| A4galt | 38.5 | −1.26 | −1.89 | 0.000339 | 0.003419 |
| Unc5a | 20.3 | −1.26 | −2.62 | 0.000665 | 0.006038 |
| Dock4 | 301.1 | −1.27 | −1.39 | 2.01E−10 | 1.28E−08 |
| C1qc | 6828 | −1.27 | −1.42 | 4.33E−08 | 1.52E−06 |
| Zfp366 | 189.3 | −1.27 | −1.44 | 6.67E−08 | 2.26E−06 |
| Samd4 | 57.2 | −1.27 | −1.61 | 1.4E−05 | 0.000235 |
| Cd209d | 96.9 | −1.27 | −1.89 | 0.000206 | 0.002258 |
| Ifi207 | 440.3 | −1.28 | −1.42 | 6.19E−09 | 2.71E−07 |
| Glis3 | 183.7 | −1.28 | −1.48 | 3.46E−07 | 9.69E−06 |
| Gpc4 | 55.1 | −1.28 | −1.58 | 6.38E−06 | 0.00012 |
| Tgfb2 | 26.2 | −1.28 | −1.82 | 7.16E−05 | 0.000937 |
| Ffar1 | 97.9 | −1.28 | −1.83 | 0.000194 | 0.002157 |
| Epha2 | 215.5 | −1.29 | −1.42 | 1.19E−09 | 6.2E−08 |
| Cd5l | 2824.6 | −1.29 | −1.41 | 1.31E−09 | 6.73E−08 |
| Nxpe5 | 131.7 | −1.29 | −1.49 | 3.09E−07 | 8.77E−06 |
| Gm33280 | 43.4 | −1.29 | −1.78 | 7E−05 | 0.000918 |
| Gm15448 | 29.6 | −1.29 | −1.86 | 0.000127 | 0.00152 |
| Tlr13 | 195.6 | −1.3 | −1.4 | 5.26E−12 | 5.13E−10 |
| Kcnk13 | 70 | −1.3 | −1.51 | 2.63E−07 | 7.54E−06 |
| Rasal2 | 84.5 | −1.3 | −1.53 | 4.32E−07 | 1.17E−05 |
| Cd300c | 47.9 | −1.3 | −1.67 | 1.67E−05 | 0.000273 |
| Gm14137 | 13.8 | −1.3 | −3.35 | 0.000624 | 0.005734 |
| 6430548M08Rik | 386.7 | −1.31 | −1.4 | 5.27E−13 | 6.58E−11 |
| C2 | 119.2 | −1.31 | −1.41 | 2.4E−11 | 1.95E−09 |
| Fgd4 | 96.4 | −1.31 | −1.63 | 4.58E−06 | 8.9E−05 |
| Gm14221 | 27.8 | −1.31 | −2.05 | 0.00018 | 0.002021 |
| Gm28050 | 11.2 | −1.31 | −2.15 | 0.000277 | 0.002883 |
| Il22ra2 | 7.5 | −1.31 | −3.06 | 0.000546 | 0.005125 |
| Rims3 | 1029.1 | −1.32 | −1.41 | 7.3E−14 | 1.16E−11 |
| I830077J02Rik | 399 | −1.32 | −1.41 | 1.46E−12 | 1.59E−10 |
| Cfp | 5197 | −1.32 | −1.44 | 4.78E−10 | 2.76E−08 |
| Sirpb1c | 21.5 | −1.32 | −1.75 | 2.9E−05 | 0.000432 |
| Adam11 | 386.6 | −1.33 | −1.39 | 0 | 1E−15 |
| Hpgds | 565.3 | −1.33 | −1.43 | 5.79E−12 | 5.61E−10 |
| Hdac9 | 184.1 | −1.33 | −1.49 | 8.39E−09 | 3.53E−07 |
| Clec4a1 | 389.4 | −1.33 | −1.52 | 6.73E−08 | 2.27E−06 |
| Gm10552 | 10.1 | −1.33 | −2.86 | 0.000376 | 0.003714 |
| Abcg3 | 1117.1 | −1.34 | −1.42 | 6E−15 | 1.18E−12 |
| Slc7a7 | 700.4 | −1.34 | −1.45 | 1.16E−11 | 1.03E−09 |
| Amz1 | 508.4 | −1.34 | −1.46 | 4.31E−11 | 3.29E−09 |
| Pirb | 1339.8 | −1.34 | −1.49 | 2.08E−09 | 1.01E−07 |
| Cd14 | 1110.8 | −1.34 | −1.51 | 5.3E−09 | 2.36E−07 |
| Cyp27a1 | 464.9 | −1.34 | −1.52 | 1.71E−08 | 6.82E−07 |
| Clec4b1 | 78.8 | −1.34 | −1.73 | 1.22E−05 | 0.000208 |
| Rasgef1b | 1868 | −1.35 | −1.41 | 0 | 1E−15 |
| Jup | 1184.5 | −1.35 | −1.44 | 1.31E−13 | 1.98E−11 |
| Grk3 | 1230.7 | −1.35 | −1.46 | 3.6E−12 | 3.63E−10 |
| C1qa | 7016.9 | −1.35 | −1.51 | 8.15E−09 | 3.43E−07 |
| Dgki | 117.3 | −1.35 | −1.56 | 3.91E−08 | 1.38E−06 |
| Agap1 | 495.9 | −1.36 | −1.51 | 6.16E−10 | 3.46E−08 |
| Timd4 | 240.3 | −1.36 | −1.53 | 7.81E−09 | 3.33E−07 |
| Ighv1-7 | 114.3 | −1.36 | −1.76 | 1.71E−05 | 0.000278 |
| Tlr8 | 86.3 | −1.37 | −1.62 | 2.21E−07 | 6.48E−06 |
| Zfp608 | 304 | −1.38 | −1.52 | 1.86E−10 | 1.19E−08 |
| Kif26a | 36.7 | −1.38 | −2.16 | 8.08E−05 | 0.001037 |
| Reln | 18.1 | −1.38 | −2.59 | 0.000204 | 0.002246 |
| Dhtkd1 | 7 | −1.38 | −3.09 | 0.000253 | 0.002684 |
| Gm15848 | 16.2 | −1.38 | −3.38 | 0.00027 | 0.002823 |
| Prkar1b | 244.4 | −1.39 | −1.54 | 8.05E−11 | 5.67E−09 |
| Slc16a9 | 181.6 | −1.39 | −1.59 | 1.74E−08 | 6.93E−07 |
| Gfra4 | 95 | −1.39 | −1.61 | 2.35E−07 | 6.87E−06 |
| Pid1 | 185.8 | −1.39 | −1.68 | 7.4E−07 | 1.86E−05 |
| Apoc1 | 69.6 | −1.39 | −1.83 | 1.3E−05 | 0.00022 |
| Scn4a | 77.9 | −1.39 | −2.02 | 7.44E−05 | 0.000968 |
| Ptgs1 | 1227.5 | −1.4 | −1.51 | 1.12E−12 | 1.26E−10 |
| AC125351.1 | 47.4 | −1.4 | −1.8 | 2.46E−06 | 5.24E−05 |
| Fstl4 | 14.3 | −1.4 | −2.76 | 0.000124 | 0.001486 |
| Plbd1 | 1881.3 | −1.41 | −1.54 | 2.06E−11 | 1.72E−09 |
| Gm15931 | 220.2 | −1.41 | −1.6 | 2.87E−09 | 1.36E−07 |
| Tcf7l2 | 320.1 | −1.41 | −1.64 | 2.26E−08 | 8.71E−07 |
| Capn9 | 80.5 | −1.41 | −1.69 | 1.32E−07 | 4.12E−06 |
| March1 | 226 | −1.41 | −1.68 | 2.64E−07 | 7.58E−06 |
| Mcc | 21.2 | −1.41 | −2.32 | 6.53E−05 | 0.000866 |
| Etv1 | 13.7 | −1.41 | −2.38 | 6.82E−05 | 0.000895 |
| AC160028.2 | 11.6 | −1.41 | −3.13 | 0.000179 | 0.002013 |
| Nav1 | 825.1 | −1.42 | −1.59 | 2.42E−10 | 1.5E−08 |
| Igf1 | 580.6 | −1.42 | −1.61 | 3.17E−09 | 1.47E−07 |
| Cpne8 | 29 | −1.42 | −1.91 | 1.06E−05 | 0.000185 |
| Csf1r | 9890.8 | −1.43 | −1.54 | 1.16E−13 | 1.79E−11 |
| Hpgd | 485.4 | −1.43 | −1.57 | 1.74E−11 | 1.47E−09 |
| Tmem26 | 319.6 | −1.43 | −1.58 | 5.08E−11 | 3.83E−09 |
| Prss29 | 62.2 | −1.43 | −1.75 | 1.22E−07 | 3.86E−06 |
| Ighv5-2 | 17.8 | −1.43 | −3.08 | 0.000154 | 0.001777 |
| Cd209b | 9.6 | −1.43 | −3.28 | 0.00016 | 0.001835 |
| Axl | 7411.5 | −1.44 | −1.56 | 4.08E−13 | 5.33E−11 |
| Tbc1d9 | 512.6 | −1.44 | −1.57 | 1.2E−12 | 1.33E−10 |
| Plpp3 | 349.2 | −1.44 | −1.62 | 4.78E−10 | 2.76E−08 |
| Clec4a3 | 317.3 | −1.44 | −1.61 | 4.9E−10 | 2.82E−08 |
| Scnn1a | 79.5 | −1.44 | −1.72 | 1.08E−07 | 3.49E−06 |
| Jhy | 47.7 | −1.44 | −2.65 | 9.59E−05 | 0.001207 |
| Nr1d1 | 927.5 | −1.45 | −1.54 | 0 | 1.35E−13 |
| Itga9 | 882.5 | −1.45 | −1.6 | 8.13E−12 | 7.66E−10 |
| Slc45a3 | 1350.4 | −1.45 | −1.59 | 2.01E−11 | 1.68E−09 |
| Clec4n | 902.8 | −1.45 | −1.62 | 1.39E−10 | 9.13E−09 |
| Lrp4 | 160.7 | −1.45 | −1.7 | 9.85E−09 | 4.07E−07 |
| Cacna1e | 284.9 | −1.45 | −1.73 | 1.14E−07 | 3.62E−06 |
| Mmp12 | 54 | −1.45 | −2.08 | 1.42E−05 | 0.000237 |
| P2ry13 | 230.8 | −1.46 | −1.59 | 5.57E−13 | 6.92E−11 |
| Nuak1 | 190.3 | −1.46 | −1.6 | 6.64E−13 | 7.9E−11 |
| Adap2 | 260.1 | −1.46 | −1.72 | 4.52E−08 | 1.58E−06 |
| Adgrg6 | 43.3 | −1.46 | −1.97 | 4.05E−06 | 8.05E−05 |
| Kcnj10 | 1374.3 | −1.47 | −1.58 | 4E−15 | 8.36E−13 |
| Ppp1r9a | 128.9 | −1.47 | −1.8 | 1.43E−07 | 4.44E−06 |
| Rab30 | 116 | −1.47 | −2.11 | 1.86E−05 | 0.000297 |
| Gfra2 | 1060.6 | −1.48 | −1.62 | 4.86E−12 | 4.82E−10 |
| St6galnac2 | 280.3 | −1.48 | −1.68 | 7.6E−10 | 4.15E−08 |
| 2900052N01Rik | 111.1 | −1.48 | −1.76 | 1.26E−08 | 5.15E−07 |
| Slc8a1 | 117.6 | −1.48 | −1.88 | 7.25E−07 | 1.83E−05 |
| Ighv1-39 | 28.6 | −1.48 | −2.7 | 9.84E−05 | 0.001231 |
| Gpr137b | 186 | −1.49 | −1.71 | 1.37E−09 | 7.01E−08 |
| Trpm2 | 1260.6 | −1.5 | −1.66 | 3.19E−12 | 3.31E−10 |
| Aatk | 130.6 | −1.5 | −1.7 | 7.89E−11 | 5.6E−09 |
| A530064D06Rik | 30 | −1.5 | −1.98 | 7.1E−07 | 1.8E−05 |
| Pyroxd2 | 242.8 | −1.51 | −1.68 | 9.76E−12 | 9.04E−10 |
| Hcar2 | 808.2 | −1.51 | −1.74 | 8.34E−10 | 4.5E−08 |
| Catsperg2 | 14.8 | −1.51 | −2.17 | 9.58E−06 | 0.00017 |
| Slc40a1 | 4625.9 | −1.53 | −1.66 | 1E−14 | 1.89E−12 |
| Mmp19 | 385.3 | −1.53 | −2.04 | 1.82E−06 | 4.01E−05 |
| Mpzl1 | 469.8 | −1.55 | −1.7 | 2.45E−13 | 3.38E−11 |
| Dnah2 | 262.4 | −1.55 | −1.85 | 3.81E−09 | 1.74E−07 |
| Igkv6-15 | 223 | −1.55 | −2.65 | 3.22E−05 | 0.00047 |
| Cadm1 | 1067.9 | −1.56 | −1.72 | 1.47E−13 | 2.19E−11 |
| Ear2 | 200.6 | −1.56 | −1.76 | 4.64E−11 | 3.53E−09 |
| Cd302 | 387 | −1.57 | −1.73 | 3.57E−13 | 4.7E−11 |
| Lilra6 | 252.1 | −1.58 | −1.75 | 2.95E−13 | 3.94E−11 |
| Cd163 | 1248.1 | −1.58 | −1.77 | 1.19E−11 | 1.05E−09 |
| Lilra5 | 286.1 | −1.58 | −1.8 | 2.95E−11 | 2.34E−09 |
| Treml4 | 1601.2 | −1.59 | −1.7 | 0 | 2E−15 |
| Slc11a1 | 1813.6 | −1.59 | −1.72 | 4E−15 | 8.92E−13 |
| Itgb5 | 955.2 | −1.6 | −1.73 | 1E−15 | 2.17E−13 |
| Adgre1 | 1926.6 | −1.6 | −1.75 | 1.8E−14 | 3.26E−12 |
| Rgl3 | 91.8 | −1.6 | −1.86 | 2.53E−10 | 1.57E−08 |
| Ccr3 | 902.9 | −1.61 | −1.75 | 2E−15 | 5.62E−13 |
| Siglec1 | 622.7 | −1.61 | −1.78 | 1.01E−12 | 1.15E−10 |
| Slco2b1 | 818.9 | −1.62 | −1.75 | 1E−15 | 1.75E−13 |
| Apod | 9.3 | −1.62 | −3.32 | 2E−05 | 0.000315 |
| C6 | 1221.3 | −1.63 | −1.78 | 1.5E−14 | 2.85E−12 |
| Sirpb1b | 32.1 | −1.64 | −2.12 | 2.61E−08 | 9.73E−07 |
| Sirpb1a | 105.6 | −1.65 | −1.88 | 3.33E−12 | 3.42E−10 |
| Lhfp | 173.9 | −1.66 | −1.92 | 3.96E−11 | 3.03E−09 |
| Hmox1 | 16492.8 | −1.66 | −2.02 | 3.06E−09 | 1.43E−07 |
| Gm2762 | 38.5 | −1.66 | −1.94 | 2.82E−08 | 1.04E−06 |
| Pilra | 854.5 | −1.67 | −1.82 | 1E−15 | 1.75E−13 |
| Angptl7 | 118.5 | −1.67 | −1.89 | 2.22E−12 | 2.38E−10 |
| Matn2 | 73.1 | −1.67 | −2 | 5.51E−10 | 3.12E−08 |
| Mertk | 1388.9 | −1.67 | −2.06 | 6.31E−09 | 2.76E−07 |
| Cxcl1 | 159.6 | −1.67 | −2.1 | 1.42E−08 | 5.76E−07 |
| Adamdec1 | 659.5 | −1.68 | −1.83 | 0 | 1.2E−13 |
| Tenm4 | 307.5 | −1.68 | −1.89 | 1.99E−13 | 2.82E−11 |
| Bank1 | 715.4 | −1.68 | −1.96 | 5.8E−11 | 4.31E−09 |
| Gm5150 | 271.8 | −1.69 | −1.83 | 0 | 9E−15 |
| Igkv3-7 | 151.3 | −1.69 | −3.52 | 8.26E−06 | 0.000149 |
| Aspa | 38.6 | −1.7 | −2.48 | 1.27E−07 | 3.99E−06 |
| Gdf15 | 168.7 | −1.71 | −2.33 | 5.3E−08 | 1.82E−06 |
| Adam22 | 142.7 | −1.72 | −2.24 | 2.59E−08 | 9.71E−07 |
| Vcam1 | 9147.6 | −1.73 | −1.91 | 6E−15 | 1.2E−12 |
| Fjx1 | 45.8 | −1.73 | −3.32 | 1.23E−06 | 2.86E−05 |
| Paqr9 | 427.9 | −1.74 | −1.96 | 4E−14 | 6.63E−12 |
| Slc22a23 | 202.4 | −1.75 | −1.94 | 0 | 7.9E−14 |
| Dlc1 | 173.9 | −1.75 | −2.01 | 6.48E−13 | 7.83E−11 |
| Sema6a | 113.4 | −1.75 | −2.11 | 8.35E−11 | 5.81E−09 |
| Snx31 | 36.9 | −1.75 | −2.37 | 2.42E−08 | 9.17E−07 |
| Mrc1 | 3441.3 | −1.76 | −1.96 | 3E−15 | 6.66E−13 |
| Pira2 | 53.5 | −1.76 | −2.34 | 7.75E−09 | 3.32E−07 |
| Siglece | 886.5 | −1.77 | −1.91 | 0 | 0 |
| Il1a | 348.3 | −1.77 | −2.22 | 7.34E−10 | 4.02E−08 |
| Cecr2 | 73.9 | −1.77 | −2.86 | 9.93E−07 | 2.39E−05 |
| Pilrb1 | 346.4 | −1.78 | −2.02 | 4E−14 | 6.68E−12 |
| Stra6l | 77.8 | −1.78 | −2.07 | 1.68E−12 | 1.8E−10 |
| Sash1 | 878.3 | −1.8 | −2.01 | 1E−15 | 1.46E−13 |
| Hfe | 584 | −1.81 | −2.02 | 2E−15 | 5.33E−13 |
| Kirrel3 | 96.4 | −1.82 | −2.06 | 1E−14 | 1.9E−12 |
| Gm14548 | 229.3 | −1.82 | −2.13 | 2.57E−12 | 2.7E−10 |
| Gpd1 | 933.9 | −1.84 | −2.05 | 1E−15 | 1.85E−13 |
| Ace | 93.4 | −1.84 | −2.73 | 5.03E−08 | 1.75E−06 |
| Pilrb2 | 349.2 | −1.85 | −2.1 | 4E−15 | 9.43E−13 |
| Spic | 2050.5 | −1.86 | −2.04 | 0 | 1E−15 |
| Lrp1 | 4560.8 | −1.86 | −2.08 | 0 | 6.6E−14 |
| Slc15a2 | 279.3 | −1.89 | −2.18 | 1.7E−14 | 3.03E−12 |
| Vstm4 | 221 | −1.89 | −2.18 | 2E−14 | 3.63E−12 |
| Abcc3 | 2372 | −1.9 | −2.04 | 0 | 0 |
| Cd209a | 127.2 | −1.9 | −2.44 | 1.68E−10 | 1.09E−08 |
| Cd300e | 272.8 | −1.91 | −2.34 | 7.55E−12 | 7.15E−10 |
| Sned1 | 230.5 | −1.92 | −2.29 | 9.22E−13 | 1.07E−10 |
| Myo18b | 126.8 | −1.96 | −2.34 | 1.63E−13 | 2.4E−11 |
| Ptprm | 389.7 | −1.98 | −2.21 | 0 | 0 |
| Kcnj16 | 221.3 | −1.98 | −2.22 | 0 | 4E−15 |
| Gm6377 | 147.2 | −1.99 | −2.51 | 8.92E−12 | 8.32E−10 |
| Mybpc2 | 29.1 | −1.99 | −3.53 | 2.54E−08 | 9.54E−07 |
| Sema6d | 939.7 | −2.01 | −2.3 | 0 | 2.2E−14 |
| Cbr2 | 76.5 | −2.01 | −2.9 | 8.13E−10 | 4.42E−08 |
| Itgad | 5960.5 | −2.04 | −2.25 | 0 | 0 |
| Adam23 | 301.5 | −2.04 | −2.27 | 0 | 0 |
| Postn | 510.9 | −2.04 | −2.28 | 0 | 1E−15 |
| Cbln1 | 33.6 | −2.04 | −2.89 | 1.25E−10 | 8.28E−09 |
| Rnf150 | 123.1 | −2.07 | −2.32 | 0 | 0 |
| Hs3st2 | 297.9 | −2.13 | −2.53 | 0 | 1.3E−13 |
| Fcna | 5828.3 | −2.2 | −2.39 | 0 | 0 |
| Col14a1 | 699.7 | −2.25 | −2.55 | 0 | 0 |
| Akr1b7 | 148.5 | −2.26 | −2.71 | 0 | 9E−15 |
| Wisp2 | 41.1 | −2.27 | −3.56 | 1.66E−11 | 1.41E−09 |
| Adgre4 | 851.8 | −2.37 | −2.61 | 0 | 0 |
| Nfasc | 204.4 | −2.38 | −2.98 | 0 | 1.5E−14 |

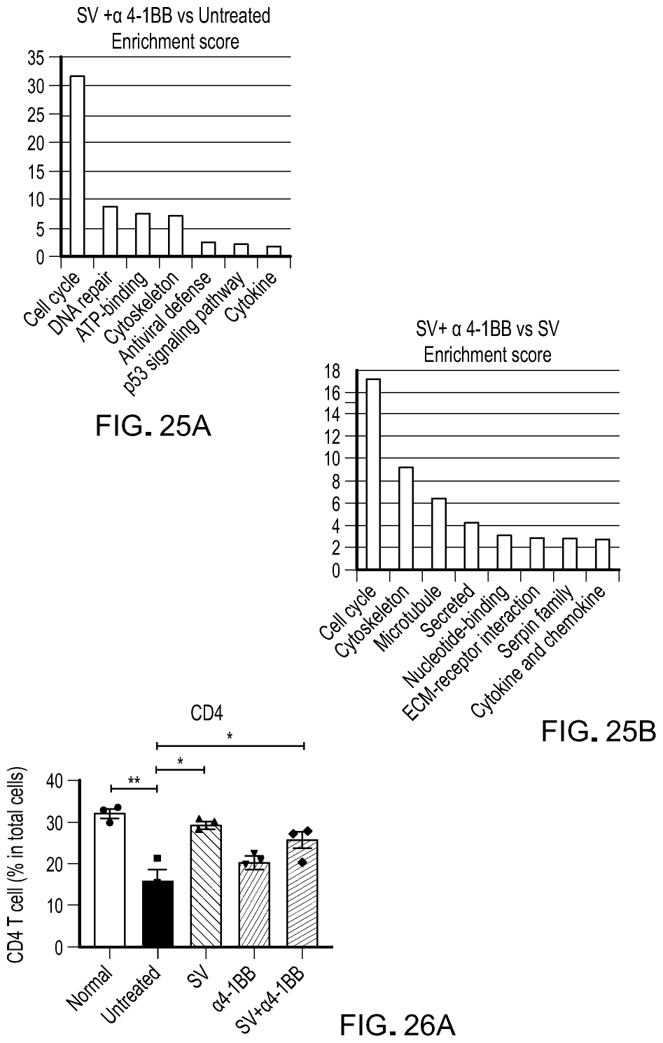

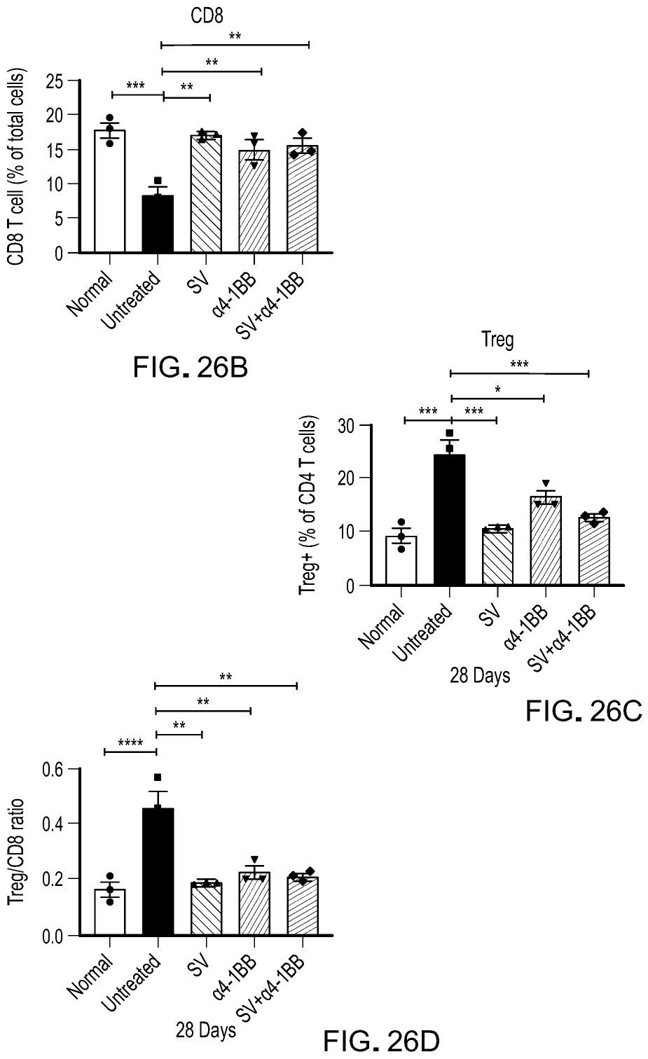

Next, NIH DAVID analysis using the upregulated gene list was run. In both comparisons, cell cycle genes upregulation is the highest enrichment cluster [although SV+α4-1BB mAb vs. SV has a lower enrichment score compared with SV plus α4-1BB mAb vs. untreated samples (FIGS. 23B and 25). This indicates that SV+α4-1BB mAb induced more potent T cell cycle progression compared with SV only. T cell proliferation is critical for an effective anti-tumor response against A20 lymphoma. The CD4/CD8 T cell ratio in untreated mice decreased markedly by day 28 after tumor inoculation (FIG. 26A-26B). In addition, Treg/CD8 T cell ratio increased by day 28, indicating impairment of T cell function (FIG. 26C-26D). In other groups the T cell ratio remained constant due to proliferation.

CD69 is the earliest marker of immune system activation. SV plus α4-1BB mAb treatment synergistically upregulated CD69 on day 2 (FIG. 23D). Additionally, KEGG GSEA indicates that T cell receptor signaling gene sets were enriched when comparing SV+α4-1BB vs untreated samples (enrichment score=0.35, Normalized Enrichment Score (NES)=1.56, FDR q value=0.17, nominal p value=0) (FIG. 23E).

SV Plus α4-1BB mAb Stimulated Cytotoxic T Cell Function

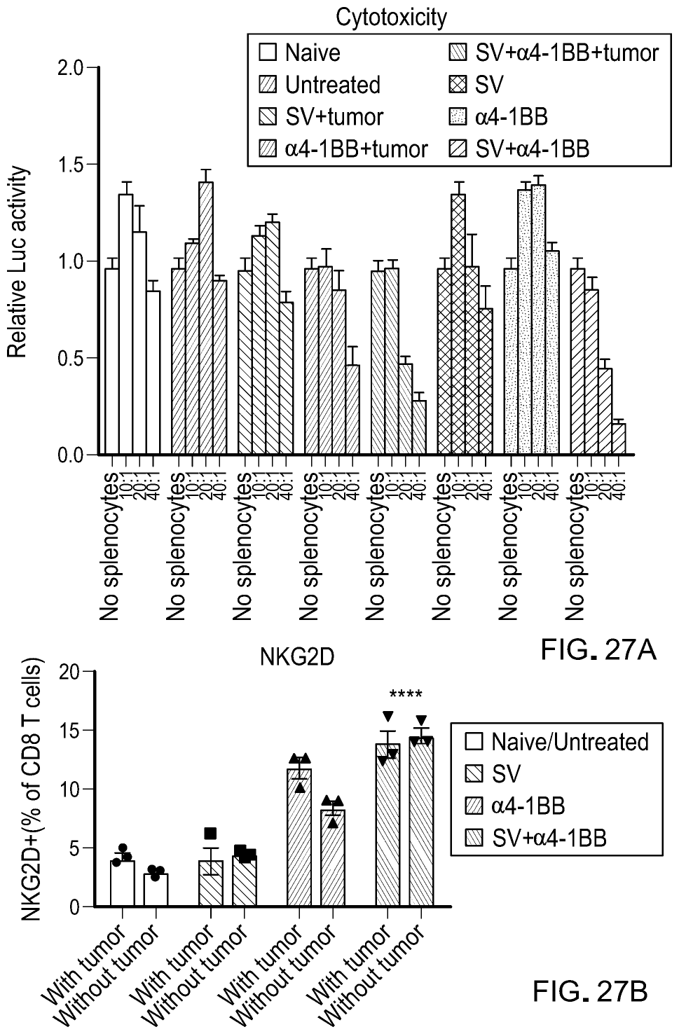

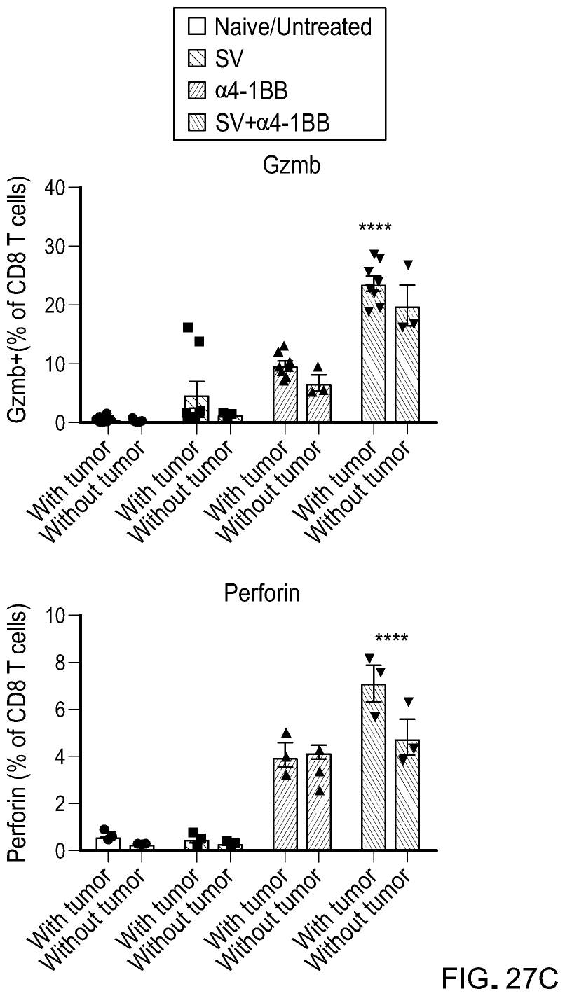

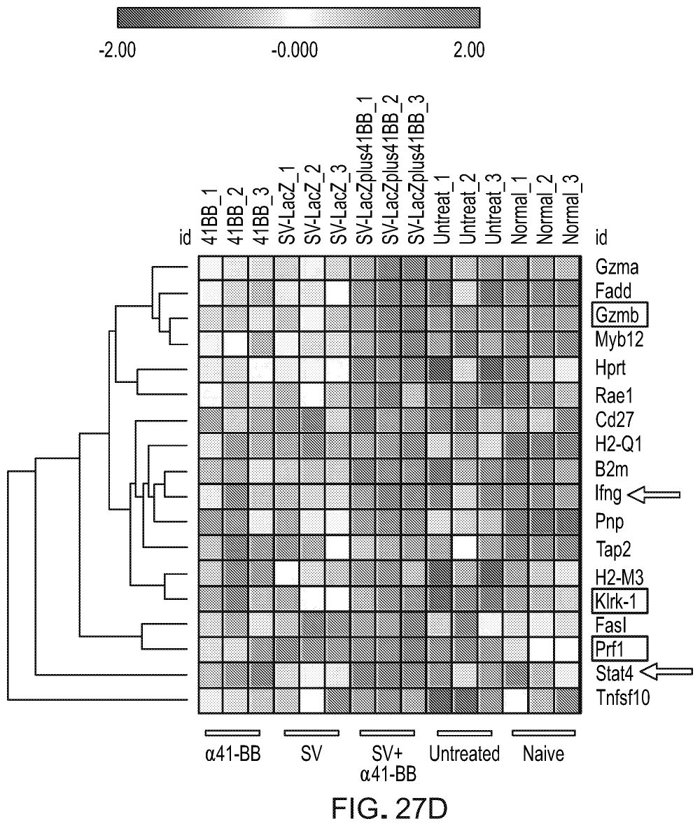

To investigate the antitumor cytotoxicity of SV/α4-1BB treated splenocytes, f-Luc A20 lymphoma cells were co-cultured with splenocytes on day 7. The ratios explored between splenocytes and tumor cell were 40:1, 20:1, 10:1. SV plus α4-1BB treated splenocytes demonstrated the highest cytotoxicity among all groups, as calculated by the reduction of f-Luc activity (FIG. 27A). To understand if this response is induced by TAA or anti-viral immunity, the same experiment was performed using mice under treatment but without tumor inoculation. We found that SV plus α4-1BB achieves the same effect as the combination treatment with tumor inoculation. This indicates that anti-tumor response on day 7 was not tumor specific. Accordingly, NKG2D, granzyme B and perforin were highly expressed in CD8 T cells from α4-1BB treated mice. In addition, SV plus α4-1BB in combination induced the highest expression of NKG2D and granzyme B in CD8 T cells. NKG2D, granzyme B and perforin upregulation was tumor independent because the same pattern was observed in all treatments without tumor inoculation (FIG. 27B-27C). Correspondingly, IPA indicates that gene sets of cytotoxic T cell development are significantly upregulated in SV plus α4-1BB mAb. These genes include Gzmb (granzyme B), Prfl (perforin) and Klrkl (NKG2D) (FIG. 27D). These data indicate that SV plus α4-1BB mAb markedly enhanced cytotoxic T cell activity.

SV Plus α4-1BB mAb Induced IFNγ Production from T Cells

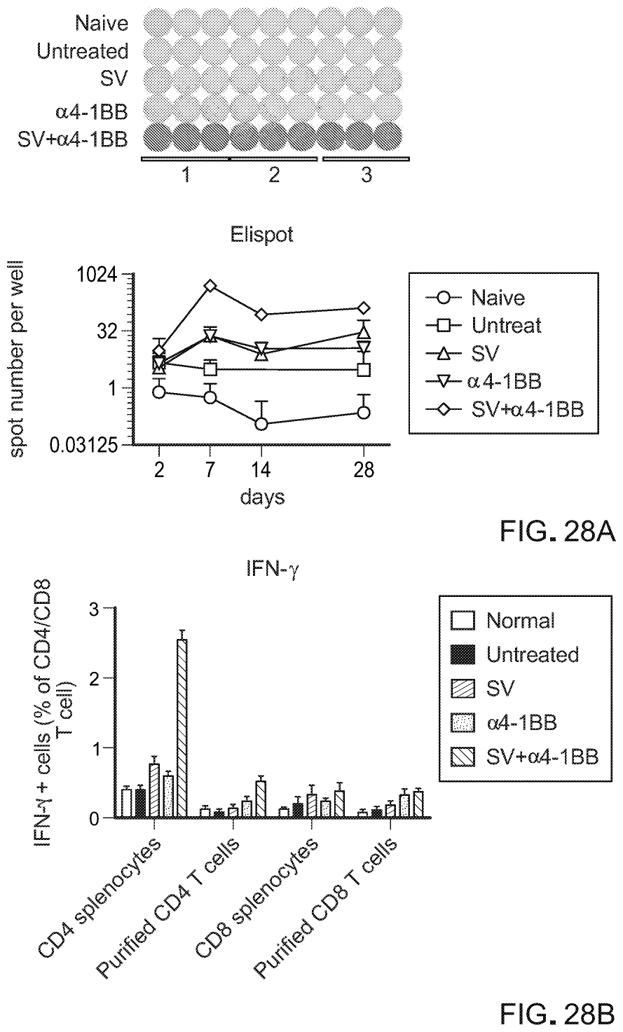

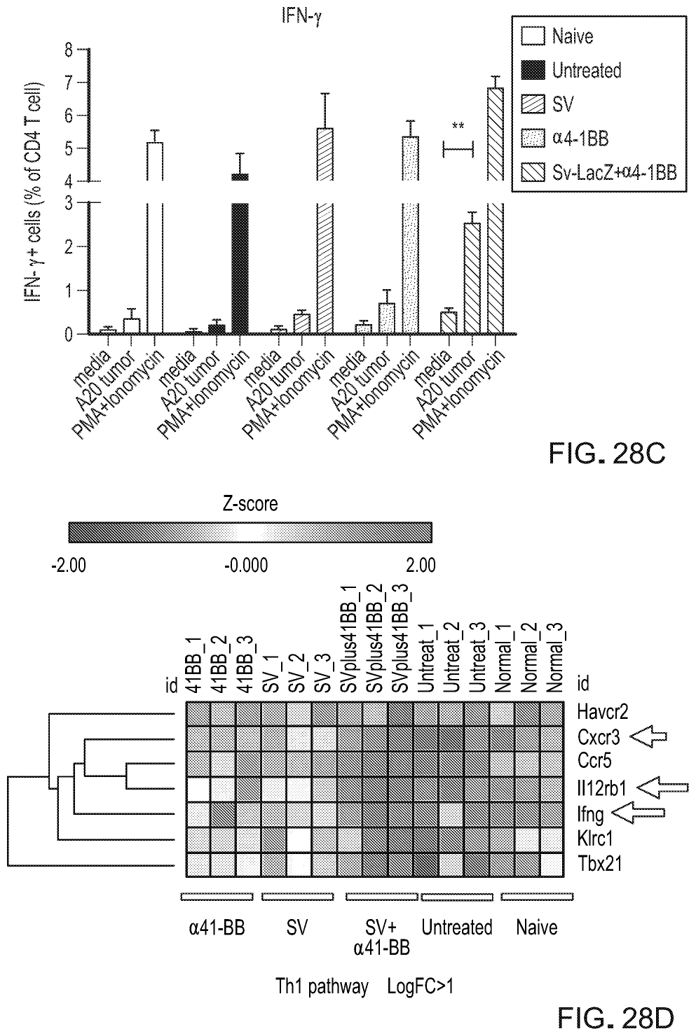

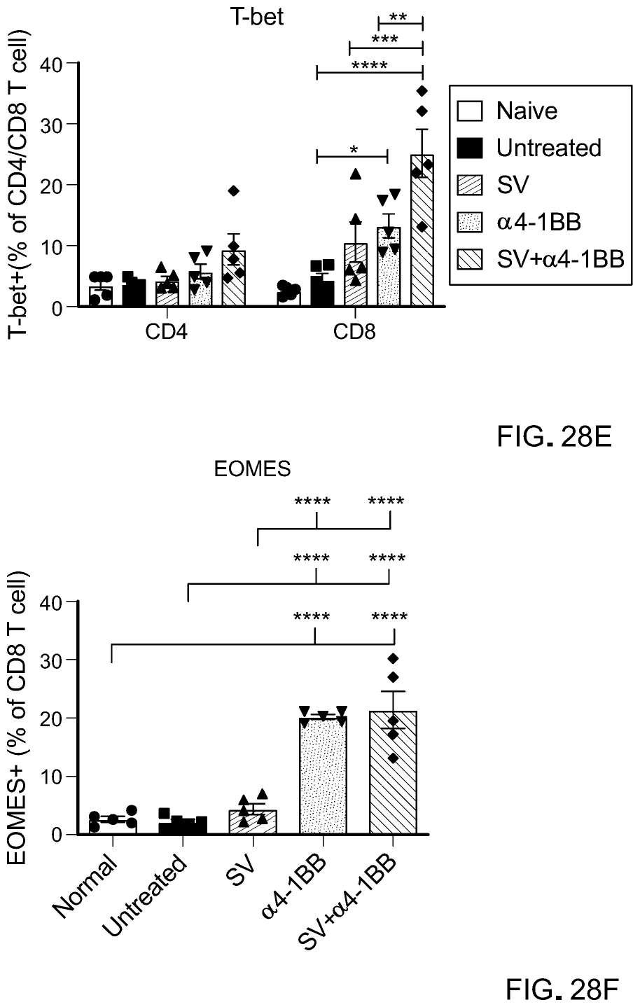

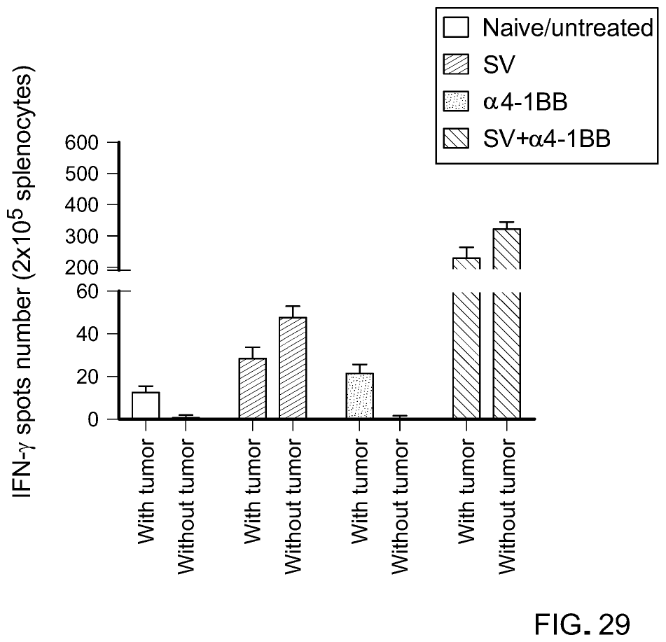

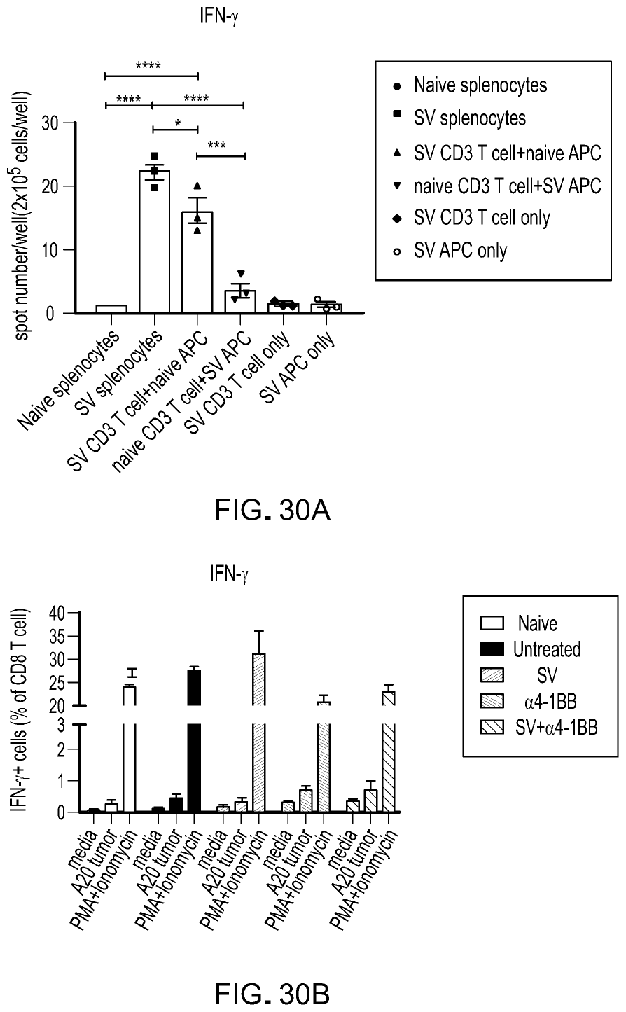

Other upregulated genes in the SV plus α4-1BB mAb combined treatment include STAT4 (FIG. 27D) and IL12rbl (FIG. 28D), which are required for the development of Th1 cells from naïve CD4+ T cells and IFNγ production (FIG. 27D) in response to IL-12 [Jacobson N G et al., J Exp Med. 1995]. Consistent with this observation, splenocytes from SV plus α4-1BB mAb treatment produced significantly higher number of IFNγ spots compared with other groups, reaching peak production on day 7 (FIG. 28A, upper panel). After day 7, the response dampened but still remained at the highest level compared with other groups (FIG. 28A, lower panel). This is in line with increased IFNγ RNA levels. To identify if TAA or viral antigen induces IFNγ production on day 7, the same experiment was performed in mice not inoculated with tumor cells. For both SV or SV plus α4-1BB treatment, the presence or absence of tumor did not significantly affect IFNγ levels (FIG. 29), confirming that IFNγ production on day 7 was mainly an anti-viral response. To identify whether T cells or antigen presentation cells (APCs) play the major role in IFNγ production, we harvested SV treated splenic T cells and naive T cells respectively. T cells from SV treated mice were co-cultured with naive APCs. Conversely, APCs from SV treated mice were cultured with naive T cells. T cells from SV treated mice produced IFNγ when co-cultured with naive APC. Naive T cells produce much less IFNγ spots when cultured with SV infected APC. However, neither T cell nor APC alone could produce elevated numbers of IFNγ spots. These observations indicate that T cells play the dominant role in IFNγ production during SV infection (FIG. 30A). APCs are essential for helping T cells to produce IFNγ.

Next, to identify whether CD4 or CD8 T cells produce IFNγ, flow cytometric analysis was performed for cytokine analysis. Among splenocytes, 2-2.5% SV plus α4-1BB mAb treated CD4 T cells produced IFNγ, which is significantly higher than other groups. Very low percentages of CD8 T cells produced IFNγ in all groups (FIG. 28B). There were much less IFNγ producing T cells after removing APC (FIG. 28B). Also, there was no difference among all groups for IFNγ production. This suggests that T cell-APC interaction is essential for IFNγ production. To test the antitumor IFNγ production activity of the purified T cells, they were co-cultured for 5 h with A20 cells, which express major histocompatibility complex (MHC) I and II molecules [Pizzoferrato E et al., Int J Cancer, 2004]. Only CD4 T cells from the SV plus α4-1BB mAb group produced IFNγ after co-culture (FIGS. 28C and 30B). This indicates that SV plus α4-1BB mAb induces anti-tumor IFNγ production activity. Besides IFNγ, several Th1 associated genes were also upregulated in the T cells from SV plus α4-1BB mAb treated groups. These include Ccr5, Cxcr3, Havcr2 (Tim3), IL12rbl and Klrc1 (FIG. 4d). T-bet is the key transcription factor which is essential for type 1 immune response (IFNγ production, T cell cytotoxicity) and memory T cell differentiation. In correspondence with the IFNγ expression findings, it was observed that SV plus α4-1BB mAb coordinately upregulates T-bet in T cells on day 7 (FIG. 28E). This suggests that SV helps α4-1BB boost the type 1 immune response, which is critical for controlling tumor growth. SV or α4-1BB mAb alone could not induce high IFNγ production due to low T-bet upregulation. Eomesodermin (EOMES), another important transcription factor, is upregulated in activated T cells and is essential for memory CD8 T cell development. Both α4-1BB mAb and SV plus α4-1BB mAb induced high expression of EOMES on day 7 (FIG. 28F). The lack of both T-bet and EOMES results in a lower expression of CXCR3 in T cells and a drastic decrease in the number of tumor-infiltrating T cells [28]. The data disclosed herein are consistent with these observations. Elevated CXCR3 (FIG. 28D), T-bet and EOMES (FIGS. 28E and 28F) in T cells of the combined SV plus α4-1BB mAb treated animals, were found.

SV and α4-1BB mAb Stimulated Chemotaxis, Adhesion and Enhanced T Cell Infiltration and Activation in Tumor

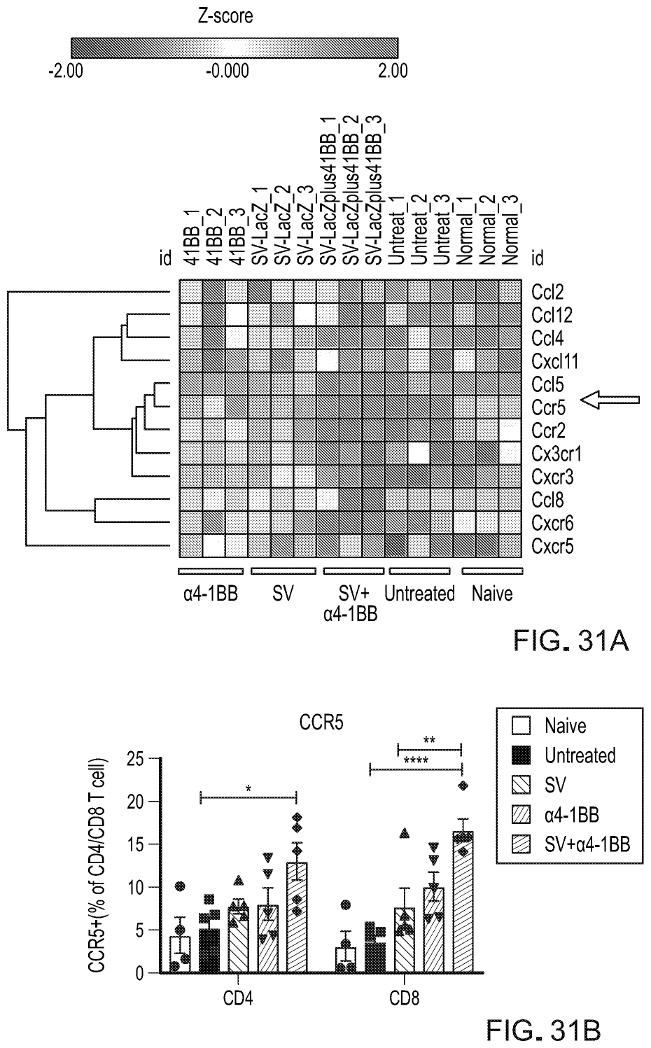

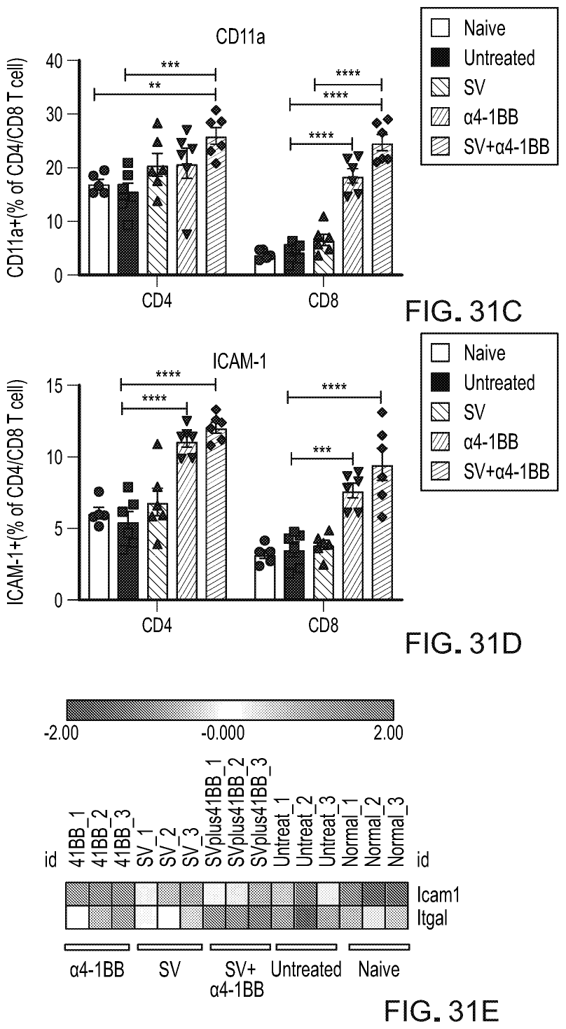

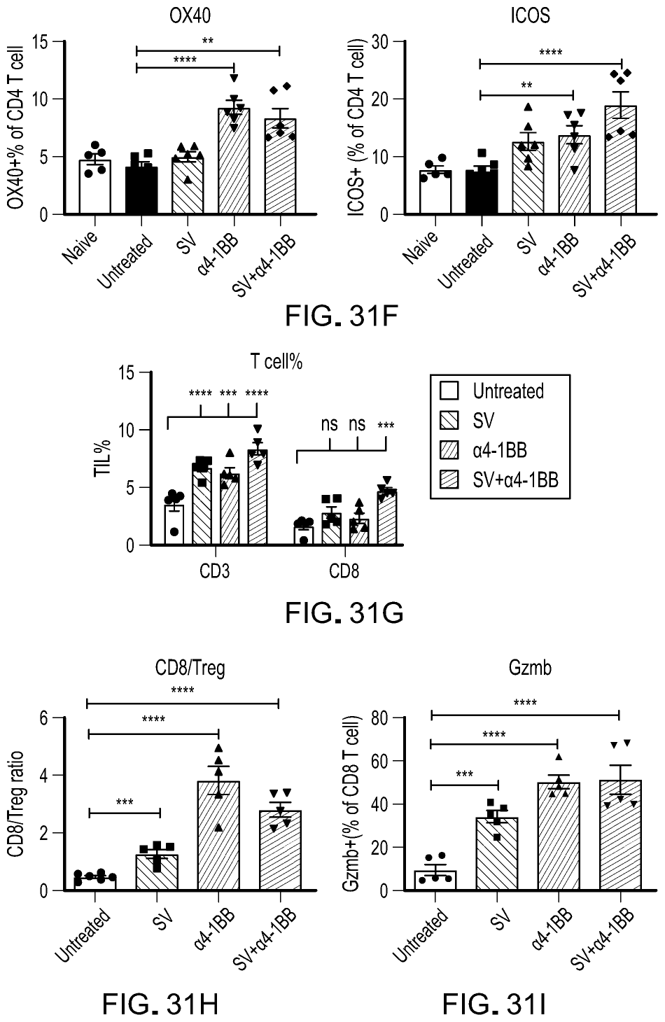

Through RNA-Seq, a series of chemokines and chemokine receptors have been identified to be upregulated in SV plus α4-1BB mAb (FIG. 31A). Among those molecules, CCR5 upregulation was confirmed by flow cytometry (FIG. 31B). CCR5 potentiates CD4 T helper cell functions boosting overall anti-tumor responses [Gonzalez-Martin A et al., Oncoimmunology, 2012]. SV plus α4-1BB significantly was found to upregulate CD11a and ICAM-1(CD54). These two adhesion molecules are highly expressed on activated T cells. LFA-1 (CD11a/CD18)-ICAM-1 interaction is essential for the formation of immune synapses between T cell and APC [Walling B L et al., Front Immunol, 2018]. LFA-1 and ICAM-1 are also required for T cell-T cell homotypic aggregation and activation [Sabatos C A, et al., Immunity, 2008; Gerard A, et al., Nat Immunol. 2013]. α4-1BB mAb stimulation induced significant upregulation of CD11a and ICAM-1 in both CD4 and CD8 T cells whereas SV does not (FIGS. 31C-31E). In addition, T cell costimulatory molecule, OX40, was also significantly upregulated in T cells of mice treated with α4-1BB. (FIG. 31F, left). OX40 engagement promotes effector T cell function and survival [33 Croft M, et al., Immunol Rev. 2009]. ICOS, another CD4 T cell costimulatory molecule, was upregulated in SV or α4-1BB alone but upregulated most in the SV plus α4-1BB combination treatment, suggesting a synergistic effect exists (FIG. 31F, right).

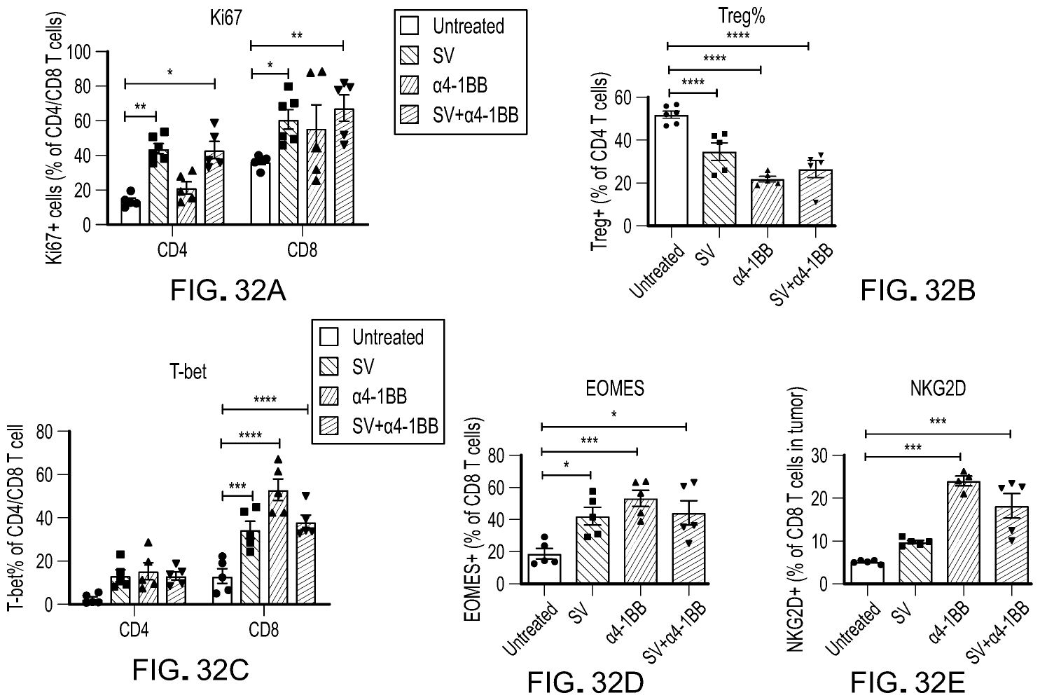

TIL play a critical anti-tumor role and is an important marker for prognosis. Compared with untreated, the percentage of CD3 and CD8 T cells were increased about 2 fold after combination treatment (FIG. 31G). Ki67 were upregulated in those T cells which indicated active division (FIG. 32A). For untreated TIL, the frequency of Foxp3+ Treg cells was the highest (FIG. 32B) and CD8/Treg ratio was the lowest (FIG. 31H). Treatment enhanced the T-bet and EOMES expression in T cells (FIG. 32C-32D). NKG2D and granzyme B were highly upregulated in tumor infiltrating CD8 T cells (FIGS. 31I, and 32E). Overall, these data indicate that combination treatment enhanced T cell infiltration, division, activation, cytotoxicity and downregulated the inhibitory Treg population.

SV and α4-1BB mAb Synergistically Enhanced Oxidative Phosphorylation

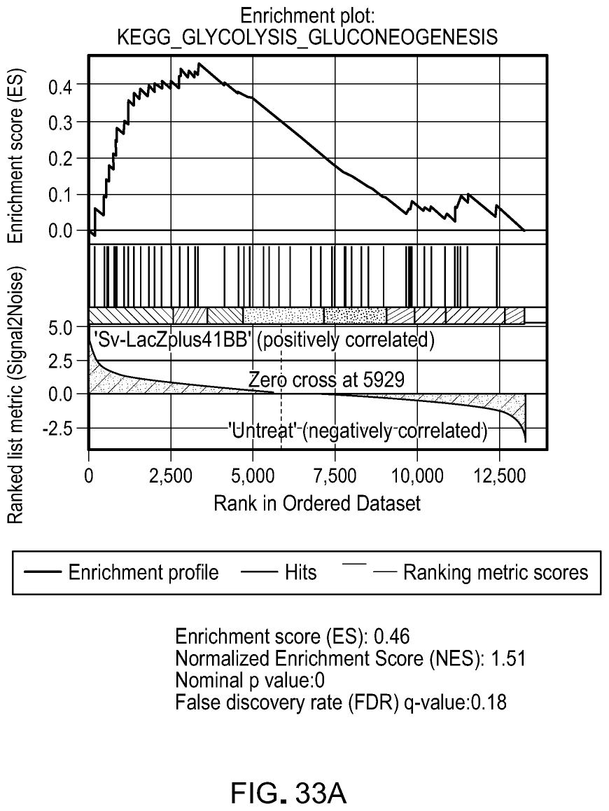

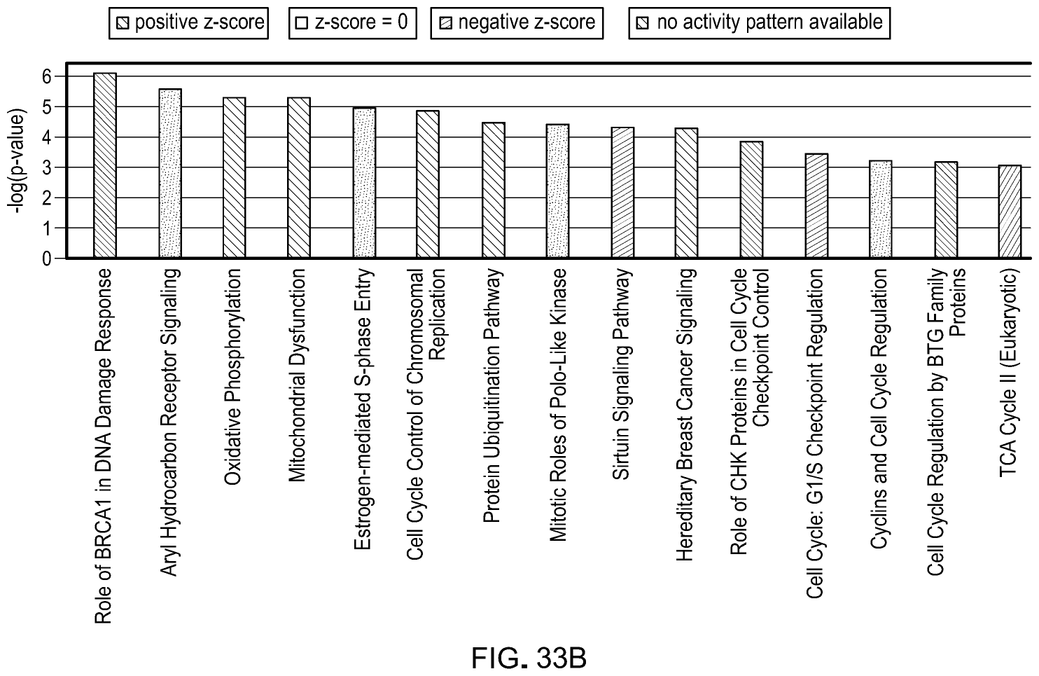

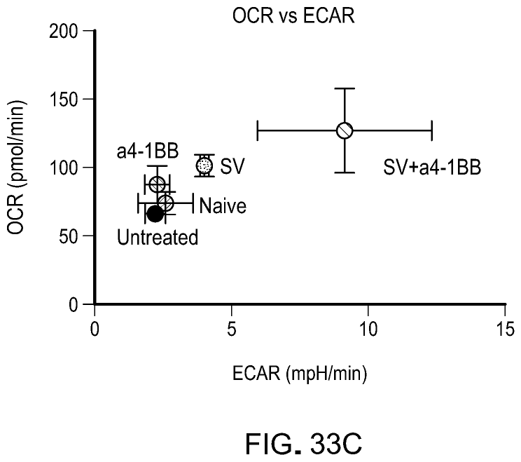

T cell activation requires a quick consumption of energy through both enhanced glycolysis and oxidative phosphorylation [Wahl D R et al., Immunol Rev., 2012]. Metabolic switch is a major feature of T cell activation and memory T cell development [van der Windt G J et al., Immunol Rev., 2012]. GSEA KEGG analysis identified that the glycolysis gene set is upregulated in SV plus α4-1BB vs. untreated samples (FIG. 33A). This process quickly produces ATP and supports T cell migration and cytotoxicity in hypoxic or acidic microenvironments. IP A confirms that SV plus α4-1BB mAb synergistically enhanced oxidative phosphorylation (FIG. 33B).

Both oxygen consumption rate (OCR, represents oxidative phosphorylation) and extracellular acidification rate (ECAR, represents glycolysis) of all groups (FIG. 33C) was assessed. Compared with other groups, SV plus α4-1BB significantly increased both OCR and ECAR. This indicates that both glycolysis and oxidative phosphorylation are activated in T cells of animals treated with SV plus α4-1BB.

SV Plus Low Dose α4-1BB mAb Cured A20 Tumor Bearing Mice

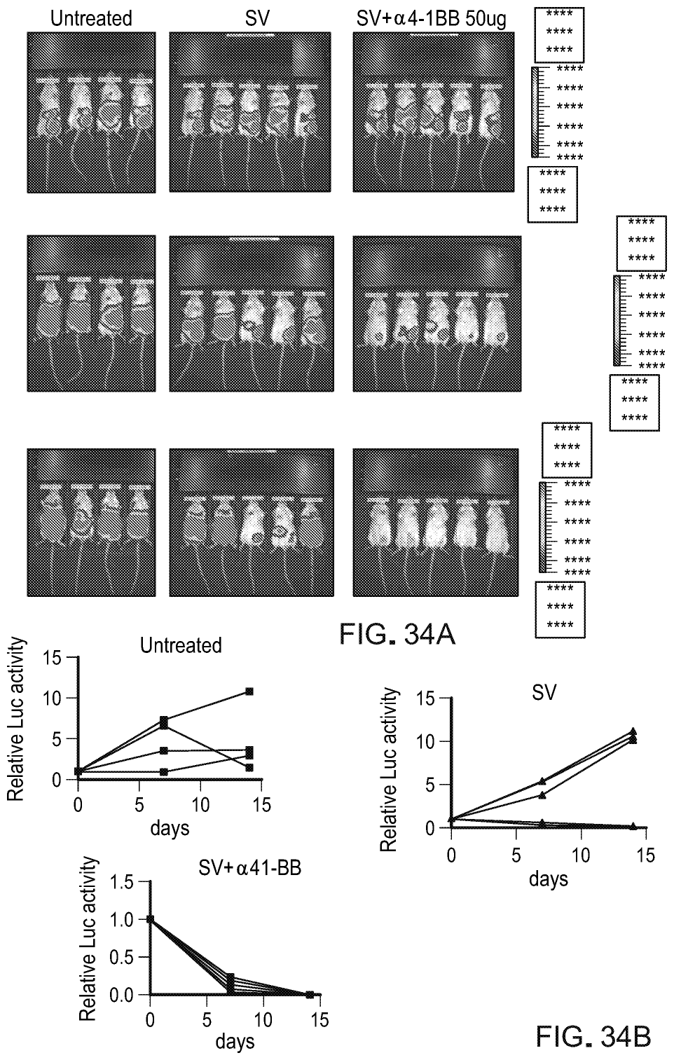

To reduce the potential risk of cytotoxicity and expense of treatment with SV vectors plus α4-1BB, the study disclosed herein explored whether low doses of α4-1BB mAb and fewer injections would be as effective in curing tumor bearing mice as the higher doses and frequencies used in our initial studies. As demonstrated (FIGS. 34A and 34B), A20 tumor bearing mice can be completely cured by SV (3 times per week for 3 weeks) plus a low dose of α4-1BB mAb (50 g per week for 3 weeks). This reduces both the SV and α4-1BB mAb dosing requirements. The reduced dose of α4-1BB mAb would be helpful, as well, in preventing the α4-1BB mAb induced liver toxicity reported by some investigators [Bartkowiak T, et al., Clin Cancer Res., 2018].

All Tumor Cured Mice Acquired Long Lasting Antitumor Immunity

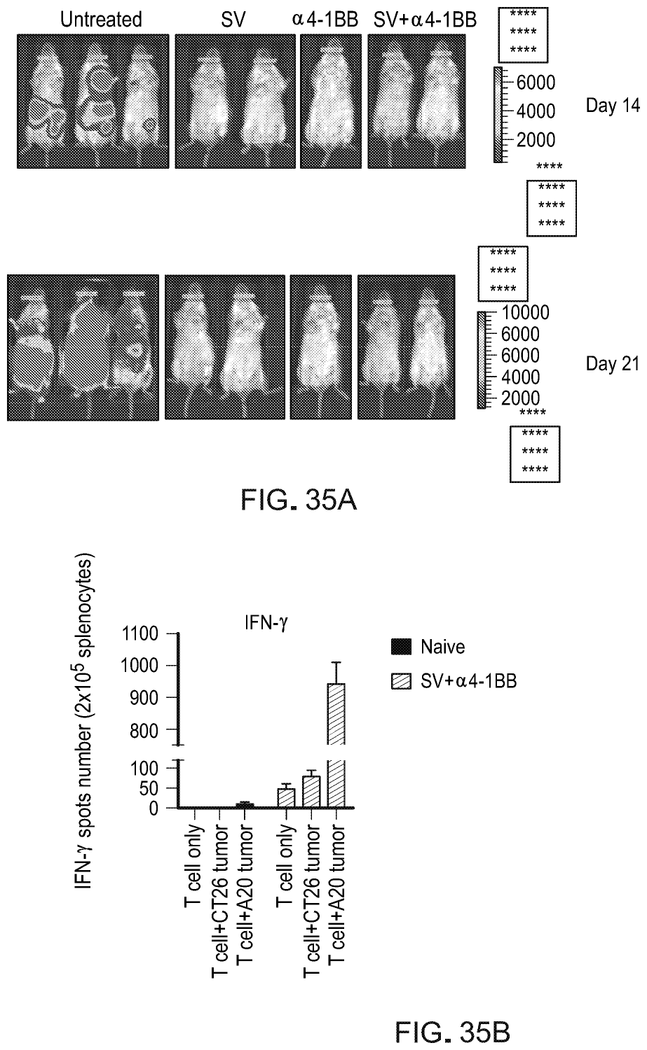

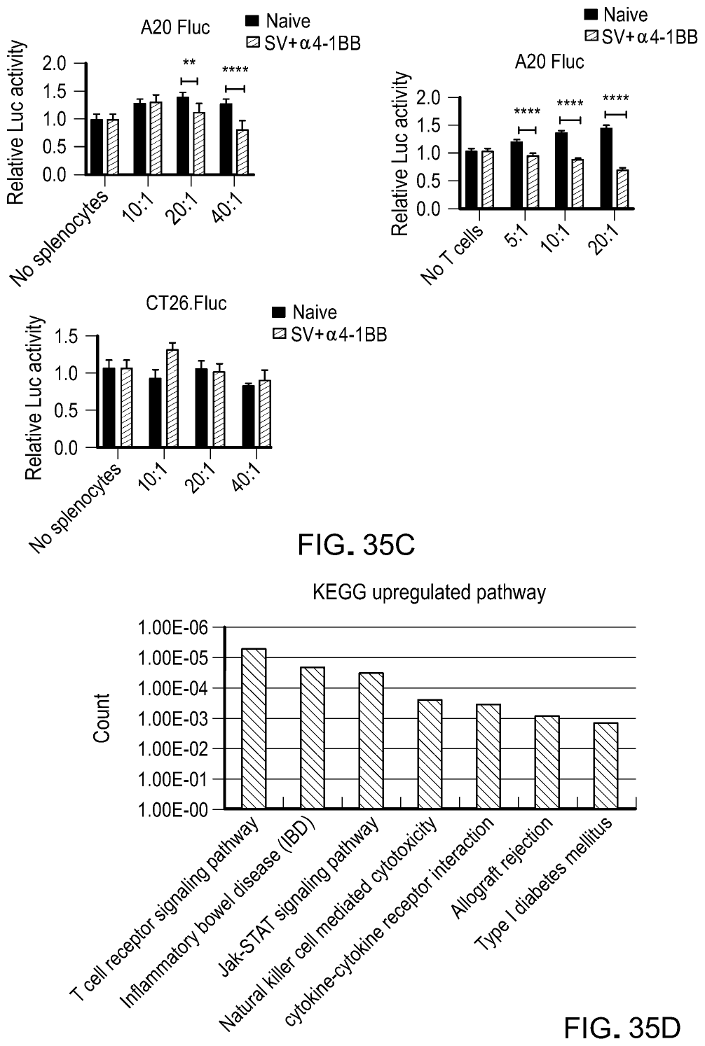

To investigate the memory response to A20 lymphoma, naive and tumor cured mice were inoculated with 3×10⁶ A20 tumor cells. Only mice that had survived more than 4 months after 1st time of tumor challenge were chosen. In all tumor cured mice, we found that A20 lymphoma was completely rejected whereas naive mice were susceptible to A20 inoculation (FIG. 35A).

To confirm anti-tumor specificity has been elicited, IFNγ production of purified T cells in the presence or absence of tumor cells was measured by Elispot assay. T cells were isolated from naive and cured mice under SV plus α4-1BB treatment (4 months after treatment finished). Isolated T cells were co-cultured with A20 and CT26 tumor cells respectively. Co-culturing with A20 cells dramatically enhanced IFNγ production, whereas co-culturing with CT26 cells only slightly enhanced IFNγ production (FIG. 35B).

Next, cytotoxicity to both naive and cured mice under SV plus α4-1BB treatment (the same method as FIG. 27A) was measured. Compared with naive, cured mice had enhanced cytotoxicity to A20 lymphoma cells, but not to CT26 tumor cells. To confirm that this is mediated by T cells, the same experiment was done using purified T cells. Cured mice had enhanced cytotoxicity compared with naive mice (FIG. 35C).

To better understand differences between this memory T cell response and the initial treatment responses as observed on day 7, RNA-Seq was performed by using purified splenic T cells from all re-challenged groups. In T cells of these re-challenged mice we found only a few differentially expressed genes among the three treated groups (Table 5), indicating that tumor cured mice develop a very similar T cell gene expression profile regardless of treatment method. Compared with untreated, KEGG analysis indicates that TCR signaling is the highest upregulated pathway in SV plus α4-1BB group (FIG. 35D), indicating that continuously enhanced TCR signaling is critical for maintaining antitumor immunity.

TABLE 5
The SD expressed gene lists among all tumor cured mice groups.

| | | | | | |
|---|---|---|---|---|---|
| gene | baseMean | log2FC | log2FCunshrunk | pvalue | padj |

SV + α4-1BB recha vs SV rechal

| | | | | | |
|---|---|---|---|---|---|
| Arl5c | 1644.9 | −0.7 | −0.71 | 4.57E−07 | 0.014007 |

SV + α4-1BB rechal vs α4-1BB rechal

| | | | | | |
|---|---|---|---|---|---|
| Prdm16 | 86.7 | −1.85 | −2.48 | 4.63E−07 | 0.001563 |
| Scamp5 | 106.1 | −1.59 | −1.99 | 3.98E−06 | 0.010743 |
| Klri1 | 117.3 | −1.57 | −1.98 | 5.79E−06 | 0.013016 |
| Ighv2-2 | 457.3 | −1.52 | −1.94 | 1.05E−05 | 0.020265 |
| Kcnj10 | 439.7 | −1.45 | −1.76 | 1.24E−05 | 0.020949 |
| Slc40a1 | 1111.8 | −1.41 | −1.69 | 1.53E−05 | 0.022994 |
| Trbv29 | 826.4 | −1.3 | −1.36 | 6.35E−12 | 8.57E−08 |
| Tmcc3 | 416.3 | −1.23 | −1.35 | 1.28E−07 | 0.000575 |
| Abcc3 | 1020.9 | −1.2 | −1.36 | 2.12E−05 | 0.02863 |
| Arl5c | 1644.9 | −0.76 | −0.77 | 2.43E−08 | 0.000164 |

SV rechal vs. α4-1BB rechal

| | | | | | |
|---|---|---|---|---|---|
| Gzmk | 1000.8 | −1.78 | −2.44 | 1.81E−06 | 0.033784 |
| Penk | 2127.9 | −1.35 | −1.6 | 6.37E−06 | 0.038894 |
| Smoc2 | 154.2 | −1.85 | −2.98 | 8.65E−06 | 0.038894 |
| Spag6 | 6.7 | −1.59 | −7.14 | 8.74E−06 | 0.038894 |
| Ighv3-5 | 85.6 | −1.86 | −3.04 | 1.08E−05 | 0.038894 |
| Wipf3 | 150.6 | −1.84 | −5.01 | 1.25E−05 | 0.038894 |

The conventional view of oncolytic virus therapy against tumors is that it requires selective infection of cancer cells resulting in the induction of cancer cell lysis and apoptosis. TAAs, released from dead tumor cells, attract and further stimulate an antitumor immune response. The study described herein found that encoding a TAA is not necessary for SV vectors plus α4-1BB mAb therapy to be fully successful. SV vectors lacking an A20 lymphoma TAA were able to treat A20 lymphoma and, in combination with α4-1BB mAb, eradicated the growing tumors. This is particularly important when effective immune reactive TAAs are unknown. It is possible that the immunotherapeutic response of SV vectors plus α4-1BB mAb is independent of whether a tumor is “cold” (i.e., having few TAAs or mutation-specific neoantigens capable of promoting robust T cell activation) or “hot.”

The study describe herein showed that both NKG2D (KLRK1) and granzyme B are highly expressed under combination treatment. This massive nonspecific activation is critical for controlling tumor growth at an early time point (day 7). This step is also important for inducing anti-tumor specificity that is mediated by TAAs released from dead tumor cells due to nonspecific killing. After tumor regression, T cells from treated animals were able maintain the ability to produce IFNγ and acquired immunological memory to rapidly reject A20 lymphoma rechallenges. IFNγ production from purified T cells of cured mice was significantly enhanced after encountering A20 tumor cells. This demonstrates that anti-tumor specificity is fully established in cured mice. Upregulated molecular pathways of responsive T cells induced by SV vectors and a 4-1 in mAbs alone and in combination were identified and compared in the study described herein. The combination of SV and α4-1BB mAb has a synergistic effect and represents a potent and robust therapeutic treatment able to cure B lymphomas and provide long term protection in a preclinical model.

In conclusion, SV vectors in combination with α4-1BB mAb completely eradicated a B-cell lymphoma in a preclinical mouse model, a result that could not be achieved with either treatment alone. Tumor elimination involves a synergistic effect of the combination that significantly boosts T cell cytotoxicity, IFN-γ production, migration, tumor infiltration and oxidative phosphorylation. In addition, all mice that survived after treatment developed long lasting antitumor immunity. The studies disclosed herein provides a novel, alternative method for B cell lymphoma treatment and describes a rationale to help translate SV vectors plus agonistic mAbs into clinical applications.

### Example 4: Sindbis Viral Vector Expressed NY-ESO-1 and IL-12 Enhances Survival of Subjects with Established Tumors

The study described herein investigates the effect of administering a tumor associated antigen and an immunostimulatory molecule, as expressed by a Sindbis viral vector on anti-tumor response and survival in a subject with an established tumor. Previous studies, demonstrated vectors encoding TAAs, such as NY-ESO-I, could cure CT26-NY-ESO-1 tumors [Galon J, et al., Nature reviews Drug discovery 2019; Gupta S, et al., Frontiers in oncology 2017]. However, while this approach has been effective in enhancing the immune response to and clearance of established tumors of colon and prostate cancers, the efficacy in curing other cancers, e.g. ovarian cancer has been limited. Therefore, an approach of administering a combination of a SV expressed immunostimulatory molecule, IL-12 along with the SV-NY-ESO-1, to a subject with an established tumor was tested.

Combination of NY-ESO-1 and IL-12 Expressed by Separate Sindbis Viral Vectors Enhances Survival of Subjects with Established Tumors

The study described herein investigates the effect of administering IL-12 and NY-ESO-1, both expressed by separate Sindbis viral vectors, on established tumors. C57/B16 albino (female) mice re-injected with Alm5-2Fluc-17 ovarian cancer cells to establish a tumor (FIG. 19), and treated with either a SV vector expressing IL-12 (SV-IL-12), a SV vector expressing NY-ESO-1 (SVNYESO) or a 50% mix of a SV-IL-12 and a SVNYESO (SV-NYESO_SV-IL12).

A Sindbis replicon expressing NYESO-1 cDNA (SV-NYESO1) was made by PCR amplification of the NYESO-1 gene from the pReceiver-M02 plasmid. Expression of the NYESO-1 gene was confirmed by western blot. NYESO-1 was detected by western blot following standard protocol, using as a primary antibody the anti-NYESO-1 clone E978 (Upstate) at a dilution 1/5,000 in Tris-buffered saline-Tween (TBS-T) with 5% non-fat milk. SV.IL12 plasmid used in this study has been published in 2002 [Tseng J C et al., J Natl Cancer Inst. 2002]. To construct a Sindbis viral vector containing genes for interleukin 12 (IL-12), the Sindbis viral vector SinRep/2PSG was first constructed, which contains a secondary subgenomic promoter that is responsive to the Sindbis replicase. Two DNA oligonucleotide primers (sequence 5′ CGCGTAAAGCATCTCTACGGTGGTCCTAATAGTGCATG-3′; SEQ ID NO: 29) and its complementary strand 5′CACTATTAGGACCACCGTCGAGATGCTTTA-3′; SEQ ID NO: 30) containing the subgenomic promoter sequence were annealed and ligated into the MluI and SphI sites of the SinRep plasmid. The murine IL-12 α subunit gene (mp35; ATCC 87596) and the IL-12 β subunit gene (mp40; ATCC 87595) were subcloned into the MluI and the StuI sites of SinRep/2PSG, respectively, to produce the Sin-Rep/IL12 plasmid.

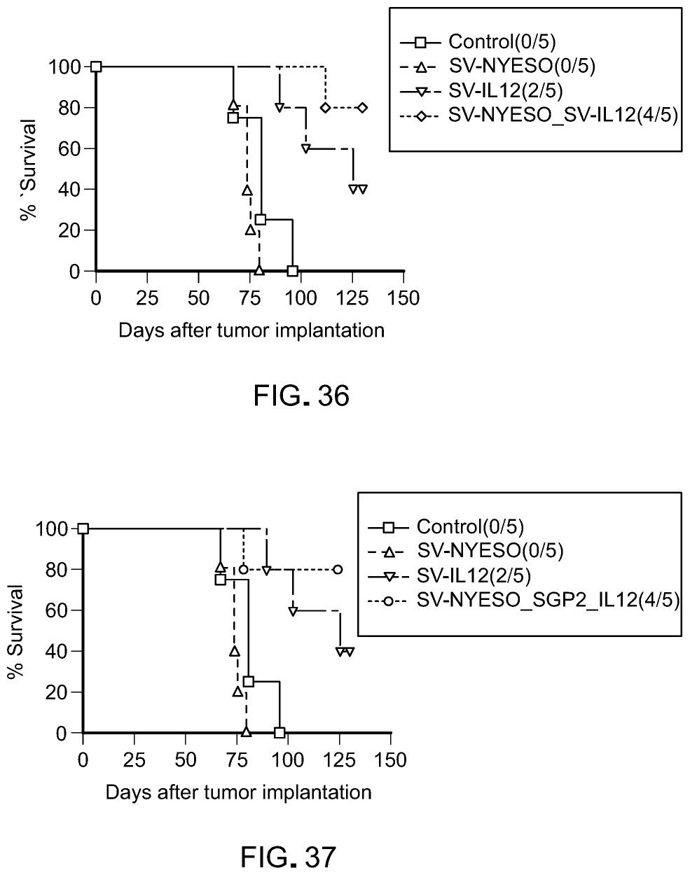

As expected the SV-IL-12 treatment group showed a better percentage survival of mice with tumor over the SVNYESO treatment group and the untreated (control) group. However, a synergistically higher showed enhanced percentage survival rate was observed in the SV-NYESO_SV-IL12 in comparison to the SV-IL-12 treatment group (FIG. 36). The results described herein clearly show the possibility of using a combination of SV vectors expressing IL-12 and NY-ESO-1, for treatment of cancers that may be resistant to treatment with a SV expressing a tumor associated antigen.

Combination of NY-ESO-1 and IL-12 Expressed by the Same Sindbis Viral Vectors Enhances Survival of Subjects with Established Tumors

The study described herein investigates the effect of administering IL-12 and NY-ESO-1, both expressed by the same Sindbis viral vector, on established tumors. C57/B16 albino (female) mice re-injected with Alm5-2Fluc-17 ovarian cancer cells to establish a tumor (FIG. 19), and treated with either a SV vector expressing IL-12 (SV-IL-12), a SV vector expressing NY-ESO-1 (SVNYESO) or a Sindbis viral vector that expresses both IL-12 and NYESO (SV-NYESO_SGP2_IL12). As shown in FIG. 36, the SV-IL-12 treatment group showed a better percentage survival of mice with tumor over the SVNYESO treatment group and the untreated (control) group. As expected, the SVNYESO treatment group and the untreated (control) group showed similar survival rate, thereby showing that certain tumors are resistant to treatment with a SV expressing a tumor associated antigen (TAA) like NY-ESO-1. A synergistically higher enhanced percentage survival rate was observed in the SV-NYESO_SGP2_IL12 treatment group, in comparison to the SV-IL-12 treatment group (FIG. 37).

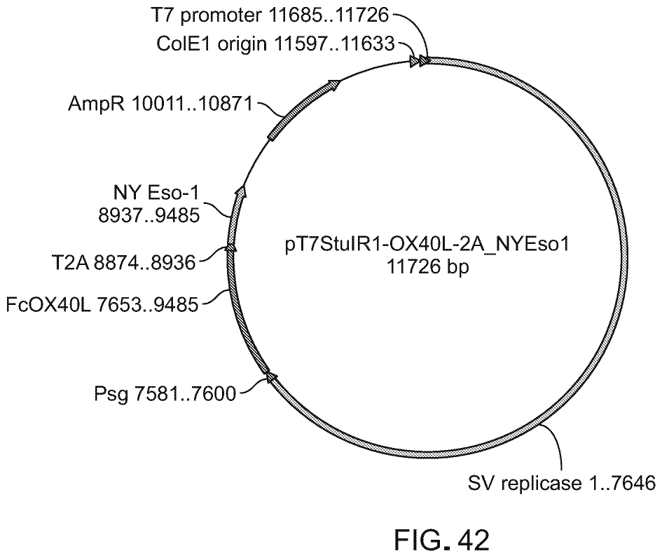

The study described herein, provides plasmid constructs for expressing NY-ESO-1, IL-12 and anti-OX40 in a SV vector. The study described herein, provides plasmid constructs encoding IL-12 α and b subunits (FIG. 38), anti-OX40 IgG2a heavy and light chains (FIG. 39), a single chain antibody to OX40 (OX40 ScFv) (FIG. 40), a human NY-ESO-1 (FIG. 41) and an OX40 ligand fused to a Fc peptide (OX40L-Fc T2A) and a NY-ESO-1 with a termination peptide sequence T2A in between (FIG. 42).

In summary, the results of the study described herein clearly show the possibility of using a SV vectors expressing both IL-12 and NY-ESO-1, for treatment of cancers that may be resistant to treatment with a SV expressing a tumor associated antigen.

What we claim is:

1. A method for treating cancer in a subject in need thereof comprising administering to the subject a therapeutically effective amount of (a) a Sindbis viral vector and (b) an antibody directed against a co-stimulatory molecule, or a nucleic acid encoding same; or an antibody to an immune system agonist molecule, or a nucleic acid encoding same.

2. The method of claim 1, wherein the antibody is selected from the group consisting of anti-OX40 antibody, anti-4-1BB antibody, anti-CD28 antibody, anti-GITR antibody, anti-CD137 antibody, anti-cd37 antibody, and anti-HVEM antibody.

3. The method of claim 1, wherein the Sindbis viral vector comprises at least one nucleic acid encoding an immunostimulatory or an immunomodulatory protein.

4. The method of claim 2, wherein the immunostimulatory or immunomodulatory protein is IL-I, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35 or IL-36.

5. The method of claim 3, wherein the immunostimulatory or immunomodulatory protein is IL-12.

6. The method of claim 1, wherein the Sindbis viral vector and the antibody induce an immune response in a tumor associated antigen (TAA) nonspecific manner.

7. A method for treating cancer in a subject in need thereof comprising administering to the subject a therapeutically effective amount of (a) a Sindbis viral vector comprising a nucleic acid encoding interleukin-12 and (b) an anti-OX40 monoclonal antibody or a nucleic acid encoding same, thereby treating cancer in the subject.

8. The method of claim 7, wherein the Sindbis viral vector comprising the nucleic acid encoding interleukin-12 further comprises the nucleic acid encoding the anti-OX40 monoclonal antibody.

9. The method of claim 7, comprising administering a Sindbis viral vector comprising the nucleic acid encoding interleukin-12 and administering a Sindbis viral vector comprising the nucleic acid encoding the anti-OX40 monoclonal antibody.

10. The method of claim 7, wherein the nucleic acid encoding interleukin-12 comprises the nucleic acid sequence encoding interleukin 12 alpha subunit of SEQ ID NO: 1, the nucleic acid encoding interleukin 12 beta subunit of SEQ ID NO: 2, or a combination thereof.

11. The method of claim 7, wherein the nucleic acid encoding the anti-OX40 monoclonal antibody comprises
a nucleic acid sequence that is at least 70%, 75%, 80%, 85%, 90%, 95%, 96%, 97%, 98%, or 99% identical to the nucleic acid sequence selected from of SEQ ID NO:6, SEQ ID NO:10, SEQ ID NO:32, SEQ ID NO:36, SEQ ID NO:40, SEQ ID NO:44 and SEQ ID NO:48 encoding an anti-OX40 antibody heavy chain,
a nucleic acid sequence that is at least 70%, 75%, 80%, 85%, 90%, 95%, 96%, 97%, 98%, 99% or 100% identical to the nucleic acid sequence of SEQ ID NO:8, SEQ ID NO:12, SEQ ID NO:34, SEQ ID NO:38, SEQ ID NO:42, SEQ ID NO:46 and SEQ ID NO:50 encoding an anti-OX40 antibody light chain,
or any combination of heavy chain nucleic acid sequence or light chain nucleic acid sequence thereof.

12. The method of claim 7, wherein the nucleic acid encoding the anti-OX40 monoclonal antibody comprises a nucleic acid encoding an anti-OX40 antibody heavy chain amino acid sequence, an anti-OX40 antibody light chain amino acid sequence, that binds to a target antigen of the amino acid sequence of SEQ ID NO: 13, or a combination thereof.

13. The method of claim 7, wherein the Sindbis viral vector and the anti-OX40 monoclonal antibody are administered systemically.

14. The method of claim 7, wherein the Sindbis viral vector and the anti-OX40 monoclonal antibody are administered parenterally.

15. The method of claim 7, wherein the Sindbis viral vector and the anti-OX40 monoclonal antibody are administered intraperitoneally.

16. The method of claim 7, wherein the Sindbis viral vector is replication defective.

17. The method of claim 7, wherein the cancer comprises a solid tumor.

18. The method of claim 7, wherein the cancer is colon cancer, prostate cancer or ovarian cancer.

19. A Sindbis viral vector comprising a nucleic acid encoding interleukin-12 and a nucleic acid encoding an anti-OX40 monoclonal antibody.

20. The Sindbis viral vector of claim 19, wherein the nucleic acid encoding interleukin-12 comprises the nucleic acid sequence encoding interleukin 12 alpha subunit of SEQ ID NO: 1, the nucleic acid encoding interleukin 12 beta subunit of SEQ ID NO: 2, or a combination thereof.

21. The Sindbis viral vector of claim 19, wherein the nucleic acid encoding the anti-OX40 monoclonal antibody comprises
a nucleic acid sequence that is at least 70%, 75%, 80%, 85%, 90%, 95%, 96%, 97%, 98%, or 99% identical to the nucleic acid sequence selected from of SEQ ID NO:6, SEQ ID NO:10, SEQ ID NO:32, SEQ ID NO:36, SEQ ID NO:40, SEQ ID NO:44 and SEQ ID NO:48 encoding an anti-OX40 antibody heavy chain,
a nucleic acid sequence that is at least 70%, 75%, 80%, 85%, 90%, 95%, 96%, 97%, 98%, 99% or 100% identical to the nucleic acid sequence of SEQ ID NO:8, SEQ ID NO:12, SEQ ID NO:34, SEQ ID NO:38, SEQ ID NO:42, SEQ ID NO:46 and SEQ ID NO:50 encoding an anti-OX40 antibody light chain,
or any combination of heavy chain nucleic acid sequence or light chain nucleic acid sequence thereof.

22. A composition comprising (a) a Sindbis viral vector comprising a nucleic acid encoding interleukin-12 and (b) an anti-OX40 monoclonal antibody or a nucleic acid encoding same.

23. A composition comprising (a) a Sindbis viral vector comprising a nucleic acid encoding interleukin-12 and (b) a Sindbis viral vector comprising a nucleic acid encoding an anti-OX40 monoclonal antibody.

24. A method for treating cancer in a subject in need thereof comprising administering to the subject a therapeutically effective amount of (a) a Sindbis viral vector comprising a nucleic acid encoding interleukin-12 and a Sindbis viral vector comprising a nucleic acid encoding NY-ESO-1 or (b) a Sindbis viral vector comprising a nucleic acid encoding interleukin-12 and a nucleic acid encoding NY-ESO-1, thereby treating cancer in the subject.

25. The method of claim 24, wherein the nucleic acid encoding interleukin-12 comprises the nucleic acid sequence encoding interleukin 12 alpha subunit of SEQ ID NO: 1, the nucleic acid encoding interleukin 12 beta subunit of SEQ ID NO: 2, or a combination thereof.

26. The method of claim 24, wherein the nucleic acid encoding NY-ESO-1 comprises a nucleic acid sequence of SEQ ID NO: 14.

27. The method of claim 24, wherein the (a) a Sindbis viral vector comprising a nucleic acid encoding interleukin-12 and a Sindbis viral vector comprising a nucleic acid encoding NY-ESO-1 or (b) a Sindbis viral vector comprising a nucleic acid encoding interleukin-12 and a nucleic acid encoding NY-ESO-1 are administered systemically.

28. The method of claim 24, wherein the (a) a Sindbis viral vector comprising a nucleic acid encoding interleukin-12 and a Sindbis viral vector comprising a nucleic acid encoding NY-ESO-1 or (b) a Sindbis viral vector comprising a nucleic acid encoding interleukin-12 and a nucleic acid encoding NY-ESO-1 are administered parenterally.

29. The method of claim 24, wherein the (a) a Sindbis viral vector comprising a nucleic acid encoding interleukin-12 and a Sindbis viral vector comprising a nucleic acid encoding NY-ESO-1 or (b) a Sindbis viral vector comprising a nucleic acid encoding interleukin-12 and a nucleic acid encoding NY-ESO-1 are administered intraperitoneally.

30. The method of claim 24, wherein the Sindbis viral vector is replication defective.

31. The method of claim 24, wherein the cancer comprises a solid tumor.

32. The method of claim 24, wherein the cancer is colon cancer, prostate cancer or ovarian cancer.

33. A Sindbis viral vector comprising a nucleic acid encoding interleukin-12 and a nucleic acid encoding an NY-ESO-1.

34. The Sindbis viral vector of claim 33, wherein the nucleic acid encoding interleukin-12 comprises the nucleic acid sequence encoding interleukin 12 alpha subunit of SEQ ID NO: 1, the nucleic acid encoding interleukin 12 beta subunit of SEQ ID NO: 2, or a combination thereof.

35. The Sindbis viral vector of claim 33, wherein the nucleic acid encoding the NY-ESO-1 comprises a nucleic acid sequence of SEQ ID NO: 14.

36. A composition comprising (a) a Sindbis viral vector comprising a nucleic acid encoding interleukin-12 and (b) a Sindbis viral vector comprising a nucleic acid encoding an NY-ESO-1.

37. A method for treating cancer in a subject in need thereof comprising administering to the subject a therapeutically effective amount of (a) a Sindbis viral vector comprising a nucleic acid encoding NY-ESO-1 and (b) an anti-OX40 monoclonal antibody or a nucleic acid encoding same, thereby treating cancer in the subject.

38. The method of claim 37, wherein the Sindbis viral vector comprising the nucleic acid encoding NY-ESO-1 further comprises the nucleic acid encoding the anti-OX40 monoclonal antibody.

39. The method of claim 37, comprising administering a Sindbis viral vector comprising the nucleic acid encoding NY-ESO-1 and administering a Sindbis viral vector comprising the nucleic acid encoding the anti-OX40 monoclonal antibody.

40. The method of claim 37, wherein the nucleic acid encoding NY-ESO-1 comprises a nucleic acid sequence of SEQ ID NO: 14.

41. The method of claim 37, wherein the nucleic acid encoding the anti-OX40 monoclonal antibody comprises
a nucleic acid sequence that is at least 70%, 75%, 80%, 85%, 90%, 95%, 96%, 97%, 98%, or 99% identical to the nucleic acid sequence selected from of SEQ ID NO:6, SEQ ID NO:10, SEQ ID NO:32, SEQ ID NO:36, SEQ ID NO:40, SEQ ID NO:44 and SEQ ID NO:48 encoding an anti-OX40 antibody heavy chain,
a nucleic acid sequence that is at least 70%, 75%, 80%, 85%, 90%, 95%, 96%, 97%, 98%, 99% or 100% identical to the nucleic acid sequence of SEQ ID NO:8, SEQ ID NO:12, SEQ ID NO:34, SEQ ID NO:38, SEQ ID NO:42, SEQ ID NO:46 and SEQ ID NO:50 encoding an anti-OX40 antibody light chain,
or any combination of heavy chain nucleic acid sequence or light chain nucleic acid sequence thereof.

42. The method of claim 37, wherein the nucleic acid encoding the anti-OX40 monoclonal antibody comprises a nucleic acid encoding an anti-OX40 antibody heavy chain amino acid sequence, an anti-OX40 antibody light chain amino acid sequence, that binds to a target antigen of the amino acid sequence of SEQ ID NO: 13, or a combination thereof.

43. The method of claim 37, wherein the Sindbis viral vector and the anti-OX40 monoclonal antibody are administered systemically.

44. The method of claim 37, wherein the Sindbis viral vector and the anti-OX40 monoclonal antibody are administered parenterally.

45. The method of claim 37, wherein the Sindbis viral vector and the anti-OX40 monoclonal antibody are administered intraperitoneally.

46. The method of claim 37, wherein the Sindbis viral vector is replication defective.

47. The method of claim 37, wherein the cancer comprises a solid tumor.

48. The method of claim 37, wherein the cancer is colon cancer, prostate cancer or ovarian cancer.

49. A Sindbis viral vector comprising a nucleic acid encoding NY-ESO-1 and a nucleic acid encoding an anti-OX40 monoclonal antibody.

50. The Sindbis viral vector of claim 49, wherein the nucleic acid encoding the NY-ESO-1 comprises the nucleic acid sequence of SEQ ID NO: 14.

51. The Sindbis viral vector of claim 49, wherein the nucleic acid encoding the anti-OX40 monoclonal antibody comprises
a nucleic acid sequence that is at least 70%, 75%, 80%, 85%, 90%, 95%, 96%, 97%, 98%, or 99% identical to the nucleic acid sequence selected from of SEQ ID NO:6, SEQ ID NO:10, SEQ ID NO:32, SEQ ID NO:36, SEQ ID NO:40, SEQ ID NO:44 and SEQ ID NO:48 encoding an anti-OX40 antibody heavy chain,
a nucleic acid sequence that is at least 70%, 75%, 80%, 85%, 90%, 95%, 96%, 97%, 98%, 99% or 100% identical to the nucleic acid sequence of SEQ ID NO:8, SEQ ID NO:12, SEQ ID NO:34, SEQ ID NO:38, SEQ ID NO:42, SEQ ID NO:46 and SEQ ID NO:50 encoding an anti-OX40 antibody light chain,
or any combination of heavy chain nucleic acid sequence or light chain nucleic acid sequence thereof.

52. The Sindbis viral vector of claim 49, wherein the nucleic acid encoding the anti-OX40 monoclonal antibody comprises a nucleic acid encoding an anti-OX40 antibody heavy chain amino acid sequence, an anti-OX40 antibody light chain amino acid sequence, that binds to a target antigen of the amino acid sequence of SEQ ID NO: 13, or a combination thereof.

53. A composition comprising (a) a Sindbis viral vector comprising a nucleic acid encoding NY-ESO-1 and (b) an anti-OX40 monoclonal antibody or a nucleic acid encoding same.

54. A composition comprising (a) a Sindbis viral vector comprising a nucleic acid encoding NY-ESO-1 and (b) a Sindbis viral vector comprising a nucleic acid encoding an anti-OX40 monoclonal antibody.

55. A method for treating cancer in a subject in need thereof comprising administering to the subject a therapeutically effective amount (a) a Sindbis viral vector and (b) an anti-4-1BB monoclonal antibody or a nucleic acid encoding same, thereby treating cancer in the subject.

56. The method of claim 55, wherein the Sindbis viral vector and the anti-4-1BB monoclonal antibody are administered systemically.

57. The method of claim 55, wherein the Sindbis viral vector and the anti-4-1BB monoclonal antibody are administered parenterally.

58. The method of claim 55, wherein the Sindbis viral vector and the anti-4-1BB monoclonal antibody are administered intraperitoneally.

59. The method of claim 55, wherein the Sindbis viral vector is replication defective.

60. The method of claim 55, wherein the cancer is a hematologic cancer.

61. The method of claim 55, wherein the cancer is a B cell lymphoma.
